SEQUENCE LISTING (602 sequences)

SEQ ID NO: 1 - protein, 736 aa, synthetic construct
  REGION 1..736: note = AAV6 WT capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, synthetic construct
  REGION 1..736: note = mT640 // Ark313
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSVVNPAEGLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - DNA, 4683 bp, synthetic construct
  misc_feature 1..4683: note = AAV6 WT capsid
  source 1..4683: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 4 - DNA, 2208 bp, synthetic construct
  misc_feature 1..2208: note = mT640 // Ark313
  source 1..2208: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccgtggtcaacccggccgagggcttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggactgaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctg

SEQ ID NO: 5 - protein, 7 aa, synthetic construct
  REGION 1..7: note = 454-460 region of mT640-01
VVNPAEG

SEQ ID NO: 6 - protein, 7 aa, synthetic construct
  REGION 1..7: note = 454-460 region of mT640-02
VVNPADA

SEQ ID NO: 7 - protein, 7 aa, synthetic construct
  REGION 1..7: note = 454-460 region of mT640-03
IVNPQVE

SEQ ID NO: 8 - protein, 7 aa, synthetic construct
  REGION 1..7: note = 454-460 region of mT640-05
VVNPAEL

SEQ ID NO: 9 - protein, 7 aa, synthetic construct
  REGION 1..7: note = 454-460 region of mT640-05
VVNPDDG

SEQ ID NO: 10 - protein, 7 aa, synthetic construct
  REGION 1..7: note = 454-460 region of mT640-06
VVNPAET

SEQ ID NO: 11 - protein, 7 aa, synthetic construct
  REGION 1..7: note = 454-460 region of mT640-07
VLNPAEG

SEQ ID NO: 12 - protein, 7 aa, synthetic construct
  REGION 1..7: note = 454-460 region of mT640-08
IINPLPS

SEQ ID NO: 13 - protein, 7 aa, synthetic construct
  REGION 1..7: note = 454-460 region of mT640-09
VVNPAEA

SEQ ID NO: 14 - protein, 7 aa, synthetic construct
  REGION 1..7: note = 454-460 region of mT640-10
IVNPHSI

SEQ ID NO: 15 - protein, 7 aa, synthetic construct
  REGION 1..7: note = 454-460 region of mT640-11
LVNPAEG

SEQ ID NO: 16 - protein, 7 aa, synthetic construct
  REGION 1..7: note = 454-460 region of mT640-12
VVNPDEG

SEQ ID NO: 17 - protein, 7 aa, synthetic construct
  REGION 1..7: note = 454-460 region of mT640-13
VVNPMQA

SEQ ID NO: 18 - protein, 7 aa, synthetic construct
  REGION 1..7: note = 454-460 region of mT640-14
VVNPGEG

SEQ ID NO: 19 - protein, 7 aa, synthetic construct
  REGION 1..7: note = 454-460 region of mT640-15
SDNPSWG

SEQ ID NO: 20 - protein, 7 aa, synthetic construct
  REGION 1..7: note = 454-460 region of mT640-16
GTNPPCA

SEQ ID NO: 21 - protein, 7 aa, synthetic construct
  REGION 1..7: note = 454-460 region of mT640-17
GGNPVEV

SEQ ID NO: 22 - protein, 7 aa, synthetic construct
  REGION 1..7: note = 454-460 region of mT640-18
SLNPSGG

SEQ ID NO: 23 - protein, 7 aa, synthetic construct
  REGION 1..7: note = 454-460 region of mT640-19
ASNPSGY

SEQ ID NO: 24 - protein, 7 aa, synthetic construct
  REGION 1..7: note = 454-460 region of mT640-20
SANPSHA

SEQ ID NO: 25 - protein, 7 aa, synthetic construct
  REGION 1..7: note = 454-460 region of mT640-21
RDNPSSG

SEQ ID NO: 26 - protein, 7 aa, synthetic construct
  REGION 1..7: note = 454-460 region of mT640-22
TTNPIVS

SEQ ID NO: 27 - protein, 7 aa, synthetic construct
  REGION 1..7: note = 454-460 region of mT640-23
APNPGSA

SEQ ID NO: 28 - protein, 7 aa, synthetic construct
  REGION 1..7: note = 454-460 region of mT640-24
GTNPVGT

SEQ ID NO: 29 - protein, 7 aa, synthetic construct
  REGION 1..7: note = 454-460 region of mT640-24
GTNPVGT

SEQ ID NO: 30 - protein, 7 aa, synthetic construct
  REGION 1..7: note = 454-460 region of mT640-25
EGNPPTA

SEQ ID NO: 31 - protein, 7 aa, synthetic construct
  REGION 1..7: note = 454-460 region of mT640-26
GSNPAEG

SEQ ID NO: 32 - protein, 7 aa, synthetic construct
  REGION 1..7: note = 454-460 region of mT640-27
WENPGGG

SEQ ID NO: 33 - protein, 7 aa, synthetic construct
  REGION 1..7: note = 454-460 region of mT640-29
LRNPMPL

SEQ ID NO: 34 - protein, 7 aa, synthetic construct
  REGION 1..7: note = 454-460 region of mT640-30
VDNPAEC

SEQ ID NO: 35 - protein, 7 aa, synthetic construct
  REGION 1..7: note = 454-460 region of mT640-31
GTNPLAT

SEQ ID NO: 36 - protein, 7 aa, synthetic construct
  REGION 1..7: note = 454-460 region of mT640-32
NANPGEG

SEQ ID NO: 37 - protein, 7 aa, synthetic construct
  REGION 1..7: note = 454-460 region of mT640-33
VPNPSEL

SEQ ID NO: 38 - protein, 7 aa, synthetic construct
  REGION 1..7: note = 454-460 region of mT640-34
VFNPADG

SEQ ID NO: 39 - protein, 7 aa, synthetic construct
  REGION 1..7: note = 454-460 region of mT640-35
SANPGWG

SEQ ID NO: 40 - protein, 7 aa, synthetic construct
  REGION 1..7: note = 454-460 region of mT640-36
VVMPDVK

SEQ ID NO: 41 - protein, 7 aa, synthetic construct
  REGION 1..7: note = 454-460 region of mT640-37
LLNREAT

SEQ ID NO: 42 - protein, 7 aa, synthetic construct
  REGION 1..7: note = 454-460 region of mT640-38
ATHVHAS

SEQ ID NO: 43 - protein, 7 aa, synthetic construct
  REGION 1..7: note = 454-460 region of mT640-39
VLRATEE

SEQ ID NO: 44 - protein, 7 aa, synthetic construct
  REGION 1..7: note = 454-460 region of mT640-40
VIERADQ

SEQ ID NO: 45 - protein, 7 aa, synthetic construct
  REGION 1..7: note = 454-460 region of mT640-41
IVNPGCG

SEQ ID NO: 46 - protein, 7 aa, synthetic construct
  REGION 1..7: note = 454-460 region of mT640-42
VVNPDHT

SEQ ID NO: 47 - protein, 7 aa, synthetic construct
  REGION 1..7: note = 454-460 region of mT640-43
KLLPVGE

SEQ ID NO: 48 - protein, 7 aa, synthetic construct
  REGION 1..7: note = 454-460 region of mT640-44
TLMSSTA

SEQ ID NO: 49 - protein, 7 aa, synthetic construct
  REGION 1..7: note = 454-460 region of mT640-45
TLLPSSS

SEQ ID NO: 50 - protein, 7 aa, synthetic construct
  REGION 1..7: note = 454-460 region of mT640-46
RLVPNSD

SEQ ID NO: 51 - protein, 7 aa, synthetic construct
  REGION 1..7: note = 454-460 region of mT640-47
VVNPYQA

SEQ ID NO: 52 - protein, 7 aa, synthetic construct
  REGION 1..7: note = 454-460 region of mT640-48
LLMDKPS

SEQ ID NO: 53 - protein, 7 aa, synthetic construct
  REGION 1..7: note = 454-460 region of mT640-49
KQEKVEG

SEQ ID NO: 54 - protein, 7 aa, synthetic construct
  REGION 1..7: note = 454-460 region of mT640-50
PDHARST

SEQ ID NO: 55 - protein, 7 aa, synthetic construct
  REGION 1..7: note = 454-460 region of mT640-51
RNEPSSA

SEQ ID NO: 56 - protein, 7 aa, synthetic construct
  REGION 1..7: note = 454-460 region of mT640-52
QREERSS

SEQ ID NO: 57 - protein, 7 aa, synthetic construct
  REGION 1..7: note = 454-460 region of mT640-53
VEVGDMQ

SEQ ID NO: 58 - protein, 7 aa, synthetic construct
  REGION 1..7: note = 454-460 region of mT640-54
IVNADSL

SEQ ID NO: 59 - protein, 7 aa, synthetic construct
  REGION 1..7: note = 454-460 region of mT640-55
EPTKVSR

SEQ ID NO: 60 - protein, 7 aa, synthetic construct
  REGION 1..7: note = 454-460 region of mT640-56
GERRHCE

SEQ ID NO: 61 - protein, 7 aa, synthetic construct
  REGION 1..7: note = 454-460 region of mT640-57
IVNPQVA

SEQ ID NO: 62 - protein, 7 aa, synthetic construct
  REGION 1..7: note = 454-460 region of mT640-58
LPSESFS

SEQ ID NO: 63 - protein, 7 aa, synthetic construct
  REGION 1..7: note = 454-460 region of mT640-59
PNANQQY

SEQ ID NO: 64 - protein, 7 aa, synthetic construct
  REGION 1..7: note = 454-460 region of mT640-60
RGEMSAG

SEQ ID NO: 65 - protein, 7 aa, synthetic construct
  REGION 1..7: note = 454-460 region of mT640-61
KLLPSLD

SEQ ID NO: 66 - protein, 7 aa, synthetic construct
  REGION 1..7: note = 454-460 region of mT640-62
NASVNSA

SEQ ID NO: 67 - protein, 7 aa, synthetic construct
  REGION 1..7: note = 454-460 region of mT640-63
TESGALS

SEQ ID NO: 68 - protein, 7 aa, synthetic construct
  REGION 1..7: note = 454-460 region of mT640-64
TILPSVS

SEQ ID NO: 69 - protein, 7 aa, synthetic construct
  REGION 1..7: note = 454-460 region of mT640-65
TTTSSVG

SEQ ID NO: 70 - protein, 7 aa, synthetic construct
  REGION 1..7: note = 454-460 region of mT640-66
RGDSPES

SEQ ID NO: 71 - protein, 7 aa, synthetic construct
  REGION 1..7: note = 454-460 region of mT640-67
TDKDGMS

SEQ ID NO: 72 - protein, 7 aa, synthetic construct
  REGION 1..7: note = 454-460 region of mT640-68
SPSKYQR

SEQ ID NO: 73 - protein, 7 aa, synthetic construct
  REGION 1..7: note = 454-460 region of mT640-69
IGERKAE

SEQ ID NO: 74 - protein, 7 aa, synthetic construct
  REGION 1..7: note = 454-460 region of mT640-70
NTVDDAR

SEQ ID NO: 75 - protein, 7 aa, synthetic construct
  REGION 1..7: note = 454-460 region of mT640-71
TFALKAE

SEQ ID NO: 76 - protein, 7 aa, synthetic construct
  REGION 1..7: note = 454-460 region of mT640-72
QMVRQAE

SEQ ID NO: 77 - protein, 7 aa, synthetic construct
  REGION 1..7: note = 454-460 region of mT640-73
TVLSRSD

SEQ ID NO: 78 - protein, 7 aa, synthetic construct
  REGION 1..7: note = 454-460 region of mT640-74
TSEKGCA

SEQ ID NO: 79 - protein, 7 aa, synthetic construct
  REGION 1..7: note = 454-460 region of mT640-75
ALWSEDV

SEQ ID NO: 80 - protein, 7 aa, synthetic construct
  REGION 1..7: note = 454-460 region of mT640-76
SGSVSAS

SEQ ID NO: 81 - protein, 7 aa, synthetic construct
  REGION 1..7: note = 454-460 region of mT640-77
AIDQGNP

SEQ ID NO: 82 - protein, 7 aa, synthetic construct
  REGION 1..7: note = 454-460 region of mT640-78
TQHTAGS

SEQ ID NO: 83 - protein, 7 aa, synthetic construct
  REGION 1..7: note = 454-460 region of mT640-79
AILKDKE

SEQ ID NO: 84 - protein, 7 aa, synthetic construct
  REGION 1..7: note = 454-460 region of mT640-80
SLLPSQQ

SEQ ID NO: 85 - protein, 7 aa, synthetic construct
  REGION 1..7: note = 454-460 region of mT640-81
LRAQDSE

SEQ ID NO: 86 - protein, 7 aa, synthetic construct
  REGION 1..7: note = 454-460 region of mT640-82
QVEGRVG

SEQ ID NO: 87 - protein, 7 aa, synthetic construct
  REGION 1..7: note = 454-460 region of mT640-83
NVERAGT

SEQ ID NO: 88 - protein, 7 aa, synthetic construct
  REGION 1..7: note = 454-460 region of mT640-84
MGEHAYG

SEQ ID NO: 89 - protein, 7 aa, synthetic construct
  REGION 1..7: note = 454-460 region of mT640-85
TVIQRSD

SEQ ID NO: 90 - protein, 7 aa, synthetic construct
  REGION 1..7: note = 454-460 region of mT640-86
TLLPEQR

SEQ ID NO: 91 - protein, 7 aa, synthetic construct
  REGION 1..7: note = 454-460 region of mT640-87
GSAQNND

SEQ ID NO: 92 - protein, 7 aa, synthetic construct
  REGION 1..7: note = 454-460 region of mT640-88
GGDTGAR

SEQ ID NO: 93 - protein, 7 aa, synthetic construct
  REGION 1..7: note = 454-460 region of mT640-89
AHNPGTG

SEQ ID NO: 94 - protein, 7 aa, synthetic construct
  REGION 1..7: note = 454-460 region of mT640-90
ALRRLEE

SEQ ID NO: 95 - protein, 7 aa, synthetic construct
  REGION 1..7: note = 454-460 region of mT640-91
LTNGGMT

SEQ ID NO: 96 - protein, 7 aa, synthetic construct
  REGION 1..7: note = 454-460 region of mT640-92
SSVSSWP

SEQ ID NO: 97 - protein, 7 aa, synthetic construct
  REGION 1..7: note = 454-460 region of mT640-93
KGTDCHV

SEQ ID NO: 98 - protein, 7 aa, synthetic construct
  REGION 1..7: note = 454-460 region of mT640-94
SSNVQQA

SEQ ID NO: 99 - protein, 7 aa, synthetic construct
  REGION 1..7: note = 454-460 region of mT640-95
SDLKVGQ

SEQ ID NO: 100 - protein, 7 aa, synthetic construct
  REGION 1..7: note = 454-460 region of mT640-96
STLDNTS

SEQ ID NO: 101 - protein, 7 aa, synthetic construct
  REGION 1..7: note = 454-460 region of mT640-97
DTMSRAY

SEQ ID NO: 102 - protein, 7 aa, synthetic construct
  REGION 1..7: note = 454-460 region of mT640-98
VLNPSSQ

SEQ ID NO: 103 - protein, 7 aa, synthetic construct
  REGION 1..7: note = 454-460 region of mT640-99
VLTRNVE

SEQ ID NO: 104 - protein, 7 aa, synthetic construct
  REGION 1..7: note = 454-460 region of mT640-100
TFSSQQT

SEQ ID NO: 105 - protein, 7 aa, synthetic construct
  REGION 1..7: note = 454-460 region of mT640-101
SLLSTSS

SEQ ID NO: 106 - protein, 7 aa, synthetic construct
  REGION 1..7: note = 454-460 region of mT640-102
PTFNEIN

SEQ ID NO: 107 - protein, 7 aa, synthetic construct
  REGION 1..7: note = 454-460 region of mT640-103
EGKKGQL

SEQ ID NO: 108 - protein, 7 aa, synthetic construct
  REGION 1..7: note = 454-460 region of mT640-104
SSSSLVG

SEQ ID NO: 109 - protein, 7 aa, synthetic construct
  REGION 1..7: note = 454-460 region of mT640-105
DTDSGSR

SEQ ID NO: 110 - protein, 7 aa, synthetic construct
  REGION 1..7: note = 454-460 region of mT640-106
DGKHEWG

SEQ ID NO: 111 - protein, 7 aa, synthetic construct
  REGION 1..7: note = 454-460 region of mT640-107
LSDISHN

SEQ ID NO: 112 - protein, 7 aa, synthetic construct
  REGION 1..7: note = 454-460 region of mT640-108
REASGVG

SEQ ID NO: 113 - protein, 7 aa, synthetic construct
  REGION 1..7: note = 454-460 region of mT640-109
VERESGG

SEQ ID NO: 114 - protein, 7 aa, synthetic construct
  REGION 1..7: note = 454-460 region of mT640-110
EGATSCR

SEQ ID NO: 115 - protein, 7 aa, synthetic construct
  REGION 1..7: note = 454-460 region of mT640-111
SSGSDLA

SEQ ID NO: 116 - protein, 7 aa, synthetic construct
  REGION 1..7: note = 454-460 region of mT640-112
SSSADRD

SEQ ID NO: 117 - protein, 7 aa, synthetic construct
  REGION 1..7: note = 454-460 region of mT640-113
TESKASN

SEQ ID NO: 118 - protein, 7 aa, synthetic construct
  REGION 1..7: note = 454-460 region of mT640-114
VINPGVA

SEQ ID NO: 119 - protein, 7 aa, synthetic construct
  REGION 1..7: note = 454-460 region of mT640-115
VLSKDSL

SEQ ID NO: 120 - protein, 7 aa, synthetic construct
  REGION 1..7: note = 454-460 region of mT640-116
EGCKAAG

SEQ ID NO: 121 - protein, 7 aa, synthetic construct
  REGION 1..7: note = 454-460 region of mT640-117
LKLETTV

SEQ ID NO: 122 - protein, 7 aa, synthetic construct
  REGION 1..7: note = 454-460 region of mT640-118
GGSQSVA

SEQ ID NO: 123 - protein, 7 aa, synthetic construct
  REGION 1..7: note = 454-460 region of mT640-119
TTPVNTT

SEQ ID NO: 124 - protein, 7 aa, synthetic construct
  REGION 1..7: note = 454-460 region of mT640-120
RELEKQL

SEQ ID NO: 125 - protein, 7 aa, synthetic construct
  REGION 1..7: note = 454-460 region of mT640-121
KVQLDQL

SEQ ID NO: 126 - protein, 7 aa, synthetic construct
  REGION 1..7: note = 454-460 region of mT640-122
KVEGSQP

SEQ ID NO: 127 - protein, 7 aa, synthetic construct
  REGION 1..7: note = 454-460 region of mT640-123
VTSTRSE

SEQ ID NO: 128 - protein, 7 aa, synthetic construct
  REGION 1..7: note = 454-460 region of mT640-124
LWRADCT

SEQ ID NO: 129 - protein, 7 aa, synthetic construct
  REGION 1..7: note = 454-460 region of mT640-125
VVRGTED

SEQ ID NO: 130 - protein, 7 aa, synthetic construct
  REGION 1..7: note = 454-460 region of mT640-126
SAKVAPS

SEQ ID NO: 131 - protein, 7 aa, synthetic construct
  REGION 1..7: note = 454-460 region of mT640-127
LDMASNL

SEQ ID NO: 132 - protein, 7 aa, synthetic construct
  REGION 1..7: note = 454-460 region of mT640-128
ETRTSPY

SEQ ID NO: 133 - protein, 7 aa, synthetic construct
  REGION 1..7: note = 454-460 region of mT640-129
TLLPSEL

SEQ ID NO: 134 - protein, 7 aa, synthetic construct
  REGION 1..7: note = 454-460 region of mT640-130
TILQSNA

SEQ ID NO: 135 - protein, 7 aa, synthetic construct
  REGION 1..7: note = 454-460 region of mT640-131
DLLGAGQ

SEQ ID NO: 136 - protein, 7 aa, synthetic construct
  REGION 1..7: note = 454-460 region of mT640-132
ETRLKEG

SEQ ID NO: 137 - protein, 7 aa, synthetic construct
  REGION 1..7: note = 454-460 region of mT640-133
SMSVPVG

SEQ ID NO: 138 - protein, 7 aa, synthetic construct
  REGION 1..7: note = 454-460 region of mT640-134
SASPEVR

SEQ ID NO: 139 - protein, 7 aa, synthetic construct
  REGION 1..7: note = 454-460 region of mT640-135
TREADGS

SEQ ID NO: 140 - protein, 7 aa, synthetic construct
  REGION 1..7: note = 454-460 region of mT640-136
TVVKEGA

SEQ ID NO: 141 - protein, 7 aa, synthetic construct
  REGION 1..7: note = 454-460 region of mT640-137
PDRSDVR

SEQ ID NO: 142 - protein, 7 aa, synthetic construct
  REGION 1..7: note = 454-460 region of mT640-138
NTVPSQL

SEQ ID NO: 143 - protein, 7 aa, synthetic construct
  REGION 1..7: note = 454-460 region of mT640-139
QAENKLG

SEQ ID NO: 144 - protein, 7 aa, synthetic construct
  REGION 1..7: note = 454-460 region of mT640-140
PDGSRQA

SEQ ID NO: 145 - protein, 7 aa, synthetic construct
  REGION 1..7: note = 454-460 region of mT640-141
SESGDVR

SEQ ID NO: 146 - protein, 7 aa, synthetic construct
  REGION 1..7: note = 454-460 region of mT640-142
KDHTSQA

SEQ ID NO: 147 - protein, 7 aa, synthetic construct
  REGION 1..7: note = 454-460 region of mT640-143
ASNVNVG

SEQ ID NO: 148 - protein, 7 aa, synthetic construct
  REGION 1..7: note = 454-460 region of mT640-144
KLHNVVD

SEQ ID NO: 149 - protein, 7 aa, synthetic construct
  REGION 1..7: note = 454-460 region of mT640-145
GEKLRAS

SEQ ID NO: 150 - protein, 7 aa, synthetic construct
  REGION 1..7: note = 454-460 region of mT640-146
EDRARGG

SEQ ID NO: 151 - protein, 7 aa, synthetic construct
  REGION 1..7: note = 454-460 region of mT640-147
KNGVCSE

SEQ ID NO: 152 - protein, 7 aa, synthetic construct
  REGION 1..7: note = 454-460 region of mT640-148
FMSAGAE

SEQ ID NO: 153 - protein, 7 aa, synthetic construct
  REGION 1..7: note = 454-460 region of mT640-149
HVGREVG

SEQ ID NO: 154 - protein, 7 aa, synthetic construct
  REGION 1..7: note = 454-460 region of mT640-150
RFSADVD

SEQ ID NO: 155 - protein, 7 aa, synthetic construct
  REGION 1..7: note = 454-460 region of mT640-151
SQLSTCH

SEQ ID NO: 156 - protein, 7 aa, synthetic construct
  REGION 1..7: note = 454-460 region of mT640-152
SARLSTE

SEQ ID NO: 157 - protein, 7 aa, synthetic construct
  REGION 1..7: note = 454-460 region of mT640-153
EVNVPIT

SEQ ID NO: 158 - protein, 7 aa, synthetic construct
  REGION 1..7: note = 454-460 region of mT640-154
TFASEGS

SEQ ID NO: 159 - protein, 6 aa, synthetic construct
  REGION 1..6: note = 454-460 region of mT640-155
GPVCPA

SEQ ID NO: 160 - protein, 7 aa, synthetic construct
  REGION 1..7: note = 454-460 region of mT640-156
GVGRDGE

SEQ ID NO: 161 - protein, 7 aa, synthetic construct
  REGION 1..7: note = 454-460 region of mT640-157
NSACHQS

SEQ ID NO: 162 - protein, 7 aa, synthetic construct
  REGION 1..7: note = 454-460 region of mT640-158
VESRLGG

SEQ ID NO: 163 - protein, 7 aa, synthetic construct
  REGION 1..7: note = 454-460 region of mT640-159
SLVKAAE

SEQ ID NO: 164 - protein, 7 aa, synthetic construct
  REGION 1..7: note = 454-460 region of mT640-160
STSPDPL

SEQ ID NO: 165 - protein, 7 aa, synthetic construct
  REGION 1..7: note = 454-460 region of mT640-161
STDAHAL

SEQ ID NO: 166 - protein, 7 aa, synthetic construct
  REGION 1..7: note = 454-460 region of mT640-162
GNSLDGG

SEQ ID NO: 167 - protein, 7 aa, synthetic construct
  REGION 1..7: note = 454-460 region of mT640-163
FDAPTSR

SEQ ID NO: 168 - protein, 7 aa, synthetic construct
  REGION 1..7: note = 454-460 region of mT640-164
GDTKGLG

SEQ ID NO: 169 - protein, 7 aa, synthetic construct
  REGION 1..7: note = 454-460 region of mT640-165
EGMPKVD

SEQ ID NO: 170 - protein, 7 aa, synthetic construct
  REGION 1..7: note = 454-460 region of mT640-166
ERSPDSV

SEQ ID NO: 171 - protein, 7 aa, synthetic construct
  REGION 1..7: note = 454-460 region of mT640-167
SVQREGG

SEQ ID NO: 172 - protein, 7 aa, synthetic construct
  REGION 1..7: note = 454-460 region of mT640-168
SNGSVGT

SEQ ID NO: 173 - protein, 7 aa, synthetic construct
  REGION 1..7: note = 454-460 region of mT640-169
PAVLQND

SEQ ID NO: 174 - protein, 7 aa, synthetic construct
  REGION 1..7: note = 454-460 region of mT640-171
HTTATAD

SEQ ID NO: 175 - protein, 7 aa, synthetic construct
  REGION 1..7: note = 454-460 region of mT640-172
LDSASAR

SEQ ID NO: 176 - protein, 7 aa, synthetic construct
  REGION 1..7: note = 454-460 region of mT640-173
ALTVDRG

SEQ ID NO: 177 - protein, 7 aa, synthetic construct
  REGION 1..7: note = 454-460 region of mT640-174
SVLTSHG

SEQ ID NO: 178 - protein, 7 aa, synthetic construct
  REGION 1..7: note = 454-460 region of mT640-175
QQASNAG

SEQ ID NO: 179 - protein, 7 aa, synthetic construct
  REGION 1..7: note = 454-460 region of mT640-176
IKNIGTA

SEQ ID NO: 180 - protein, 7 aa, synthetic construct
  REGION 1..7: note = 454-460 region of mT640-177
NSRDCDS

SEQ ID NO: 181 - protein, 7 aa, synthetic construct
  REGION 1..7: note = 454-460 region of mT640-178
SLITSGV

SEQ ID NO: 182 - protein, 7 aa, synthetic construct
  REGION 1..7: note = 454-460 region of mT640-179
TFTSNLE

SEQ ID NO: 183 - protein, 7 aa, synthetic construct
  REGION 1..7: note = 454-460 region of mT640-180
KADVSCG

SEQ ID NO: 184 - protein, 7 aa, synthetic construct
  REGION 1..7: note = 454-460 region of mT640-181
PERTSQA

SEQ ID NO: 185 - protein, 7 aa, synthetic construct
  REGION 1..7: note = 454-460 region of mT640-182
STHGLAD

SEQ ID NO: 186 - protein, 7 aa, synthetic construct
  REGION 1..7: note = 454-460 region of mT640-183
ATLAWET

SEQ ID NO: 187 - protein, 7 aa, synthetic construct
  REGION 1..7: note = 454-460 region of mT640-184
VSSSVLE

SEQ ID NO: 188 - protein, 7 aa, synthetic construct
  REGION 1..7: note = 454-460 region of mT640-185
PSDFTRD

SEQ ID NO: 189 - protein, 7 aa, synthetic construct
  REGION 1..7: note = 454-460 region of mT640-186
ARSEQPG

SEQ ID NO: 190 - protein, 7 aa, synthetic construct
  REGION 1..7: note = 454-460 region of mT640-187
TLLPSHG

SEQ ID NO: 191 - protein, 7 aa, synthetic construct
  REGION 1..7: note = 454-460 region of mT640-188
EEKVRVG

SEQ ID NO: 192 - protein, 7 aa, synthetic construct
  REGION 1..7: note = 454-460 region of mT640-189
PEANKQQ

SEQ ID NO: 193 - protein, 7 aa, synthetic construct
  REGION 1..7: note = 454-460 region of mT640-190
VQDAKAG

SEQ ID NO: 194 - protein, 7 aa, synthetic construct
  REGION 1..7: note = 454-460 region of mT640-191
MESGGMG

SEQ ID NO: 195 - protein, 7 aa, synthetic construct
  REGION 1..7: note = 454-460 region of mT640-192
KSSPEGG

SEQ ID NO: 196 - protein, 7 aa, synthetic construct
  REGION 1..7: note = 454-460 region of mT640-193
GLSSTQS

SEQ ID NO: 197 - protein, 7 aa, synthetic construct
  REGION 1..7: note = 454-460 region of mT640-194
QSSNEPR

SEQ ID NO: 198 - protein, 7 aa, synthetic construct
  REGION 1..7: note = 454-460 region of mT640-195
TEADDRM

SEQ ID NO: 199 - protein, 7 aa, synthetic construct
  REGION 1..7: note = 454-460 region of mT640-196
SHVLEGS

SEQ ID NO: 200 - protein, 6 aa, synthetic construct
  REGION 1..6: note = 454-460 region of mT640-197
ADDGTK

SEQ ID NO: 201 - protein, 7 aa, synthetic construct
  REGION 1..7: note = 454-460 region of mT640-198
REESFGP

SEQ ID NO: 202 - protein, 7 aa, synthetic construct
  REGION 1..7: note = 454-460 region of mT640-199
EPDVRVR

SEQ ID NO: 203 - protein, 7 aa, synthetic construct
  REGION 1..7: note = 454-460 region of mT640-200
ETSASGR

SEQ ID NO: 204 - protein, 7 aa, synthetic construct
  REGION 1..7: note = 454-460 region of mT640-201
VTLTLPL

SEQ ID NO: 205 - protein, 7 aa, synthetic construct
  REGION 1..7: note = 454-460 region of mT640-202
EVSARSS

SEQ ID NO: 206 - protein, 7 aa, synthetic construct
  REGION 1..7: note = 454-460 region of mT640-203
LLKADHT

SEQ ID NO: 207 - protein, 7 aa, synthetic construct
  REGION 1..7: note = 454-460 region of mT640-204
AVMRPEA

SEQ ID NO: 208 - protein, 7 aa, synthetic construct
  REGION 1..7: note = 454-460 region of mT640-205
LISCENE

SEQ ID NO: 209 - protein, 7 aa, synthetic construct
  REGION 1..7: note = 454-460 region of mT640-206
CEKSIPT

SEQ ID NO: 210 - protein, 7 aa, synthetic construct
  REGION 1..7: note = 454-460 region of mT640-207
GERNCPC

SEQ ID NO: 211 - protein, 7 aa, synthetic construct
  REGION 1..7: note = 454-460 region of mT640-208
AGDGGSG

SEQ ID NO: 212 - protein, 7 aa, synthetic construct
  REGION 1..7: note = 454-460 region of mT640-209
LMSTSLE

SEQ ID NO: 213 - protein, 7 aa, synthetic construct
  REGION 1..7: note = 454-460 region of mT640-210
SPTAEAQ

SEQ ID NO: 214 - protein, 7 aa, synthetic construct
  REGION 1..7: note = 454-460 region of mT640-211
SHVIGEC

SEQ ID NO: 215 - protein, 7 aa, synthetic construct
  REGION 1..7: note = 454-460 region of mT640-212
HGAYIPE

SEQ ID NO: 216 - protein, 7 aa, synthetic construct
  REGION 1..7: note = 454-460 region of mT640-213
QAQDAGT

SEQ ID NO: 217 - protein, 7 aa, synthetic construct
  REGION 1..7: note = 454-460 region of mT640-214
EVPTKSL

SEQ ID NO: 218 - protein, 7 aa, synthetic construct
  REGION 1..7: note = 454-460 region of mT640-215
SSGSGLG

SEQ ID NO: 219 - protein, 7 aa, synthetic construct
  REGION 1..7: note = 454-460 region of mT640-216
THQKSEE

SEQ ID NO: 220 - protein, 7 aa, synthetic construct
  REGION 1..7: note = 454-460 region of mT640-217
HSDVCKG

SEQ ID NO: 221 - protein, 7 aa, synthetic construct
  REGION 1..7: note = 454-460 region of mT640-218
SSDVYAG

SEQ ID NO: 222 - protein, 7 aa, synthetic construct
  REGION 1..7: note = 454-460 region of mT640-219
GVNAGAT

SEQ ID NO: 223 - protein, 7 aa, synthetic construct
  REGION 1..7: note = 454-460 region of mT640-220
TTNVRAE

SEQ ID NO: 224 - protein, 7 aa, synthetic construct
  REGION 1..7: note = 454-460 region of mT640-221
DSQKMDL

SEQ ID NO: 225 - protein, 7 aa, synthetic construct
  REGION 1..7: note = 454-460 region of mT640-222
VDIRSAS

SEQ ID NO: 226 - protein, 7 aa, synthetic construct
  REGION 1..7: note = 454-460 region of mT640-223
TANLSQL

SEQ ID NO: 227 - protein, 7 aa, synthetic construct
  REGION 1..7: note = 454-460 region of mT640-224
GKDGGVA

SEQ ID NO: 228 - protein, 7 aa, synthetic construct
  REGION 1..7: note = 454-460 region of mT640-225
LKNQSCD

SEQ ID NO: 229 - protein, 7 aa, synthetic construct
  REGION 1..7: note = 454-460 region of mT640-226
NVTLNSS

SEQ ID NO: 230 - protein, 7 aa, synthetic construct
  REGION 1..7: note = 454-460 region of mT640-227
GSQTGTE

SEQ ID NO: 231 - protein, 7 aa, synthetic construct
  REGION 1..7: note = 454-460 region of mT640-228
DTAKSGQ

SEQ ID NO: 232 - protein, 7 aa, synthetic construct
  REGION 1..7: note = 454-460 region of mT640-229
VEVGGAA

SEQ ID NO: 233 - protein, 7 aa, synthetic construct
  REGION 1..7: note = 454-460 region of mT640-230
SANIGET

SEQ ID NO: 234 - protein, 7 aa, synthetic construct
  REGION 1..7: note = 454-460 region of mT640-231
MDGSSVR

SEQ ID NO: 235 - protein, 7 aa, synthetic construct
  REGION 1..7: note = 454-460 region of mT640-232
ADLSGRE

SEQ ID NO: 236 - protein, 7 aa, synthetic construct
  REGION 1..7: note = 454-460 region of mT640-233
RTESASA

SEQ ID NO: 237 - protein, 7 aa, synthetic construct
  REGION 1..7: note = 454-460 region of mT640-234
LLNGVIP

SEQ ID NO: 238 - protein, 7 aa, synthetic construct
  REGION 1..7: note = 454-460 region of mT640-235
VLDGRSA

SEQ ID NO: 239 - protein, 7 aa, synthetic construct
  REGION 1..7: note = 454-460 region of mT640-236
AAEGSGK

SEQ ID NO: 240 - protein, 7 aa, synthetic construct
  REGION 1..7: note = 454-460 region of mT640-237
VDVKRSG

SEQ ID NO: 241 - protein, 7 aa, synthetic construct
  REGION 1..7: note = 454-460 region of mT640-238
VGRVESE

SEQ ID NO: 242 - protein, 7 aa, synthetic construct
  REGION 1..7: note = 454-460 region of mT640-239
CNMTLEA

SEQ ID NO: 243 - protein, 7 aa, synthetic construct
  REGION 1..7: note = 454-460 region of mT640-240
RDSTIAE

SEQ ID NO: 244 - protein, 7 aa, synthetic construct
  REGION 1..7: note = 454-460 region of mT640-241
EVRGCTE

SEQ ID NO: 245 - protein, 7 aa, synthetic construct
  REGION 1..7: note = 454-460 region of mT640-242
QDGVKWT

SEQ ID NO: 246 - protein, 7 aa, synthetic construct
  REGION 1..7: note = 454-460 region of mT640-243
TILIRED

SEQ ID NO: 247 - protein, 7 aa, synthetic construct
  REGION 1..7: note = 454-460 region of mT640-244
SSVEEGR

SEQ ID NO: 248 - protein, 7 aa, synthetic construct
  REGION 1..7: note = 454-460 region of mT640-245
TLTSQSV

SEQ ID NO: 249 - protein, 7 aa, synthetic construct
  REGION 1..7: note = 454-460 region of mT640-246
IDCTDTE

SEQ ID NO: 250 - protein, 7 aa, synthetic construct
  REGION 1..7: note = 454-460 region of mT640-247
QVDAARA

SEQ ID NO: 251 - protein, 7 aa, synthetic construct
  REGION 1..7: note = 454-460 region of mT640-249
DAGCKAG

SEQ ID NO: 252 - protein, 7 aa, synthetic construct
  REGION 1..7: note = 454-460 region of mT640-250
TTGACHE

SEQ ID NO: 253 - protein, 7 aa, synthetic construct
  REGION 1..7: note = 454-460 region of mT640-251
LRDGAGE

SEQ ID NO: 254 - protein, 7 aa, synthetic construct
  REGION 1..7: note = 454-460 region of mT640-252
SLGTASG

SEQ ID NO: 255 - protein, 7 aa, synthetic construct
  REGION 1..7: note = 454-460 region of mT640-253
NAIASPV

SEQ ID NO: 256 - protein, 7 aa, synthetic construct
  REGION 1..7: note = 454-460 region of mT640-254
SRDTVPA

SEQ ID NO: 257 - protein, 7 aa, synthetic construct
  REGION 1..7: note = 454-460 region of mT640-255
GNSLSPS

SEQ ID NO: 258 - protein, 7 aa, synthetic construct
  REGION 1..7: note = 454-460 region of mT640-256
NLDSWSG

SEQ ID NO: 259 - protein, 7 aa, synthetic construct
  REGION 1..7: note = 454-460 region of mT640-257
EGALKGH

SEQ ID NO: 260 - protein, 7 aa, synthetic construct
  REGION 1..7: note = 454-460 region of mT640-258
IRPDSCD

SEQ ID NO: 261 - protein, 7 aa, synthetic construct
  REGION 1..7: note = 454-460 region of mT640-259
NADVLDG

SEQ ID NO: 262 - protein, 7 aa, synthetic construct
  REGION 1..7: note = 454-460 region of mT640-260
QDKTCSG

SEQ ID NO: 263 - protein, 7 aa, synthetic construct
  REGION 1..7: note = 454-460 region of mT640-261
AQGSYGE

SEQ ID NO: 264 - protein, 7 aa, synthetic construct
  REGION 1..7: note = 454-460 region of mT640-262
EERKEPK

SEQ ID NO: 265 - protein, 7 aa, synthetic construct
  REGION 1..7: note = 454-460 region of mT640-263
SLSSSLE

SEQ ID NO: 266 - protein, 7 aa, synthetic construct
  REGION 1..7: note = 454-460 region of mT640-264
TETGRQV

SEQ ID NO: 267 - protein, 7 aa, synthetic construct
  REGION 1..7: note = 454-460 region of mT640-265
NNAGTDG

SEQ ID NO: 268 - protein, 7 aa, synthetic construct
  REGION 1..7: note = 454-460 region of mT640-266
ETTTKSN

SEQ ID NO: 269 - protein, 7 aa, synthetic construct
  REGION 1..7: note = 454-460 region of mT640-267
QQAPSPG

SEQ ID NO: 270 - protein, 7 aa, synthetic construct
  REGION 1..7: note = 454-460 region of mT640-268
HTGGDAG

SEQ ID NO: 271 - protein, 7 aa, synthetic construct
  REGION 1..7: note = 454-460 region of mT640-269
TISTGSE

SEQ ID NO: 272 - protein, 7 aa, synthetic construct
  REGION 1..7: note = 454-460 region of mT640-270
KAVGSEE

SEQ ID NO: 273 - protein, 7 aa, synthetic construct
  REGION 1..7: note = 454-460 region of mT640-271
RESRSVE

SEQ ID NO: 274 - protein, 7 aa, synthetic construct
  REGION 1..7: note = 454-460 region of mT640-272
REGEAGP

SEQ ID NO: 275 - protein, 7 aa, synthetic construct
  REGION 1..7: note = 454-460 region of mT640-273
PNSSTSS

SEQ ID NO: 276 - protein, 7 aa, synthetic construct
  REGION 1..7: note = 454-460 region of mT640-274
VTLPDSR

SEQ ID NO: 277 - protein, 7 aa, synthetic construct
  REGION 1..7: note = 454-460 region of mT640-275
LPDRGWA

SEQ ID NO: 278 - protein, 7 aa, synthetic construct
  REGION 1..7: note = 454-460 region of mT640-276
GTVMNID

SEQ ID NO: 279 - protein, 7 aa, synthetic construct
  REGION 1..7: note = 454-460 region of mT640-277
STNPLSV

SEQ ID NO: 280 - protein, 4 aa, synthetic construct
  REGION 1..4: note = 454-460 region of mT640-278
VVAS

SEQ ID NO: 281 - protein, 7 aa, synthetic construct
  REGION 1..7: note = 454-460 region of mT640-279
VLTKDPL

SEQ ID NO: 282 - protein, 7 aa, synthetic construct
  REGION 1..7: note = 454-460 region of mT640-280
ALTSAAN

SEQ ID NO: 283 - protein, 7 aa, synthetic construct
  REGION 1..7: note = 454-460 region of mT640-281
VVKSRAK

SEQ ID NO: 284 - protein, 7 aa, synthetic construct
  REGION 1..7: note = 454-460 region of mT640-282
MHSSNGT

SEQ ID NO: 285 - protein, 7 aa, synthetic construct
  REGION 1..7: note = 454-460 region of mT640-283
TSVKNCP

SEQ ID NO: 286 - protein, 7 aa, synthetic construct
  REGION 1..7: note = 454-460 region of mT640-284
VELKTGV

SEQ ID NO: 287 - protein, 7 aa, synthetic construct
  REGION 1..7: note = 454-460 region of mT640-285
TPTQGGD

SEQ ID NO: 288 - protein, 7 aa, synthetic construct
  REGION 1..7: note = 454-460 region of mT640-286
QNGGCVP

SEQ ID NO: 289 - protein, 7 aa, synthetic construct
  REGION 1..7: note = 454-460 region of mT640-287
GSGRDGQ

SEQ ID NO: 290 - protein, 7 aa, synthetic construct
  REGION 1..7: note = 454-460 region of mT640-288
KIVPSHQ

SEQ ID NO: 291 - protein, 7 aa, synthetic construct
  REGION 1..7: note = 454-460 region of mT640-289
ANTGHST

SEQ ID NO: 292 - protein, 7 aa, synthetic construct
  REGION 1..7: note = 454-460 region of mT640-290
KLVASPS

SEQ ID NO: 293 - protein, 7 aa, synthetic construct
  REGION 1..7: note = 454-460 region of mT640-291
DTGIRDS

SEQ ID NO: 294 - protein, 7 aa, synthetic construct
  REGION 1..7: note = 454-460 region of mT640-292
RGEGTHV

SEQ ID NO: 295 - protein, 7 aa, synthetic construct
  REGION 1..7: note = 454-460 region of mT640-293
ASDAFRE

SEQ ID NO: 296 - protein, 7 aa, synthetic construct
  REGION 1..7: note = 454-460 region of mT640-294
DSCGTSH

SEQ ID NO: 297 - protein, 7 aa, synthetic construct
  REGION 1..7: note = 454-460 region of mT640-295
AHTEFNG

SEQ ID NO: 298 - protein, 7 aa, synthetic construct
  REGION 1..7: note = 454-460 region of mT640-296
PGGADRG

SEQ ID NO: 299 - protein, 5 aa, synthetic construct
  REGION 1..5: note = 454-460 region of mT640-297
QSCAA

SEQ ID NO: 300 - protein, 7 aa, synthetic construct
  REGION 1..7: note = 454-460 region of mT640-298
GLNECVE

SEQ ID NO: 301 - protein, 7 aa, synthetic construct
  REGION 1..7: note = 454-460 region of mT640-299
LMSAQTT

SEQ ID NO: 302 - protein, 7 aa, synthetic construct
  REGION 1..7: note = 454-460 region of mT640-300
GNMVGLS

SEQ ID NO: 303 - protein, 7 aa, synthetic construct
  REGION 1..7: note = 454-460 region of mT640-301
QKEFGMD

SEQ ID NO: 304 - protein, 7 aa, synthetic construct
  REGION 1..7: note = 454-460 region of mT640-302
GGTVGDQ

SEQ ID NO: 305 - protein, 7 aa, synthetic construct
  REGION 1..7: note = 454-460 region of mT640-303
STVREGR

SEQ ID NO: 306 - protein, 7 aa, synthetic construct
  REGION 1..7: note = 454-460 region of mT640-304
RFSPEPQ

SEQ ID NO: 307 - protein, 7 aa, synthetic construct
  REGION 1..7: note = 454-460 region of mT640-305
NSGVEST

SEQ ID NO: 308 - protein, 7 aa, synthetic construct
  REGION 1..7: note = 454-460 region of mT640-306
GAKMDSD

SEQ ID NO: 309 - protein, 7 aa, synthetic construct
  REGION 1..7: note = 454-460 region of mT640-307
RETKAEV

SEQ ID NO: 310 - protein, 7 aa, synthetic construct
  REGION 1..7: note = 454-460 region of mT640-308
LGSSLDC

SEQ ID NO: 311 - protein, 7 aa, synthetic construct
  REGION 1..7: note = 454-460 region of mT640-309
MSHTEGG

SEQ ID NO: 312 - protein, 7 aa, synthetic construct
  REGION 1..7: note = 454-460 region of mT640-310
REAREAH

SEQ ID NO: 313 - protein, 7 aa, synthetic construct
  REGION 1..7: note = 454-460 region of mT640-311
STDSAHR

SEQ ID NO: 314 - protein, 7 aa, synthetic construct
  REGION 1..7: note = 454-460 region of mT640-312
NGALGAG

SEQ ID NO: 315 - protein, 7 aa, synthetic construct
  REGION 1..7: note = 454-460 region of mT640-313
TEVGRGC

SEQ ID NO: 316 - protein, 7 aa, synthetic construct
  REGION 1..7: note = 454-460 region of mT640-314
GETNGAT

SEQ ID NO: 317 - protein, 7 aa, synthetic construct
  REGION 1..7: note = 454-460 region of mT640-315
NTSPASL

SEQ ID NO: 318 - protein, 7 aa, synthetic construct
  REGION 1..7: note = 454-460 region of mT640-316
QLSAEGV

SEQ ID NO: 319 - protein, 7 aa, synthetic construct
  REGION 1..7: note = 454-460 region of mT640-317
QATCDSS

SEQ ID NO: 320 - protein, 7 aa, synthetic construct
  REGION 1..7: note = 454-460 region of mT640-318
KDTCHDS

SEQ ID NO: 321 - protein, 7 aa, synthetic construct
  REGION 1..7: note = 454-460 region of mT640-319
SNERACG

SEQ ID NO: 322 - protein, 7 aa, synthetic construct
  REGION 1..7: note = 454-460 region of mT640-320
PENMSGP

SEQ ID NO: 323 - protein, 7 aa, synthetic construct
  REGION 1..7: note = 454-460 region of mT640-321
VSIAEDV

SEQ ID NO: 324 - protein, 7 aa, synthetic construct
  REGION 1..7: note = 454-460 region of mT640-322
KEGVGPG

SEQ ID NO: 325 - protein, 7 aa, synthetic construct
  REGION 1..7: note = 454-460 region of mT640-323
SPSDKVL

SEQ ID NO: 326 - protein, 7 aa, synthetic construct
  REGION 1..7: note = 454-460 region of mT640-324
STALVTT

SEQ ID NO: 327 - protein, 7 aa, synthetic construct
  REGION 1..7: note = 454-460 region of mT640-325
GSHRQEE

SEQ ID NO: 328 - protein, 7 aa, synthetic construct
  REGION 1..7: note = 454-460 region of mT640-326
GVALKGE

SEQ ID NO: 329 - protein, 7 aa, synthetic construct
  REGION 1..7: note = 454-460 region of mT640-327
TADMCPR

SEQ ID NO: 330 - protein, 7 aa, synthetic construct
  REGION 1..7: note = 454-460 region of mT640-328
KTVLDQT

SEQ ID NO: 331 - protein, 7 aa, synthetic construct
  REGION 1..7: note = 454-460 region of mT640-329
GAGVAAS

SEQ ID NO: 332 - protein, 7 aa, synthetic construct
  REGION 1..7: note = 454-460 region of mT640-330
QERLNSV

SEQ ID NO: 333 - protein, 7 aa, synthetic construct
  REGION 1..7: note = 454-460 region of mT640-331
NGDKRQE

SEQ ID NO: 334 - protein, 7 aa, synthetic construct
  REGION 1..7: note = 454-460 region of mT640-332
GGDQGAQ

SEQ ID NO: 335 - protein, 7 aa, synthetic construct
  REGION 1..7: note = 454-460 region of mT640-333
STDRNVM

SEQ ID NO: 336 - protein, 7 aa, synthetic construct
  REGION 1..7: note = 454-460 region of mT640-334
SDRLTVG

SEQ ID NO: 337 - protein, 7 aa, synthetic construct
  REGION 1..7: note = 454-460 region of mT640-335
SDLGKGE

SEQ ID NO: 338 - protein, 7 aa, synthetic construct
  REGION 1..7: note = 454-460 region of mT640-336
GLEGNSS

SEQ ID NO: 339 - protein, 7 aa, synthetic construct
  REGION 1..7: note = 454-460 region of mT640-337
GQDSDRN

SEQ ID NO: 340 - protein, 7 aa, synthetic construct
  REGION 1..7: note = 454-460 region of mT640-338
KPDGREA

SEQ ID NO: 341 - protein, 7 aa, synthetic construct
  REGION 1..7: note = 454-460 region of mT640-339
DPSGNGA

SEQ ID NO: 342 - protein, 7 aa, synthetic construct
  REGION 1..7: note = 454-460 region of mT640-340
PSEGQGG

SEQ ID NO: 343 - protein, 7 aa, synthetic construct
  REGION 1..7: note = 454-460 region of mT640-341
SGTVMQG

SEQ ID NO: 344 - protein, 7 aa, synthetic construct
  REGION 1..7: note = 454-460 region of mT640-342
PTASVST

SEQ ID NO: 345 - protein, 7 aa, synthetic construct
  REGION 1..7: note = 454-460 region of mT640-343
PISESSN

SEQ ID NO: 346 - protein, 7 aa, synthetic construct
  REGION 1..7: note = 454-460 region of mT640-344
SSDGLNN

SEQ ID NO: 347 - protein, 7 aa, synthetic construct
  REGION 1..7: note = 454-460 region of mT640-345
SEAQASV

SEQ ID NO: 348 - protein, 7 aa, synthetic construct
  REGION 1..7: note = 454-460 region of mT640-346
SGEPRGS

SEQ ID NO: 349 - protein, 7 aa, synthetic construct
  REGION 1..7: note = 454-460 region of mT640-347
SSESKCS

SEQ ID NO: 350 - protein, 7 aa, synthetic construct
  REGION 1..7: note = 454-460 region of mT640-348
ASSRETT

SEQ ID NO: 351 - protein, 7 aa, synthetic construct
  REGION 1..7: note = 454-460 region of mT640-349
SSEPGVR

SEQ ID NO: 352 - protein, 7 aa, synthetic construct
  REGION 1..7: note = 454-460 region of mT640-350
ESMSKPW

SEQ ID NO: 353 - protein, 7 aa, synthetic construct
  REGION 1..7: note = 454-460 region of mT640-351
STDLRQH

SEQ ID NO: 354 - protein, 7 aa, synthetic construct
  REGION 1..7: note = 454-460 region of mT640-352
EGVGREG

SEQ ID NO: 355 - protein, 7 aa, synthetic construct
  REGION 1..7: note = 454-460 region of mT640-353
KSGKEVE

SEQ ID NO: 356 - protein, 7 aa, synthetic construct
  REGION 1..7: note = 454-460 region of mT640-354
GWNKGTE

SEQ ID NO: 357 - protein, 7 aa, synthetic construct
  REGION 1..7: note = 454-460 region of mT640-355
IPLCARV

SEQ ID NO: 358 - protein, 7 aa, synthetic construct
  REGION 1..7: note = 454-460 region of mT640-356
TALNAEK

SEQ ID NO: 359 - protein, 7 aa, synthetic construct
  REGION 1..7: note = 454-460 region of mT640-357
SAGLCGQ

SEQ ID NO: 360 - protein, 7 aa, synthetic construct
  REGION 1..7: note = 454-460 region of mT640-358
VVNPKVE

SEQ ID NO: 361 - protein, 7 aa, synthetic construct
  REGION 1..7: note = 454-460 region of mT640-359
LLLPVGE

SEQ ID NO: 362 - protein, 7 aa, synthetic construct
  REGION 1..7: note = 454-460 region of mT640-360
VVRVEGA

SEQ ID NO: 363 - protein, 7 aa, synthetic construct
  REGION 1..7: note = 454-460 region of mT640-361
VLSAELV

SEQ ID NO: 364 - protein, 7 aa, synthetic construct
  REGION 1..7: note = 454-460 region of mT640-362
VLTQQCT

SEQ ID NO: 365 - protein, 7 aa, synthetic construct
  REGION 1..7: note = 454-460 region of mT640-363
VVGIDSA

SEQ ID NO: 366 - protein, 7 aa, synthetic construct
  REGION 1..7: note = 454-460 region of mT640-364
VLGQDAS

SEQ ID NO: 367 - protein, 7 aa, synthetic construct
  REGION 1..7: note = 454-460 region of mT640-365
ALLPQQG

SEQ ID NO: 368 - protein, 7 aa, synthetic construct
  REGION 1..7: note = 454-460 region of mT640-366
VAEARGG

SEQ ID NO: 369 - protein, 7 aa, synthetic construct
  REGION 1..7: note = 454-460 region of mT640-367
VLASSHT

SEQ ID NO: 370 - protein, 7 aa, synthetic construct
  REGION 1..7: note = 454-460 region of mT640-368
EAGACSR

SEQ ID NO: 371 - protein, 7 aa, synthetic construct
  REGION 1..7: note = 454-460 region of mT640-369
MFKTTEG

SEQ ID NO: 372 - protein, 7 aa, synthetic construct
  REGION 1..7: note = 454-460 region of mT640-370
GLFPGSE

SEQ ID NO: 373 - protein, 7 aa, synthetic construct
  REGION 1..7: note = 454-460 region of mT640-371
RLLPETA

SEQ ID NO: 374 - protein, 7 aa, synthetic construct
  REGION 1..7: note = 454-460 region of mT640-372
GLVKTAS

SEQ ID NO: 375 - protein, 7 aa, synthetic construct
  REGION 1..7: note = 454-460 region of mT640-373
ADCEENR

SEQ ID NO: 376 - protein, 7 aa, synthetic construct
  REGION 1..7: note = 454-460 region of mT640-374
DGSGSEE

SEQ ID NO: 377 - protein, 7 aa, synthetic construct
  REGION 1..7: note = 454-460 region of mT640-375
EERSKAH

SEQ ID NO: 378 - protein, 7 aa, synthetic construct
  REGION 1..7: note = 454-460 region of mT640-376
PGQTLQS

SEQ ID NO: 379 - protein, 7 aa, synthetic construct
  REGION 1..7: note = 454-460 region of mT640-377
TLISREG

SEQ ID NO: 380 - protein, 7 aa, synthetic construct
  REGION 1..7: note = 454-460 region of mT640-378
RDPGPAA

SEQ ID NO: 381 - protein, 7 aa, synthetic construct
  REGION 1..7: note = 454-460 region of mT640-379
GNAINVD

SEQ ID NO: 382 - protein, 7 aa, synthetic construct
  REGION 1..7: note = 454-460 region of mT640-380
TLSKTVS

SEQ ID NO: 383 - protein, 7 aa, synthetic construct
  REGION 1..7: note = 454-460 region of mT640-381
ERSRECG

SEQ ID NO: 384 - protein, 7 aa, synthetic construct
  REGION 1..7: note = 454-460 region of mT640-382
TGSGGAS

SEQ ID NO: 385 - protein, 7 aa, synthetic construct
  REGION 1..7: note = 454-460 region of mT640-383
EESARKL

SEQ ID NO: 386 - protein, 7 aa, synthetic construct
  REGION 1..7: note = 454-460 region of mT640-384
ASTNIDY

SEQ ID NO: 387 - protein, 7 aa, synthetic construct
  REGION 1..7: note = 454-460 region of mT640-385
GLENEMT

SEQ ID NO: 388 - protein, 7 aa, synthetic construct
  REGION 1..7: note = 454-460 region of mT640-386
SADGGSG

SEQ ID NO: 389 - protein, 7 aa, synthetic construct
  REGION 1..7: note = 454-460 region of mT640-387
SSGECAR

SEQ ID NO: 390 - protein, 7 aa, synthetic construct
  REGION 1..7: note = 454-460 region of mT640-388
TERVSHH

SEQ ID NO: 391 - protein, 7 aa, synthetic construct
  REGION 1..7: note = 454-460 region of mT640-389
IEGVGGG

SEQ ID NO: 392 - protein, 7 aa, synthetic construct
  REGION 1..7: note = 454-460 region of mT640-390
SSTLTGA

SEQ ID NO: 393 - protein, 7 aa, synthetic construct
  REGION 1..7: note = 454-460 region of mT640-391
FSTQSAQ

SEQ ID NO: 394 - protein, 7 aa, synthetic construct
  REGION 1..7: note = 454-460 region of mT640-392
GDSEGWQ

SEQ ID NO: 395 - protein, 7 aa, synthetic construct
  REGION 1..7: note = 454-460 region of mT640-393
NLVDATP

SEQ ID NO: 396 - protein, 7 aa, synthetic construct
  REGION 1..7: note = 454-460 region of mT640-394
DRDGETG

SEQ ID NO: 397 - protein, 7 aa, synthetic construct
  REGION 1..7: note = 454-460 region of mT640-395
ANQRPEA

SEQ ID NO: 398 - protein, 7 aa, synthetic construct
  REGION 1..7: note = 454-460 region of mT640-396
GGAANTG

SEQ ID NO: 399 - protein, 7 aa, synthetic construct
  REGION 1..7: note = 454-460 region of mT640-397
EGGVQER

SEQ ID NO: 400 - protein, 7 aa, synthetic construct
  REGION 1..7: note = 454-460 region of mT640-398
NTNKATE

SEQ ID NO: 401 - protein, 7 aa, synthetic construct
  REGION 1..7: note = 454-460 region of mT640-399
EARAEAR

SEQ ID NO: 402 - protein, 7 aa, synthetic construct
  REGION 1..7: note = 454-460 region of mT640-400
SLESDCG

SEQ ID NO: 403 - protein, 7 aa, synthetic construct
  REGION 1..7: note = 454-460 region of mT640-401
GDARNRE

SEQ ID NO: 404 - protein, 7 aa, synthetic construct
  REGION 1..7: note = 454-460 region of mT640-402
TSHQSAE

SEQ ID NO: 405 - protein, 7 aa, synthetic construct
  REGION 1..7: note = 454-460 region of mT640-403
GVSGSPG

SEQ ID NO: 406 - protein, 7 aa, synthetic construct
  REGION 1..7: note = 454-460 region of mT640-404
VTSTEPR

SEQ ID NO: 407 - protein, 7 aa, synthetic construct
  REGION 1..7: note = 454-460 region of mT640-405
SQILGSA

SEQ ID NO: 408 - protein, 7 aa, synthetic construct
  REGION 1..7: note = 454-460 region of mT640-406
LGETNAG

SEQ ID NO: 409 - protein, 7 aa, synthetic construct
  REGION 1..7: note = 454-460 region of mT640-407
ESRLSAS

SEQ ID NO: 410 - protein, 7 aa, synthetic construct
  REGION 1..7: note = 454-460 region of mT640-408
STATTVG

SEQ ID NO: 411 - protein, 7 aa, synthetic construct
  REGION 1..7: note = 454-460 region of mT640-409
MLHQEAA

SEQ ID NO: 412 - protein, 7 aa, synthetic construct
  REGION 1..7: note = 454-460 region of mT640-410
GGGEAGC

SEQ ID NO: 413 - protein, 7 aa, synthetic construct
  REGION 1..7: note = 454-460 region of mT640-411
ATAVQCN

SEQ ID NO: 414 - protein, 7 aa, synthetic construct
  REGION 1..7: note = 454-460 region of mT640-412
TDSANPA

SEQ ID NO: 415 - protein, 7 aa, synthetic construct
  REGION 1..7: note = 454-460 region of mT640-413
DASTTTG

SEQ ID NO: 416 - protein, 7 aa, synthetic construct
  REGION 1..7: note = 454-460 region of mT640-414
SDKVNAS

SEQ ID NO: 417 - protein, 7 aa, synthetic construct
  REGION 1..7: note = 454-460 region of mT640-415
EGTQSGS

SEQ ID NO: 418 - protein, 7 aa, synthetic construct
  REGION 1..7: note = 454-460 region of mT640-416
RVEQGVG

SEQ ID NO: 419 - protein, 7 aa, synthetic construct
  REGION 1..7: note = 454-460 region of mT640-417
SASGLEG

SEQ ID NO: 420 - protein, 7 aa, synthetic construct
  REGION 1..7: note = 454-460 region of mT640-418
GHGQQSD

SEQ ID NO: 421 - protein, 7 aa, synthetic construct
  REGION 1..7: note = 454-460 region of mT640-419
ASLGPQG

SEQ ID NO: 422 - protein, 7 aa, synthetic construct
  REGION 1..7: note = 454-460 region of mT640-420
SSDSLRS

SEQ ID NO: 423 - protein, 7 aa, synthetic construct
  REGION 1..7: note = 454-460 region of mT640-421
ESVKTAQ

SEQ ID NO: 424 - protein, 7 aa, synthetic construct
  REGION 1..7: note = 454-460 region of mT640-422
KAELGTA

SEQ ID NO: 425 - protein, 7 aa, synthetic construct
  REGION 1..7: note = 454-460 region of mT640-423
GDTRPNC

SEQ ID NO: 426 - protein, 7 aa, synthetic construct
  REGION 1..7: note = 454-460 region of mT640-424
AQELGHG

SEQ ID NO: 427 - protein, 7 aa, synthetic construct
  REGION 1..7: note = 454-460 region of mT640-425
DGLSRST

SEQ ID NO: 428 - protein, 4 aa, synthetic construct
  REGION 1..4: note = 454-460 region of mT640-426
EAVH

SEQ ID NO: 429 - protein, 7 aa, synthetic construct
  REGION 1..7: note = 454-460 region of mT640-427
STAPDPR

SEQ ID NO: 430 - protein, 7 aa, synthetic construct
  REGION 1..7: note = 454-460 region of mT640-428
SSSEINP

SEQ ID NO: 431 - protein, 7 aa, synthetic construct
  REGION 1..7: note = 454-460 region of mT640-429
KRSDVPT

SEQ ID NO: 432 - protein, 7 aa, synthetic construct
  REGION 1..7: note = 454-460 region of mT640-430
SSSSRQL

SEQ ID NO: 433 - protein, 7 aa, synthetic construct
  REGION 1..7: note = 454-460 region of mT640-431
LFTTSRR

SEQ ID NO: 434 - protein, 7 aa, synthetic construct
  REGION 1..7: note = 454-460 region of mT640-432
PKNPEEV

SEQ ID NO: 435 - protein, 7 aa, synthetic construct
  REGION 1..7: note = 454-460 region of mT640-433
VGNPALT

SEQ ID NO: 436 - protein, 7 aa, synthetic construct
  REGION 1..7: note = 454-460 region of mT640-434
IVARAGD

SEQ ID NO: 437 - protein, 7 aa, synthetic construct
  REGION 1..7: note = 454-460 region of mT640-435
LLSKEQI

SEQ ID NO: 438 - protein, 7 aa, synthetic construct
  REGION 1..7: note = 454-460 region of mT640-436
LAPRDRE

SEQ ID NO: 439 - protein, 7 aa, synthetic construct
  REGION 1..7: note = 454-460 region of mT640-437
VVMTESR

SEQ ID NO: 440 - protein, 7 aa, synthetic construct
  REGION 1..7: note = 454-460 region of mT640-438
VLQQASA

SEQ ID NO: 441 - protein, 7 aa, synthetic construct
  REGION 1..7: note = 454-460 region of mT640-439
VAERERG

SEQ ID NO: 442 - protein, 7 aa, synthetic construct
  REGION 1..7: note = 454-460 region of mT640-440
VIRSRIR

SEQ ID NO: 443 - protein, 7 aa, synthetic construct
  REGION 1..7: note = 454-460 region of mT640-441
TSSICAE

SEQ ID NO: 444 - protein, 7 aa, synthetic construct
  REGION 1..7: note = 454-460 region of mT640-442
EYVRPGE

SEQ ID NO: 445 - protein, 7 aa, synthetic construct
  REGION 1..7: note = 454-460 region of mT640-443
SDGFGVL

SEQ ID NO: 446 - protein, 7 aa, synthetic construct
  REGION 1..7: note = 454-460 region of mT640-444
CQIPEGV

SEQ ID NO: 447 - protein, 7 aa, synthetic construct
  REGION 1..7: note = 454-460 region of mT640-445
TLLTVPC

SEQ ID NO: 448 - protein, 7 aa, synthetic construct
  REGION 1..7: note = 454-460 region of mT640-446
EERRSSP

SEQ ID NO: 449 - protein, 7 aa, synthetic construct
  REGION 1..7: note = 454-460 region of mT640-447
HTTQTVH

SEQ ID NO: 450 - protein, 7 aa, synthetic construct
  REGION 1..7: note = 454-460 region of mT640-448
VDAYEVQ

SEQ ID NO: 451 - protein, 7 aa, synthetic construct
  REGION 1..7: note = 454-460 region of mT640-449
SLLPRES

SEQ ID NO: 452 - protein, 7 aa, synthetic construct
  REGION 1..7: note = 454-460 region of mT640-450
SISVRES

SEQ ID NO: 453 - protein, 7 aa, synthetic construct
  REGION 1..7: note = 454-460 region of mT640-451
LGVGVVG

SEQ ID NO: 454 - protein, 7 aa, synthetic construct
  REGION 1..7: note = 454-460 region of mT640-452
MTMTTSA

SEQ ID NO: 455 - protein, 7 aa, synthetic construct
  REGION 1..7: note = 454-460 region of mT640-453
ERLQKAE

SEQ ID NO: 456 - protein, 7 aa, synthetic construct
  REGION 1..7: note = 454-460 region of mT640-454
DSSTRTL

SEQ ID NO: 457 - protein, 7 aa, synthetic construct
  REGION 1..7: note = 454-460 region of mT640-455
LTNGGCH

SEQ ID NO: 458 - protein, 7 aa, synthetic construct
  REGION 1..7: note = 454-460 region of mT640-456
APTQTVS

SEQ ID NO: 459 - protein, 7 aa, synthetic construct
  REGION 1..7: note = 454-460 region of mT640-457
NREDTTH

SEQ ID NO: 460 - protein, 7 aa, synthetic construct
  REGION 1..7: note = 454-460 region of mT640-458
TFNIAPS

SEQ ID NO: 461 - protein, 7 aa, synthetic construct
  REGION 1..7: note = 454-460 region of mT640-459
VDPAGQR

SEQ ID NO: 462 - protein, 7 aa, synthetic construct
  REGION 1..7: note = 454-460 region of mT640-460
AQHLNGE

SEQ ID NO: 463 - protein, 7 aa, synthetic construct
  REGION 1..7: note = 454-460 region of mT640-461
TKGSETW

SEQ ID NO: 464 - protein, 7 aa, synthetic construct
  REGION 1..7: note = 454-460 region of mT640-462
EALLKPY

SEQ ID NO: 465 - protein, 7 aa, synthetic construct
  REGION 1..7: note = 454-460 region of mT640-463
TKGSETW

SEQ ID NO: 466 - protein, 7 aa, synthetic construct
  REGION 1..7: note = 454-460 region of mT640-464
EGTVGAR

SEQ ID NO: 467 - protein, 7 aa, synthetic construct
  REGION 1..7: note = 454-460 region of mT640-465
HQDLCDK

SEQ ID NO: 468 - protein, 7 aa, synthetic construct
  REGION 1..7: note = 454-460 region of mT640-466
IDTGAHR

SEQ ID NO: 469 - protein, 7 aa, synthetic construct
  REGION 1..7: note = 454-460 region of mT640-467
QEKGSSS

SEQ ID NO: 470 - protein, 7 aa, synthetic construct
  REGION 1..7: note = 454-460 region of mT640-468
GDRGDSS

SEQ ID NO: 471 - protein, 7 aa, synthetic construct
  REGION 1..7: note = 454-460 region of mT640-469
TEGKCER

SEQ ID NO: 472 - protein, 7 aa, synthetic construct
  REGION 1..7: note = 454-460 region of mT640-470
DSAGGGA

SEQ ID NO: 473 - protein, 7 aa, synthetic construct
  REGION 1..7: note = 454-460 region of mT640-471
QGGPSSG

SEQ ID NO: 474 - protein, 7 aa, synthetic construct
  REGION 1..7: note = 454-460 region of mT640-472
GSNTCVG

SEQ ID NO: 475 - protein, 7 aa, synthetic construct
  REGION 1..7: note = 454-460 region of mT640-473
TGPSMGG

SEQ ID NO: 476 - protein, 7 aa, synthetic construct
  REGION 1..7: note = 454-460 region of mT640-474
AHDRAGA

SEQ ID NO: 477 - protein, 7 aa, synthetic construct
  REGION 1..7: note = 454-460 region of mT640-475
GEVHRAS

SEQ ID NO: 478 - protein, 7 aa, synthetic construct
  REGION 1..7: note = 454-460 region of mT640-476
GGNVGEV

SEQ ID NO: 479 - protein, 7 aa, synthetic construct
  REGION 1..7: note = 454-460 region of mT640-477
DRTSDIA

SEQ ID NO: 480 - protein, 7 aa, synthetic construct
  REGION 1..7: note = 454-460 region of mT640-478
LGMENPP

SEQ ID NO: 481 - protein, 7 aa, synthetic construct
  REGION 1..7: note = 454-460 region of mT640-479
GDSGAMQ

SEQ ID NO: 482 - protein, 7 aa, synthetic construct
  REGION 1..7: note = 454-460 region of mT640-480
REAGLDG

SEQ ID NO: 483 - protein, 7 aa, synthetic construct
  REGION 1..7: note = 454-460 region of mT640-481
MASNGYD

SEQ ID NO: 484 - protein, 7 aa, synthetic construct
  REGION 1..7: note = 454-460 region of mT640-482
SVSAWSE

SEQ ID NO: 485 - protein, 7 aa, synthetic construct
  REGION 1..7: note = 454-460 region of mT640-483
GQGEGSG

SEQ ID NO: 486 - protein, 7 aa, synthetic construct
  REGION 1..7: note = 454-460 region of mT640-484
KAGLAAD

SEQ ID NO: 487 - protein, 7 aa, synthetic construct
  REGION 1..7: note = 454-460 region of mT640-485
VDSARTS

SEQ ID NO: 488 - protein, 7 aa, synthetic construct
  REGION 1..7: note = 454-460 region of mT640-486
CQVDGSQ

SEQ ID NO: 489 - protein, 7 aa, synthetic construct
  REGION 1..7: note = 454-460 region of mT640-487
PTVVGQG

SEQ ID NO: 490 - protein, 7 aa, synthetic construct
  REGION 1..7: note = 454-460 region of mT640-488
TAKLVPE

SEQ ID NO: 491 - protein, 7 aa, synthetic construct
  REGION 1..7: note = 454-460 region of mT640-489
STMKEES

SEQ ID NO: 492 - protein, 7 aa, synthetic construct
  REGION 1..7: note = 454-460 region of mT640-490
LHDVDSS

SEQ ID NO: 493 - protein, 7 aa, synthetic construct
  REGION 1..7: note = 454-460 region of mT640-491
GNVAVER

SEQ ID NO: 494 - protein, 7 aa, synthetic construct
  REGION 1..7: note = 454-460 region of mT640-492
ATSDPGS

SEQ ID NO: 495 - protein, 7 aa, synthetic construct
  REGION 1..7: note = 454-460 region of mT640-493
VSDNRAG

SEQ ID NO: 496 - protein, 7 aa, synthetic construct
  REGION 1..7: note = 454-460 region of mT640-494
AGQLDSG

SEQ ID NO: 497 - protein, 7 aa, synthetic construct
  REGION 1..7: note = 454-460 region of mT640-495
KVDAANG

SEQ ID NO: 498 - protein, 7 aa, synthetic construct
  REGION 1..7: note = 454-460 region of mT640-496
KLSPSQG

SEQ ID NO: 499 - protein, 7 aa, synthetic construct
  REGION 1..7: note = 454-460 region of mT640-497
DPRKDCT

SEQ ID NO: 500 - protein, 7 aa, synthetic construct
  REGION 1..7: note = 454-460 region of mT640-498
SPTSIGT

SEQ ID NO: 501 - protein, 7 aa, synthetic construct
  REGION 1..7: note = 454-460 region of mT640-499
MSEHVNG

SEQ ID NO: 502 - protein, 7 aa, synthetic construct
  REGION 1..7: note = 454-460 region of mT640-500
LEGHGAG

SEQ ID NO: 503 - protein, 7 aa, synthetic construct
  REGION 1..7: note = 454-460 region of mT640-501
VEVNDRN

SEQ ID NO: 504 - protein, 7 aa, synthetic construct
  REGION 1..7: note = 454-460 region of mT640-502
SASLTCG

SEQ ID NO: 505 - protein, 7 aa, synthetic construct
  REGION 1..7: note = 454-460 region of mT640-503
VDTMRHG

SEQ ID NO: 506 - protein, 7 aa, synthetic construct
  REGION 1..7: note = 454-460 region of mT640-504
TSGQLQS

SEQ ID NO: 507 - protein, 7 aa, synthetic construct
  REGION 1..7: note = 454-460 region of mT640-505
LNALSAT

SEQ ID NO: 508 - protein, 7 aa, synthetic construct
  REGION 1..7: note = 454-460 region of mT640-506
TINVSPE

SEQ ID NO: 509 - protein, 7 aa, synthetic construct
  REGION 1..7: note = 454-460 region of mT640-507
NLSSGSD

SEQ ID NO: 510 - protein, 7 aa, synthetic construct
  REGION 1..7: note = 454-460 region of mT640-508
ESGPRGS

SEQ ID NO: 511 - protein, 7 aa, synthetic construct
  REGION 1..7: note = 454-460 region of mT640-509
VMNKAEE

SEQ ID NO: 512 - protein, 7 aa, synthetic construct
  REGION 1..7: note = 454-460 region of mT640-510
DSSPLGR

SEQ ID NO: 513 - protein, 7 aa, synthetic construct
  REGION 1..7: note = 454-460 region of mT640-511
AREGVPG

SEQ ID NO: 514 - protein, 7 aa, synthetic construct
  REGION 1..7: note = 454-460 region of mT640-512
GQERATC

SEQ ID NO: 515 - protein, 7 aa, synthetic construct
  REGION 1..7: note = 454-460 region of mT640-513
ESAAGVR

SEQ ID NO: 516 - protein, 7 aa, synthetic construct
  REGION 1..7: note = 454-460 region of mT640-514
SGVAGSA

SEQ ID NO: 517 - protein, 7 aa, synthetic construct
  REGION 1..7: note = 454-460 region of mT640-515
LERSQEG

SEQ ID NO: 518 - protein, 7 aa, synthetic construct
  REGION 1..7: note = 454-460 region of mT640-516
SSGHGVG

SEQ ID NO: 519 - protein, 7 aa, synthetic construct
  REGION 1..7: note = 454-460 region of mT640-517
SSSQEHR

SEQ ID NO: 520 - protein, 7 aa, synthetic construct
  REGION 1..7: note = 454-460 region of mT640-518
ASDSVGS

SEQ ID NO: 521 - protein, 7 aa, synthetic construct
  REGION 1..7: note = 454-460 region of mT640-519
ADHTASH

SEQ ID NO: 522 - protein, 7 aa, synthetic construct
  REGION 1..7: note = 454-460 region of mT640-520
GEEGRGR

SEQ ID NO: 523 - protein, 7 aa, synthetic construct
  REGION 1..7: note = 454-460 region of mT640-521
QASAEVR

SEQ ID NO: 524 - protein, 7 aa, synthetic construct
  REGION 1..7: note = 454-460 region of mT640-522
PESGGLG

SEQ ID NO: 525 - protein, 7 aa, synthetic construct
  REGION 1..7: note = 454-460 region of mT640-523
KSSPESL

SEQ ID NO: 526 - protein, 7 aa, synthetic construct
  REGION 1..7: note = 454-460 region of mT640-524
LNATAAT

SEQ ID NO: 527 - protein, 7 aa, synthetic construct
  REGION 1..7: note = 454-460 region of mT640-525
LEKDGWE

SEQ ID NO: 528 - protein, 7 aa, synthetic construct
  REGION 1..7: note = 454-460 region of mT640-526
DPTLPRQ

SEQ ID NO: 529 - protein, 7 aa, synthetic construct
  REGION 1..7: note = 454-460 region of mT640-527
DAAGKAH

SEQ ID NO: 530 - protein, 7 aa, synthetic construct
  REGION 1..7: note = 454-460 region of mT640-528
KFSYACF

SEQ ID NO: 531 - protein, 7 aa, synthetic construct
  REGION 1..7: note = 454-460 region of mT640-529
VFKQRFS

SEQ ID NO: 532 - protein, 7 aa, synthetic construct
  REGION 1..7: note = 454-460 region of mT640-530
GLSCAGL

SEQ ID NO: 533 - protein, 7 aa, synthetic construct
  REGION 1..7: note = 454-460 region of mT640-531
MRYWESR

SEQ ID NO: 534 - protein, 7 aa, synthetic construct
  REGION 1..7: note = 454-460 region of mT640-532
STQVSDA

SEQ ID NO: 535 - protein, 7 aa, synthetic construct
  REGION 1..7: note = 454-460 region of mT640-533
MSCETAT

SEQ ID NO: 536 - protein, 7 aa, synthetic construct
  REGION 1..7: note = 454-460 region of mT640-534
HTWNGLG

SEQ ID NO: 537 - protein, 7 aa, synthetic construct
  REGION 1..7: note = 454-460 region of mT640-535
EASEHKY

SEQ ID NO: 538 - protein, 7 aa, synthetic construct
  REGION 1..7: note = 454-460 region of mT640-536
VTRQTCD

SEQ ID NO: 539 - protein, 7 aa, synthetic construct
  REGION 1..7: note = 454-460 region of mT640-537
VLVTLGS

SEQ ID NO: 540 - protein, 4 aa, synthetic construct
  REGION 1..4: note = 454-460 region of mT640-538
LANG

SEQ ID NO: 541 - protein, 7 aa, synthetic construct
  REGION 1..7: note = 454-460 region of mT640-539
LIDASFN

SEQ ID NO: 542 - protein, 7 aa, synthetic construct
  REGION 1..7: note = 454-460 region of mT640-540
VAERKSV

SEQ ID NO: 543-544 - skipped (2 entries)

SEQ ID NO: 545 - protein, 7 aa, synthetic construct
  REGION 1..7: note = 454-460 region of mT640
GSAQNKD

SEQ ID NO: 546 - DNA, 25 bp, synthetic construct
  misc_feature 1..25: note = AAV2 inverted terminal repeat region #1 - (ITR) 5
  source 1..25: mol_type = other DNA
aacatgctacgcagagagggagtgg

SEQ ID NO: 547 - DNA, 29 bp, synthetic construct
  misc_feature 1..29: note = AAV2 inverted terminal repeat region #2 - (ITR) 5?
  source 1..29: mol_type = other DNA
catgagacaaggaacccctagtgatggag

SEQ ID NO: 548 - DNA, 332 bp, synthetic construct
  misc_feature 1..332: note = gRNA
  source 1..332: mol_type = other DNA
ggtttccgcttccaccttctccctcttctcgctcgtcctccggtagcagccgccagcacagtgtcggctgctgggcggggtgtcggcggatcggtcggttggtttcggcttcggcgtctctgcggttgagttgggctgtcatggccgagttggatccattcggcgctcccgccggtgcgcccggtggccccgctctaggaaacggggtggcgggcgccggcgaagaagacccggccgcggccttcttggcgcagcaagagagtgagattgcgggcatcgagaacgatgaggccttcgccatcctggacggcggcgcccccgggccgcagcca

SEQ ID NO: 549 - DNA, 20 bp, synthetic construct
  misc_feature 1..20: note = Clta gRNA 5'
  source 1..20: mol_type = other DNA
atggccgagttggatccatt

SEQ ID NO: 550 - DNA, 22 bp, synthetic construct
  misc_feature 1..22: note = GFP Forward 5'
  source 1..22: mol_type = other DNA
ttgtggctcaccaacccaaccg

SEQ ID NO: 551 - DNA, 22 bp, synthetic construct
  misc_feature 1..22: note = GFP Reverse 5'
  source 1..22: mol_type = other DNA
cactcagaagccggcagtctgc

SEQ ID NO: 552 - DNA, 22 bp, synthetic construct
  source 1..22: mol_type = other DNA
agagttccgctcttgcctgtca

SEQ ID NO: 553 - DNA, 22 bp, synthetic construct
  source 1..22: mol_type = other DNA
gtctgtgggccaagaggactga

SEQ ID NO: 554 - DNA, 20 bp, synthetic construct
  source 1..20: mol_type = other DNA
gggcacaagctggagtacaa

SEQ ID NO: 555 - DNA, 20 bp, synthetic construct
  source 1..20: mol_type = other DNA
acgttgtggcggatcttgaa

SEQ ID NO: 556 - DNA, 21 bp, synthetic construct
  source 1..21: mol_type = other DNA
ggactatcatatgcttaccgt

SEQ ID NO: 557 - DNA, 20 bp, synthetic construct
  source 1..20: mol_type = other DNA
ggtgtttcgtcctttccaca

SEQ ID NO: 558 - DNA, 25 bp, synthetic construct
  source 1..25: mol_type = other DNA
aacatgctacgcagagagggagtgg

SEQ ID NO: 559 - DNA, 28 bp, synthetic construct
  source 1..28: mol_type = other DNA
catgagacaaggaacccctagtgatgga

SEQ ID NO: 560 - DNA, 21 bp, synthetic construct
  source 1..21: mol_type = other DNA
gggaagagctcacgtccctga

SEQ ID NO: 561 - DNA, 21 bp, synthetic construct
  source 1..21: mol_type = other DNA
gagcgccgaatggatccaact

SEQ ID NO: 562 - DNA, 21 bp, synthetic construct
  source 1..21: mol_type = other DNA
ttgtggctcaccaacccaacc

SEQ ID NO: 563 - DNA, 22 bp, synthetic construct
  source 1..22: mol_type = other DNA
gttccgcgtaagagcaccgacc

SEQ ID NO: 564 - DNA, 22 bp, synthetic construct
  source 1..22: mol_type = other DNA
ccaggctgtttgaaatatcacg

SEQ ID NO: 565 - DNA, 20 bp, synthetic construct
  source 1..20: mol_type = other DNA
tccaccctgtagcctcaaag

SEQ ID NO: 566 - DNA, 20 bp, synthetic construct
  source 1..20: mol_type = other DNA
atggtcattcgttgtgctga

SEQ ID NO: 567 - DNA, 22 bp, synthetic construct
  source 1..22: mol_type = other DNA
agtcttgtaaggcaaagggtga

SEQ ID NO: 568 - DNA, 19 bp, synthetic construct
  source 1..19: mol_type = other DNA
ctatgtagcccgctctcgg

SEQ ID NO: 569 - DNA, 20 bp, synthetic construct
  source 1..20: mol_type = other DNA
cccctcacctttggcaactt

SEQ ID NO: 570 - DNA, 75 bp, synthetic construct
  source 1..75: mol_type = other DNA
cctgtattacctgaacagaactcagaatcagtccnnsnnsnnsnnsnnsnnsnnsttgctgtttagccgggggtc

SEQ ID NO: 571 - DNA, 25 bp, synthetic construct
  source 1..25: mol_type = other DNA
ccatgagaggagacgggtgaaagtg

SEQ ID NO: 572 - DNA, 23 bp, synthetic construct
  source 1..23: mol_type = other DNA
gcacggttcaagtcttctcggac

SEQ ID NO: 573 - DNA, 34 bp, synthetic construct
  source 1..34: mol_type = other DNA
ggactgattctgagttctgttcaggtaatacagg

SEQ ID NO: 574 - DNA, 53 bp, synthetic construct
  source 1..53: mol_type = other DNA
ccctacacgacgctcttccgatctnnnnnctggaccggctgatgaatcctctc

SEQ ID NO: 575 - DNA, 66 bp, synthetic construct
  source 1..66: mol_type = other DNA
gactggagttcagacgtgtgctcttccgatctnnnnntatacgtctctgtcttgccacaccattcc

SEQ ID NO: 576 - DNA, 80 bp, synthetic construct
  source 1..80: mol_type = other DNA
aatgatacggcgaccaccgagatctacactctttccctacacgacgctcttccgatctttgtggaaaggacgaaacaccg

SEQ ID NO: 577 - DNA, 88 bp, synthetic construct
  source 1..88: mol_type = other DNA
caagcagaagacggcatacgagatnnnnnnnngtgactggagttcagacgtgtgctcttccgatctctaaagcgcatgctccagactg

SEQ ID NO: 578 - DNA, 21 bp, synthetic construct
  source 1..21: mol_type = other DNA
gtttatgcctgtgtcagtggt

SEQ ID NO: 579 - DNA, 21 bp, synthetic construct
  source 1..21: mol_type = other DNA
ttttccaattggccccgctat

SEQ ID NO: 580 - DNA, 19 bp, synthetic construct
  source 1..19: mol_type = other DNA
agtgcttcagccgctaccc

SEQ ID NO: 581 - DNA, 20 bp, synthetic construct
  source 1..20: mol_type = other DNA
gttgtactccagcttgtgcc

SEQ ID NO: 582 - RNA, 20 nt, synthetic construct
  source 1..20: mol_type = other RNA
tatggattccaagagcaatg

SEQ ID NO: 583 - RNA, 20 nt, synthetic construct
  source 1..20: mol_type = other RNA
ctggtgggtgaatggcaagg

SEQ ID NO: 584 - RNA, 20 nt, synthetic construct
  source 1..20: mol_type = other RNA
atggccgagttggatccatt

SEQ ID NO: 585 - RNA, 20 nt, synthetic construct
  source 1..20: mol_type = other RNA
gacaagcaccagaaagacca

SEQ ID NO: 586 - RNA, 20 nt, synthetic construct
  source 1..20: mol_type = other RNA
actctggatagcatacaggc

SEQ ID NO: 587 - RNA, 20 nt, synthetic construct
  source 1..20: mol_type = other RNA
ttgcagcgagtgttggcctg

SEQ ID NO: 588 - RNA, 20 nt, synthetic construct
  source 1..20: mol_type = other RNA
ggaaatattgcagcgagtgt

SEQ ID NO: 589 - RNA, 20 nt, synthetic construct
  source 1..20: mol_type = other RNA
ttgcattgcggacagctgca

SEQ ID NO: 590 - RNA, 19 nt, synthetic construct
  source 1..19: mol_type = other RNA
atagtctccttcttgcatt

SEQ ID NO: 591 - RNA, 20 nt, synthetic construct
  source 1..20: mol_type = other RNA
agaatccgaagctgttcagt

SEQ ID NO: 592 - RNA, 20 nt, synthetic construct
  source 1..20: mol_type = other RNA
gacatccaactgaacagctt

SEQ ID NO: 593 - RNA, 20 nt, synthetic construct
  source 1..20: mol_type = other RNA
ggccacggagcgagacatct

SEQ ID NO: 594 - DNA, 351 bp, synthetic construct
  source 1..351: mol_type = other DNA
aaatggctgttcctgatcactccagctcccgatgctcggcgactcccagcttcccctagggtggcacttccgctggctcttccttctgctcccagtcacgagttgtccccagggctcctcccctttgccagctgattgggcagctcccgtcttctgattggctgttctttttgtggctcaccaacccaaccggatatagaagcagggtttccgcttccaccttctccctcttctcgctcgtcctccggtagcagccgccagcacagtgtcggctgctgggcggggtgtcggcggatcggtcggttggtttcggcttcggcgtctctgcggttgagttgggctgtcatggcc

SEQ ID NO: 595 - DNA, 303 bp, synthetic construct
  source 1..303: mol_type = other DNA
gagttggatccattcggcgctcccgccggtgcgcccggtggccccgctctaggaaacggggtggcgggcgccggcgaagaagacccggccgcggccttcttggcgcagcaagagagtgagattgcgggcatcgagaacgatgaggccttcgccatcctggacggcggcgcccccgggccgcagccacacggcgagccgccgggaggtccgggtgagagggcagcggcgttcgggcgggaggacttgtgggcacactcgggtcggtgctcttacgcggaacttgggcttctggctgaagcttta

SEQ ID NO: 596 - DNA, 497 bp, synthetic construct
  source 1..497: mol_type = other DNA
aagatcctgttaagtagaaaaatgacaagatggtttagtggttccaccttcctttctggttttcccagatgccagaaaggaccccgtggagagggactactgaaagctagagatcttggctggcactcatgacttgtgcctgtccctaagcctgtgtccatcatgggcagctagatcctaggctgtcatttccaatgtgaagtctgtgaacaagactggctagtccagagagttccgctcttgcctgtcactggcatctgagttctgacctaagttaggacccccaaaaaatcttctaacatcagtcctcttggcccacagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagc

SEQ ID NO: 597 - DNA, 500 bp, synthetic construct
  source 1..500: mol_type = other DNA
aatggggccattgcctggagcaaccagacaagcttcacctgccaagatatcttcaaagagaccaacgccacctaccccagttcaggtgagtggtgccccagaagtctcctcctggctcaaatctatggtttccaaaagctgctttgggtcatctcaccctcatcattaggcagcaaaatccacatcctgtgtcactaaggagtcctaacgggtcaccaaagcagacaagagaacatgacagaggacattggcccagcattggtctcacaattctgaggctcaccttcctgtcttctcttgagccaagtgccttctctgttctcccaggcacagtttccattcccttcctgcccatcccacccccacccacccccagccgcctgccctgacttctatgtctgccaagtagtgtgagtacagaagtcagatgaggaggagggagtgccactctaatccgggaggaccatccgtcagcagagggacatgtaaatgacaccagg

SEQ ID NO: 598 - DNA, 687 bp, synthetic construct
  source 1..687: mol_type = other DNA
agcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcact

SEQ ID NO: 599 - DNA, 1476 bp, synthetic construct
  source 1..1476: mol_type = other DNA
atggcctcaccgttgacccgctttctgtcgctgaacctgctgctgctgggtgagtcgattatcctggggagtggagaagctgaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgc

SEQ ID NO: 600 - DNA, 22 bp, synthetic construct
  source 1..22: mol_type = other DNA
ttgtggaaaggacgaaacaccg

SEQ ID NO: 601 - DNA, 22 bp, synthetic construct
  source 1..22: mol_type = other DNA
ctaaagcgcatgctccagactg

SEQ ID NO: 602 - RNA, 19 nt, synthetic construct
  source 1..19: mol_type = other RNA
agtacccctaggtatgggg

(19) United States
(12) Patent Application Publication
(10) Pub. No.: US 2024/0366788 A1
(43) Pub. Date: Nov. 7, 2024

(54) ADENO-ASSOCIATED VIRUS COMPOSITIONS AND METHODS OF USE THEREOF

(71) Applicants: Duke University, Durham, NC (US); The Regents of the University of California, Oakland, CA (US)

(72) Inventors: Aravind Asokan, Durham, NC (US); Jonathan Ian Ark, Durham, NC (US); Lawrence Patrick Havlik, Durham, NC (US); Justin Eyquem, Oakland, CA (US); William Nyberg, Oakland, CA (US)

(21) Appl. No.: 18/290,799

(22) PCT Filed: Jul. 22, 2022
(86) PCT No.: PCT/US2022/074039

§ 371 Date: Jan. 21, 2024

Related U.S. Application Data
(60) Provisional application No. 63/225,087, filed on Jul. 23, 2021.

(51) Int. Cl. A61K 48/00; A61K 35/17; A61P 37/04; C07K 14/005; C12N 5/0783; C12N 9/22; C12N 15/11; C12N 15/86

(52) U.S. Cl. CPC A61K 48/005; A61K 35/17; A61P 37/04; C07K 14/005; C12N 5/0636; C12N 9/22; C12N 15/111; C12N 15/86; C12N 2310/20; C12N 2750/14122; C12N 2750/14143

(57) ABSTRACT

Disclosed herein are adeno-associated virus (AAV) vectors comprising capsid protein variants. Also disclosed herein are pharmaceutical compositions comprising these AAV vectors and capsid protein variants as well as methods of making such vectors and capsid protein variants. Disclosed herein are methods of using the disclosed AAV vectors and disclosed capsid protein variants.

### I. CROSS-REFERENCE TO RELATED APPLICATIONS

This application claims priority to U.S. Provisional Application No. 63/225,087 filed 23 Jul. 2021, which is incorporated herein in its entirety.

### II. STATEMENT REGARDING FEDERALLY SPONSORED RESEARCH OR DEVELOPMENT

This invention was made with government support under Federal Grant No. R01HL089221 awarded by the National Institutes of Health. The Federal Government has certain rights to this invention.

### III. FIELD OF THE DISCLOSURE

The present disclosure relates to modified capsid proteins from adeno-associated virus (AAV) and virus capsids and virus vectors comprising the same. In particular, the disclosure relates to modified AAV capsid proteins and capsids comprising the same that can be incorporated into virus vectors to enable expression in any cell or tissue type in a mammalian subject.

### IV. INCORPORATION BY REFERENCE OF SEQUENCE LISTING PROVIDED ELECTRONICALLY

The Sequence Listing submitted on 22 Jul. 2022 as an .xml file is incorporated by reference herein in its entirety. The electronic file is 728 kilobytes in size and titled “POL_21-2016-WO_Sequence_Listing”.

### V. BACKGROUND

Adeno-associated virus (AAV) vectors have become a leading platform for gene delivery for the treatment of a variety of diseases. Although there has been clinical success using AAV gene therapies, limitations and challenges associated with use of this gene delivery platform remain. Gene therapy with vectors (viral or non-viral) is sometimes complicated because of an immune response against the vector carrying the gene. Viral vectors are the most likely to induce an immune response, especially those like adenovirus and AAV that express immunogenic epitopes within the organism. Immunity against vectors and their contents can substantially reduce the efficiency of gene therapy. A strong immune response against the constituents of the vector or the transgene leads to rejection of the cells infected by the vector and, therefore, to a reduction in the duration of expression of the therapeutic protein. Due to the many different and complex roles they play in host immune responses, immune cells have been identified as important targets for treatment of immunodeficiencies and cancer and for the development of cell-based immune-mediated therapeutics, such as chimeric antigen receptor (CAR) T cells. As such, immune cells, such as T cells and NK cells, can be important targets for AAV-mediated gene therapies. Such efforts have been hampered, however, in that AAV has generally been considered inefficient at transducing T cells. Further, because immune cells are ubiquitously found throughout the body in blood and other lymphoid and non-lymphoid tissues, AAV-mediated gene therapies targeting immune cells would require systemic delivery at high doses, further triggering the undesired immune responses to the AAV vectors. As such, there is a need in the field for improved AAV vectors for therapeutic gene delivery, particularly for use in AAV-mediated immune cell gene therapies. Additionally, there is a need to develop AAV-based gene therapies that can selectively and specifically target tissues of interest, including tissues that have been difficult to target using known AAV serotypes, including multiple immune cell types such as T cells and NK cells.

The compositions and methods disclosed herein demonstrate that Ark313, a synthetic AAV that exhibits high transduction efficiency in murine T cells, can be used for nucleofection-free DNA delivery, CRISPR/Cas9-mediated gene knockouts, and targeted integration of large transgenes with efficiencies up to 50%. Thus, Ark313 enables pre-clinical modeling of Trac-targeted CAR- and transgenic T cell receptor (TCR-T) cells in immunocompetent models.

### VI. BRIEF SUMMARY OF THE DISCLOSURE

The present disclosure provides, at least in part, methods and compositions comprising an adeno-associated virus (AAV) capsid protein, comprising one or more amino acid substitutions, wherein the substitutions introduce into an AAV vector comprising these modified capsid proteins one or more improved functionalities such as, but not limited to, the ability to evade host antibodies, selective tropism, and/or higher transduction efficiency.

Disclosed herein isolated nucleic acid molecule, comprising: a sequence encoding an adeno-associated virus (AAV) capsid protein variant, wherein the encoded AAV capsid protein variant comprises the sequence of SEQ ID NO:01, wherein the amino acids 454-460 of the capsid protein variant comprise the sequence set forth in any one of SEQ ID NO:05-SEQ ID NO:545.

Disclosed herein is an isolated nucleic acid molecule, comprising: a nucleotide sequence encoding an adeno-associated virus (AAV) capsid protein variant, wherein the encoded AAV capsid protein variant comprises the sequence of SEQ ID NO:02.

Disclosed herein is an isolated nucleic acid molecule, comprising: the nucleotide sequence set forth in SEQ ID NO:04.

Disclosed herein is an AAV capsid protein variant comprising the sequence of SEQ ID NO:01, wherein amino acids 454-460 of the capsid protein variant comprise the sequence set forth in any one of SEQ ID NO:05-SEQ ID NO:545.

Disclosed herein is an AAV capsid protein variant comprising a sequence having at least 90% identity to the sequence set forth in SEQ ID NO:01, wherein amino acids 454-460 of the capsid protein variant comprise the sequence set forth in any one of SEQ ID NO:05-SEQ ID NO:545.

Disclosed herein is an AAV capsid protein variant comprising the sequence set forth in SEQ ID NO:02 or a sequence having at least 90% identity to the sequence set forth in SEQ ID NO:02.

Disclosed herein is a recombinant AAV (rAAV) vector comprising a vector genome, wherein the vector genome is encapsidated by an AAV capsid comprising a disclosed AAV capsid protein variant.

Disclosed herein is pharmaceutical composition comprising a disclosed rAAV vector and at least one pharmaceutically acceptable carrier.

Disclosed herein is a method of delivering a transgene to a target cell in a subject, the method comprising administering to the subject a therapeutically effective amount of a disclosed rAAV vector or a disclosed pharmaceutical composition.

Disclosed herein is a method of alleviating and/or treating a disease or a condition in a subject in need thereof, the method comprising administering to the subject a therapeutically effective amount of a disclosed rAAV vector or a disclosed pharmaceutical composition.

Disclosed herein is a method of alleviating and/or treating a disease or a condition in a subject in need thereof, the method comprising administering to the subject one or more cells that have been contacted ex vivo with a disclosed rAAV vector or a disclosed pharmaceutical composition.

Disclosed herein is an AAV capsid library, comprising: a first AAV capsid protein comprising the sequence set forth in SEQ ID NO:01, and one or more capsid protein variants comprising the sequence set forth in SEQ ID NO:01, wherein amino acids 454-460 of the capsid protein variant comprise the sequence set forth in any one of SEQ ID NO:05-SEQ ID NO:545.

An aspect of the present disclosure provides for recombinant AAV vectors that may comprise a capsid protein variant, wherein the capsid protein may comprise a peptide having a sequence of any one of SEQ ID NO:05-SEQ ID NO:545. In an aspect, recombinant AAV vectors herein may comprise an AAV capsid protein variant, wherein the AAV capsid variant can have at least 90% identity to the sequence of SEQ ID NO:01, wherein the amino acids corresponding to amino acids 454-460 of SEQ ID NO:01 can be substituted with a peptide having a sequence of any one of SEQ ID NO:05-SEQ ID NO:545. In an aspect, recombinant AAV vectors herein may comprise an AAV capsid protein variant, wherein the AAV capsid variant can have the sequence of SEQ ID NO:02 or a sequence with at least 90% or at least 95% identity thereto.

In an aspect, recombinant AAV vectors herein may comprise a vector genome. In an aspect, vector genomes disclosed herein can be encapsidated by an AAV capsid comprising any AAV capsid protein variant disclosed herein. In an aspect, recombinant AAV vectors herein may comprise a first inverted terminal repeat (ITR) and a second ITR. In an aspect, vector genomes disclosed herein may comprise a transgene located between the first ITR and the second ITR.

In an aspect, recombinant AAV vectors herein may comprise a transgene that can encode a therapeutic RNA. In an aspect, transgenes disclosed herein may encode a therapeutic protein. In an aspect, transgenes disclosed herein may encode a gene-editing molecule. In an aspect, gene-editing molecules disclosed herein may comprise a nuclease. In an aspect, nucleases disclosed herein may comprise a Cas9 nuclease. In an aspect, gene-editing molecules disclosed herein may comprise a single guide RNA (sgRNA).

Another aspect of the present disclosure provides for AAV capsid protein variants that may comprise a peptide having the sequence of any one of SEQ ID NO:05-SEQ ID NO:545.

In an aspect, AAV capsid protein variants herein may comprise an amino acid sequence having at least 90% identity to the sequence of SEQ ID NO:01, wherein the amino acids corresponding to amino acids 454-460 of SEQ ID NO:01 can be substituted with a peptide having a sequence of any one of SEQ ID NO:05-SEQ ID NO:545. In an aspect, AAV capsid protein variants herein may comprise an amino acid sequence of SEQ ID NO:02 or a sequence with at least 90% or at least 95% identity thereto.

In an aspect, AAV capsid protein variants herein may comprise about 60 copies of the AAV capsid protein variant, or fragments thereof. In an aspect, recombinant AAV vectors herein can be arranged with T=1 icosahedral symmetry.

In an aspect, recombinant AAV vectors herein may comprise any AAV capsid variant disclosed herein and/or any AAV capsid disclosed herein.

Another aspect of the present disclosure provides for pharmaceutical compositions that may comprise any recombinant AAV vectors disclosed herein and at least one pharmaceutically acceptable carrier.

Another aspect of the present disclosure provides for methods of using compositions disclosed herein. In an aspect, the disclosure provides for methods of introducing a recombinant AAV vector into a target cell. In an aspect, methods herein may comprise contacting a target cell with any recombinant AAV vector disclosed herein and/or any pharmaceutical composition disclosed herein. In an aspect, methods herein may comprise delivering a transgene to a target cell in a subject. In an aspect, methods herein may comprise administering to a subject described herein any recombinant AAV vector disclosed herein and/or any pharmaceutical composition disclosed herein. In an aspect, methods herein may target an immune cell. In an aspect, methods herein may target a T cell, a NK cell, or a combination thereof. In an aspect, methods herein may comprise contacting a cell in vitro, ex vivo and/or in vivo.

In an aspect, methods herein may comprise treating a subject in need thereof by administering to the subject an effective amount of any recombinant AAV vector disclosed herein and/or any pharmaceutical composition disclosed herein. In an aspect, a subject in need of treatment may comprise a mammal. In an aspect, a subject in need of treatment can be a human. In an aspect, a subject in need of treatment can be a mouse.

An aspect of the disclosure provides for kits, wherein a kit can comprise any of the compositions or AAV vectors disclosed herein and at least one container.

### VII. BRIEF DESCRIPTION OF THE DRAWINGS

The following drawings form part of the present specification and are included to further demonstrate certain aspects of the present disclosure, which can be better understood by reference to the drawings in combination with the detailed description of specific embodiments presented herein.

FIG. 1 illustrates bubble plots showing analysis of library diversity, directed evolution and enrichment of AAVs comprising capsid proteins with novel peptide substitutions such as those, for example, described herein. Each bubble represents a unique amino acid variant represented within the sequencing, y-axis depicts the log of percent reads for each amino acid variant detected and the x-axis is dimensionless. Bubble size represents enrichment of unique amino acid variants from the parental library as calculated by percent reads in the evolved library over the percent reads in the unselected library for each detected variant.

FIG. 2A-FIG. 2E illustrate GFP reporter gene expression in C57/B6 mouse T cells.

FIG. 2A depicts a schematic representation of the AAV vectors used for transient GFP expression in mouse T cells, where 1×10¹ mouse T cells were incubated with WT AAV6 or Ark313 in FBS containing media for 48 hours prior to analysis by flow cytometry. FIG. 2B depicts an image showing GFP positive cells at each MOI comparing AAV6 WT to Ark313.

FIG. 2C depicts a graph showing percentage of in GFP positive T cells at each MOI comparing WT to 313 AAV6. FIG. 2D-FIG. 2E depict a graph showing percentage of GFP positive human CD4 and CD8 T cells after infection with AAV6 WT (FIG. 2D) and Ark313 (FIG. 2E).

FIG. 3A-FIG. 3F illustrate delivery of a donor template with AAV6 mutant (Ark313) improved gene targeting in mouse T cells C57/B6 mouse T cells. FIG. 3A shows the genomic sequence of Clta exon1 targeted by a gRNA underlined and marked in orange followed by a PAM sequence marked in red (SEQ ID NO:548). FIG. 3B shows mouse T cells electroporated with Cas9 and Clta gRNA (Clta RNP). FIG. 3C shows a schematic representation of a donor template to insert a GFP in the first exon of the Clta gene. FIG. 3D shows GFP expression in mouse T cells that were electroporated with the Clta RNP and incubated with the indicated AAV6 (WT or Ark313) overnight. FIG. 3E shows percentage of GFP positive mouse T cells.

FIG. 3F shows validation of the Clta gene targeting by PCR analysis with primers flanking the integration site.

FIG. 4A-FIG. 4D illustrate GFP reporter gene expression (FIG. 4A-FIG. 4B) and MFI (FIG. 4C-FIG. 4D) in T cells harvested from mice after in vivo injection of either WT AAV6 or Ark313.

FIG. 5A-FIG. 5D illustrate native tdTomato fluorescence in mouse immune cells after intravenous administration of AAV6 or Ark313.

FIG. 6A-FIG. 6B illustrate use of Ark313 to generate CAR T cells in ex vivo mouse cells. FIG. 6A shows a schematic representation of donor template to insert a CAR in the first exon of the TRAC gene. FIG. 6B shows percentage of CAR positive mouse T cells as assessed by flow cytometry.

FIG. 7A-FIG. 7I show that structure-guided evolution identified an AAV capsid variant with murine T cell tropism. FIG. 7A shows the directed evolution of a pooled library of AAV6 variants. The library was evolved for three cycles with CD3/CD28 bead-activated primary T cells from C57BL/6J mice. FIG. 7B shows the sequencing analysis of the parental and evolved libraries. Bubble plots depict the enrichment of capsid mutants with each bubble representing a unique amino acid sequence. Bubble size was proportional to enrichment in the evolved library. FIG. 7C shows the sequence logo of the 7-mer sequence in the top 1,000 (>500-fold enrichment) expressed capsids in the evolved library. FIG. 7D shows the packaging yield of AAV6 (n=20) and Ark313 (n=11) presented as viral genomes per liter (vg/L) of media used to produce virus. Viral genomes were quantified by qPCR. FIG. 7E shows the number of viral genomes bound to the murine T cell surface following a 1 hr incubation at 4° C. to arrest cellular uptake with the indicated AAV capsid, measured by qPCR. The bar graph depicts the mean±SEM from four independent experiments. FIG. 7F shows the percentage of internalized viral genomes after reactivation by a 1 hr incubation at 37° C. of membrane-bound AAV. The bar graph depicts the mean±SEM from four independent experiments. FIG. 7G shows that scAAV-CBh-GFP was packaged into AAV6 and into Ark313. Transduction efficiencies were determined by flow cytometry at 48 hr after transduction. FIG. 7H shows flow cytometry analysis of EGFP expression following transduction of human T cells with AAV6 or Ark313 at the indicated MOIs. The left side of FIG. 7H shows fluorescence histograms while the right side of FIG. 7H shows the MFI of transduced cells. FIG. 7I shows a flow cytometry analysis of EGFP expression following transduction of murine T cells with AAV6 or Ark313 at the indicated MOIs. The left side of FIG. 7I shows fluorescence histograms while the right side of FIG. 7I shows the MFI of transduced cells. In FIG. 7D-FIG. 7F, the statistical significance was assessed using unpaired t-tests (ns=not significant; *p<0.05; ***p<0.001).

FIG. 8A shows the heatmap for the average distribution of amino acids at each of the 7 positions of the parental and murine T cell-evolved AAV capsid libraries. FIG. 8B shows the top-ranked fifteen (15) 7-mer sequences in the parental vs. evolved AAV capsid libraries.

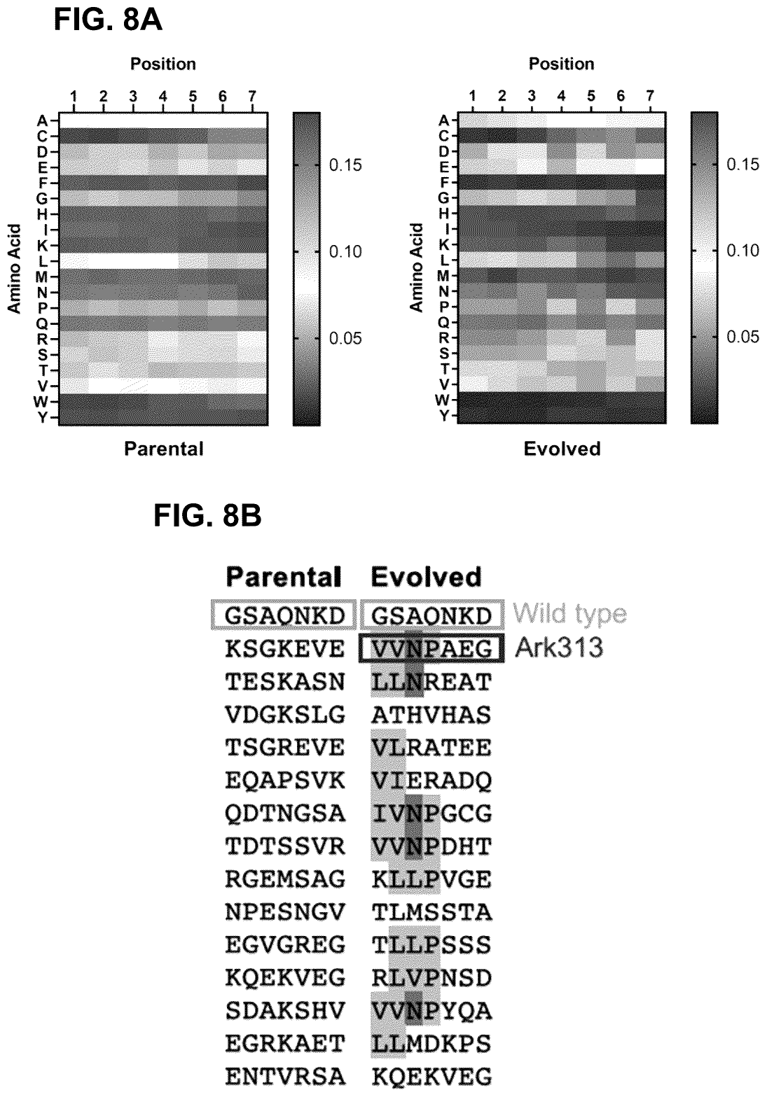

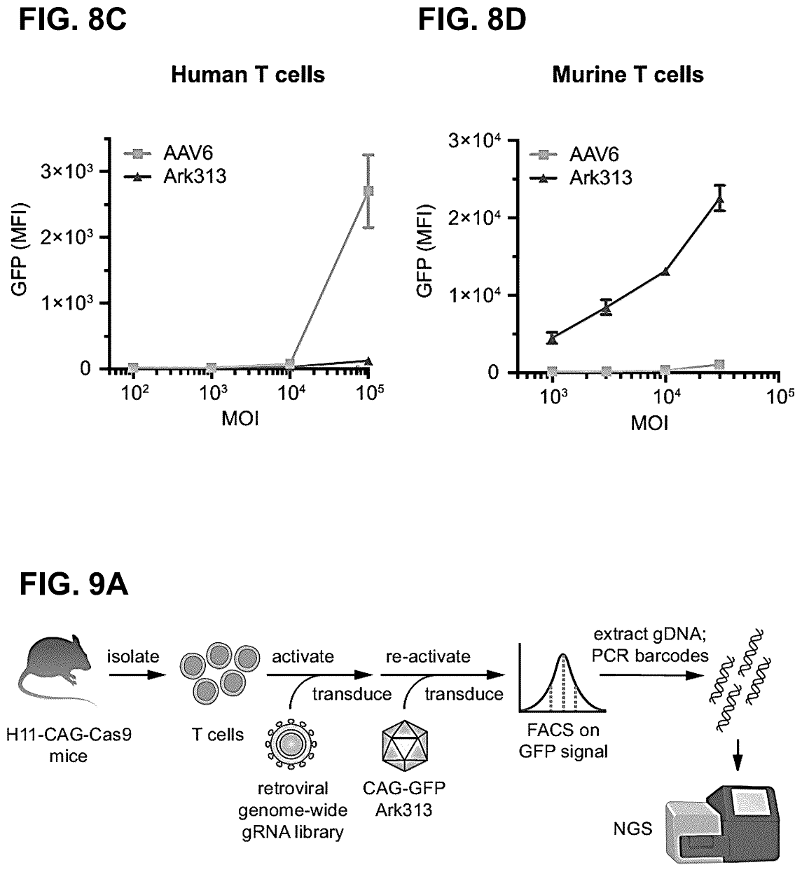

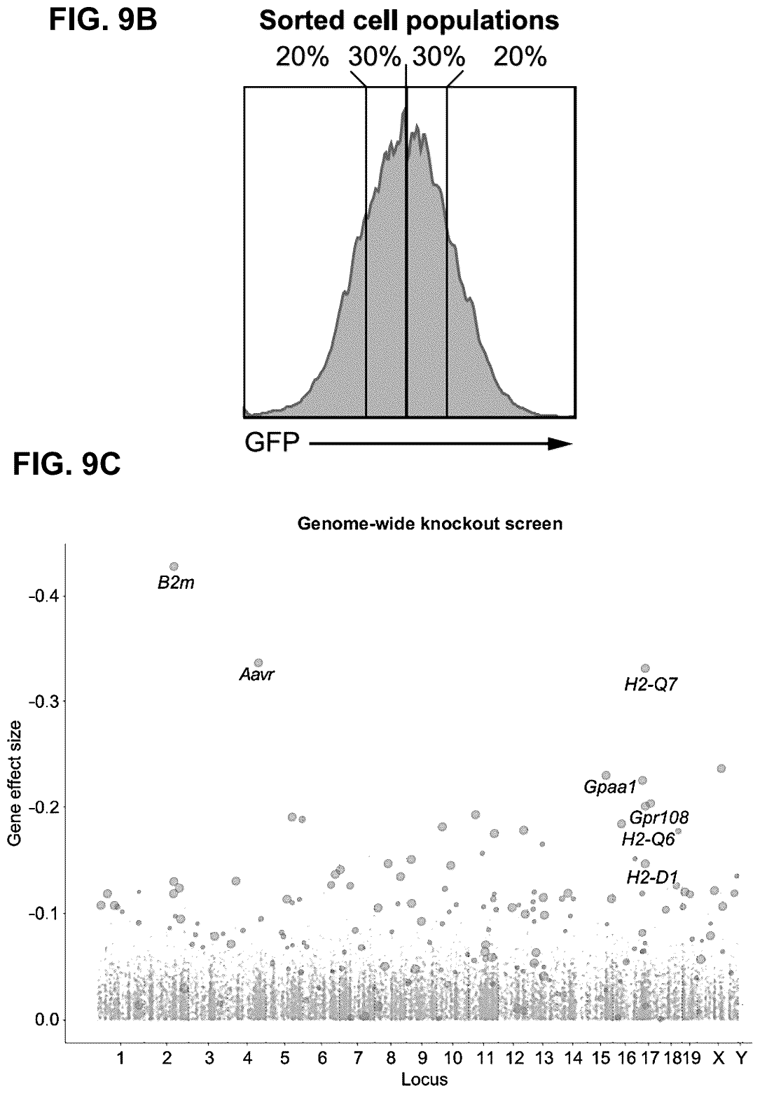

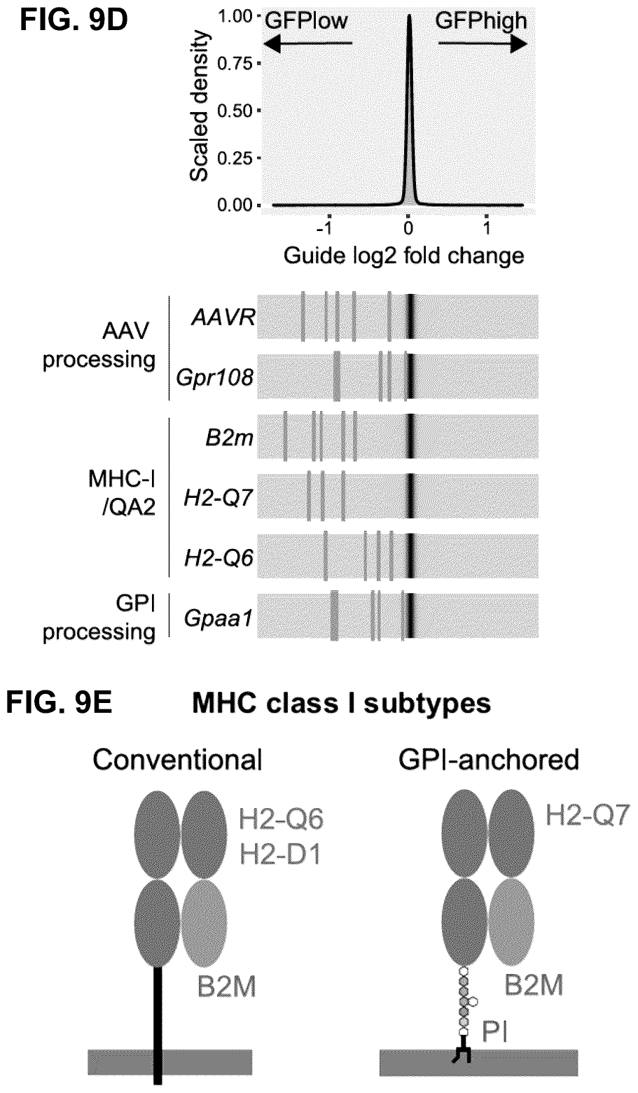

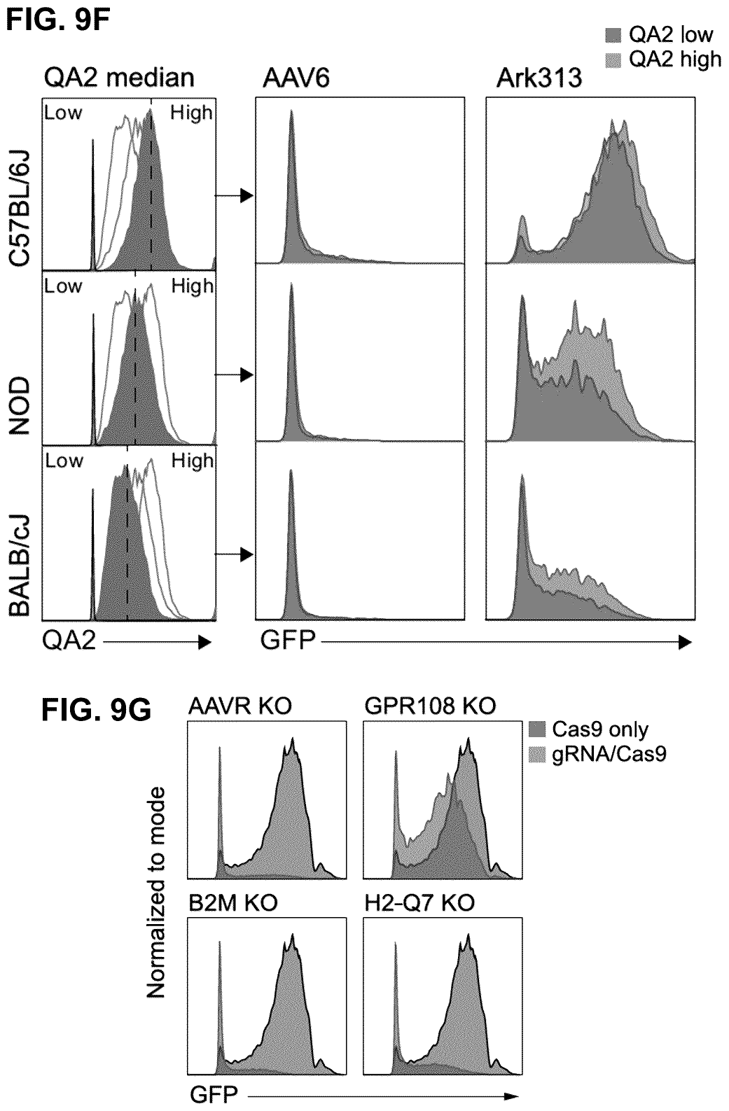

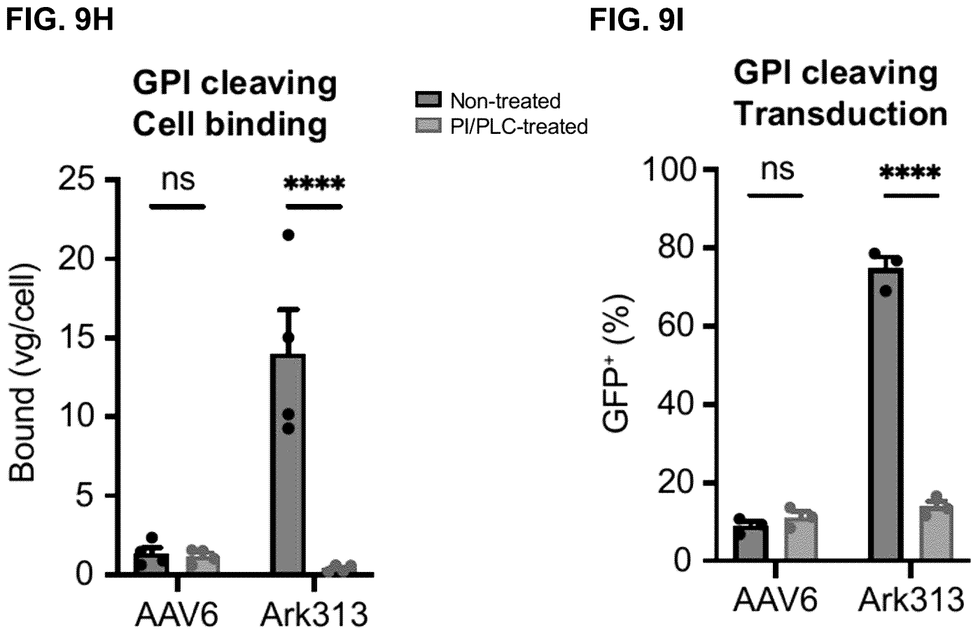

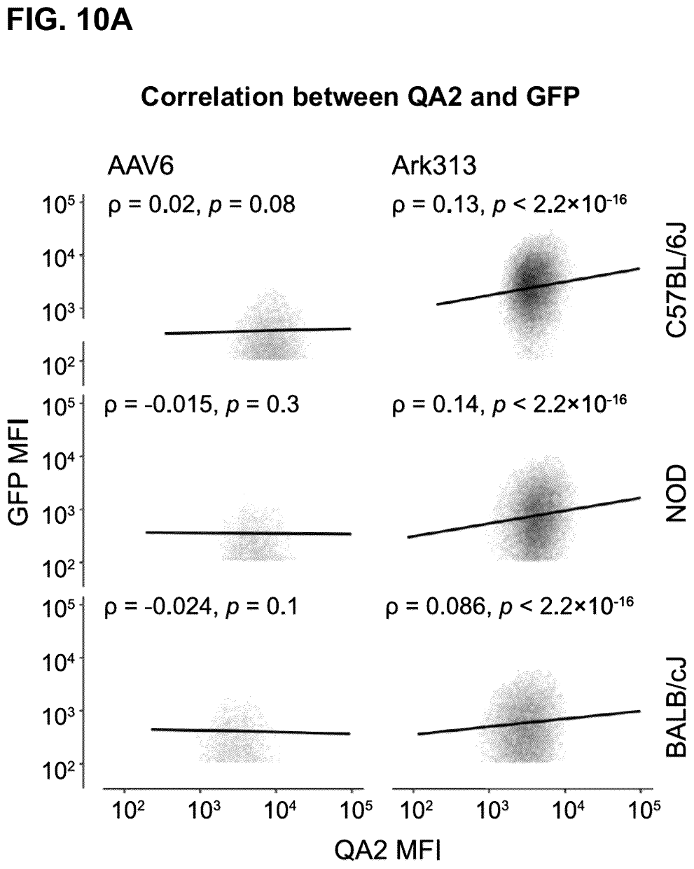

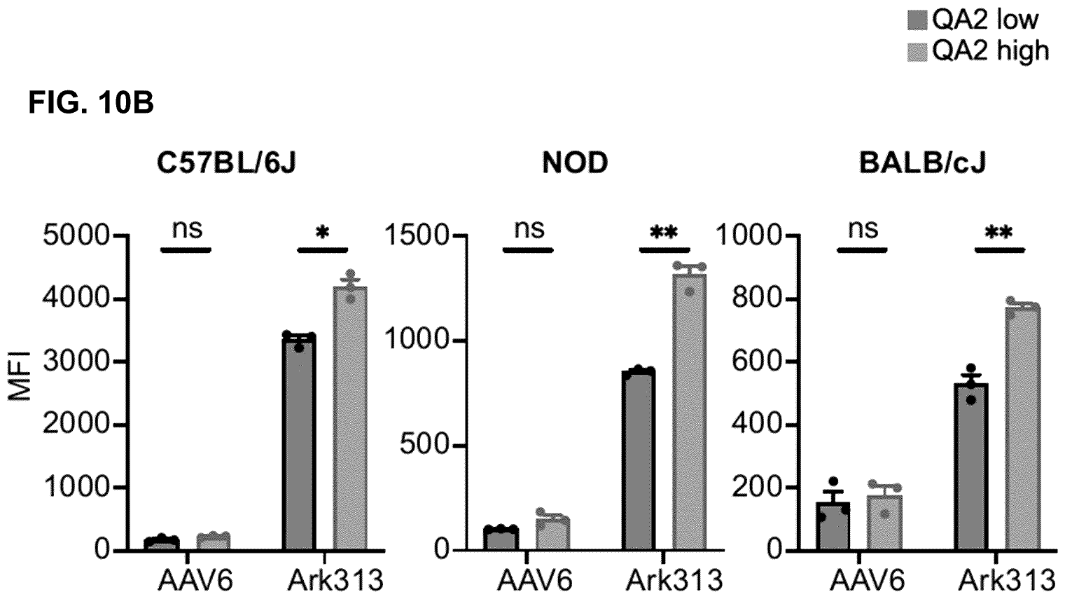

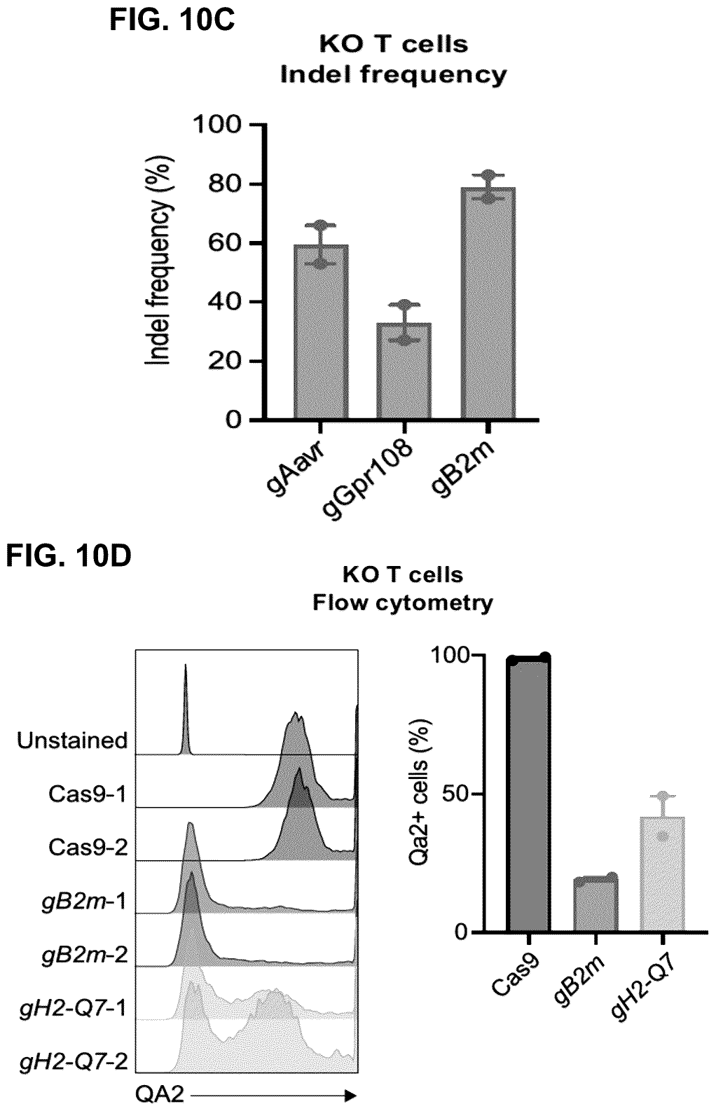

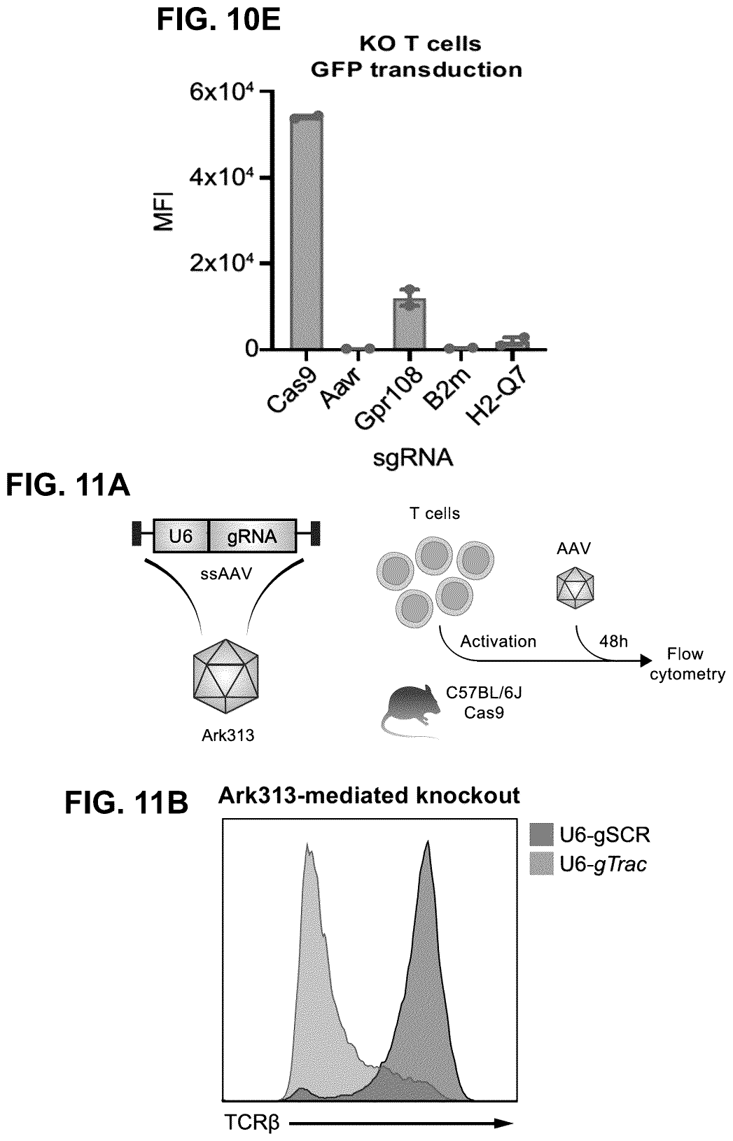

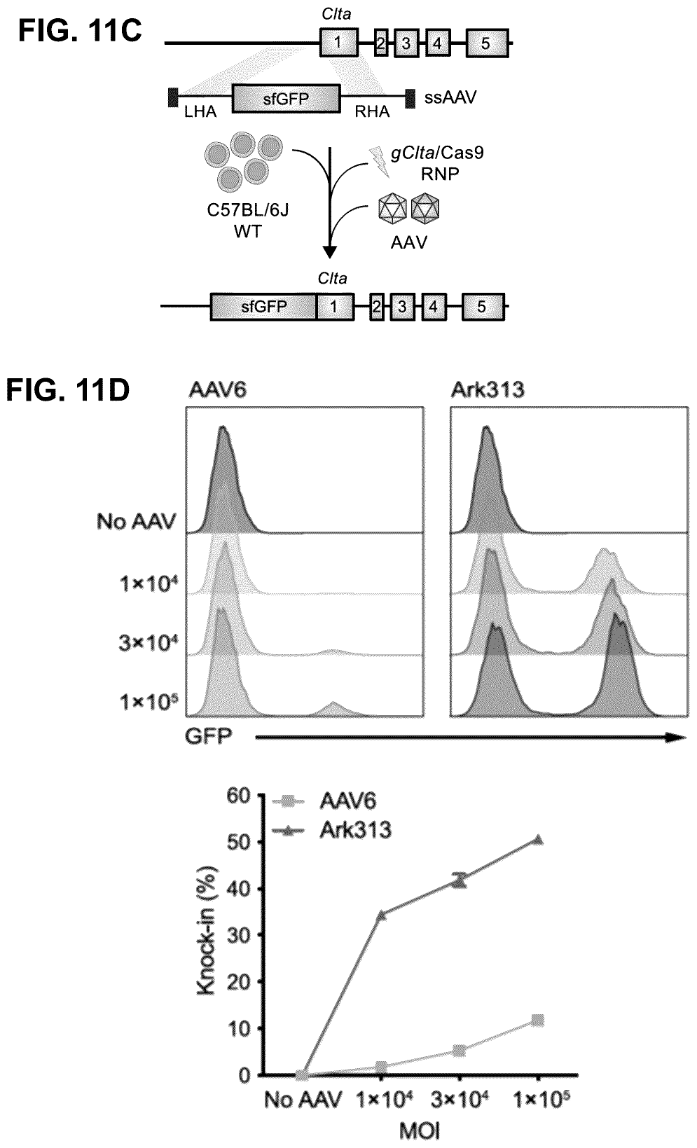

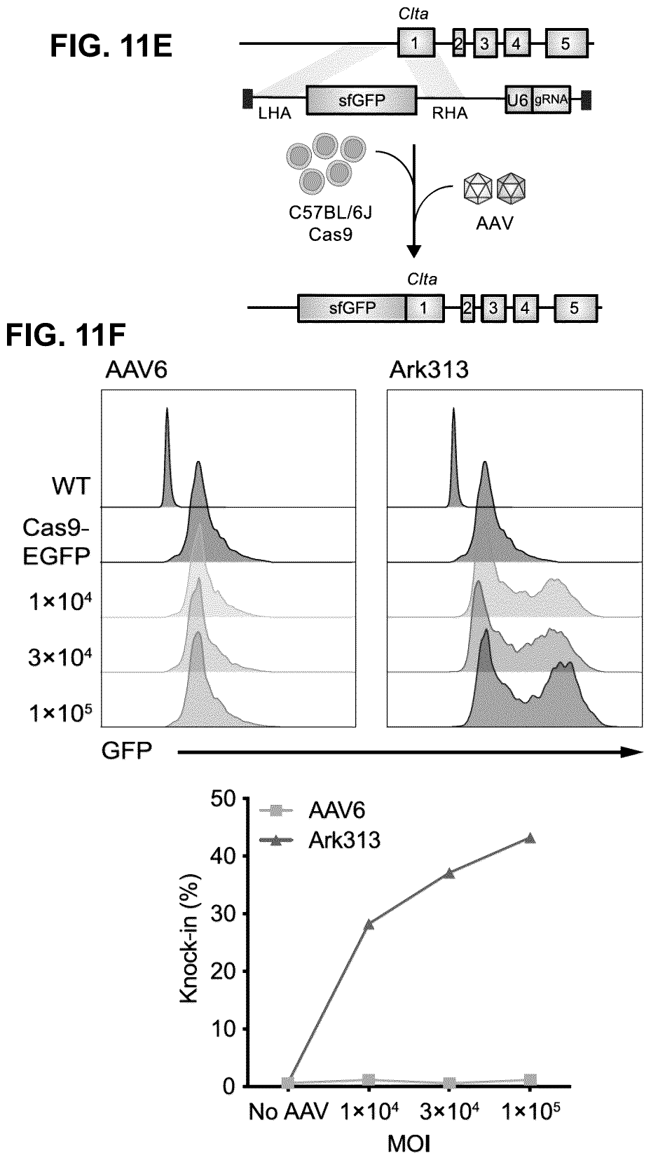

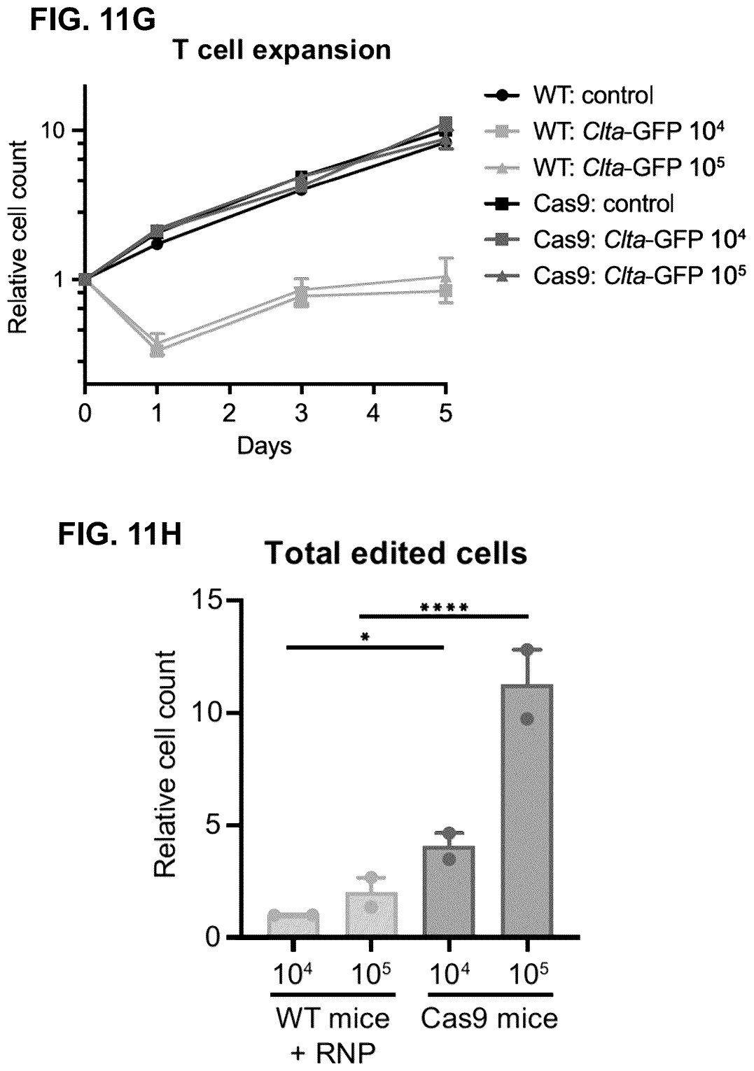

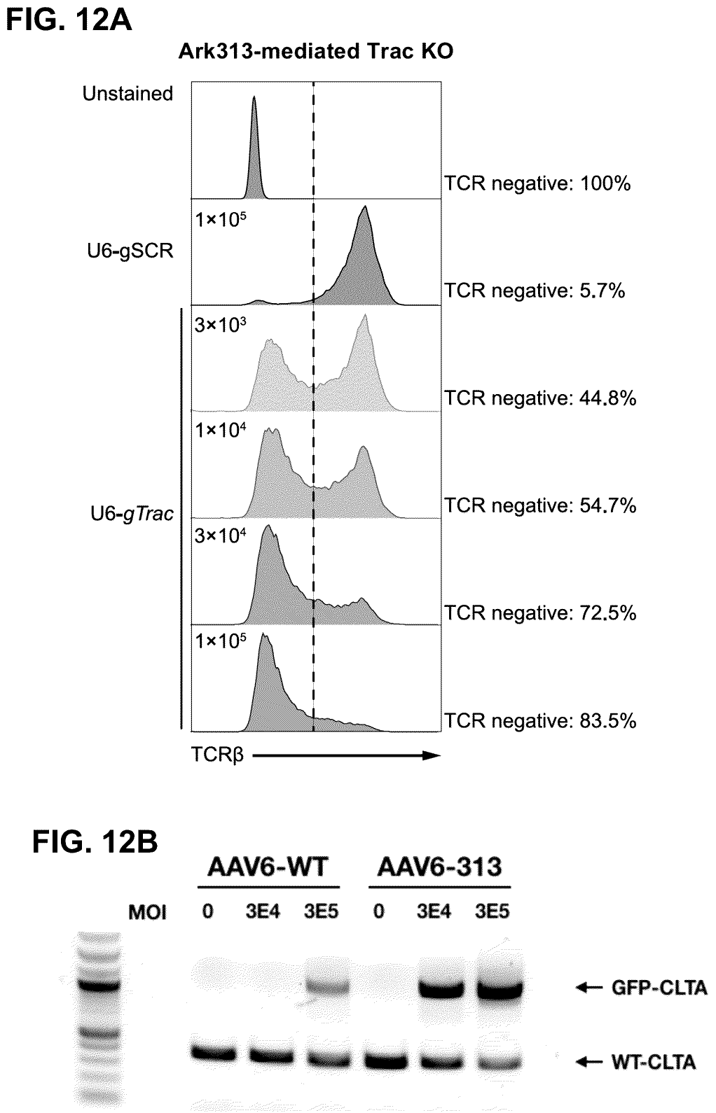

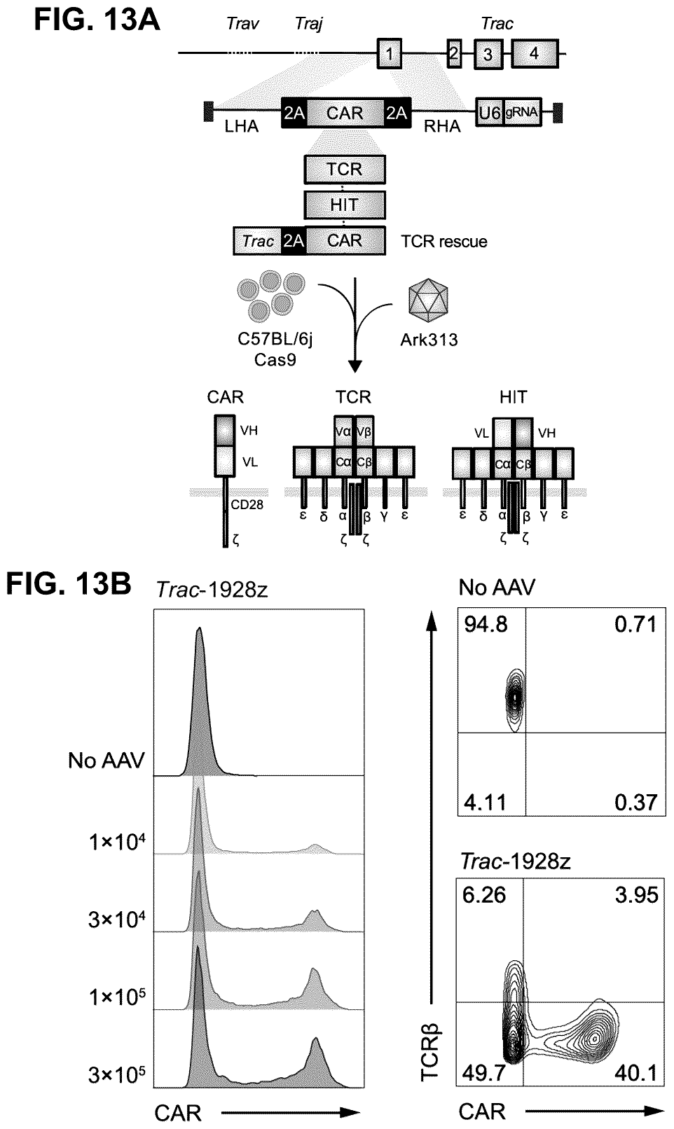

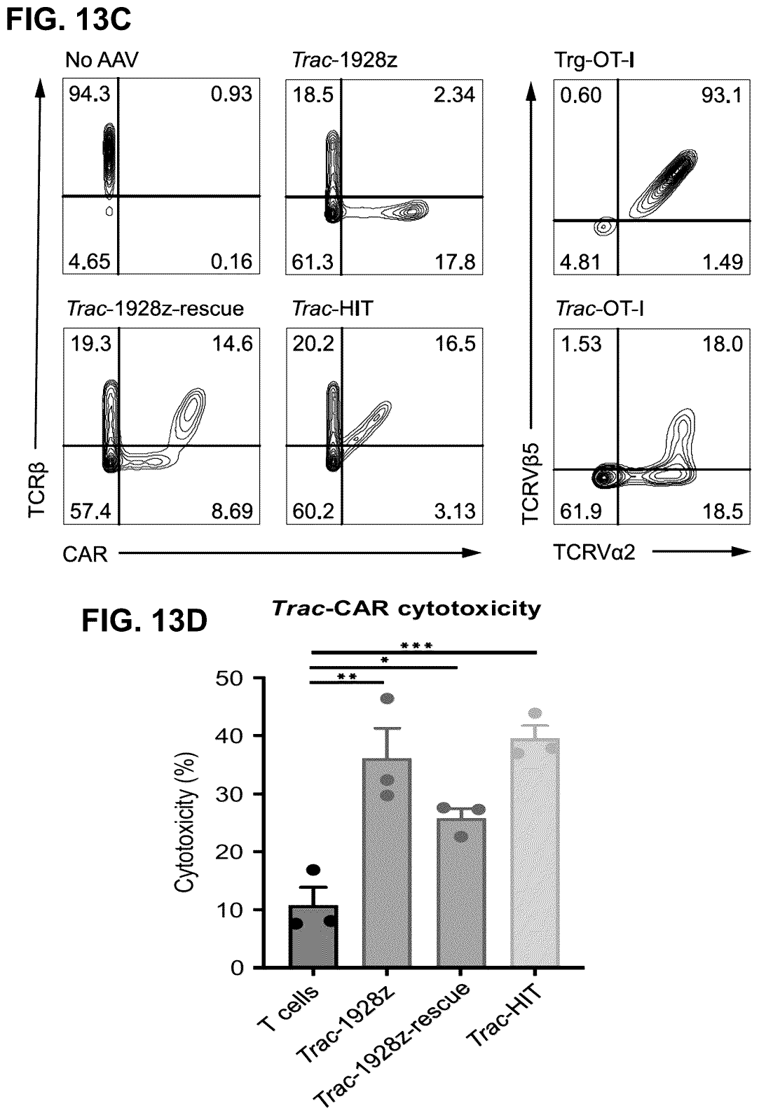

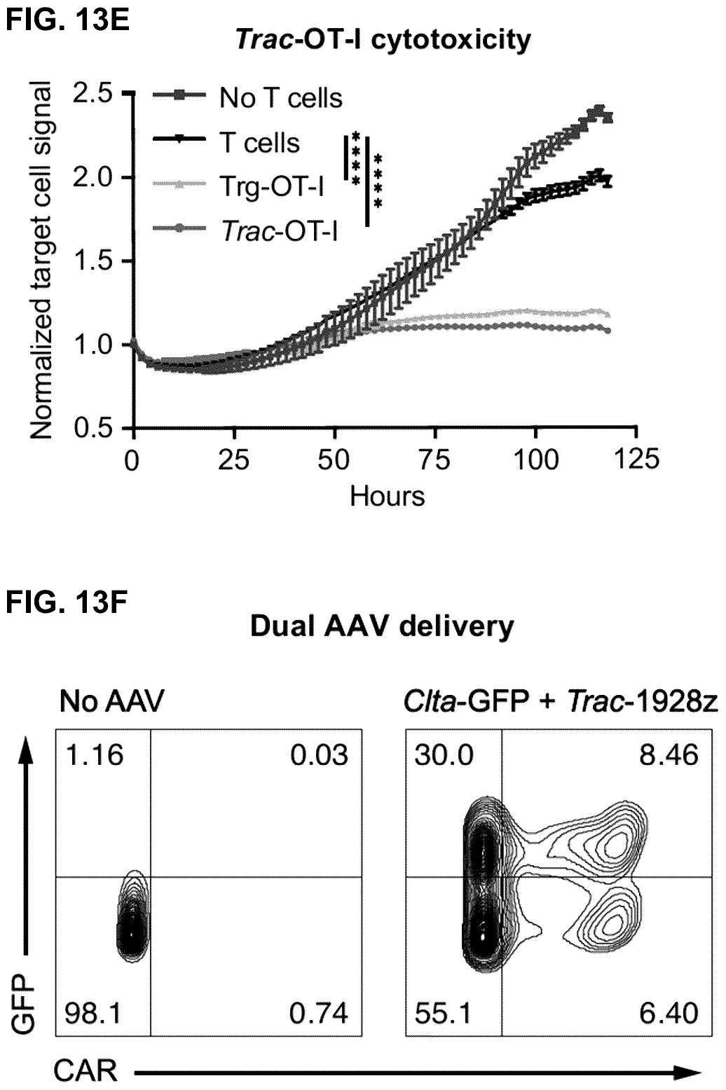

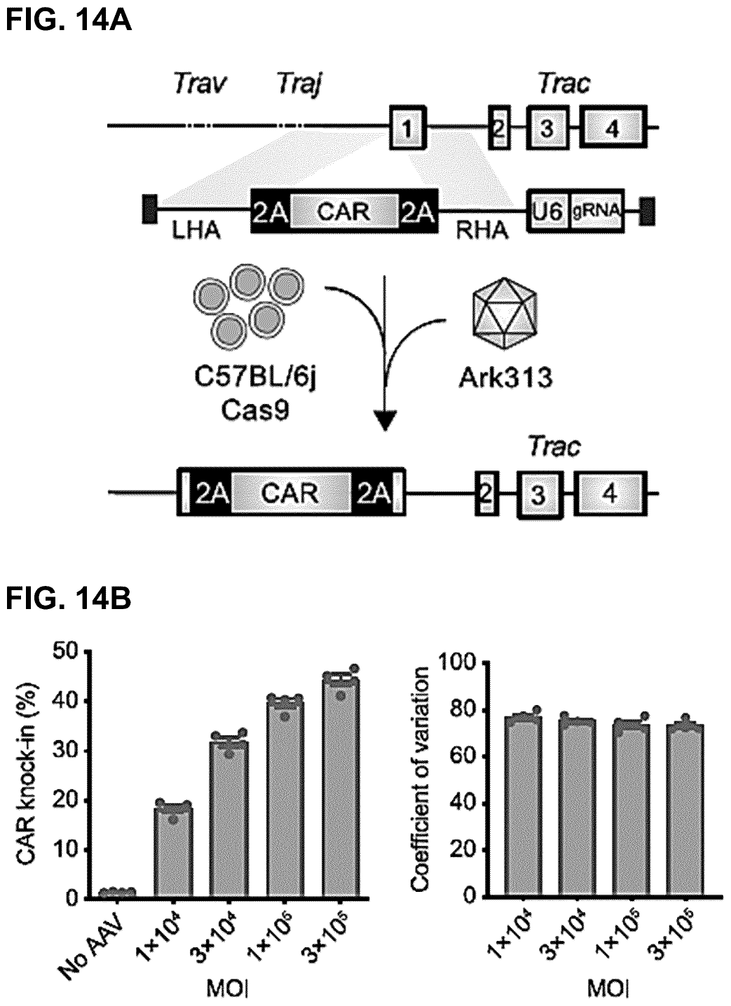

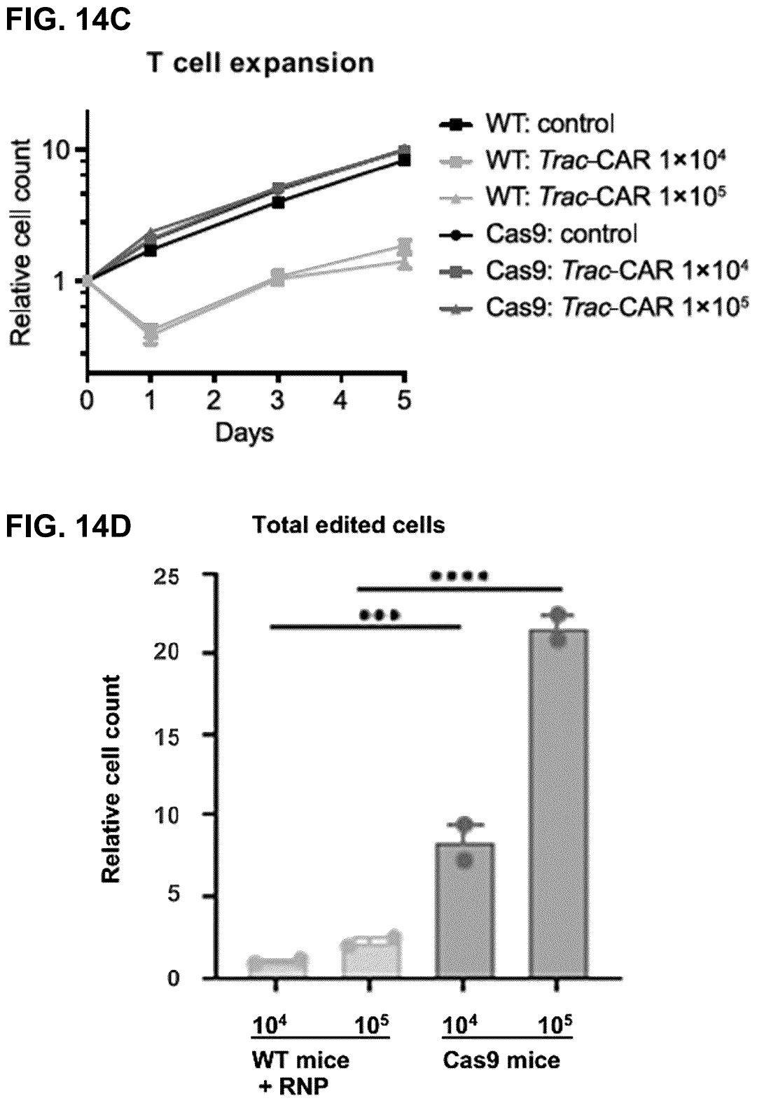

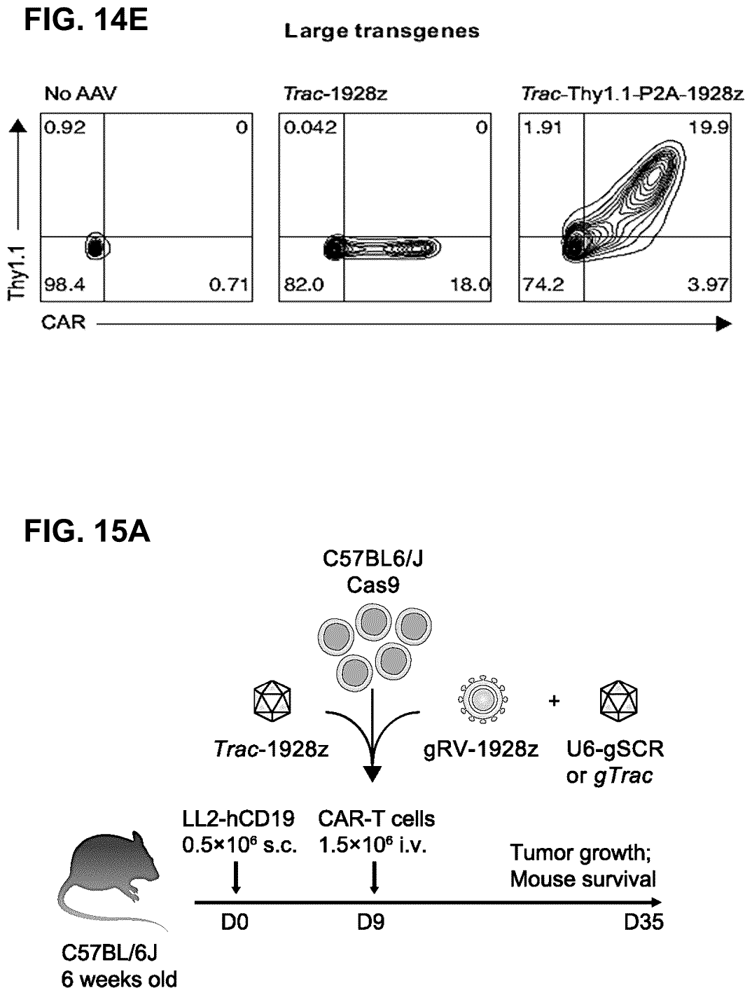

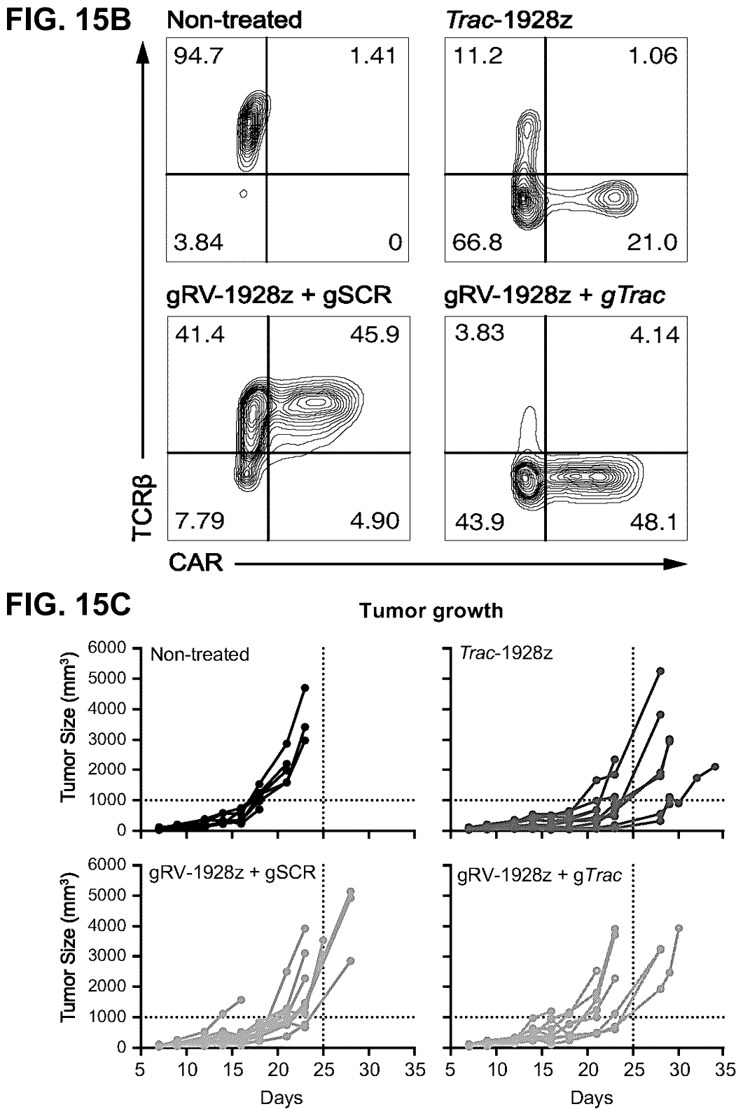

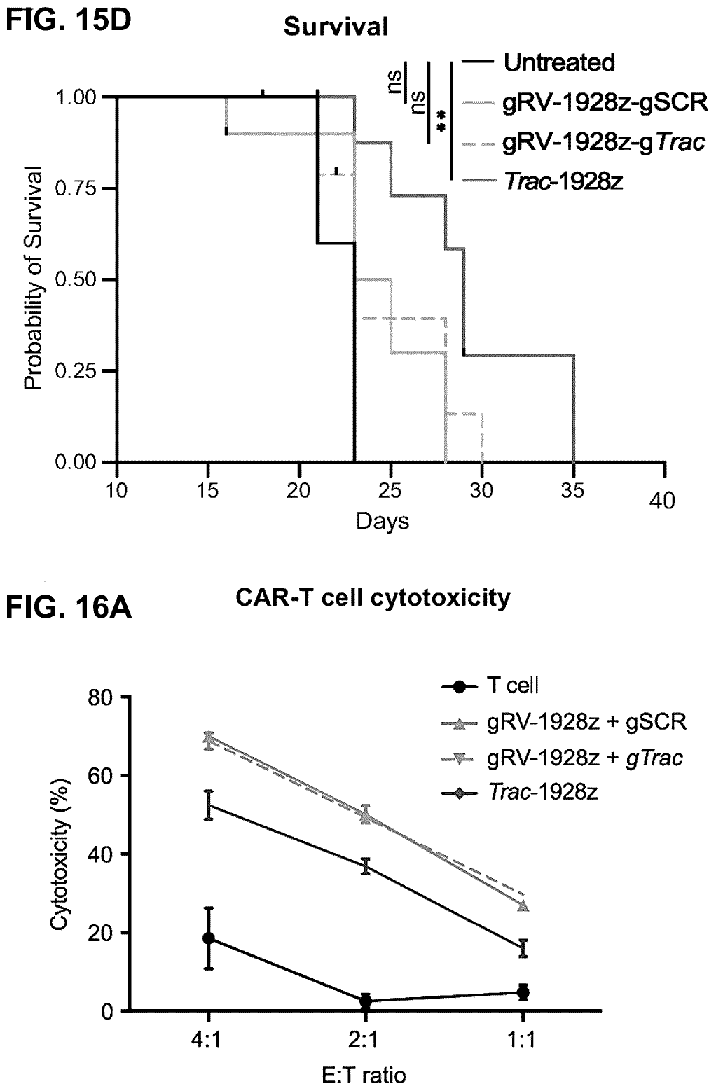

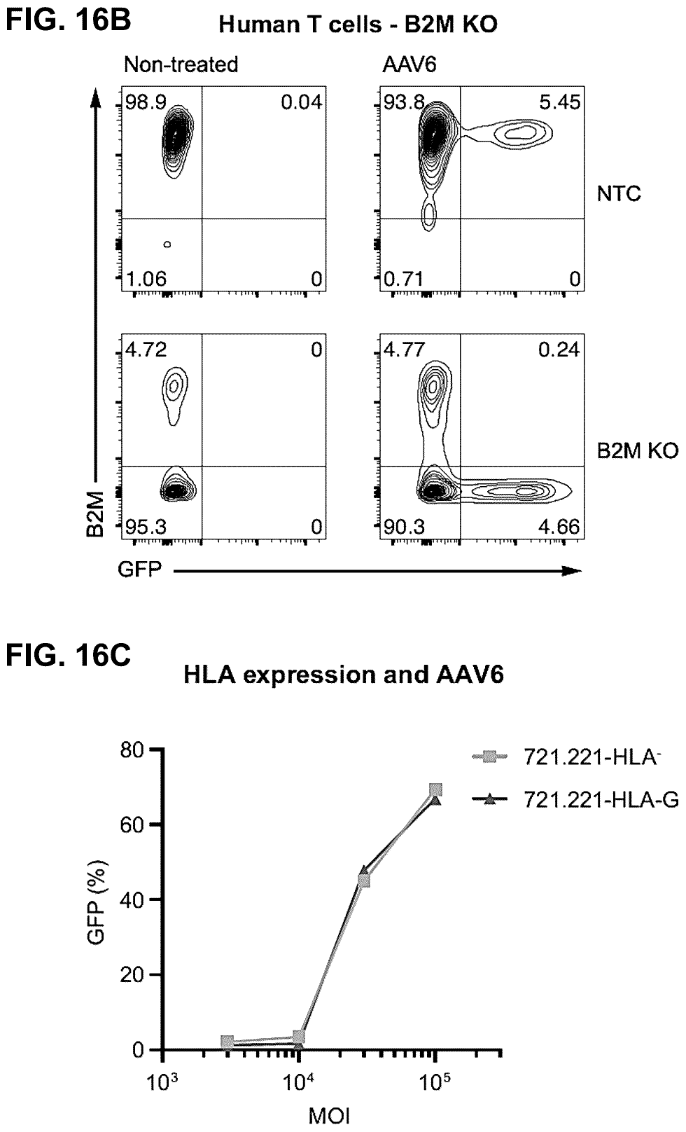

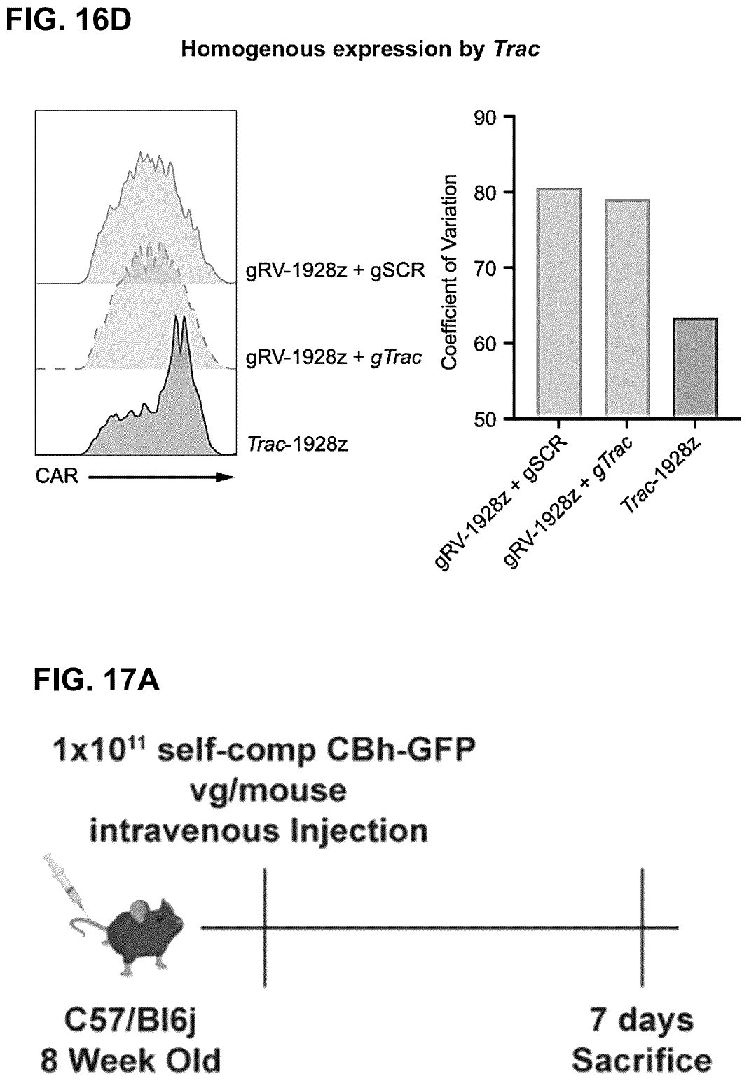

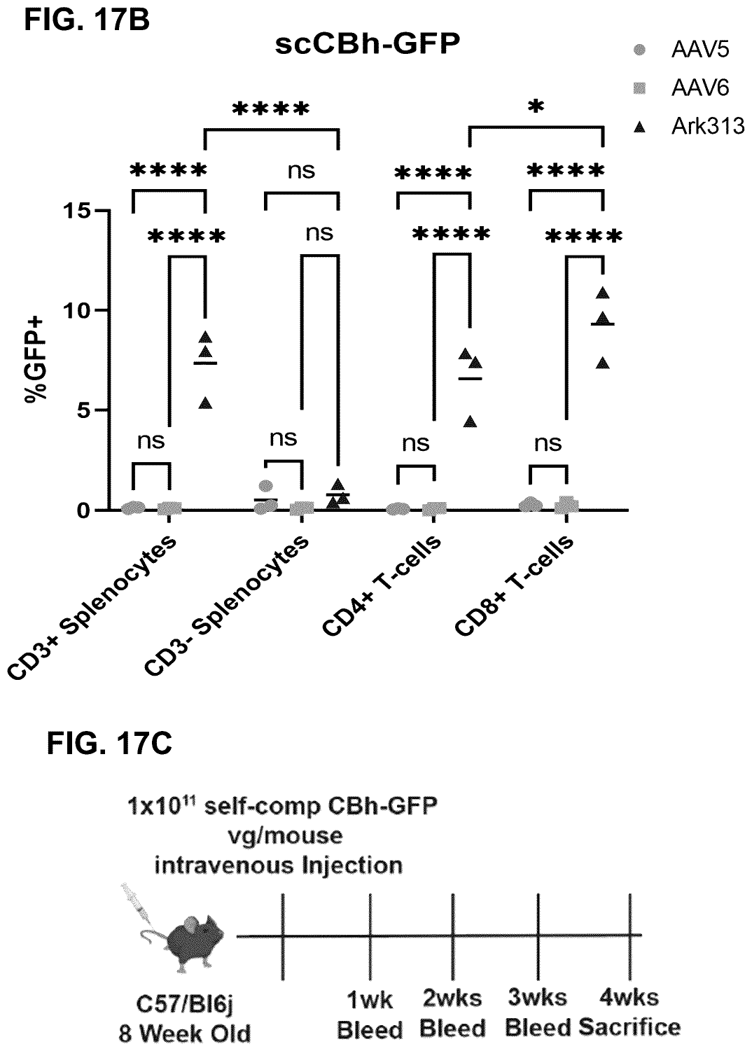

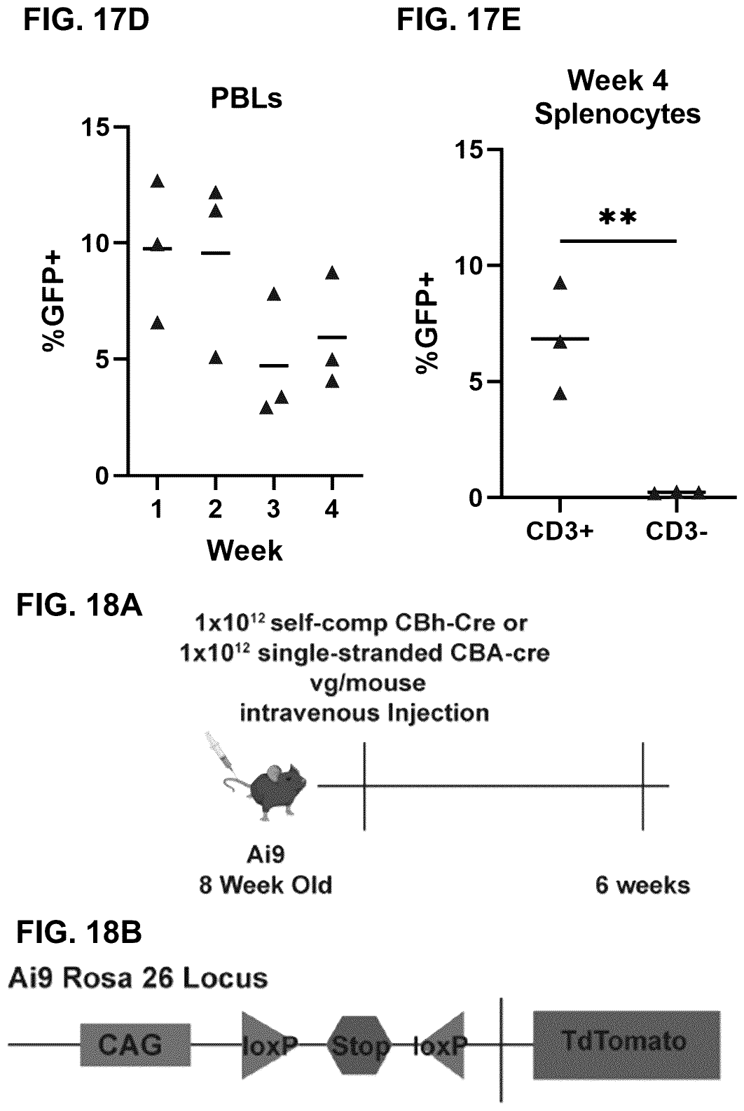

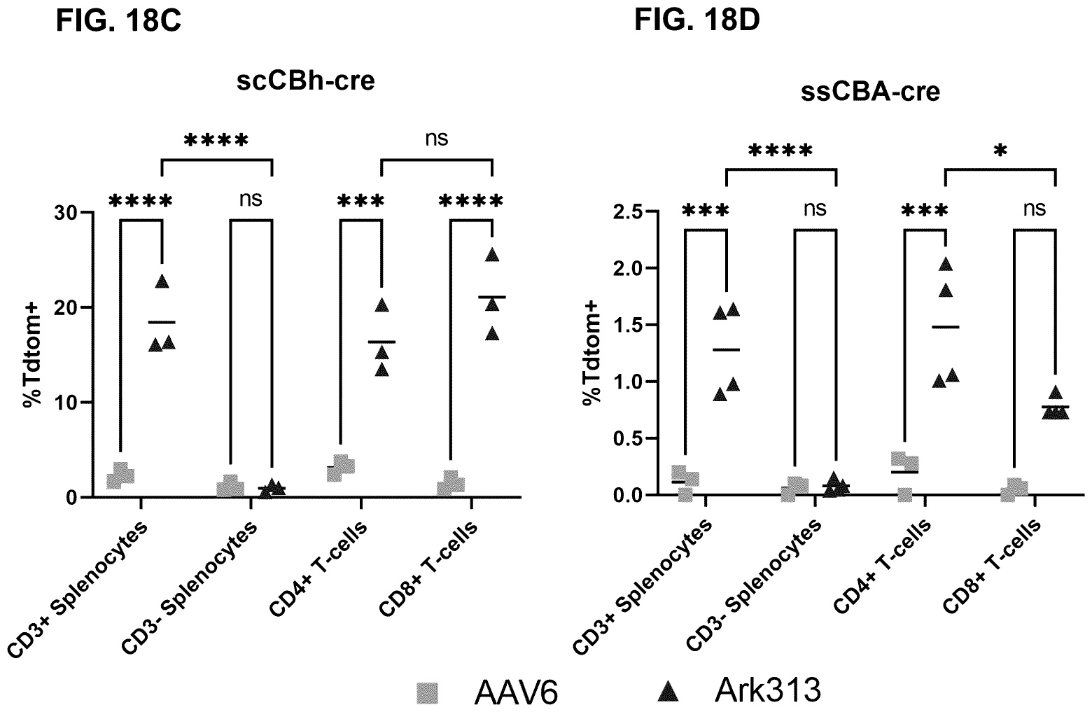

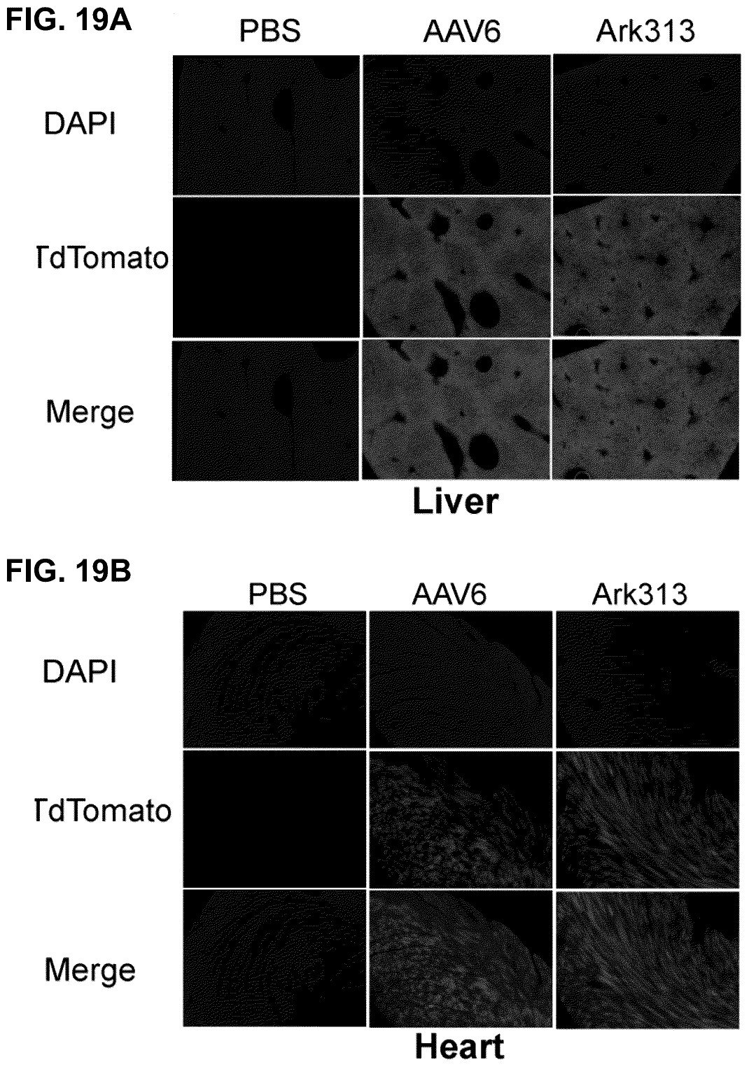

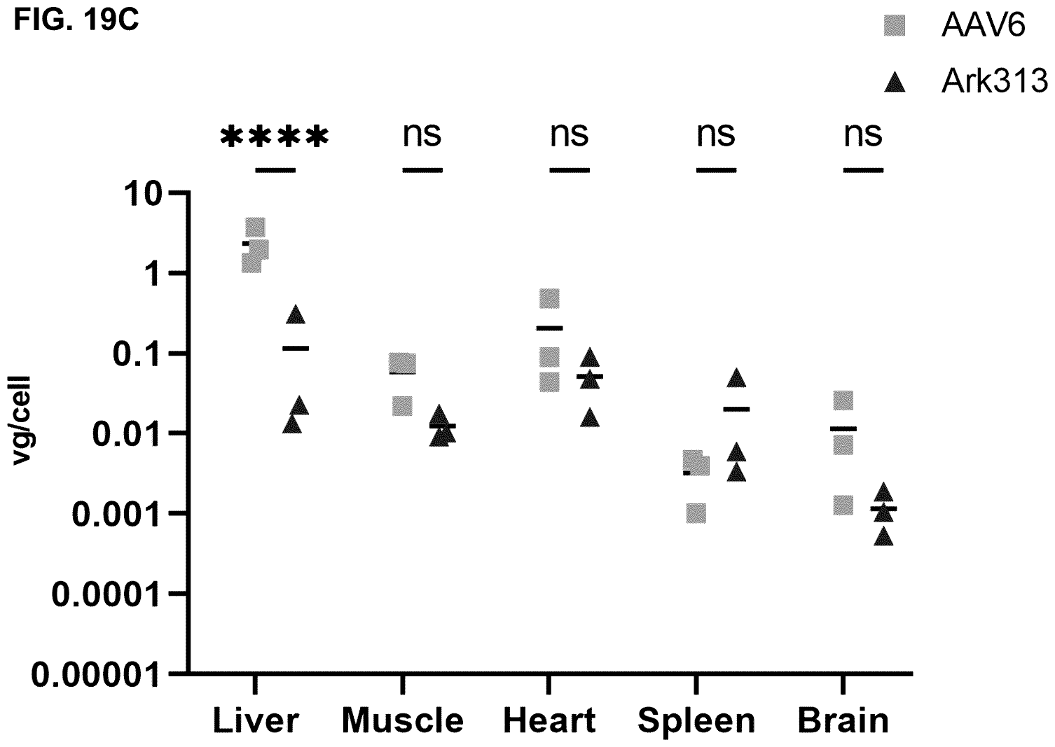

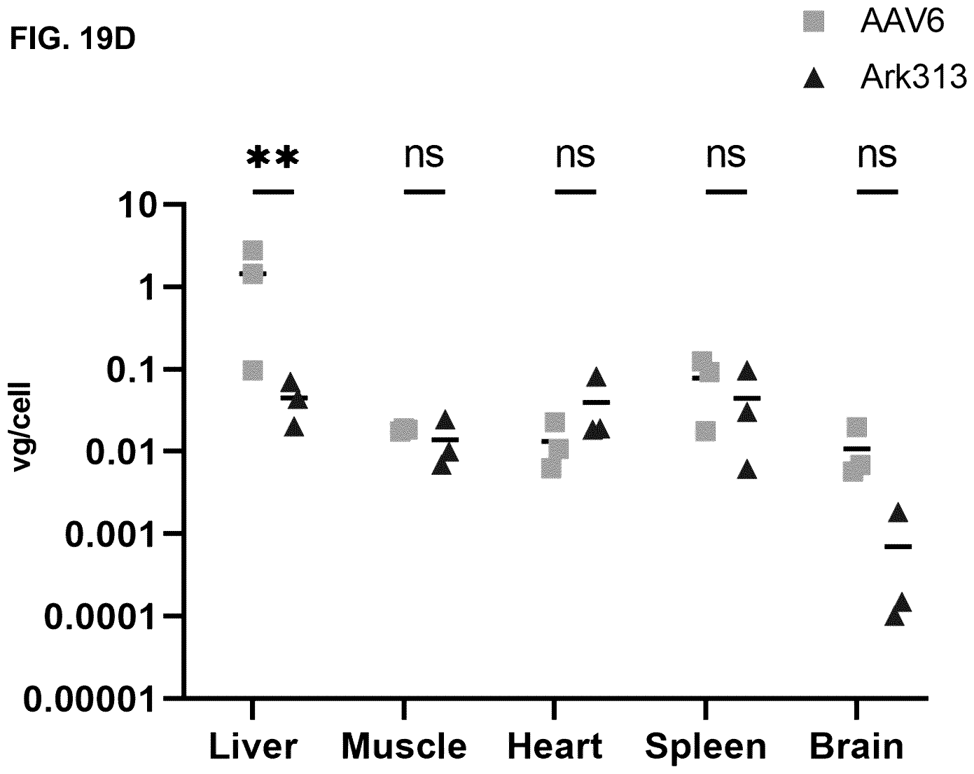

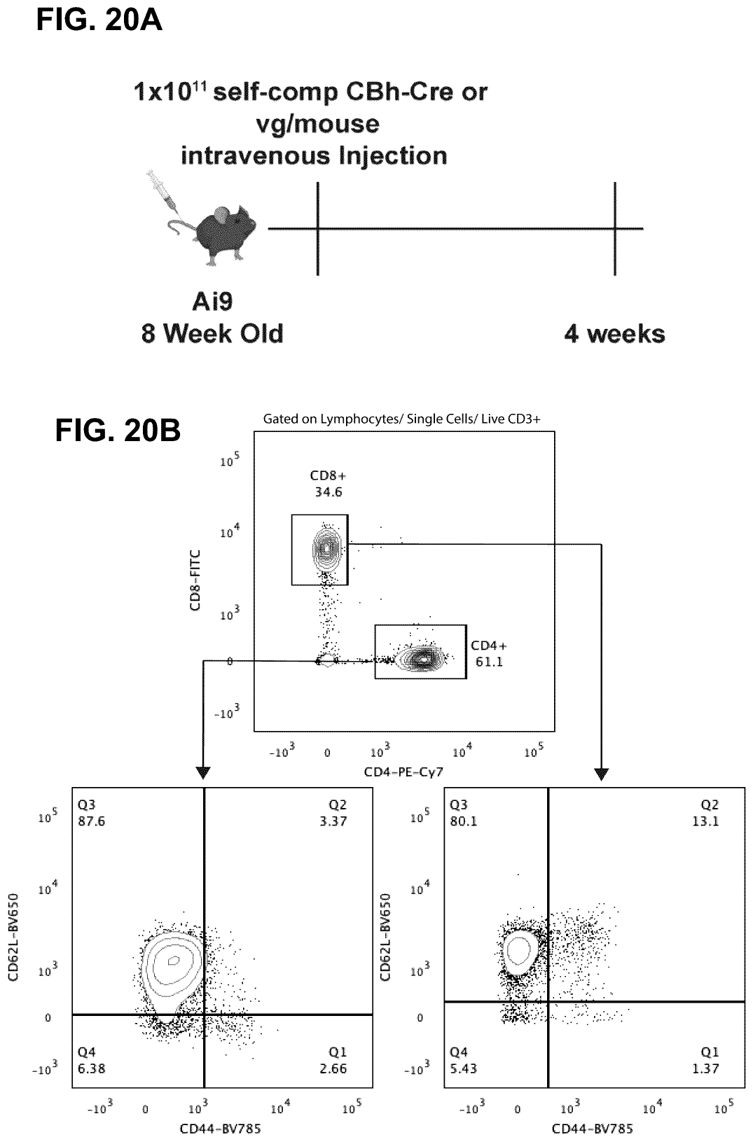

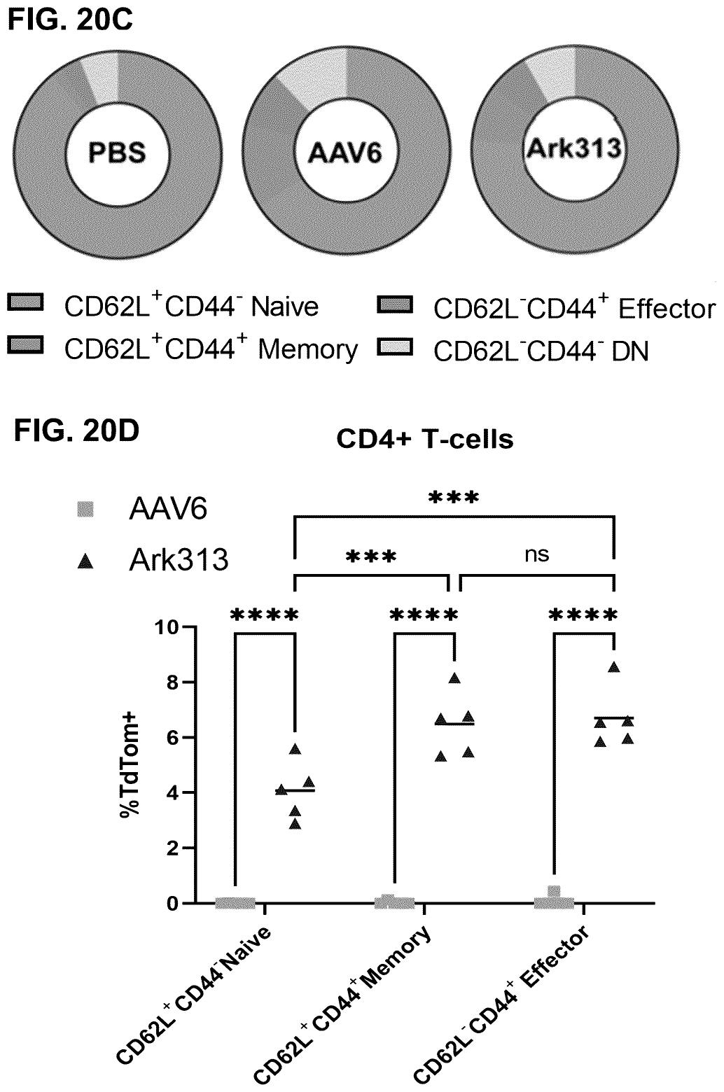

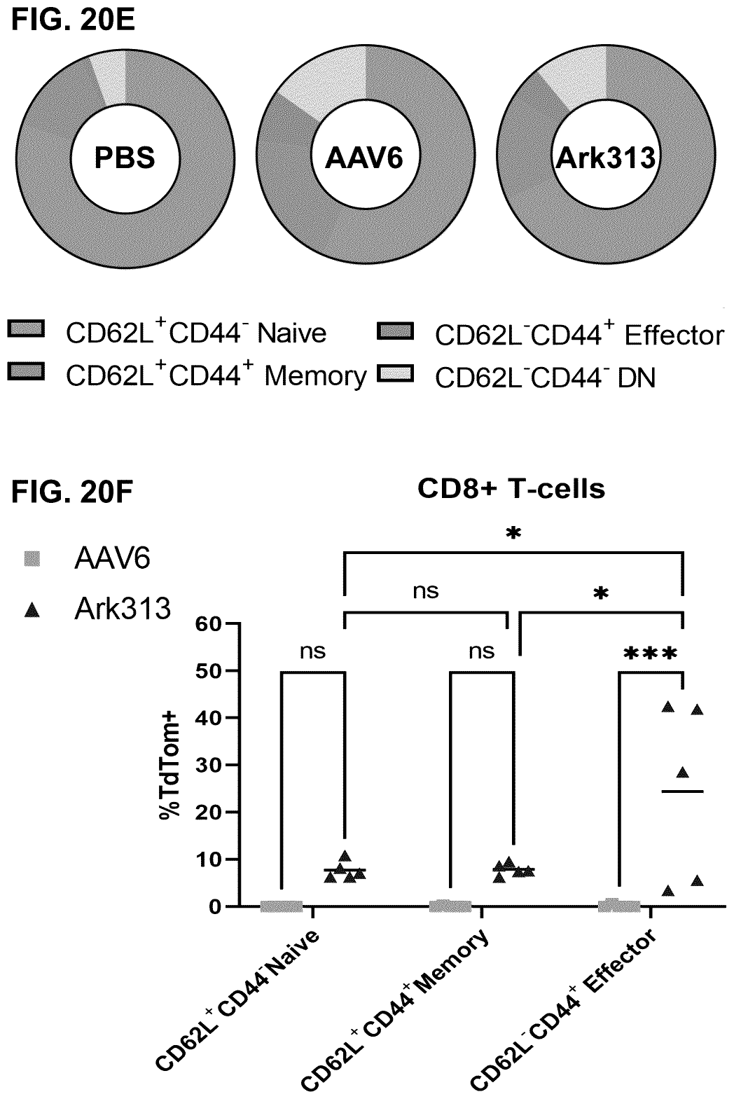

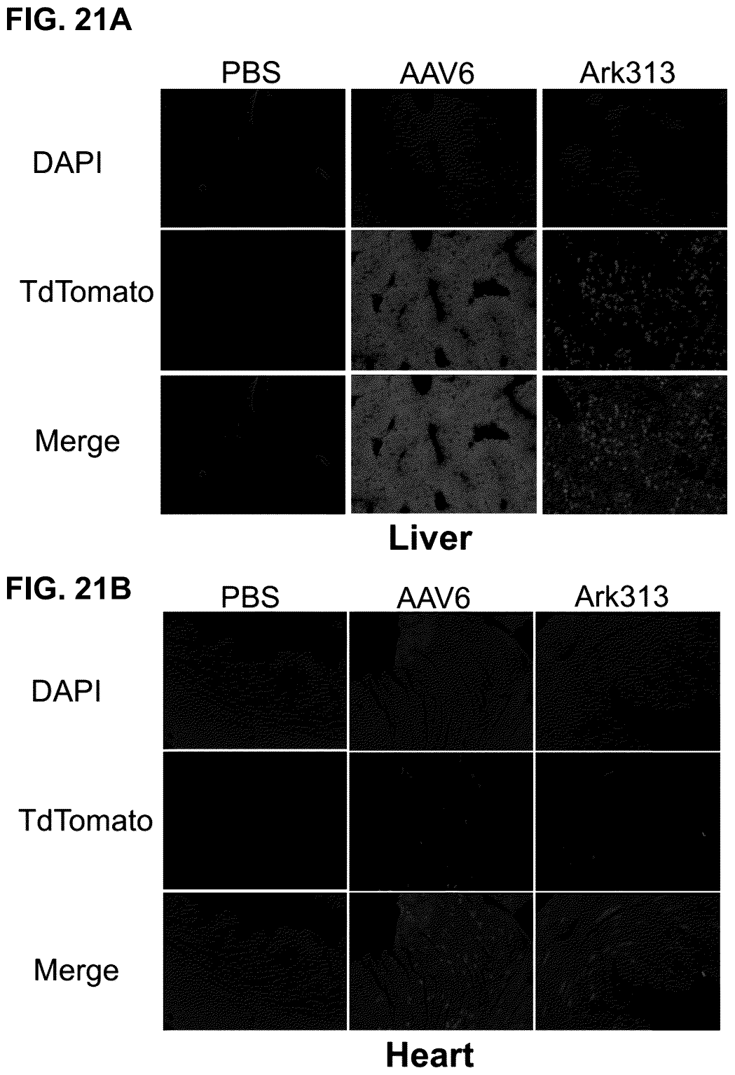

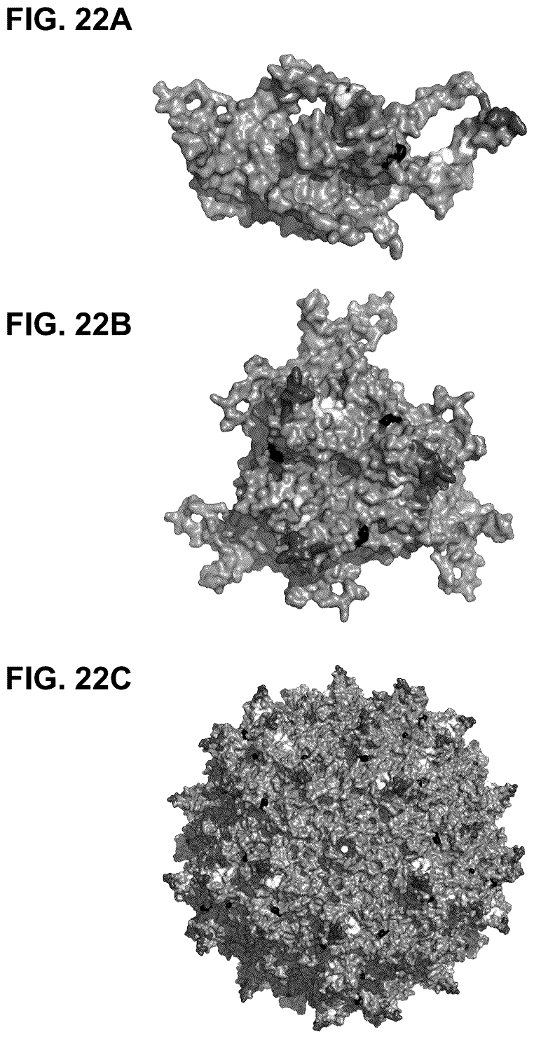

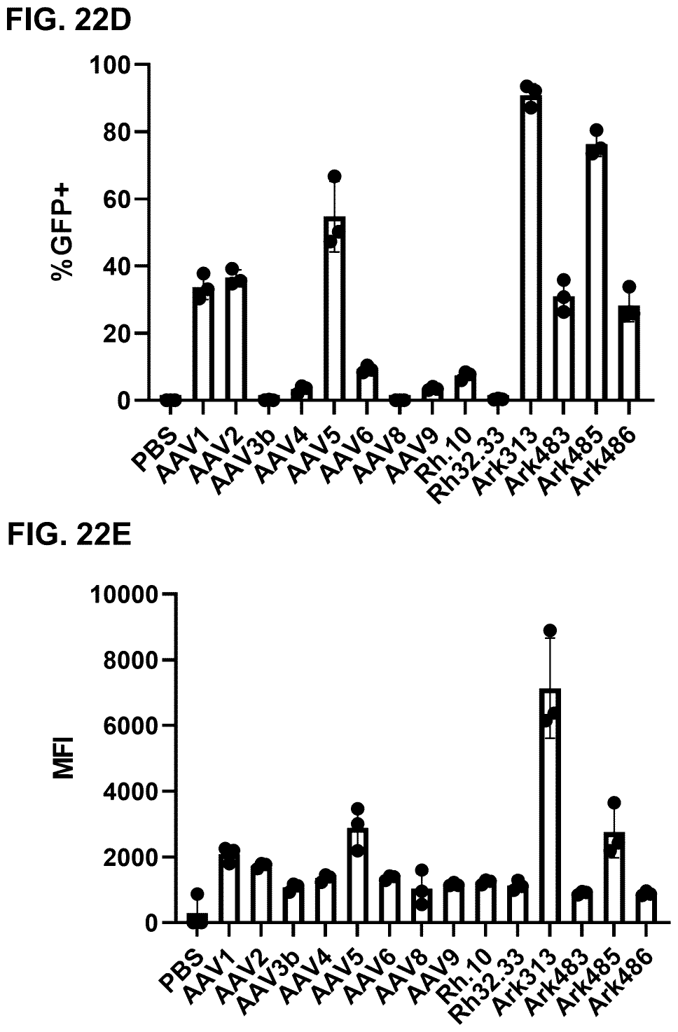

The AAV6 WT sequence and the Ark313 sequence are boxed. FIG. 8C shows the flow cytometry analysis of GFP expression in AAV-transduced activated human T cells using scAAV-CBh-GFP in either AAV6 or Ark313 at the indicated MOI. MFI was determined by flow cytometry at 48 hr after transduction and is shown as the mean±SEM from three human donors. FIG. 8D presents the flow cytometry analysis of GFP expression in AAV-transduced activated murine T cells using scAAV-CBh-GFP in either AAV6 or Ark313 at the indicated MOI. MFI was determined by flow cytometry at 48 hr after transduction and is shown as mean±SEM from three mouse donors.

FIG. 9A-FIG. 9I show that the genome-wide CRISPR-Cas9 knockout screen identified essential host factors for Ark313 infection. FIG. 9A shows the schematic of a genome-wide knockout screen to identify genes associated with Ark313 uptake and processing in primary murine T cells. FIG. 9B shows Cas9-expressing C57BL/6J T cells isolated from spleens, activated with CD3/CD28 beads, and transduced with the gRNA library. Three days later, T cells were re-activated for 24 hr and transduced with scAAV(Ark313)-CAG-GFP. At 48 hr after Ark313 transduction, live cells were gated on BFP expression and subsequently sorted into four bins based on GFP expression. Genomic DNA was extracted from cells in each bin, and amplicon libraries were prepared and sequenced to determine sgRNA enrichment. FIG. 9C provides a Manhattan plot depicting genes ranked by gene effect size from waterbear analysis. Positive regulators of Ark313 transduction are plotted and larger circle sizes indicate lower FDR values. FIG. 9D shows the distribution of log₂ fold change (LFC) values of GFP-positive vs. GFP-negative cells for 90,230 guides in the library (top). LFC for up to five sgRNAs targeting six depleted genes (red lines), overlaid on a gray gradient for the overall distribution (bottom). Values are the average of two technical replicates. FIG. 9E provides an illustration of transmembrane MHC class Ib and GPI-anchored MHC class Ib. FIG. 9F shows T cells from C57BL/6J, NOD, and BALB/cJ mice that were activated and then transduced with either AAV6 or Ark313 scAAV-CAG-GFP an MOI of 5×10⁴. At 48 hr after transduction, cells were stained for QA2 expression (the QA2 antibody binds to both H2-Q7 and H2-Q6) and analyzed for GFP expression by flow cytometry. For each sample, cells were gated as QA2-high or QA2-low based on the median expression of QA2, and GFP expression was analyzed within each subpopulation. FIG. 9G shows the arrayed validation of hits for the regulation of Ark313 infection. C57BL/6J T cells were nucleofected with RNPs targeting eitherAavr, Gpr108, B2m, or H2-Q7 for knockout, transduced with Ark313 scAAV-CAG-GFP at an MOI of 3×10⁴, and analyzed by flow cytometry at 48 hr after transduction. Cells nucleofected with Cas9 only (without a gRNA) were used as a negative control. FIG. 9H shows murine T cells that were treated with PI/PLC to catalyze GPI cleavage, then transduced with scAAV-CBh-GFP in either AAV6 or Ark313. Surface-bound viral genomes bound to the murine T cell were measured by qPCR following a 1 hr incubation at 4° C. to arrest cellular uptake with the indicated AAV capsid, measured qPCR. Results are the mean±SEM from four independent experiments. FIG. 9I shows murine T cells that were treated with phosphatidylinositol-specific phospholipase C (PI/PLC) to catalyze GPI cleavage, then transduced with scAAV-CBh-GFP in either AAV6 or Ark313. GFP signal was analyzed by flow cytometry at 48 hr to determine transduction. Results are the mean±SEM from three independent experiments. In FIG. 9H-FIG. 9I, significance was assessed using two-way ANOVA and Tukey's multiple comparison test (ns=not significant; ****p<0.0001). FIG. 10A shows the correlation of QA2 and GFP MFI, among GFP positive cells, in T cells from C57BL/6J, NOD, and BALB/cJ mice that were activated and transduced with scAAV-CAG-GFP in either AAV6 or Ark313 at an MOI of 1×10⁵. Cells were stained for QA2 expression (the QA2 antibody binds to both H2-Q7 and H2-Q6) and analyzed for GFP expression at 48 hr after transduction by flow cytometry. Statistics were assessed using Spearman's correlation test. FIG. 10B shows T-cells from C57BL/6J, NOD, and BALB/cJ mice that were activated and transduced with scAAV-CAG-GFP in either AAV6 or Ark313. Cells were stained for QA2 expression (the QA2 antibody binds to both H2-Q7 and H2-Q6) and analyzed for GFP expression at 48 hr after transduction by flow cytometry. For each sample, cells were gated as QA2-high or QA2-low based on the median expression of QA2. GFP expression was analyzed within each subpopulation. cvMFI was determined by flow cytometry. Results are the mean±SEM from three technical replicates. Significance was assessed using multiple unpaired t-tests and the Holm-Sidak method for multiple comparison correction (ns=not significant; *p<0.05; **p<0.01. FIG. 10C shows Indel frequencies. C57BL/6J T cells were electroporated with Cas9-RNPs targeting either Aavr, Gpr108, or B2m. Each gene was targeted independently with two sgRNAs. Indel frequencies were determined by genomic DNA PCR followed by Sanger sequencing and ICE analysis. Results are the mean±SEM for each set of two sgRNAs. FIG. 10D shows a flow cytometry analysis of QA2 expression in murine T cells electroporated with RNPs containing two independent sgRNAs for B2m and H2-Q7. Cells electroporated with Cas9 only (without sgRNA) were used as a control. The left side of FIG. 10D shows QA2 expression in each condition. The right side of FIG. 10E shows summary of QA2-positive cells for each condition. The results are the mean±SEM for each set of two sgRNAs. FIG. 10E shows C57BL/6J T cells that were electroporated with two RNPs targeting either Aavr, Gpr108, B2m, or H2-Q7. After knockout, cells were transduced with Ark313 scAAV-CAG-GFP at a MOI of 5×10⁴ and analyzed by flow cytometry 48 hr later. Cells electroporated with Cas9 only were used as control. MFI was determined by flow cytometry. Results are the mean±SEM for two independent sgRNAs. FIG. 11A-FIG. 11H shows that Ark313 enabled efficient gene targeting in primary murine T cells. FIG. 11A shows the schematic of gene knockout by delivering a gRNA to Cas9-expressing T cells using Ark313. FIG. 11B shows a flow cytometry analysis of TCRP expression in Cas9-expressing T cells following transduction with Trac gRNA or scramble gRNA using Ark313 at an MOI of 10¹. FIG. 11C shows the integration of GFP HDRT at the Clta locus to generate a GFP-Clta fusion using Cas9-RNP nucleofection and AAV transduction. FIG. 11D shows GFP integration that was analyzed by flow cytometry. Knock-in efficiency was compared for AAV6 and Ark313 across a range of MOIs. The left side of FIG. 11D shows representative histograms from one experiment while the right side of FIG. 11D shows the summary from three independent experiments. FIG. 11E shows GFP integration at Clta in Rosa26-Cas9-EGFP T cells using single-AAV co-delivery of HDRT and gRNA. FIG. 11F shows the integration of GFP at Clta that was analyzed by flow cytometry. Knock-in efficiency was compared between AAV6 and Ark313 across a range of MOIs. The left side of FIG. 11F shows representative histograms from one experiment while the right side of FIG. 11F shows the summary from four independent experiments. FIG. 11G shows the proliferation of wildtype T cells nucleofected with Cas9-RNP and transduced with AAV compared to AAV-transduced Cas9-expressing T cells. Results are the mean±SEM from two mouse donors (n=2). FIG. 11H shows the normalized yield of Clta-GFP edited cells after five days of expansion post-transduction, comparing Cas9-RNP-nucleofected and AAV-transduced WT cells to AAV-transduced Cas9-expressing T cells. Results are the mean±SEM from two mouse donors (n=2). Significance was assessed using one-way ANOVA and the Sidak's multiple comparison test. *p<0.05; ****p<0.0001.

FIG. 12A shows the flow cytometry analysis of TCRβ expression in Cas9-expressing T cells following transduction with Trac gRNA or scramble gRNA using Ark313 at indicated MOI. Percent TCR negative cells indicated for each condition. FIG. 12B shows the results of PCR on genomic DNA extracted from murine T cells for the Clta-GFP knock-in condition. T cells were electroporated with Clta-targeting RNP and incubated with AAV to target a GFP fusion to Clta, using either AAV6 or Ark313 at the indicated MOI. PCR primers were designed to generate a −400 bp band for the WT Clta locus and a −1100 bp band for the Clta-GFP fusion locus. In FIG. 12B, ns=not significant while *p<0.05; **p<0.01; ***p<0.001; and ****p <0.0001.

FIG. 13A-FIG. 13F show that Trac was an ideal integration locus for experimental T cell immunology. FIG. 13A shows a schematic for targeted integration of a TCR, HIT, or CAR transgene at the Trac locus using co-delivery of HDRT and gRNA in Ark313. FIG. 13B shows the integration of a murine 1928z CAR at the Trac locus by Ark313-mediated delivery to Cas9-expressing T cells. The left side of FIG. 13B shows flow cytometry analysis of CAR expression after transduction at different MOIs. The right side of FIG. 13B shows representative TCRβ and CAR flow cytometry plots for transduction with Trac-1928z Ark313. FIG. 13C shows representative TCRβ and CAR flow cytometry plots after transduction with the indicated Ark313 HDRT at an MOI of 3×10⁴(left side). Edited cells express either 1928z receptor, 1928z receptor with rescued TCR expression, or a HIT receptor. The right side of FIG. 13C shows expression of Trac-targeted OT-I TCR T cells in comparison to T cells isolated from transgenic OT-I TCR mice. FIG. 13D shows cytotoxicity determined based on the luciferase signal after a 24-hr co-culture of T cells with luciferase-expressing hCD19-expressing LL2 cells. Results are the mean±SEM from three technical replicates. Significance was assessed using one-way ANOVA and Dunnett's multiple comparisons test. FIG. 13E shows an incucyte analysis of Trac-OT-I TCR T cells co-cultured with OVA-expressing mCherry-positive B78 cells. Results are the mean±SEM from three technical replicates. Significance was assessed using repeated-measures one-way ANOVA and Dunnett's multiple comparisons test. FIG. 13F shows efficacy of dual-gene targeting in murine T cells. GFP and CAR flow cytometry plots of Cas9-expressing T cells transduced with GFP-Clta and Trac-1928z Ark313 viruses at an MOI of 1×10⁵ for each AAV. In FIG. 13D-FIG. 13E, *p<0.05; **p<0.01; ***p<0.001; and ****p<0.0001.

FIG. 14A shows the schematic of targeted integration of a CAR transgene at the Trac locus using co-delivery of the HDRt and gRNA in Ark313. FIG. 14B shows integration of a 1928z CAR at the Trac locus by Ark313-mediated delivery to Cas9-expressing T cells. The left side of FIG. 14B shows knock-in percentage at the indicated MOI while the right side shows the coefficient of variation for CAR expression. CAR expression determined by flow cytometry. Results are the mean±SEM from four independent replicates. FIG. 14C shows the proliferation of wildtype T cells nucleofected with Cas9-RNP and transduced with AAV compared to AAV-transduced Cas9-expressing T cells. Results are the mean±SEM from two independent experiments. FIG. 14D shows normalized yield of Trac-1928z cells after five days of expansion, comparing Cas9-RNP-nucleofected and AAV-transduced WT cells to AAV-transduced Cas9-expressing T cells. Results are the mean±SEM from two mouse donors. Significance was assessed using one-way ANOVA and Sidak's multiple comparisons test (***p<0.001; ****p<0.0001). FIG. 14E shows integration of 1928z or Thy1.1-P2A-1928z at Trac using Ark313-mediated delivery to Cas9-expressing T cells. Knock-in efficiency was determined by flow cytometry for CAR and Thy1.1 expression.

FIG. 15A-FIG. 15D show that targeting a CAR to the Trac locus using Ark313 enhanced tumor control in an immunocompetent solid tumor mouse model. FIG. 15A shows a schematic of the syngeneic solid tumor model. hCD19-expressing LL2 cells were injected subcutaneously into C57BL/6J mice. The tumor-bearing mice were treated with either Ark313 Trac-1928z T cells or gRV-1928z T cells. The gRV-1928z T cells were co-transduced with Ark313 expressing either a SCR or a Trac targeting gRNA to generate TCR⁺ and TCR-CAR T cells. FIG. 15B shows TCRβ and CAR flow cytometry plots of engineered T cells using the indicated methods. FIG. 15C shows tumor growth in non-treated (n=6) mice and in mice treated with 1.5×10⁶ Trac-1928z T cells (n=9), gRV-1928z-gSCR T cells (n=10), or gRV-1928z-gTrac T cells (n=10). FIG. 15D shows a Kaplan-Meier survival analysis of mice injected with hCD19-expressing LL2 cells. Comparison of non-treated mice to mice injected with either Trac-1928z T cells (n=9), gRV-1928z-gSCR T cells (n=10), or gRV-1928z-gTrac T cells (n=10). Significance was assessed using a log-rank (Mantel-Cox) test (ns=not significant; **p<0.01).

FIG. 16A shows a cytotoxicity assay of murine CAR-T cell co-culture with hCD19-expressing LL2 cells. Cytotoxicity was determined based on luciferase signal after 24 hr of co-culture at the indicated effector cell to tumor cell ratios (E:T). Results are the mean±SEM from three technical replicates. FIG. 16B shows that activated human T cells were nucleofected with either a non-targeting control (NTC) sgRNA or B2M targeting sgRNA. Cells were transduced with scAAV-CAG-GFP AAV6 at an MOI of 5×10⁵ and analyzed by flow cytometry for GFP and B2M expression after 48 hr. FIG. 16C shows human 721.221 HLA negative cells or 721.221 cells engineered to express HLA-G were transduced with scAAV-CAG-GFP AAV6 at indicated MOIs and analyzed by flow cytometry for GFP expression after 48 hr. FIG. 16D shows flow cytometry analysis of CAR-expressing murine T cells engineered with the indicated methods and then cells were gated on CAR expression for subsequent analysis. The left side shows CAR expression using each indicated engineering method while the right side shows coefficient of variation for CAR expression with each method.

FIG. 17A-FIG. 17E provide details regarding thein vivo transduction of T-cells with Ark313. FIG. 17A is a schematic showing that 8-week-old C57BL/6J mice were injected with either AAV5, AAV6 or Ark313 at 1×10¹¹ vg/mouse packaging a sc-CBh-GFP transgene. One week later, mice were sacrificed and splenocytes analyzed by flow cytometry. FIG. 17B shows that while neither AAV5 or AAV6 were able to appreciably transduce CD3+ splenocytes at this dose, Ark313 did transduce up to 10.2% of spleen resident T-cells with little off target effects for CD3-splenocytes. Ark313 was capable of transducing both CD4+ and CD8+ T-cells equally in vivo. Significance was assessed using two-way ANOVA and Tukey's multiple comparison test (ns=not significant; ****p<0.0001). FIG. 17C is a schematic showing that as T-cells are a dividing population, transgene expression from Ark313 packaging a sc-CBh-GFP cassette injected at 1×10¹¹ vg/mouse was tracked over a 4-week period within circulating CD3+ peripheral blood leukocyte (PBLs). FIG. 17D shows that Ark313 transduced T-cells were detectable in circulating PBLs up to 4 weeks post injection. FIG. 17E shows that mice were sacrificed and splenocytes were analyzed by flow cytometry. Up to 9.3% of CD3+ splenocytes were GFP+ with negligible transduction of the CD3-population. Significance was assessed using an unpaired t-test (**p<0.01).

FIG. 18A-FIG. 18D show an vivo comparison of self-complementary and single-stranded AAV transgenes. FIG. 18A shows a schematic of the experimental timing while FIG. 18B shows the Ai9 mouse model, which has a cre activatable TdTomato signal. A self-complementary CBh driven cre (sc-CBh-cre) or a single stranded CBA driven cre (ss-CBA-cre) were packaged in either AAV6 or Ark313 and injected at a 1×10¹² vg/mouse. Mice were sacrificed and splenocytes analyzed by flow cytometry 6 weeks post injection. FIG. 18C shows that Ark313 packaging sc-CBh-cre transgene attained up to 22.8% transduction of CD3+ T-cells with no significant difference in CD3-splenocytes transduced between AAV6 and Ark313. No significant difference in transduction of CD4+vs. CD8+ T-cells was observed. Significance was assessed using two-way ANOVA and the Sidak's multiple comparison test (ns=not significant; ***p<0.001; ****p<0.0001). FIG. 18D shows that when packaging ss-CBA-cre, Ark313 transduced up to 1.6% of CD3+ splenocytes while AAV6 was unable to appreciably do so. CD4+ T-cells were significantly more transduced than CD8+ T-cells when transduced by a single-stranded cassette in vivo. Significance was assessed using two-way ANOVA and the Sidak's multiple comparison test (ns=not significant; *p<0.05; ***p<0.001; ****p<0.0001).

FIG. 19A-FIG. 19D shows the biodistribution of Ark313 in Ai9 mice. FIG. 19A and FIG. 19B show Ai9 mice that were injected with either AAV6 or Ark313 at 1×10¹² vg/mouse packaging a sc-CBh-cre or a ss-CBA-cre cassette and sacrificed 6 weeks post injection. The liver and heart were sectioned and imaged for native fluorescence. FIG. 19A shows that while the sc-Cbh-cre injected group showed no difference in transduction in the liver or heart between Ark313 or AAV6, FIG. 19B shows that ss-CBA-cre cohort showed a decrease in TdTomato+ signal within the liver in Ark313 injected mice. FIG. 19C and FIG. 19D show that genomic and viral DNA were extracted from liver, muscle, heart, spleen and brain and quantitated by qPCR. Ark313 showed no significant difference to AAV6 in vg/cell in muscle, heart, spleen and brain but was significantly reduced in the liver for both the sc-CBh-cre cohort (FIG. 19C) and ss-CBA-cre cohort (FIG. 19D). Significance was assessed using two-way ANOVA and the Sidak's multiple comparison test (ns=not significant; **p<0.01; ****p<0.0001).

FIG. 20A-FIG. 20F show that Ark313 significantly infects memory and effector T-cells over naive T-cell in vivo. FIG. 20A shows a schematic of the experiment in which either AAV6 or Ark313 packaging a sc-CBh-cre cassette was injected in Ai9 mice at 1×10¹¹ vg/mouse and sacrificed 4 weeks post injection. Splenocytes were harvested and analyzed by flow cytometry for T-cell activation markers CD62L and CD44. FIG. 20B shows the gating strategy for naive, memory, and effector CD4+ and CD8+ T-cells. FIG. 20C shows that mice injected with AAV6 and Ark313 displayed an increase of both CD4+ memory and effector T-cells over PBS injected controls. FIG. 20D shows that TdTomato+ expression was quantified in naive/memory/effector CD4+ T-cells. While up 5.6% of naive CD4+ T-cells were transduced by Ark313, both memory and effector CD4+ subsets had a significant increase in amount of TdTomato+ cells over naive T-cells. Significance was assessed using two-way ANOVA and the Sidak's multiple comparison test (ns=not significant; ***p<0.001; ****p <0.0001). FIG. 20E shows that mice injected with AAV6 and Ark313 displayed an increase of CD8+ effector T-cells over PBS injected controls. FIG. 20F shows that Ark313 transduced up to 10.8% of naive CD8+ T-cells and 9.5% of memory CD8+ T-cells. Interestingly CD8+ effector T-cells were significantly more transduced T-cells than both naive and memory CD8+ T-cells, with up to 42.5% TdTomato+ in some cases. Significance was assessed using two-way ANOVA and the Sidak's multiple comparison test (ns=not significant; *p<0.05; ***p <0.001).

FIG. 21A (liver) and FIG. 21B (heart) show the effect of Ark313 as a single-stranded vector.

FIG. 22A-FIG. 22E show the evolution of the capsid mutant Ark313. FIG. 22A shows the monomer, FIG. 22B shows the trimer, and FIG. 22C shows the assembled capsid, which has a demonstrated importance for tissue tropism and cell entry. Ark313, the most enriched variant within from the evolution, outperformed all other serotypes whether natural of engineered by both % GFP+(FIG. 22D) and median fluorescence intensity (FIG. 22E).

### VIII. DETAILED DESCRIPTION

Adeno-associated virus (AAV) vectors have become a leading platform for therapeutic gene delivery. Unfortunately, AAV-based gene therapies are sometimes be less effective than desired because of, for example, difficulties in optimizing administration routes to target a cell or tissue of interest and the subject's immune responses against the vector carrying the therapeutic gene (e.g., a transgene of interest). Host-derived pre-existing antibodies generated upon natural encounter of AAV or recombinant AAV vectors prevent first time as well as repeat administration of AAV vectors as vaccines and/or for gene therapy. Serological studies reveal a high prevalence of antibodies in the human population worldwide with about 67% of people having antibodies against AAV1, 72% against AAV2, and about 40% against AAV5 through AAV9. In gene therapy, pre-existing antibodies in the subject cause problems because certain clinical scenarios involving gene silencing or tissue degeneration require multiple AAV vector administrations to sustain long term expression of the transgene.

Known AAV serotypes each have a specific tissue tropism, and there are some tissues (e.g., immune cells) that cannot be easily targeted using these AAVs. Delivery of therapeutic genes using AAV vectors for treating disorders like immunodeficiencies and some cancers can be particularly difficult as AAV-mediated gene therapies targeting immune cells would require systemic delivery at high doses, thus triggering a subject's immune response against the vector carrying the therapeutic gene. To circumvent these issues, recombinant AAV vectors which evade antibody recognition and/or selectively target tissues of the immune system are needed.

Aspects provided in the present disclosure will help a) expand the eligible cohort of patients suitable for AAV-based gene therapy and b) allow multiple, repeat administrations of AAV-based gene therapy vectors. Additionally, there is a need to develop AAV-based gene therapies that are able to selectively and specifically target tissues of interest, including tissues that are have been difficult to target using known AAV serotypes such as immune cells like T cells and NK cells. The present disclosure is based, at least in part, on the novel discovery that capsid antigenicity and functional properties of AAV capsids and capsid proteins, such as tropism and transduction, overlap in a structural context and can be modified to impart improved functionality.

### A. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can comprise more than one element.

“About” is used to provide flexibility to a numerical range endpoint by providing that a given value can be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Moreover, the present disclosure also contemplates that In an aspect, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rhl0, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-:375-383; and Table 1).

The genomic sequences of various serotypes of AAV and the autonomous parvoviruses, as well as the sequences of the native terminal repeats (TRs), Rep proteins, and capsid subunits are known in the art. Such sequences can be found in the literature or in public databases such as GenBank, such as, for example, GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences.

TABLE 1
Identification of Various AAV Serotypes and Clades

| | GenBank |
|---|---|
| | Accession |
| | Number |
| Complete | |
| Genomes | |
| Adeno-associated | NC_00207, |
| virus 1 | AF063497 |
| Adeno-associated | NC 001401 |
| virus 2 | |
| Adeno-associated | NC 001729 |
| virus 3 | |
| Adeno-associated | NC 001863 |
| virus 3B | |
| Adeno-associated | NC 001829 |
| virus 4 | |
| Adeno-associated | Y18065, |
| virus 5 | AF085716 |
| Adeno-associated | NC 001862 |
| virus 6 | |
| Avian AAV | AY186198, |
| ATCC VR-865 | AY629583, |
| | NC 004828 |
| Avian AAV strain | NC_006263, |
| DA-1 | AY629583 |
| Bovine AAV | NC_005889, |
| | AY388617, |
| | AAR26465 |
| AAV11 | AAT46339, |
| | AY631966 |
| AAV12 | AB116639, |
| | DQ813647 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 (AAV9) | AY530579 |
| Hu31 | AY530596 |
| Hu32 | AY530597 |
| HSC1 | M1332400.1 |
| HSC2 | M1332401.1 |
| HSC3 | M1332402.1 |
| HSC4 | M1332403.1 |
| HSC5 | M1332405.1 |
| HSC6 | M1332404.1 |
| HSC7 | M1332407.1 |
| HSC8 | M1332408.1 |
| HSC9 | M1332409.1 |
| HSC11 | M1332406.1 |
| HSC12 | M1332410.1 |
| HSC13 | M1332411.1 |
| HSC14 | M1332412.1 |
| HSC15 | M1332413.1 |
| HSC16 | M1332414.1 |
| HSC17 | M1332415.1 |
| Hu68 | |
| Clonal Isolate | |
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |
| Others | |
| Rh74 | |
| Bearded Dragon AAV | |
| Snake AAV | NC_006148.1 |

The terms “heterologous nucleotide sequence” and “heterologous nucleic acid” are used interchangeably herein and refer to a sequence that is not naturally occurring in the virus. Generally, the heterologous nucleic acid comprises an open reading frame that encodes a polypeptide or nontranslated RNA of interest (e.g., for delivery to a cell or subject).

A “polynucleotide” or “nucleotide” as used herein refers to a sequence of nucleotide bases, and can be RNA, DNA or DNA-RNA hybrid sequences (including both naturally occurring and non-naturally occurring nucleotide), but in representative aspects are either single or double stranded DNA sequences.

As used herein, the term “peptide” refers to a short amino acid sequence. The term peptide can be used to refer to portion or region of an AAV capsid amino acid sequence. The peptide can be a peptide that naturally occurs in a native AAV capsid, or a peptide that does not naturally occur in a native AAV capsid. Naturally occurring AAV peptides in an AAV capsid can be substituted by non-naturally occurring peptides. For example, a non-naturally occurring peptide can be substituted into an AAV capsid to provide a modified capsid, such that the naturally-occurring peptide is replaced by the non-naturally occurring peptide. As used herein, the term “polypeptide” encompasses both peptides and proteins, unless indicated otherwise.

As used herein, the term “amino acid” encompasses any naturally occurring amino acid, modified forms thereof, and synthetic amino acids. Alternatively, an amino acid herein can be a modified amino acid residue and/or can be an amino acid that is modified by post-translation modification (e.g., acetylation, amidation, formylation, hydroxylation, methylation, phosphorylation or sulfatation). Naturally occurring, levorotatory (L-) amino acids are shown in Table 2.

TABLE 2
Listing of Amino Acids and Corresponding Codes

| Amino Acid Residue | 3-Letter Code | 1-Letter Code |
|---|---|---|
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) and/or can be an amino acid that is modified by post-translation modification (e.g., acetylation, amidation, formylation, hydroxylation, methylation, phosphorylation or sulfatation).

TABLE 3
Listing of Modified Amino Acid Residues

| Modified Amino Acid Residue | Abbreviation |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

As used herein, the terms “virus vector,” “vector” ˜ or “gene delivery vector” ˜ refer to a virus (e.g., AAV) particle that functions as a nucleic acid delivery vehicle, and which comprises the vector genome (e.g., viral DNA or vDNA) packaged within a virion. Alternatively, in some contexts, the term “vector” can be used to refer to the vector genome/vDNA alone.

A “rAAV vector genome” or “rAAV genome” as used herein is an AAV genome (i.e., vDNA) that comprises one or more heterologous nucleic acid sequences. rAAV vectors generally require only the terminal repeat(s) (TR(s)) in cis to generate virus. All other viral sequences are dispensable and can be supplied in trans (Muzyczka, (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences can be provided in trans (e.g., from a vector, such as a plasmid, or by stably integrating the sequences into a packaging cell). In an aspect, a disclosed rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” can be from any AAV, including but not limited to serotypes 1, 2, 3, 4, 5, 6, 7, 8, 9, 10, 11, 12, 13 or any other AAV now known or later discovered (see, e.g., Table 1). An AAV terminal repeat need not have the native terminal repeat sequence (e.g., a native AAV TR sequence can be altered by insertion, deletion, truncation and/or missense mutations), as long as the terminal repeat mediates the desired functions, e.g., replication, virus packaging, integration, and/or provirus rescue, and the like.

An AAV vector typically comprises a protein-based capsid, and a nucleic acid encapsidated by the capsid. The nucleic acid can be, for example, a vector genome comprising a transgene flanked by inverted terminal repeats. The AAV “capsid” is a near-spherical protein shell that comprises individual “capsid proteins” or “subunits.” AAV capsids typically comprise about 60 capsid protein subunits, associated and arranged with T=1 icosahedral symmetry. When an AAV vector is described herein as comprising an AAV capsid protein, it will be understood that the AAV vector comprises a capsid, wherein the capsid comprises one or more AAV capsid proteins (i.e., subunits). Also described herein are “viral-like particles” or “virus-like particles,” which refers to a capsid that does not comprise any vector genome or nucleic acid comprising a transgene.

The virus vectors of the present disclosure can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al. (2000) Molecular Therapy. 2:619.

The virus vectors of the present disclosure can further be duplexed parvovirus particles as described in international patent publication WO 01/92551 (the disclosure of which is incorporated herein by reference in its entirety). Thus, in an aspect, double stranded (duplex) genomes can be packaged into a disclosed virus capsids. Further, the viral capsid or genomic elements can contain other modifications, including insertions, deletions and/or substitutions.

The term “self-complimentary AAV” or “scAAV” refers to a recombinant AAV vector which forms a dimeric inverted repeat DNA molecule that spontaneously anneals, resulting in earlier and more robust transgene expression compared with conventional single-strand (ss) AAV genomes. See, e.g., McCarty, D. M., et al., Gene Therapy 8, 1248-1254 (2001). Unlike conventional ssAAV, scAAV can bypass second-strand synthesis, the rate-limiting step for gene expression. Moreover, double-stranded scAAV is less prone to DNA degradation after viral transduction, thereby increasing the number of copies of stable episomes. Notably, scAAV can typically only hold a genome that is about 2.4 kb, half the size of a conventional AAV vector. In an aspect, the AAV vectors described herein are self-complementary AAVs.

A “therapeutic polypeptide” or “therapeutic protein” is a polypeptide or protein that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In an aspect, the subject can comprise a human. In an aspect, the subject can comprise a mouse. In an aspect, the subject can comprise a human in need of one or more gene therapies.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

Unless otherwise defined, all technical terms used herein have the same meaning as commonly understood by one of ordinary skill in the art to which this disclosure belongs.

### B. Adeno-Associated Virus (AAV)

Adeno-associated virus (AAV), a member of the Parvovirus family, is a small, non-enveloped virus. Wildtype AAV is composed of an icosahedral protein capsid which encloses a single-stranded DNA genome. In wildtype AAVs, inverted terminal repeats (ITRs) flank the coding nucleotide sequences (e.g., a polynucleotides) for the non-structural proteins (encoded by Rep genes) and the structural proteins (encoded by capsid genes or Cap genes). Rep genes encode the non-structural proteins that regulate functions comprising the replication of the AAV genome. Cap genes encode the structural proteins, VP1, VP2 and/or VP3 that assemble to form the capsid.

The present disclosure provides recombinant AAV capsid proteins (VP1, VP2 and/or VP3) comprising a modification (e.g., a substitution) in the amino acid sequence relative to a wildtype capsid protein, and AAV capsids and AAV vectors comprising the modified AAV capsid protein. As detailed herein, modifications of disclosed capsid proteins can confer one or more desirable properties to virus vectors comprising the modified AAV capsid protein variants herein, including without limitation, the ability to evade neutralizing antibodies and/or the ability to specifically and selectively target a cell or tissue of interest. Thus, the present disclosure addresses some of the limitations associated with conventional AAV vectors.

In an aspect, AAV vectors herein can be engineered to include one or more capsid protein variants. In an aspect, AAV vectors herein can be engineered to include at least one or more amino acid substitutions, wherein the one or more substitutions can modify one or more antigenic sites on the AAV capsid protein. The modification of the one or more antigenic sites can result in inhibition of binding by an antibody to the one or more antigenic sites and/or inhibition of neutralization of infectivity of a virus particle comprising said a capsid protein variant herein.

Accordingly, in an aspect herein, the present disclosure provides an adeno-associated virus (AAV) capsid protein variant, comprising one or more amino acid modifications (e.g., substitutions and/or deletions), wherein the one or more modifications modify one or more antigenic sites on the AAV capsid protein. In an aspect, modification of the one or more antigenic sites can result in inhibition of binding by an antibody to the one or more antigenic sites and/or inhibition of neutralization of infectivity of a virus particle comprising said AAV capsid protein. In an aspect, the modified antigenic site can prevent antibodies from binding or recognizing or neutralizing AAV capsids. In an aspect, the antibody can be an IgG (including IgG1, IgG2a, IgG2b, IgG3), IgM, IgE or IgA. In an aspect, the modified antigenic site can prevent binding, recognition, or neutralization of AAV capsids by antibodies from different animal species, wherein the animal is human, canine, porcine, bovine, non-human primate, rodent (e.g., mouse), feline or equine.

In an aspect, modification of the one or more antigenic sites can result in tropism of the AAV vectors herein to one or more cell types, one or more tissue types, or any combination thereof. As used herein, “tropism” refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest. In an aspect, modification of the one or more antigenic sites can result in AAV vectors herein that can exhibit tropism for one or more cell types and/or tissues throughout the body of a subject. In an aspect, modification of the one or more antigenic sites can result in AAV vectors herein that can exhibit tropism to one or more hematopoietic progenitor cells. In an aspect, modification of the one or more antigenic sites can result in AAV vectors herein that can exhibit tropism to one or more immune cell types. In an aspect, modification of the one or more antigenic sites can result in AAV vectors herein that can exhibit tropism to T-cells (CD4 T cells and/or CD8 T cells), B-cells, and/or natural killer (NK) cells. In an aspect, modification of the one or more antigenic sites can result in AAV vectors herein that can exhibit tropism to T cells and NK cells.

In an aspect, the one or more amino acid modifications (e.g., substitutions and/or deletions) within capsid protein variants herein, can be in one or more antigenic footprints identified by peptide epitope mapping and/or cryo-electron microscopy studies of AAV-antibody complexes containing AAV capsid proteins. In an aspect, the one or more antigenic sites herein that can be subject to one or more amino acid modifications can be common antigenic motifs (CAMs) as described in WO 2017/058892, which is incorporated herein by reference in its entirety.

In an aspect, the one or more antigenic sites herein that can be subject to one or more amino acid modifications can be in a variable region (VR) of an AAV capsid protein. An AAV capsid contains 60 copies (in total) of three VPs (VP1, VP2, VP3) that are encoded by the cap gene and have overlapping sequences. Each VP can contain an eight-stranded β-barrel motif (01B to 1) and/or an α-helix (αA) conserved in autonomous parvovirus capsids. Structurally variable regions (VRs) can occur in the surface loops that connect the β-strands, which cluster to produce local variations in the capsid surface. In an aspect, the one or more amino acid modifications herein that modify one or more antigenic sites in AAV capsid protein variants herein can be in VR-I, VR-II, VR-III, VR-IV, VR-V, VR-VI, VR-VII, VR-VI II, VR-IX, or any combination thereof. In an aspect, one or more antigenic sites can be in the HI loop of the AAV capsid protein variants herein.

In an aspect, AAV vectors herein can comprise (i) a AAV capsid protein variant disclosed herein, and (ii) a cargo nucleic acid encapsidated by the capsid protein. In an aspect, an AAV vector comprising an AAV capsid protein variant described herein can have a phenotype of: evading neutralizing antibodies; enhanced or maintained transduction efficiency; selective tropism to one or more cell and/or tissue types; and any combination thereof.

In an aspect, the AAV vectors disclosed herein can exhibit at least about 2-fold (for example, about 4-fold, about 5-fold, about 7-fold, about 10-fold, about 15-fold, about 16-fold, about 17-fold, about 18-fold, about 20-fold, about 25-fold, or about 30-fold, including all values and subranges that lie there between) higher transduction in an immune cell (e.g., a T cell, a NK cell) compared to parental AAV6. The disclosure provides Ark313, which demonstrated about 15-fold to about 18-fold higher transduction in immune cells (e.g., a T cell, a NK cell) compared to parental AAV6.

In an aspect, AAV capsid protein variants disclosed herein can include at least one or more amino acid substitutions wherein about 1 amino acid residue to about 50 amino acid residues (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50) can be substituted from the amino acid residues comprising an amino acid sequence of a naturally occurring capsid protein. In an aspect, AAV capsid protein variants herein can have about 7 amino acid residues substituted from the amino acid residues comprising an amino acid sequence of a naturally occurring capsid protein.

In an aspect, AAV capsid protein variants disclosed herein can have an amino acid sequence with about 85% (e.g., about 85%, 90%, 95%, 99%, 100%) similarity to a naturally occurring capsid protein. As used herein, “naturally occurring” or “wild-type” means existing in nature without modification by man. In an aspect, a naturally occurring capsid protein herein can be derived from a single species. Non-limiting examples of species that can be the origin of a naturally occurring capsid protein herein include those from a general organism such as a human, mouse, rat, guinea pig, dog, cat, horse, cow, pig, or non-human primate (e.g., monkey, chimpanzee, baboon, gorilla) bird, reptile, worm, fish, and the like. In an aspect, species that can be the origin of a naturally occurring capsid protein herein can be Mus Musculus (mouse).

In an aspect, AAV capsid protein variants having at least one amino acid substitution as disclosed herein can have an amino acid sequence with about 85% (e.g., about 85%, 90%, 95%, 99%, 100%) similarity to a naturally occurring capsid protein having an amino acid sequence referenced by GenBank Accession Numbers: NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579, and any combination thereof.

Methods of determining sequence similarity or identity between two or more amino acid sequences are known in the art. Sequence similarity or identity can be determined using standard techniques, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wl), the Best Fit sequence program described by Devereux et al., Nucl. Acid Res. 12, 387-395 (1984), or by inspection. Another suitable algorithm is the BLAST algorithm, described in Altschul et al., JMol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996). WU-BLAST-2 uses several search parameters, which are optionally set to the default values. The parameters are dynamic values and are established by the program itself depending upon the composition of the particular sequence and composition of the particular database against which the sequence of interest is being searched; however, the values can be adjusted to increase sensitivity. Further, an additional useful algorithm is gapped BLAST as reported by Altschul et al, (1997) Nucleic Acids Res. 25, 3389-3402. For purposes of the instant disclosure, unless otherwise indicated, percent identity is calculated using the Basic Local Alignment Search Tool (BLAST) available online at blast.ncbi.nlm.nih.gov/Blast.cgi. The skilled artisan will understand that other algorithms can be substituted as appropriate.

In an aspect, AAV capsid protein variants disclosed herein can have at least one amino acid substitution that can replace any seven amino acids in an AAV capsid protein from any one of the following serotypes: AAV1, AAV2, AAV3, AAV4, AAV5, AAV6, AAV7, AAV8, AAV9, AAVrh8, AAVrh10, AAV10, AAV11, AAV12, AAVrh32.22, bovine AAV, avian AAV and/or any other AAV now known or later identified. In an aspect, AAV capsid protein variants disclosed herein can have at least one amino acid substitution that can replace any seven amino acids in an AAV capsid protein from a serotype having a known tropism to one or more desired cell and/or tissue types. In an aspect, AAV capsid protein variants disclosed herein can have at least one amino acid substitution that can replace any seven amino acids in an AAV capsid protein from a serotype having a known tropism to one or more desired human cell and/or tissue types.

In an aspect, AAV capsid protein variants disclosed herein can have at least one amino acid substitution that can replace any seven amino acids in an AAV capsid protein from a serotype having tropism for immune cells (e.g., T cells, NK cells). In an aspect, AAV capsid protein variants disclosed herein can have at least one amino acid substitution that can replace any seven amino acids in an AAV capsid protein of any AAV serotype having tropism for T cells. In an aspect, AAV capsid protein variants disclosed herein can have at least one amino acid substitution that can replace any seven amino acids in an AAV capsid protein of any AAV serotype having tropism for NK cells.

In an aspect, AAV capsid protein variants herein or fragments thereof can have an amino acid sequence with about 85% (e.g., about 85%, 90%, 95%, 99%, 100%) similarity to a naturally occurring VP1 capsid protein or fragment thereof. In an aspect, capsid protein variants herein can comprise an amino acid substitution at one or more (e.g., 2, 3, 4, 5, 6, or 7) of amino acid residues 454-460 of AAV6 (VP1 numbering), in any combination, or the equivalent amino acid residues in AAV2, AAV3, AAV4, AAV5, AAV1, AAV7, AAV8, AAV9, AAV10, AAV11, AAV12, AAVrh8, AAVrh10, AAVrh32.33, bovine AAV or avian AAV.

In an aspect, capsid protein variants herein can have at least 90% (e.g., about 90%, 95%, 99%, 100%) sequence identity to the native sequence of the AAV6 capsid (SEQ ID NO:01).

In an aspect, capsid protein variants herein can have at least 90% (e.g., about 90%, 95%, 99%, 100%) sequence identity to a protein encoded by native nucleic acid sequence of the AAV6 (SEQ ID NO:02). In an aspect, capsid protein variants herein can comprise a substitution at one or more (e.g., 2, 3, 4, 5, 6, or 7) amino acid residues within a SEQ ID NO:01 (454-GSAQNKD-460 (VP1 numbering) on the capsid surface of AAV6 in any combination.

In an aspect, AAV vectors herein can comprise (i) a AAV6 capsid protein variant and (ii) a cargo nucleic acid encapsidated by the capsid protein. In an aspect, AAV vectors herein can comprise (i) a AAV6 capsid protein variant and (ii) a cargo nucleic acid encapsidated by the capsid protein wherein the capsid protein can comprise a peptide having the sequence X¹-X²-X³-X⁴-X⁵-X⁶-X⁷ (SEQ ID NO:544) at amino acids 454-460 (VP1 numbering) of a native AAV6 capsid protein, (SEQ ID NO:01), wherein the peptide does not occur in the native AAV6 capsid protein sequence.

In an aspect, AAV vectors herein can comprise an AAV6 capsid protein variant comprising a peptide having the sequence X¹-X²-X³-X⁴-X⁵-X⁶-X⁷ (SEQ ID NO:544) at amino acids 454-460 (VP1 numbering) of a native AAV6 capsid protein, (SEQ ID NO:01), wherein X¹ can be any amino acid other than G; X² can be any amino acid other than S; X³ can be any amino acid other than A; X⁴ can be any amino acid other than Q; X⁵ can be any amino acid other than N; X⁶ can be any amino acid other than K; and/or X⁷ can be any amino acid other than D. In an aspect, AAV vectors herein can comprise an AAV6 capsid protein variant comprising a peptide having the sequence X-X²-X³-X⁴-X⁵-X⁶-X⁷ (SEQ ID NO:543) at amino acids 454-460 (VP1 numbering) of a native AAV6 capsid protein, (SEQ ID NO:01), wherein X¹ can be any amino acid other than Y; X² can be any amino acid other than C; X³ can be any amino acid; X⁴ can be any amino acid; X⁵ can be any amino acid; X⁶ can be any amino acid; and/or X¹ can be any amino acid.

In an aspect, capsid protein variants herein can comprise a peptide wherein the amino acids corresponding to amino acid position 454-460 (VP1 numbering) of a native AAV6 capsid protein, (SEQ ID NO:01) can be substituted with amino acids corresponding to any one of SEQ ID NO:05-SEQ ID NO:545. Table 4 below provides amino acids corresponding to any one of SEQ ID NO:05-SEQ ID NO:545.

TABLE 4
Listing of AAV6 Capsid Variants and
Sequence Identifiers

| SEQ ID | AA |
|---|---|
| NO | sequence |
| 5 | VVNPAEG |
| 6 | VVNPADA |
| 7 | IVNPQVE |
| 8 | VVNPAEL |
| 9 | VVNPDDG |
| 10 | VVNPAET |
| 11 | VLNPAEG |
| 12 | IINPLPS |
| 13 | VVNPAEA |
| 14 | IVNPHSI |
| 15 | LVNPAEG |
| 16 | VVNPDEG |
| 17 | VVNPMQA |
| 18 | VVNPGEG |
| 19 | SDNPSWG |
| 20 | GTNPPCA |
| 21 | GGNPVEV |
| 22 | SLNPSGG |
| 23 | ASNPSGY |
| 24 | SANPSHA |
| 25 | RDNPSSG |
| 26 | TTNPIVS |
| 27 | APNPGSA |
| 28 | GTNPVGT |
| 29 | EGNPPTA |
| 30 | GSNPAEG |
| 31 | WENPGGG |
| 32 | TMNPTIA |
| 33 | LRNPMPL |
| 34 | VDNPAEC |
| 35 | GTNPLAT |
| 36 | NANPGEG |
| 37 | VPNPSEL |
| 38 | VFNPADG |
| 39 | SANPGWG |
| 40 | VVMPDVK |
| 41 | LLNREAT |
| 42 | ATHVHAS |
| 43 | VLRATEE |
| 44 | VIERADQ |
| 45 | IVNPGCG |
| 46 | VVNPDHT |
| 47 | KLLPVGE |
| 48 | TLMSSTA |
| 49 | TLLPSSS |
| 50 | RLVPNSD |
| 51 | VVNPYQA |
| 52 | LLMDKPS |
| 53 | KQEKVEG |
| 54 | PDHARST |
| 55 | RNEPSSA |
| 56 | QREERSS |
| 57 | VEVGDMQ |
| 58 | IVNADSL |
| 59 | EPTKVSR |
| 60 | GERRHCE |
| 61 | IVNPQVA |
| 62 | LPSESFS |
| 63 | PNANQQY |
| 64 | RGEMSAG |
| 65 | KLLPSLD |
| 66 | NASVNSA |
| 67 | TESGALS |
| 68 | TILPSVS |
| 69 | TTTSSVG |
| 70 | RGDSPES |
| 71 | TDKDGMS |
| 72 | SPSKYQR |
| 73 | IGERKAE |
| 74 | NTVDDAR |
| 75 | TFALKAE |
| 76 | QMVRQAE |
| 77 | TVLSRSD |
| 78 | TSEKGCA |
| 79 | ALWSEDV |
| 80 | SGSVSAS |
| 81 | AIDQGNP |
| 82 | TQHTAGS |
| 83 | AILKDKE |
| 84 | SLLPSQQ |
| 85 | LRAQDSE |
| 86 | QVEGRVG |
| 87 | NVERAGT |
| 88 | MGEHAYG |
| 89 | TVIQRSD |
| 90 | TLLPEQR |
| 91 | GSAQNND |
| 92 | GGDTGAR |
| 93 | AHNPGTG |
| 94 | ALRRLEE |
| 95 | LTNGGMT |
| 96 | SSVSSWP |
| 97 | KGTDCHV |
| 98 | SSNVQQA |
| 99 | SDLKVGQ |
| 100 | STLDNTS |
| 101 | DTMSRAY |
| 102 | VLNPSSQ |
| 103 | VLTRNVE |
| 104 | TFSSQQT |
| 105 | SLLSTSS |
| 106 | PTFNEIN |
| 107 | EGKKGQL |
| 108 | SSSSLVG |
| 109 | DTDSGSR |
| 110 | DGKHEWG |
| 111 | LSDISHN |
| 112 | REASGVG |
| 113 | VERESGG |
| 114 | EGATSCR |
| 115 | SSGSDLA |
| 116 | SSSADRD |
| 117 | TESKASN |
| 118 | VINPGVA |
| 119 | VLSKDSL |
| 120 | EGCKAAG |
| 121 | LKLETTV |
| 122 | GGSQSVA |
| 123 | TTPVNTT |
| 124 | RELEKQL |
| 125 | KVQLDQL |
| 126 | KVEGSQP |
| 127 | VTSTRSE |
| 128 | LWRADCT |
| 129 | VVRGTED |
| 130 | SAKVAPS |
| 131 | LDMASNL |
| 132 | ETRTSPY |
| 133 | TLLPSEL |
| 134 | TILQSNA |
| 135 | DLLGAGQ |
| 136 | ETRLKEG |
| 137 | SMSVPVG |
| 138 | SASPEVR |
| 139 | TREADGS |
| 140 | TVVKEGA |
| 141 | PDRSDVR |
| 142 | NTVPSQL |
| 143 | QAENKLG |
| 144 | PDGSRQA |
| 145 | SESGDVR |
| 146 | KDHTSQA |
| 147 | ASNVNVG |
| 148 | KLHNVVD |
| 149 | GEKLRAS |
| 150 | EDRARGG |
| 151 | KNGVCSE |
| 152 | FMSAGAE |
| 153 | HVGREVG |
| 154 | RFSADVD |
| 155 | SQLSTCH |
| 156 | SARLSTE |
| 157 | EVNVPIT |
| 158 | TFASEGS |
| 159 | GPVCPA |
| 160 | GVGRDGE |
| 161 | NSACHQS |
| 162 | VESRLGG |
| 163 | SLVKAAE |
| 164 | STSPDPL |
| 165 | STDAHAL |
| 166 | GNSLDGG |
| 167 | FDAPTSR |
| 168 | GDTKGLG |
| 169 | EGMPKVD |
| 170 | ERSPDSV |
| 171 | SVQREGG |
| 172 | SNGSVGT |
| 173 | PAVLQND |
| 174 | HTTATAD |
| 175 | LDSASAR |
| 176 | ALTVDRG |
| 177 | SVLTSHG |
| 178 | QQASNAG |
| 179 | IKNIGTA |
| 180 | NSRDCDS |
| 181 | SLITSGV |
| 182 | TFTSNLE |
| 183 | KADVSCG |
| 184 | PERTSQA |
| 185 | STHGLAD |
| 186 | ATLAWET |
| 187 | VSSSVLE |
| 188 | PSDFTRD |
| 189 | ARSEQPG |
| 190 | TLLPSHG |
| 191 | EEKVRVG |
| 192 | PEANKQQ |
| 193 | VQDAKAG |
| 194 | MESGGMG |
| 195 | KSSPEGG |
| 196 | GLSSTQS |
| 197 | QSSNEPR |
| 198 | TEADDRM |
| 199 | SHVLEGS |
| 200 | ADDGTK |
| 201 | REESFGP |
| 202 | EPDVRVR |
| 203 | ETSASGR |
| 204 | VTLTLPL |
| 205 | EVSARSS |
| 206 | LLKADHT |
| 207 | AVMRPEA |
| 208 | LISCENE |
| 209 | CEKSIPT |
| 210 | GERNCPC |
| 211 | AGDGGSG |
| 212 | LMSTSLE |
| 213 | SPTAEAQ |
| 214 | SHVIGEC |
| 215 | HGAYIPE |
| 216 | QAQDAGT |
| 217 | EVPTKSL |
| 218 | SSGSGLG |
| 219 | THQKSEE |
| 220 | HSDVCKG |
| 221 | SSDVYAG |
| 222 | GVNAGAT |
| 223 | TTNVRAE |
| 224 | DSQKMDL |
| 225 | VDIRSAS |
| 226 | TANLSQL |
| 227 | GKDGGVA |
| 228 | LKNQSCD |
| 229 | NVTLNSS |
| 230 | GSQTGTE |
| 231 | DTAKSGQ |
| 232 | VEVGGAA |
| 233 | SANIGET |
| 234 | MDGSSVR |
| 235 | ADLSGRE |
| 236 | RTESASA |
| 237 | LLNGVIP |
| 238 | VLDGRSA |
| 239 | AAEGSGK |
| 240 | VDVKRSG |
| 241 | VGRVESE |
| 242 | CNMTLEA |
| 243 | RDSTIAE |
| 244 | EVRGCTE |
| 245 | QDGVKWT |
| 246 | TILIRED |
| 247 | SSVEEGR |
| 248 | TLTSQSV |
| 249 | IDCTDTE |
| 250 | QVDAARA |
| 251 | DAGCKAG |
| 252 | TTGACHE |
| 253 | LRDGAGE |
| 254 | SLGTASG |
| 255 | NAIASPV |
| 256 | SRDTVPA |
| 257 | GNSLSPS |
| 258 | NLDSWSG |
| 259 | EGALKGH |
| 260 | IRPDSCD |
| 261 | NADVLDG |
| 262 | QDKTCSG |
| 263 | AQGSYGE |
| 264 | EERKEPK |
| 265 | SLSSSLE |
| 266 | TETGRQV |
| 267 | NNAGTDG |
| 268 | ETTTKSN |
| 269 | QQAPSPG |
| 270 | HTGGDAG |
| 271 | TISTGSE |
| 272 | KAVGSEE |
| 273 | RESRSVE |
| 274 | REGEAGP |
| 275 | PNSSTSS |
| 276 | VTLPDSR |
| 277 | LPDRGWA |
| 278 | GTVMNID |
| 279 | STNPLSV |
| 280 | VVAS |
| 281 | VLTKDPL |
| 282 | ALTSAAN |
| 283 | VVKSRAK |
| 284 | MHSSNGT |
| 285 | TSVKNCP |
| 286 | VELKTGV |
| 287 | TPTQGGD |
| 288 | QNGGCVP |
| 289 | GSGRDGQ |
| 290 | KIVPSHQ |
| 291 | ANTGHST |
| 292 | KLVASPS |
| 293 | DTGIRDS |
| 294 | RGEGTHV |
| 295 | ASDAFRE |
| 296 | DSCGTSH |
| 297 | AHTEFNG |
| 298 | PGGADRG |
| 299 | QSCAA |
| 300 | GLNECVE |
| 301 | LMSAQTT |
| 302 | GNMVGLS |
| 303 | QKEFGMD |
| 304 | GGTVGDQ |
| 305 | STVREGR |
| 306 | RFSPEPQ |
| 307 | NSGVEST |
| 308 | GAKMDSD |
| 309 | RETKAEV |
| 310 | LGSSLDC |
| 311 | MSHTEGG |
| 312 | REAREAH |
| 313 | STDSAHR |
| 314 | NGALGAG |
| 315 | TEVGRGC |
| 316 | GETNGAT |
| 317 | NTSPASL |
| 318 | QLSAEGV |
| 319 | QATCDSS |
| 320 | KDTCHDS |
| 321 | SNERACG |
| 322 | PENMSGP |
| 323 | VSIAEDV |
| 324 | KEGVGPG |
| 325 | SPSDKVL |
| 326 | STALVTT |
| 327 | GSHRQEE |
| 328 | GVALKGE |
| 329 | TADMCPR |
| 330 | KTVLDQT |
| 331 | GAGVAAS |
| 332 | QERLNSV |
| 333 | NGDKRQE |
| 334 | GGDQGAQ |
| 335 | STDRNVM |
| 336 | SDRLTVG |
| 337 | SDLGKGE |
| 338 | GLEGNSS |
| 339 | GQDSDRN |
| 340 | KPDGREA |
| 341 | DPSGNGA |
| 342 | PSEGQGG |
| 343 | SGTVMQG |
| 344 | PTASVST |
| 345 | PISESSN |
| 346 | SSDGLNN |
| 347 | SEAQASV |
| 348 | SGEPRGS |
| 349 | SSESKCS |
| 350 | ASSRETT |
| 351 | SSEPGVR |
| 352 | ESMSKPW |
| 353 | STDLRQH |
| 354 | EGVGREG |
| 355 | KSGKEVE |
| 356 | GWNKGTE |
| 357 | IPLCARV |
| 358 | TALNAEK |
| 359 | SAGLCGQ |
| 360 | VVNPKVE |
| 361 | LLLPVGE |
| 362 | VVRVEGA |
| 363 | VLSAELV |
| 364 | VLTQQCT |
| 365 | VVGIDSA |
| 366 | VLGQDAS |
| 367 | ALLPQQG |
| 368 | VAEARGG |
| 369 | VLASSHT |
| 370 | EAGACSR |
| 371 | MFKTTEG |
| 372 | GLFPGSE |
| 373 | RLLPETA |
| 374 | GLVKTAS |
| 375 | ADCEENR |
| 376 | DGSGSEE |
| 377 | EERSKAH |
| 378 | PGQTLQS |
| 379 | TLISREG |
| 380 | RDPGPAA |
| 381 | GNAINVD |
| 382 | TLSKTVS |
| 383 | ERSRECG |
| 384 | TGSGGAS |
| 385 | EESARKL |
| 386 | ASTNIDY |
| 387 | GLENEMT |
| 388 | SADGGSG |
| 389 | SSGECAR |
| 390 | TERVSHH |
| 391 | IEGVGGG |
| 392 | SSTLTGA |
| 393 | FSTQSAQ |
| 394 | GDSEGWQ |
| 395 | NLVDATP |
| 396 | DRDGETG |
| 397 | ANQRPEA |
| 398 | GGAANTG |
| 399 | EGGVQER |
| 400 | NTNKATE |
| 401 | EARAEAR |
| 402 | SLESDCG |
| 403 | GDARNRE |
| 404 | TSHQSAE |
| 405 | GVSGSPG |
| 406 | VTSTEPR |
| 407 | SQILGSA |
| 408 | LGETNAG |
| 409 | ESRLSAS |
| 410 | STATTVG |
| 411 | MLHQEAA |
| 412 | GGGEAGC |
| 413 | ATAVQCN |
| 414 | TDSANPA |
| 415 | DASTTTG |
| 416 | SDKVNAS |
| 417 | EGTQSGS |
| 418 | RVEQGVG |
| 419 | SASGLEG |
| 420 | GHGQQSD |
| 421 | ASLGPQG |
| 422 | SSDSLRS |
| 423 | ESVKTAQ |
| 424 | KAELGTA |
| 425 | GDTRPNC |
| 426 | AQELGHG |
| 427 | DGLSRST |
| 428 | EAVH |
| 429 | STAPDPR |
| 430 | SSSEINP |
| 431 | KRSDVPT |
| 432 | SSSSRQL |
| 433 | LFTTSRR |
| 434 | PKNPEEV |
| 435 | VGNPALT |
| 436 | IVARAGD |
| 437 | LLSKEQI |
| 438 | LAPRDRE |
| 439 | VVMTESR |
| 440 | VLQQASA |
| 441 | VAERERG |
| 442 | TSSICAE |
| 443 | EYVRPGE |
| 444 | SDGFGVL |
| 445 | CQIPEGV |
| 446 | TLLTVPC |
| 447 | EERRSSP |
| 448 | HTTQTVH |
| 449 | VDAYEVQ |
| 450 | SLLPRES |
| 451 | SISVRES |
| 452 | LGVGVVG |
| 453 | MTMTTSA |
| 454 | ERLQKAE |
| 455 | DSSTRTL |
| 456 | LTNGGCH |
| 457 | APTQTVS |
| 458 | NREDTTH |
| 459 | TFNIAPS |
| 460 | VDPAGQR |
| 461 | AQHLNGE |
| 462 | TKGSETW |
| 463 | EALLKPY |
| 464 | QQSCHGT |
| 465 | EGTVGAR |
| 466 | HQDLCDK |
| 467 | IDTGAHR |
| 468 | QEKGSSS |
| 469 | GDRGDSS |
| 470 | TEGKCER |
| 471 | DSAGGGA |
| 472 | QGGPSSG |
| 473 | GSNTCVG |
| 474 | TGPSMGG |
| 475 | AHDRAGA |
| 476 | GEVHRAS |
| 477 | GGNVGEV |
| 478 | DRTSDIA |
| 479 | LGMENPP |
| 480 | GDSGAMQ |
| 481 | REAGLDG |
| 482 | MASNGYD |
| 483 | SVSAWSE |
| 484 | GQGEGSG |
| 485 | KAGLAAD |
| 486 | VDSARTS |
| 487 | CQVDGSQ |
| 488 | PTVVGQG |
| 489 | TAKLVPE |
| 490 | STMKEES |
| 491 | LHDVDSS |
| 492 | GNVAVER |
| 493 | ATSDPGS |
| 494 | VSDNRAG |
| 495 | AGQLDSG |
| 496 | KVDAANG |
| 497 | KLSPSQG |
| 498 | DPRKDCT |
| 499 | SPTSIGT |
| 500 | MSEHVNG |
| 501 | LEGHGAG |
| 502 | VEVNDRN |
| 503 | SASLTCG |
| 504 | VDTMRHG |
| 505 | TSGQLQS |
| 506 | LNALSAT |
| 507 | TINVSPE |
| 508 | NLSSGSD |
| 509 | ESGPRGS |
| 510 | VMNKAEE |
| 511 | DSSPLGR |
| 512 | AREGVPG |
| 513 | GQERATC |
| 514 | ESAAGVR |
| 515 | SGVAGSA |
| 516 | LERSQEG |
| 517 | SSGHGVG |
| 518 | SSSQEHR |
| 519 | ASDSVGS |
| 520 | ADHTASH |
| 521 | GEEGRGR |
| 522 | QASAEVR |
| 523 | PESGGLG |
| 524 | KSSPESL |
| 525 | LNATAAT |
| 526 | LEKDGWE |
| 527 | DPTLPRQ |
| 528 | DAAGKAH |
| 529 | KFSYACF |
| 530 | VFKQRFS |
| 531 | GLSCAGL |
| 532 | MRYWESR |
| 533 | STQVSDA |
| 534 | MSCETAT |
| 535 | HTWNGLG |
| 536 | EASEHKY |
| 537 | VTRQTCD |
| 538 | VLVTLGS |
| 539 | LANG |
| 540 | LIDASFN |
| 541 | VAERKSV |
| 545 | GSAQNKD |

In an aspect, capsid protein variants herein can comprise a peptide wherein the amino acids corresponding to amino acid position 454-460 (VP1 numbering) of a native AAV6 capsid protein, (SEQ ID NO:01) can be substituted with amino acids corresponding to VVNPAEG (SEQ ID NO:05).

In an aspect, capsid protein variants herein can share at least about 85% (e.g., about 85%, 90%, 95%, 99%, or 100%) amino acid sequence similarity with any one of the sequences set forth in SEQ ID NO:01 and SEQ ID NO:02. In an aspect, capsid protein variants herein can comprise SEQ ID NO: 2 or a species equivalent thereof. In an aspect, capsid protein variants herein can be encoded from a polynucleotide sharing at least about 85% (e.g., about 85%, 90%, 95%, 99%, or 100%) nucleic acid sequence similarity with any one of the sequences set forth in SEQ ID NO:03 and SEQ ID NO:04. In an aspect, capsid protein variants herein can be encoded from a polynucleotide comprising SEQ ID NO:04 or a species equivalent thereof. Amino acid sequences of native AAV6 capsid protein (SEQ ID NO:01) and SEQ ID NO:02 (Ark313) are provided below. Nucleic acid sequences of native AAV6 capsid protein (SEQ ID NO:03) and SEQ ID NO:04 (Ark313) are provided below.

In an aspect, a disclosed wild-type AAV9 capsid protein can comprise the sequence set forth below:

(SEQ ID NO: 01)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAVFQAKKRVLEPFGLVEEGAKTAPGKKRPVE
QSPQEPDSSSGIGKTGQQPAKKRLNFGQTGDSESVPDPQPLGEPPATPA
AVGPTTMASGGGAPMADNNEGADGVGNASGNWHCDSTWLGDRVITTSTR
TWALPTYNNHLYKQISSASTGASNDNHYFGYSTPWGYFDFNRFHCHFSP
RDWQRLINNNWGFRPKRLNFKLFNIQVKEVTTNDGVTTIANNLTSTVQV
FSDSEYQLPYVLGSAHQGCLPPFPADVFMIPQYGYLTLNNGSQAVGRSS
FYCLEYFPSQMLRTGNNFTFSYTFEDVPFHSSYAHSQSLDRLMNPLIDQ
YLYYLNRTQNQSGSAQNKDLLFSRGSPAGMSVQPKNWLPGPCYRQQRVS
KTKTDNNNSNFTWTGASKYNLNGRESIINPGTAMASHKDDKDKFFPMSG
VMIFGKESAGASNTALDNVMITDEEEIKATNPVATERFGTVAVNLQSSS
TDPATGDVHVMGALPGMVWQDRDVYLQGPIWAKIPHTDGHFHPSPLMGG
FGLKHPPPQILIKNTPVPANPPAEFSATKFASFITQYSTGQVSVEIEWE
LQKENSKRWNPEVQYTSNYAKSANVDFTVDNNGLYTEPRPIGTRYLTRP
L.

In an aspect, a disclosed Ark313 AAV9 capsid protein can comprise the sequence set forth below:

(SEQ ID NO: 02)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAVFQAKKRVLEPFGLVEEGAKTAPGKKRPVE
QSPQEPDSSSGIGKTGQQPAKKRLNFGQTGDSESVPDPQPLGEPPATPA
AVGPTTMASGGGAPMADNNEGADGVGNASGNWHCDSTWLGDRVITTSTR
TWALPTYNNHLYKQISSASTGASNDNHYFGYSTPWGYFDFNRFHCHFSP
RDWQRLINNNWGFRPKRLNFKLFNIQVKEVTTNDGVTTIANNLTSTVQV
FSDSEYQLPYVLGSAHQGCLPPFPADVFMIPQYGYLTLNNGSQAVGRSS
FYCLEYFPSQMLRTGNNFTFSYTFEDVPFHSSYAHSQSLDRLMNPLIDQ
YLYYLNRTQNQSVVNPAEGLLFSRGSPAGMSVQPKNWLPGPCYRQQRVS
KTKTDNNNSNFTWTGASKYNLNGRESIINPGTAMASHKDDKDKFFPMSG
VMIFGKESAGASNTALDNVMITDEEEIKATNPVATERFGTVAVNLQSSS
TDPATGDVHVMGALPGMVWQDRDVYLQGPIWAKIPHTDGHFHPSPLMGG
FGLKHPPPQILIKNTPVPANPPAEFSATKFASFITQYSTGQVSVEIEWE
LQKENSKRWNPEVQYTSNYAKSANVDFTVDNNGLYTEPRPIGTRYLTRP
L.

In an aspect, a disclosed wild-type AAV9 capsid protein can encoded by the sequence set forth below:

(SEQ ID NO: 03)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCTGG
AGGGGTGGAGTCGTGACGTGAATTACGTCATAGGGTTAGGGAGGTCCTG
TATTAGAGGTCACGTGAGTGTTTTGCGACATTTTGCGACACCATGTGGT
CACGCTGGGTATTTAAGCCCGAGTGAGCACGCAGGGTCTCCATTTTGAA
GCGGGAGGTTTGAACGCGCAGCGCCATGCCGGGGTTTTACGAGATTGTG
ATTAAGGTCCCCAGCGACCTTGACGAGCATCTGCCCGGCATTTCTGACA
GCTTTGTGAACTGGGTGGCCGAGAAGGAATGGGAGTTGCCGCCAGATTC
TGACATGGATCTGAATCTGATTGAGCAGGCACCCCTGACCGTGGCCGAG
AAGCTGCAGCGCGACTTCCTGGTCCAGTGGCGCCGCGTGAGTAAGGCCC
CGGAGGCCCTCTTCTTTGTTCAGTTCGAGAAGGGCGAGTCCTACTTCCA
CCTCCATATTCTGGTGGAGACCACGGGGGTCAAATCCATGGTGCTGGGC
CGCTTCCTGAGTCAGATTAGGGACAAGCTGGTGCAGACCATCTACCGCG
GGATCGAGCCGACCCTGCCCAACTGGTTCGCGGTGACCAAGACGCGTAA
TGGCGCCGGAGGGGGGAACAAGGTGGTGGACGAGTGCTACATCCCCAAC
TACCTCCTGCCCAAGACTCAGCCCGAGCTGCAGTGGGCGTGGACTAACA
TGGAGGAGTATATAAGCGCGTGTTTAAACCTGGCCGAGCGCAAACGGCT
CGTGGCGCACGACCTGACCCACGTCAGCCAGACCCAGGAGCAGAACAAG
GAGAATCTGAACCCCAATTCTGACGCGCCTGTCATCCGGTCAAAAACCT
CCGCACGCTACATGGAGCTGGTCGGGTGGCTGGTGGACCGGGGCATCAC
CTCCGAGAAGCAGTGGATCCAGGAGGACCAGGCCTCGTACATCTCCTTC
AACGCCGCCTCCAACTCGCGGTCCCAGATCAAGGCCGCTCTGGACAATG
CCGGCAAGATCATGGCGCTGACCAAATCCGCGCCCGACTACCTGGTAGG
CCCCGCTCCGCCCGCCGACATTAAAACCAACCGCATTTACCGCATCCTG
GAGCTGAACGGCTACGACCCTGCCTACGCCGGCTCCGTCTTTCTCGGCT
GGGCCCAGAAAAGGTTCGGAAAACGCAACACCATCTGGCTGTTTGGGCC
GGCCACCACGGGCAAGACCAACATCGCGGAAGCCATCGCCCACGCCGTG
CCCTTCTACGGCTGCGTCAACTGGACCAATGAGAACTTTCCCTTCAACG
ATTGCGTCGACAAGATGGTGATCTGGTGGGAGGAGGGCAAGATGACGGC
CAAGGTCGTGGAGTCCGCCAAGGCCATTCTCGGCGGCAGCAAGGTGCGC
GTGGACCAAAAGTGCAAGTCGTCCGCCCAGATCGATCCCACCCCCGTGA
TCGTCACCTCCAACACCAACATGTGCGCCGTGATTGACGGGAACAGCAC
CACCTTCGAGCACCAGCAGCCGTTGCAGGACCGGATGTTCAAATTTGAA
CTCACCCGCCGTCTGGAGCATGACTTTGGCAAGGTGACAAAGCAGGAAG
TCAAAGAGTTCTTCCGCTGGGCGCAGGATCACGTGACCGAGGTGGCGCA
TGAGTTCTACGTCAGAAAGGGTGGAGCCAACAAGAGACCCGCCCCCGAT
GACGCGGATAAAAGCGAGCCCAAGCGGGCCTGCCCCTCAGTCGCGGATC
CATCGACGTCAGACGCGGAAGGAGCTCCGGTGGACTTTGCCGACAGGTA
CCAAAACAAATGTTCTCGTCACGCGGGCATGCTTCAGATGCTGTTTCCC
TGCAAAACATGCGAGAGAATGAATCAGAATTTCAACATTTGCTTCACGC
ACGGGACCAGAGACTGTTCAGAATGTTTCCCCGGCGTGTCAGAATCTCA
ACCGGTCGTCAGAAAGAGGACGTATCGGAAACTCTGTGCCATTCATCAT
CTGCTGGGGCGGGCTCCCGAGATTGCTTGCTCGGCCTGCGATCTGGTCA
ACGTGGATCTGGATGACTGTGTTTCTGAGCAATAAATGACTTAAACCAG
GTATGGCTGCCGATGGTTATCTTCCAGATTGGCTCGAGGACAACCTCTC
TGAGGGCATTCGCGAGTGGTGGGACTTGAAACCTGGAGCCCCGAAACCC
AAAGCCAACCAGCAAAAGCAGGACGACGGCCGGGGTCTGGTGCTTCCTG
GCTACAAGTACCTCGGACCCTTCAACGGACTCGACAAGGGGGAGCCCGT
CAACGCGGCGGATGCAGCGGCCCTCGAGCACGACAAGGCCTACGACCAG
CAGCTCAAAGCGGGTGACAATCCGTACCTGCGGTATAACCACGCCGACG
CCGAGTTTCAGGAGCGTCTGCAAGAAGATACGTCTTTTGGGGGCAACCT
CGGGCGAGCAGTCTTCCAGGCCAAGAAGAGGGTTCTCGAACCTTTTGGT
CTGGTTGAGGAAGGTGCTAAGACGGCTCCTGGAAAGAAACGTCCGGTAG
AGCAGTCGCCACAAGAGCCAGACTCCTCCTCGGGCATTGGCAAGACAGG
CCAGCAGCCCGCTAAAAAGAGACTCAATTTTGGTCAGACTGGCGACTCA
GAGTCAGTCCCCGACCCACAACCTCTCGGAGAACCTCCAGCAACCCCCG
CTGCTGTGGGACCTACTACAATGGCTTCAGGCGGTGGCGCACCAATGGC
AGACAATAACGAAGGCGCCGACGGAGTGGGTAATGCCTCAGGAAATTGG
CATTGCGATTCCACATGGCTGGGCGACAGAGTCATCACCACCAGCACCC
GAACATGGGCCTTGCCCACCTATAACAACCACCTCTACAAGCAAATCTC
CAGTGCTTCAACGGGGGCCAGCAACGACAACCACTACTTCGGCTACAGC
ACCCCCTGGGGGTATTTTGATTTCAACAGATTCCACTGCCATTTCTCAC
CACGTGACTGGCAGCGACTCATCAACAACAATTGGGGATTCCGGCCCAA
GAGACTCAACTTCAAGCTCTTCAACATCCAAGTCAAGGAGGTCACGACG
AATGATGGCGTCACGACCATCGCTAATAACCTTACCAGCACGGTTCAAG
TCTTCTCGGACTCGGAGTACCAGTTGCCGTACGTCCTCGGCTCTGCGCA
CCAGGGCTGCCTCCCTCCGTTCCCGGCGGACGTGTTCATGATTCCGCAG
TACGGCTACCTAACGCTCAACAATGGCAGCCAGGCAGTGGGACGGTCAT
CCTTTTACTGCCTGGAATATTTCCCATCGCAGATGCTGAGAACGGGCAA
TAACTTTACCTTCAGCTACACCTTCGAGGACGTGCCTTTCCACAGCAGC
TACGCGCACAGCCAGAGCCTGGACCGGCTGATGAATCCTCTCATCGACC
AGTACCTGTATTACCTGAACAGAACTCAGAATCAGTCCGGAAGTGCCCA
AAACAAGGACTTGCTGTTTAGCCGGGGGTCTCCAGCTGGCATGTCTGTT
CAGCCCAAAAACTGGCTACCTGGACCCTGTTACCGGCAGCAGCGCGTTT
CTAAAACAAAAACAGACAACAACAACAGCAACTTTACCTGGACTGGTGC
TTCAAAATATAACCTTAATGGGCGTGAATCTATAATCAACCCTGGCACT
GCTATGGCCTCACACAAAGACGACAAAGACAAGTTCTTTCCCATGAGCG
GTGTCATGATTTTTGGAAAGGAGAGCGCCGGAGCTTCAAACACTGCATT
GGACAATGTCATGATCACAGACGAAGAGGAAATCAAAGCCACTAACCCC
GTGGCCACCGAAAGATTTGGGACTGTGGCAGTCAATCTCCAGAGCAGCA
GCACAGACCCTGCGACCGGAGATGTGCATGTTATGGGAGCCTTACCTGG
AATGGTGTGGCAAGACAGAGACGTATACCTGCAGGGTCCTATTTGGGCC
AAAATTCCTCACACGGATGGACACTTTCACCCGTCTCCTCTCATGGGCG
GCTTTGGACTTAAGCACCCGCCTCCTCAGATCCTCATCAAAAACACGCC
TGTTCCTGCGAATCCTCCGGCAGAGTTTTCGGCTACAAAGTTTGCTTCA
TTCATCACCCAGTATTCCACAGGACAAGTGAGCGTGGAGATTGAATGGG
AGCTGCAGAAAGAAAACAGCAAACGCTGGAATCCCGAAGTGCAGTATAC
ATCTAACTATGCAAAATCTGCCAACGTTGATTTCACTGTGGACAACAAT
GGACTTTATACTGAGCCTCGCCCCATTGGCACCCGTTACCTCACCCGTC
CCCTGTAATTGTGTGTTAATCAATAAACCGGTTAATTCGTGTCAGTTGA
ACTTTGGTCTCATGTCGTTATTATCTTATCTGGTCACCATAGCAACCGG
TTACACATTAACTGCTTAGTTGCGCTTCGCGAATACCCCTAGTGATGGA
GTTGCCCACTCCCTCTATGCGCGCTCGCTCGCTCGGTGGGGCCGGCAGA
GCAGAGCTCTGCCGTCTGCGGACCTTTGGTCCGCAGGCCCCACCGAGCG
AGCGAGCGCGCATAGAGGGAGTGGGCAA.

In an aspect, a disclosed Ark313 AAV9 capsid protein can encoded by the sequence set forth below:

(SEQ ID NO: 04)
ATGGCTGCCGATGGTTATCTTCCAGATTGGCTCGAGGACAACCTCTCTG
AGGGCATTCGCGAGTGGTGGGACTTGAAACCTGGAGCCCCGAAACCCAA
AGCCAACCAGCAAAAGCAGGACGACGGCCGGGGTCTGGTGCTTCCTGGC
TACAAGTACCTCGGACCCTTCAACGGACTCGACAAGGGGGAGCCCGTCA
ACGCGGCGGATGCAGCGGCCCTCGAGCACGACAAGGCCTACGACCAGCA
GCTCAAAGCGGGTGACAATCCGTACCTGCGGTATAACCACGCCGACGCC
GAGTTTCAGGAGCGTCTGCAAGAAGATACGTCTTTTGGGGGCAACCTCG
GGCGAGCAGTCTTCCAGGCCAAGAAGAGGGTTCTCGAACCTTTTGGTCT
GGTTGAGGAAGGTGCTAAGACGGCTCCTGGAAAGAAACGTCCGGTAGAG
CAGTCGCCACAAGAGCCAGACTCCTCCTCGGGCATTGGCAAGACAGGCC
AGCAGCCCGCTAAAAAGAGACTCAATTTTGGTCAGACTGGCGACTCAGA
GTCAGTCCCCGACCCACAACCTCTCGGAGAACCTCCAGCAACCCCCGCT
GCTGTGGGACCTACTACAATGGCTTCAGGCGGTGGCGCACCAATGGCAG
ACAATAACGAAGGCGCCGACGGAGTGGGTAATGCCTCAGGAAATTGGCA
TTGCGATTCCACATGGCTGGGCGACAGAGTCATCACCACCAGCACCCGA
ACATGGGCCTTGCCCACCTATAACAACCACCTCTACAAGCAAATCTCCA
GTGCTTCAACGGGGGCCAGCAACGACAACCACTACTTCGGCTACAGCAC
CCCCTGGGGGTATTTTGATTTCAACAGATTCCACTGCCATTTCTCACCA
CGTGACTGGCAGCGACTCATCAACAACAATTGGGGATTCCGGCCCAAGA
GACTCAACTTCAAGCTCTTCAACATCCAAGTCAAGGAGGTCACGACGAA
TGATGGCGTCACGACCATCGCTAATAACCTTACCAGCACGGTTCAAGTC
TTCTCGGACTCGGAGTACCAGTTGCCGTACGTCCTCGGCTCTGCGCACC
AGGGCTGCCTCCCTCCGTTCCCGGCGGACGTGTTCATGATTCCGCAGTA
CGGCTACCTAACGCTCAACAATGGCAGCCAGGCAGTGGGACGGTCATCC
TTTTACTGCCTGGAATATTTCCCATCGCAGATGCTGAGAACGGGCAATA
ACTTTACCTTCAGCTACACCTTCGAGGACGTGCCTTTCCACAGCAGCTA
CGCGCACAGCCAGAGCCTGGACCGGCTGATGAATCCTCTCATCGACCAG
TACCTGTATTACCTGAACAGAACTCAGAATCAGTCCGTGGTCAACCCGG
CCGAGGGCTTGCTGTTTAGCCGGGGGTCTCCAGCTGGCATGTCTGTTCA
GCCCAAAAACTGGCTACCTGGACCCTGTTACCGGCAGCAGCGCGTTTCT
AAAACAAAAACAGACAACAACAACAGCAACTTTACCTGGACTGGTGCTT
CAAAATATAACCTTAATGGGCGTGAATCTATAATCAACCCTGGCACTGC
TATGGCCTCACACAAAGACGACAAAGACAAGTTCTTTCCCATGAGCGGT
GTCATGATTTTTGGAAAGGAGAGCGCCGGAGCTTCAAACACTGCATTGG
ACAATGTCATGATCACAGACGAAGAGGAAATCAAAGCCACTAACCCCGT
GGCCACCGAAAGATTTGGGACTGTGGCAGTCAATCTCCAGAGCAGCAGC
ACAGACCCTGCGACCGGAGATGTGCATGTTATGGGAGCCTTACCTGGAA
TGGTGTGGCAAGACAGAGACGTATACCTGCAGGGTCCTATTTGGGCCAA
AATTCCTCACACGGATGGACACTTTCACCCGTCTCCTCTCATGGGCGGC
TTTGGACTGAAGCACCCGCCTCCTCAGATCCTCATCAAAAACACGCCTG
TTCCTGCGAATCCTCCGGCAGAGTTTTCGGCTACAAAGTTTGCTTCATT
CATCACCCAGTATTCCACAGGACAAGTGAGCGTGGAGATTGAATGGGAG
CTGCAGAAAGAAAACAGCAAACGCTGGAATCCCGAAGTGCAGTATACAT
CTAACTATGCAAAATCTGCCAACGTTGATTTCACTGTGGACAACAATGG
ACTTTATACTGAGCCTCGCCCCATTGGCACCCGTTACCTCACCCGTCCC
CTG.

In an aspect, wherein any amino acid residue identified as X¹ through X⁷ is not substituted, the amino acid residue at the unsubstituted position can be the wild type amino acid residue of the reference amino acid sequence (e.g., wild-type AAV6 (SEQ ID NO:01)). In an aspect, capsid protein variants herein can have an amino acid substitution at residues G454, S455, A456, Q457, N458, K459, and/or D460 of SEQ ID NO:01 (AAV6 capsid protein; VP1 numbering) in any combination. In an aspect, capsid protein variants herein can have one or more of the following amino acid substitutions of SEQ ID NO:01 (AAV6 capsid protein; VP1 numbering) in any combination: G454V, S455V, A456N, Q457P, N458A, K459E, and/or D460G.

In an aspect, capsid protein variants of the present disclosure can be produced by modifying the capsid protein of any AAV capsid protein now known or later discovered using the methodology described herein. Further, the AAV capsid protein that is to be modified according to the present disclosure can be a naturally occurring AAV capsid protein (e.g., an AAV2, AAV3a or 3b, AAV4, AAV5, AAV8, AAV9, AAV10 or AAV11 capsid protein or any of the AAV shown in Table 1) but is not so limited. Those skilled in the art will understand that a variety of manipulations to the AAV capsid proteins are known in the art and the invention is not limited to modifications of naturally occurring AAV capsid proteins. For example, the capsid protein to be modified can already have one or more alterations as compared with naturally occurring AAV (e.g., is derived from a naturally occurring AAV capsid protein, e.g., AAV2, AAV3a, AAV3b, AAV4, AAV5, AAV6, AAV7, AAV8, AAV9, AAV10, AAV11, AAV12 or any other AAV now known or later discovered). Such AAV capsid proteins are also within the scope of the present disclosure.

In an aspect, disclosed herein are virus capsids which can have one or more of any of the capsid protein variants disclosed herein. In an aspect, a virus capsid herein can be a parvovirus capsid, which can further be an autonomous parvovirus capsid or a dependovirus capsid. Optionally, a virus capsid herein can be an AAV capsid. In an aspect, AAV capsids of the present disclosure can be an AAV1, AAV2, AAV3a, AAV3b, AAV4, AAV5, AAV6, AAV7, AAV8, AAV9, AAV10, AAV11, AAV12, AAVrh8, AAVrh10, AAVrh32.33, bovine AAV capsid, avian AAV capsid and/or any other AAV now known or later identified.

In an aspect, modified virus capsids herein can be used as capsid vehicles. In an aspect, molecules can be packaged by the modified virus capsids herein and transferred into a cell wherein the molecules can include heterologous DNA, RNA, polypeptides, small organic molecules, metals, or combinations of the same. Heterologous molecules are defined herein as those that are not naturally found in an AAV infection, e.g., those not encoded by a wild-type AAV genome. Further, therapeutically useful molecules for use herein can be associated with the outside of the chimeric virus capsid for transfer of the molecules into one or more host target cells. Such associated molecules can include DNA, RNA, small organic molecules, metals, carbohydrates, lipids and/or polypeptides. In an aspect, a therapeutically useful molecule herein can be covalently linked (i.e., conjugated or chemically coupled) to a capsid proteins. Methods of covalently linking molecules are known by those skilled in the art.

In an aspect, modified virus capsids herein can be used in raising antibodies against the capsid protein variants disclosed herein. As a further alternative, an exogenous amino acid sequence can be inserted into the modified virus capsid for antigen presentation to a cell, e.g., for administration to a subject to produce an immune response to the exogenous amino acid sequence.

In an aspect, modified virus capsids herein can be a targeted virus capsid, comprising a targeting sequence (e.g., substituted or inserted in the viral capsid) that can direct the virus capsid to interact with cell-surface molecules present on desired target tissue(s) (see, e.g., international patent publication WO 00/28004 and Hauck et al. (2003) J. Virology, 77:2768-2774); Shi et al. (2006) Human Gene Ther. 17:353-361 describing insertion of the integrin receptor binding motif RGD at positions 520 and/or 584 of the AAV capsid subunit; and U.S. Pat. No. 7,314,912 describing insertion of the P1 peptide containing an RGD motif following amino acid positions 447, 534, 573 and 587 of the AAV2 capsid subunit). Other positions within the AAV capsid subunit that tolerate insertions are known in the art (e.g., positions 449 and 588 described by Grifman et al. (2001) Molecular Therapy 3:964-975).

As an example, a virus capsid of the present disclosure can have relatively inefficient tropism toward certain target cells of interest (e.g., immune cells, such as T cells and NK cells). A targeting sequence can advantageously be incorporated into these low-transduction vectors to thereby confer to the virus capsid a desired tropism and, optionally, selective tropism for particular tissue(s). AAV capsid proteins, capsids and vectors comprising targeting sequences are described, for example in international patent publication WO 00/28004. As another example, one or more non-naturally occurring amino acids as described by Wang et al., Annu Rev Biophys Biomol Struct. 35:225-49 (2006) can be incorporated into an AAV capsid subunit of this disclosure at an orthogonal site as a means of redirecting a low-transduction vector to desired target tissue(s). These unnatural amino acids can advantageously be used to chemically link molecules of interest to the AAV capsid protein including without limitation: glycans (mannose-dendritic cell targeting); RGD, bombesin or a neuropeptide for targeted delivery to specific cancer cell types; RNA aptamers or peptides selected from phage display targeted to specific cell surface receptors such as growth factor receptors, integrins, and the like. Methods of chemically modifying amino acids are known in the art (see, e.g., Greg T. Hermanson, Bioconjugate Techniques, 1^{st} edition, Academic Press, 1996).

In an aspect, the targeting sequence can be a virus capsid sequence (e.g., an autonomous parvovirus capsid sequence, AAV capsid sequence, or any other viral capsid sequence) that directs infection to a particular cell type(s).

In an aspect, an exogenous targeting sequence for use herein can be any amino acid sequence encoding a peptide that alters the tropism of a virus capsid or virus vector comprising the modified AAV capsid protein. In an aspect, the targeting peptide or protein can be naturally occurring or, alternately, completely or partially synthetic. In an aspect, targeting sequences can include ligands and other peptides that bind to cell surface receptors and glycoproteins, such as RGD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., a, β or γ), neuropeptides and endorphins, and the like, and fragments thereof that retain the ability to target cells to their cognate receptors. Other illustrative peptides and proteins include, but are not limited to substance P, keratinocyte growth factor, neuropeptide Y, gastrin releasing peptide, interleukin 2, hen egg white lysozyme, erythropoietin, gonadoliberin, corticostatin, β-endorphin, leu-enkephalin, rimorphin, α-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as α-bungarotoxin, and the like) can be substituted into the capsid protein as a targeting sequence. In an aspect, a AAV capsid protein herein can be modified by substitution of a “nonclassical” import/export signal peptide (e.g., fibroblast growth factor-1 and −2, interleukin 1, HIV-1 Tat protein, herpes virus VP22 protein, and the like) as described by Cleves (Current Biology 7:R318 (1997)) into the AAV capsid protein. In an aspect, a targeting sequence for use herein can be a peptide that can be used for chemical coupling (e.g., can comprise arginine and/or lysine residues that can be chemically coupled through their R groups) to another molecule that targets entry into a cell.

In an aspect, capsid protein variants, virus capsids and/or AAV vectors disclosed herein can have equivalent or enhanced transduction efficiency relative to the transduction efficiency of the AAV serotype from which the capsid protein variant, virus capsid and/or vector originated. In an aspect, capsid protein variants, virus capsids and/or vectors disclosed herein can have reduced transduction efficiency relative to the transduction efficiency of the AAV serotype from which the capsid protein variant, virus capsid and/or vector originated. In an aspect, capsid protein variants, virus capsids and/or vectors disclosed herein can have equivalent or enhanced tropism relative to the tropism of the AAV serotype from which capsid protein variant, virus capsid and/or vector originated. In an aspect, capsid protein variants, virus capsids and/or vectors disclosed herein can have an altered or different tropism relative to the tropism of the AAV serotype from which the capsid protein variant, virus capsid and/or vector originated. In an aspect, capsid protein variants, virus capsids and/or vectors disclosed herein can have or be engineered to have tropism for immune cells (e.g., T cells, NK cells). In an aspect, capsid protein variants, virus capsids and/or vectors disclosed herein can have or be engineered to have enhanced tropism for immune cells (e.g., T cells, NK cells). In an aspect, capsid protein variants, virus capsids and/or AAV vectors disclosed herein can produce an attenuated immunological response relative to the immunological response of the AAV serotype from which the capsid protein variant, virus capsid and/or vector originated. In an aspect, capsid protein variants, virus capsids and/or AAV vectors disclosed herein can be administered to a subject in multiple dosages (e.g., about two doses, about three doses, about four doses, about 5 doses, about 10 doses, about 15 doses, about 20 doses, about 40 doses, as many doses as needed to observe one or more desired responses) relative to the number of doses that can be administered using the AAV serotype from which the capsid protein variant, virus capsid and/or vector originated.

### 1. Capsid and AAV Engineering

In an aspect, rational engineering and/or mutational methods can be used to identify capsid protein variants of AAV vectors disclosed herein. In an aspect, methods herein can be used to produce an AAV vector that evades neutralizing antibodies. In an aspect, methods herein can be used to produce an AAV vector that has improved gene transfer efficiency. In an aspect, methods herein can be used to produce an AAV vector that has improved gene transfer efficiency in more than one mammalian species. In an aspect, methods herein can be used to produce an AAV vector that specifically targets a cell or tissue of interest (e.g., immune cells, such as T cells and NK cells).

In an aspect, a recombinant AAV described herein has improved gene transfer efficiency in one or more mammalian species relative to a recombinant AAV that has a capsid protein that is otherwise identical, except it lacks the one or more amino acid substitutions. In an aspect, the improved gene transfer efficiency can occur in one more of: Mus Musculus (mouse), Sus scrofa (pig), Canis Familiaris (Dog), non-human primates (Macaca, macaque), or Homo sapiens (human). In an aspect, the improved gene transfer efficiency can occur in Mus Musculus (mouse). In an aspect, the improved gene transfer efficiency occurs in one or more of the following cell types or tissues: hematopoietic progenitor cells, T-cells (CD4 T cells and/or CD8 T cells), B-cells, natural killer (NK) cells, dendritic cells, and/or macrophages. In an aspect, the improved gene transfer efficiency occurs in T cells and/or NK cells.

Aspects of the present disclosure provide for methods of producing AAV vectors as disclosed herein. In an aspect, methods can include one or more of the following steps: (a) identifying contact amino acid residues that form a three dimensional antigenic footprint on an AAV capsid protein; (b) generating a library of AAV capsid proteins comprising amino acid substitutions of the contact amino acid residues identified in (a); (c) producing AAV particles comprising capsid proteins from the library of AAV capsid proteins of (b); (d) contacting the AAV particles of (c) with cells under conditions whereby infection and replication can occur; (e) selecting AAV particles that can complete at least one infectious cycle and replicate to titers similar to control AAV particles; (f) contacting the AAV particles selected in (e) with neutralizing antibodies and cells under conditions whereby infection and replication can occur; and (g) selecting AAV particles that are not neutralized by the neutralizing antibodies of (f). Non-limiting examples of methods for identifying contact amino acid residues include peptide epitope mapping and/or cryo-electron microscopy. One of skill in the art will appreciate that there is an ever-evolving variety of methods and protocols that can be used to generate a library of AAV capsid proteins (e.g., rational design, barcoding, direct evolution, in silico discovery).

Any method of generating a library of AAV capsid protein known in the field or to be discovered that is suited for used herein can be used and/or optimized for use according to the methods disclosed herein.

In an aspect, generating a library of AAV capsid proteins comprising amino acid substitutions of the contact amino acid residues identified in an AAV capsid protein can produce a parental AAV capsid protein library. In an aspect, methods of producing AAV vectors herein can include administering the parental AAV capsid protein library to a mammal. In an aspect, administering the parental AAV capsid protein library to a mammal can be systemic administration to the mammal. In an aspect, the parental AAV capsid protein library can be administered to a mammal having a species of Mus Musculus (mouse), Sus scrofa (pig), Canis Familiaris (Dog), Non-human primates (Macaca, macaque), or Homo sapiens (human). In an aspect, the parental AAV capsid protein library can be administered to a Mus Musculus (mouse). In an aspect, capsid proteins can be enriched by collecting from a cell and/or a tissue from the mammal after administration of the parental AAV capsid protein library. In an aspect, capsid proteins can be enriched by collecting from a cell and/or a tissue from the mammal after administration of the parental AAV capsid protein library wherein the cell and/or a tissue can comprise hematopoietic progenitor cells, T-cells (CD4 T cells and/or CD8 T cells), B-cells, natural killer (NK) cells, dendritic cells, and/or macrophages. In an aspect, capsid proteins can be collected from the mammal after about 1 days to about 1 month (e.g., about 1 day, 5 days, 1 week, 2 weeks, 3 weeks, one month) following administration of the parental AAV capsid protein library. In an aspect, capsid proteins collected from a mammal after administration of the parental AAV capsid protein library can be used to generate another AAV capsid protein library referred to as the evolved AAV capsid protein library.

In an aspect, the evolved AAV capsid protein library can be administered to a mammal having a species of Mus Musculus (mouse), Sus scrofa (pig), Canis Familiaris (Dog), Non-human primates (Macaca, macaque), or Homo sapiens (human). In an aspect, capsid proteins can be enriched by collecting from a cell and/or a tissue from the mammal after administration of the evolved AAV capsid protein library. In an aspect, capsid proteins can be enriched by collecting from a cell and/or a tissue from the mammal after administration of the evolved AAV capsid protein library wherein the cell and/or a tissue can comprise hematopoietic progenitor cells, T-cells (CD4 T cells and/or CD8 T cells), B-cells, natural killer (NK) cells, dendritic cells, and/or macrophages. In an aspect, capsid proteins can be collected and identified from the mammal after administration of the evolved AAV capsid protein library. In an aspect, capsid proteins can be collected and identified from the mammal after about 1 days to about 1 month (e.g., about 1 day, 5 days, 1 week, 2 weeks, 3 weeks, one month) following administration of the evolved AAV capsid protein library. In an aspect, capsid proteins collected and identified from a mammal after administration of the evolved AAV capsid protein library can be used to generate an additional, second evolved AAV capsid protein library. In an aspect, the second evolved AAV capsid protein library can be administered to a mammal having a species of Mus Musculus (mouse), Sus scrofa (pig), Canis Familiaris (Dog), Non-human primates (Macaca, macaque), or Homo sapiens (human).

In an aspect, methods of evolving novel strains of adeno-associated viruses can comprise passaging AAV libraries across one or multiple mammalian species, wherein the AAV libraries can comprise a plurality of recombinant AAV vectors, wherein each recombinant AAV vector can comprise a capsid protein variant comprising one or more amino acid mutations relative to a wildtype AAV capsid protein. In an aspect, each recombinant AAV vector in the AAV libraries can comprise one or more amino acid mutations relative to a wildtype AAV6 capsid protein (SEQ ID NO:01). In an aspect, the one or more amino acid mutations can be in the region corresponding to amino acids 454-460 of SEQ ID NO:01.

In an aspect, a method of evolving novel strains of AAV can comprise administering a first AAV library to a first mammalian species. The AAVs from the first AAV library present in one or more target tissues of the first mammalian species can then be sequenced, and used to generate a second AAV library. The second AAV library can subsequently be administered to a second mammalian species, wherein the first mammalian species and the second mammalian species are different. The AAVs from the second AAV library present in one or more target tissues of the second mammalian species can then be sequenced. In an aspect, the first mammalian species and the second mammalian species can be each independently selected from the group consisting of: Mus Musculus (mouse), Sus scrofa (pig), Canis Familiaris (Dog), Non-human primates (Macaca, macaque), and Homo sapiens (human). These steps can then be repeated with a third, fourth, fifth, sixth, etc. species. In an aspect, the one or more target tissues/cells of the first mammalian species, the second mammalian species (or any subsequent species) is selected from hematopoietic progenitor cells, T-cells (CD4 T cells and/or CD8 T cells), B-cells, natural killer (NK) cells, dendritic cells, macrophages, and any combination thereof.

Disclosed herein is a capsid library comprising a first capsid proteins comprising the sequence set forth in SEQ ID NO:01, and one or more capsid proteins comprising the sequence set forth in SEQ ID NO:01, wherein amino acids 454-460 of the capsid protein comprise the sequence set forth in any one of SEQ ID NO:05-SEQ ID NO:545. In an aspect, the one or more capsid proteins can comprise the sequence set forth in SEQ ID NO:02.

Disclosed herein is a capsid library comprising one or more of capsid proteins comprising the sequence set forth in SEQ ID NO:01, wherein amino acids 454-460 of the capsid protein comprise the sequence set forth in any one of SEQ ID NO:05-SEQ ID NO:545. In an aspect, the one or more capsid proteins can comprise the sequence set forth in SEQ ID NO:02.

Disclosed herein is a capsid library comprising one or more capsid proteins comprising the sequence set forth in SEQ ID NO:01, wherein amino acids 454-460 of the capsid protein comprise the sequence set forth in SEQ ID NO:545.

### 2. AAV Vectors

In an aspect, the present disclosure provides AAV vectors comprising one or more of the capsid protein variants disclosed herein. As used herein, a “vector” refers to any molecule or moiety which transports, transduces or otherwise acts as a carrier of a heterologous molecule. A “viral vector” is a vector which comprises one or more polynucleotide regions encoding or comprising a payload molecule of interest, e.g., a transgene, a polynucleotide encoding a polypeptide or multi-polypeptide or a modulatory nucleic acid. Viral vectors of the present invention can be produced recombinantly using methods known in the art. Such techniques are explained fully in the literature, such as in Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; and Cell Biology: A Laboratory Notebook (J. E. Cellis, ed., 1989) Academic Press.

In an aspect, AAV viral particles disclosed herein can have a vector genome for expressing one or more of the capsid protein variants disclosed herein. The vector genome of the AAV vector can, in an aspect, be derived from the wild type genome of a virus, such as AAV, by using molecular methods to remove the wild type genome from the virus (e.g., AAV6), and replacing with a non-native nucleic acid, such as a heterologous polynucleotide sequence (e.g., a coding sequence for a transgene of interest). Typically, for AAV vectors, one or both inverted terminal repeat (ITR) sequences of the wild type AAV genome are retained in the AAV vector whereas other parts of the wild type viral genome are replaced with a non-native sequence such as a heterologous polynucleotide sequence between the retained ITRs. The vector genomes disclosed herein can encompass AAV genome-derived backbone elements, a coding sequence for a capsid protein variant disclosed herein, and a suitable promoter in operable linkage to the coding sequence. In an aspect, vector genomes disclosed herein can further comprise regulatory sequences regulating expression and/or secretion of the encoded protein. Examples include, but are not limited to, enhancers, polyadenylation signal sites, internal ribosome entry sites (IRES), sequences encoding protein transduction domains (PTD), microRNA-target sites, or a combination thereof.

In an aspect, vector genomes described herein can be single stranded. In other examples, vector genomes disclosed herein can be double stranded. For example, a vector genome described herein can be a self-complementary AAV vector genome capable of comprising double stranded portions therein.

### 3. AAV-backbone Elements

In an aspect, vector genomes disclosed herein can have one or more AAV-genome derived backbone elements, which refer to the minimum AAV genome elements required for the bioactivity of the AAV vectors. For example, the AAV-genome derived backbone elements may include the packaging site for the vector to be assembled into an AAV viral particle, one or more of the capsid protein variants disclosed herein, elements needed for vector replication, and/or expression of a transgene-encoding sequence comprised therein in host cells.

In an aspect, vector genome backbones disclosed herein may include at least one inverted terminal repeat (ITR) sequence. In an aspect, vector genome backbones herein may include two ITR sequences. In an aspect, one ITR sequence can be 5′ of a polynucleotide sequence coding for a transgene. In an aspect, one ITR sequence can be 3′ of a polynucleotide sequence coding for a transgene. In an aspect, a polynucleotide sequence coding for a transgene herein can be flanked on either side by an ITR sequence. Accordingly, in an aspect, a vector genome can comprise a transgene located between the first ITR and the second ITR.

In an aspect, vector genomes herein may include sequences or components originating from at least one distinct AAV serotype. In an aspect, AAV vector genome backbones disclosed herein can include at least ITR sequence from one distinct AAV serotype. In an aspect, AAV vector genome backbones disclosed herein may include at least ITR sequence from one distinct human AAV serotype. Such a human AAV can be derived from any known serotype, e.g., from any one of serotypes 1-11. In an aspect, AAV serotypes used herein have a tropism for immune cells, such as but not limited to a hematopoietic progenitor cell, a T-cell (CD4 T cell and/or CD8 T cell), a B-cell, a natural killer (NK) cell, a dendritic cell, and/or a macrophage. In an aspect, AAV vector genome backbones disclosed herein may have an ITR sequence of serotype AAV6.

In an aspect, AAV vectors herein can be a pseudotyped AAV vector, (i.e., comprises sequences or components originating from at least two distinct AAV serotypes). In an aspect, a pseudotyped AAV vector herein may include an AAV genome backbone derived from one AAV serotype, and a capsid protein derived at least in part from a distinct AAV serotype. In an aspect, pseudotyped AAV vectors herein can have an AAV2 vector genome backbone and a capsid protein derived from an AAV serotype having a tropism toward immune cells (e.g., T cells, NK cells).

To analyze the success of viral vector-mediated gene transfer, it can be important to be able to monitor both the distribution of the vector and the effectiveness of vector-mediated gene expression. This can be achieved by subcloning a reporter gene into the vector genome backbone. In an aspect, AAV vector genome backbones disclosed herein may contain a reporter gene. Several reporter genes are commonly used for this purpose and include, but are not limited to, fluorescent proteins of various colors (including green fluorescent protein (GFP), red fluorescent protein (RFP)), E. coli β-galactosidase (LacZ), and various forms of luciferase (Luc). In an aspect, AAV vector backbones disclosed herein may contain GFP.

The vector constructs disclosed herein can be prepared using known techniques. (See e.g., Current Protocols in Molecular Biology, Ausubel., F. et al., eds, Wiley and Sons, New York 1995). Fragment length can be chosen so that the recombinant genome does not exceed the packaging capacity of the AAV particle. If necessary, a “stuffer” DNA sequence can be added to the construct to maintain standard AAV genome size for comparative purposes. Such a fragment can be derived from such non-viral sources, e.g., lacZ, or other genes which are known and available to those skilled in the art.

### 4. Self-Complementary AAV Viral Vectors

In an aspect, AAV vectors disclosed herein can be self-complementary AAV (scAAV) vectors. Self-complementary AAV (scAAV) vectors contain complementary sequences that are capable of spontaneously annealing (folding back on itself to form a double-stranded genome) when entering into infected cells, thus circumventing the need for converting a single-stranded DNA vector using the cell's DNA replication machinery. An AAV herein having a self-complementing genome can quickly form a double stranded DNA molecule by virtue of its partially complementing sequences (e.g., complementing coding and non-coding strands of a transgene-encoding sequence).

In an aspect, a scAAV viral vector disclosed herein can comprise a first heterologous polynucleotide sequence and a second heterologous polynucleotide sequence, which can form intrastrand base pairs. In an aspect, the first heterologous polynucleotide sequence and the second heterologous polynucleotide sequence are linked by a sequence that facilitates intrastrand base pairing, e.g., to form a hairpin DNA structure. In an aspect, the dimeric structure of a scAAV vector upon entering a cell can be stabilized by a mutation or a deletion of one of the two terminal resolution sites (trs). As trs are Rep-binding sites contained within each ITR, a mutation or a deletion of such trs can prevent cleavage of a dimeric structure of a scAAV vector by AAV Rep proteins to form monomers. In an aspect, a scAAV viral vector disclosed herein can include a truncated 5′ inverted terminal repeats (ITR), a truncated 3′ ITR, or both. In an aspect, a scAAV vector disclosed herein can comprise a truncated 3′ ITR, in which the D region or a portion thereof (e.g., the terminal resolution sequence therein) can be deleted. Such a truncated 3′ ITR can be located between the first heterologous polynucleotide sequence and a second heterologous polynucleotide sequence noted above.

In an aspect, AAV vectors disclosed herein can comprise further elements necessary for expression, such as at least one suitable promoter which controls the expression of the transgene-encoding sequence. Such a promoters can be ubiquitous, tissue-specific, strong, weak, regulated, chimeric, etc., to allow efficient and suitable production of the protein in the infected tissue. The promoter can be homologous to the encoded protein, or heterologous, including cellular, viral, fungal, plant or synthetic promoters. Most preferred promoters for use herein can be functional in human cells. Non-limiting examples of ubiquitous promoters include viral promoters, particularly the CMV promoter, the RSV promoter, the SV40 promoter, etc. and cellular promoters such as the PGK (phosphoglycerate kinase) promoter. In an aspect, viral promoters herein can be a CMV promoter, a SV40 promoter, or any combination thereof.

In an aspect, AAV vectors disclosed herein can comprise further elements necessary for expression, such as at least one suitable promoter which controls the expression of the transgene-encoding sequence after infection of the appropriate cells. Suitable promoters for use herein include, in addition to the AAV promoters, e.g. the cytomegalovirus (CMV) promoter or the chicken beta actin/cytomegalovirus hybrid promoter (CAG), an endothelial cell-specific promoter such as the VE-cadherin promoter, as well as steroid promoters and metallothionein promoters. In an aspect, the promoter used in the vectors disclosed herein can be a CAG promoter.

In an aspect, a disclosed transgene-encoding sequence can comprise a tissue specific promoter which is functionally linked to the transgene-encoding sequence to be expressed. Accordingly, the specificity of the vectors according to the disclosure for the tissue (e.g., immune cells such as T cells and NK cells) can be further increased. In an aspect, a vector disclosed herein can have a tissue-specific promoter whose activity in the specific tissue is at least about 2-fold, 5-fold, 10-fold, 20-fold, 50-fold or 100-fold higher than in a tissue which is not the specific tissue. In an aspect, a tissue specific promoter herein is a human a tissue specific promoter. In an aspect, the expression cassette can also include an enhancer element for increasing the expression levels of exogenous protein to be expressed. Furthermore, the expression cassette can further comprise polyadenylation sequences, such as the SV40 polyadenylation sequences or polyadenylation sequences of bovine growth hormone.

In an aspect, AAV vectors disclosed herein can include one or more conventional control elements which are operably linked to the transgene-encoding sequence in a manner which permits its transcription, translation and/or expression in a cell transfected with the plasmid vector or infected with the virus produced by the invention. As used herein, “operably linked” sequences can include both expression control sequences that are contiguous with the transgene-encoding sequence and expression control sequences that act in trans or at a distance to control the transgene-encoding sequence. Expression control sequences can further comprise appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and can be utilized herein.

In an aspect, an AAV vector disclosed herein can include a modified capsid, including proteins or peptides of non-viral origin or structurally modified, to alter the tropism of the vector. For example, the capsid can include a ligand of a particular receptor, or a receptor of a particular ligand, to target the vector towards cell type(s) expressing said receptor or ligand, respectively.

### 5. Serotype of AAV Viral Particles

In an aspect, AAV vectors disclosed herein can be prepared or derived from various serotypes of AAVs. The term “serotype” is a distinction with respect to an AAV having a capsid which is serologically distinct from other AAV serotypes. Serologic distinctiveness is determined on the basis of the lack of cross-reactivity between antibodies to the AAV as compared to other AAV. Cross-reactivity can be measured using methods known in the art.

For example, cross-reactivity herein can be measured using a neutralizing antibody assay. For this assay polyclonal serum is generated against a specific AAV in a rabbit or other suitable animal model using the adeno-associated viruses. In this assay, the serum generated against a specific AAV is then tested in its ability to neutralize either the same (homologous) or a heterologous AAV. The dilution that achieves 50% neutralization is considered the neutralizing antibody titer. If for two AAVs the quotient of the heterologous titer divided by the homologous titer is lower than 16 in a reciprocal manner, those two vectors are considered as the same serotype. Conversely, if the ratio of the heterologous titer over the homologous titer is 16 or more in a reciprocal manner the two AAVs are considered distinct serotypes.

In an aspect, AAV vectors herein can be mixed of at least two serotypes of AAVs or with other types of viruses to produce chimeric (e.g., pseudotyped) AAV viruses. In an aspect, AAV vectors herein can be a human serotype AAV vector. Such a human AAV can be derived from any known serotype, e.g., from any one of serotypes 1-11.

### 6. Methods of Making AAV Particles

In an aspect, AAV vector genomes described herein can be packaged into virus particles which can be used to deliver the genome for transgene-encoding sequence expression in target cells. In an aspect, AAV vector genomes disclosed herein can be packaged into particles by transient transfection, use of producer cell lines, combining viral features into Ad-AAV hybrids, use of herpesvirus systems, or production in insect cells using baculoviruses.

A method of generating a packaging cell for use herein can involve creating a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing, addition of synthetic linkers containing restriction endonuclease cleavage sites, or by direct, blunt-end ligation. The packaging cell line is then infected with a helper virus, such as adenovirus. The advantages of this method are that the cells are selectable and are suitable for large-scale production of rAAV. Examples of suitable methods herein employ adenovirus or baculovirus, rather than plasmids, to introduce rAAV genomes and/or rep and cap genes into packaging cells.

### 7. Characteristics of AAV Vectors and AAV Particles

In an aspect, AAV vectors and/or AAV particles herein can have one or more improvements compared to naturally isolated AAV vectors. As used herein, a “naturally isolated AAV vector” refers to a vector that does not comprise one or more of the capsid protein variants disclosed herein. In an aspect, AAV vectors and/or AAV particles herein can have increased gene transfer efficiency in a cell compared to naturally isolated AAV vectors. In an aspect, AAV vectors and/or AAV particles herein can have at least about 2-fold to about 50-fold (e.g., about 2-, 4-, 6-, 8-, 10-, 20-, 30-, 40-, 50-fold) increased gene transfer efficiency in a cell compared to naturally isolated AAV vectors.

In an aspect, AAV vectors and/or AAV particles herein can have increased gene transfer efficiency in the cell and/or tissue of one or more mammalian species. In an aspect, AAV vectors and/or AAV particles herein can have increased gene transfer efficiency in the cell and/or tissue of one or more of Mus Musculus (mouse), Sus scrofa (pig), Canis Familiaris (Dog), Non-human primates (Macaca, macaque), or Homo sapiens (human), and any combination thereof. In an aspect, AAV vectors and/or AAV particles herein can have increased gene transfer efficiency in a cell and/or tissue of a mammal, the cell and/or tissue comprising hematopoietic progenitor cells, T-cells (CD4 T cells and/or CD8 T cells), B-cells, natural killer (NK) cells, dendritic cells, and/or macrophages.

In an aspect, AAV vectors and/or AAV particles herein can have a higher vector titer compared to naturally isolated AAV vectors. In an aspect, AAV vectors and/or AAV particles herein can have at least about 2-fold to about 50-fold (e.g., about 2-, 4-, 6-, 8-, 10-, 20-, 30-, 40-, 50-fold) higher vector titer compared to naturally isolated AAV vectors.

In an aspect, AAV vectors and/or AAV particles herein can be less susceptible to antibody-mediated neutralization compared to naturally isolated AAV vectors. In an aspect, AAV vectors and/or AAV particles herein can be less susceptible to antibody-mediated neutralization by about 2-fold to about 50-fold (e.g., about 2-, 4-, 6-, 8-, 10-, 20-, 30-, 40-, 50-fold) compared to naturally isolated AAV vectors. In an aspect, AAV vectors and/or AAV particles herein can be less susceptible to antibody-mediated neutralization for at least about 1 hour to about 24 hours (e.g., about 1, 2, 4, 8, 12, 16, 20, 24 hours) after administration to a subject compared to naturally isolated AAV vectors.

In an aspect, AAV vectors and/or AAV particles herein can produce lower levels of anti-AAV antibodies after at least one administration to a subject herein compared to naturally isolated AAV vectors. In an aspect, AAV and/or AAV particles herein can produce about 2-fold to about 50-fold (e.g., about 2-, 4-, 6-, 8-, 10-, 20-, 30-, 40-, 50-fold) less anti-AAV antibodies after at least one administration to a subject herein compared to naturally isolated AAV vectors. In an aspect, gene therapies comprising AAV vectors and/or AAV particles herein can be administered about 2 times to about 10 times (e.g., about 2, 3, 4, 5, 6,,7, 8, 9, 10) to a subject herein without becoming susceptible to antibody-mediated neutralization.

In an aspect, AAV vectors and/or AAV particles herein can have expression in any cell or tissue type of more than one mammal. In an aspect, AAV vectors and/or AAV particles herein can have expression in any cell or tissue type of more than one mammal comprising a human, mouse, rat, guinea pig, dog, cat, horse, cow, pig, or non-human primate (e.g., monkey, chimpanzee, baboon, gorilla). In an aspect, AAV vectors and/or AAV particles herein can have expression in any cell or tissue type of a human, a mouse, a dog, and a non-human primate.

Disclosed herein is a nucleotide sequence encoding an AAV capsid protein variant, wherein the encoded AAV capsid protein variant has at least 90% identity to the sequence of SEQ ID NO:01, wherein the amino acids corresponding to amino acids 454-460 of SEQ ID NO:01 are substituted with a peptide having a sequence of any one of SEQ ID NO:05-SEQ ID NO:545. Disclosed herein is a nucleotide sequence encoding an adeno-associated virus (AAV) capsid protein variant, wherein the encoded AAV capsid protein variant has at least 90% identity to the sequence of SEQ ID NO:01, wherein one or more amino acids corresponding to amino acids 454-460 of SEQ ID NO:01 are substituted with a peptide having a sequence of any one of SEQ ID NO:05-SEQ ID NO:545. Disclosed herein is a recombinant adeno-associated virus (AAV) capsid protein variant, wherein the capsid protein variant can comprise a peptide having the sequence of any one of SEQ ID NO:05-SEQ ID NO:545. Disclosed herein is an AAV capsid protein variant, wherein the AAV capsid variant can comprise the sequence of SEQ ID NO:02 or a sequence with at least 90% or at least 95% identity thereto. Disclosed herein is an AAV capsid protein comprising the sequence of SEQ ID NO:01 or SEQ ID NO:02. Disclosed herein is an AAV capsid protein comprising the sequence of SEQ ID NO:01 or SEQ ID NO:02, wherein the sequence can comprise one or more modifications. In an aspect, a disclosed modification can comprise a substitution of an amino acid. For example, in an aspect, a disclosed AAV capsid protein can comprise the sequence of SEQ ID NO:01 and a modification at position 454, position 455, position 456, position 457, position 458, position 459, and/or position 460, or a combination thereof. In an aspect, a modification can comprise the substitution of any one of SEQ ID NO:05-SEQ ID NO:545 at positions 454-460 of SEQ ID NO:01. In an aspect, a modification can comprise the substitution of NYLEADD at positions 454-460 of SEQ ID NO:01. In an aspect, a modification can comprise the substitution of HAPRVEE at positions 454-460 of SEQ ID NO:01. Disclosed herein is a AAV capsid protein comprising the sequence of SEQ ID NO:01, wherein the amino acids corresponding to amino acids 454-460 of SEQ ID NO:01 are substituted with a peptide having a sequence of any one of SEQ ID NO:05-SEQ ID NO:545. Disclosed herein is a AAV capsid protein comprising the sequence of SEQ ID NO:01 or a fragment thereof. Disclosed herein is a AAV capsid protein comprising the sequence of SEQ ID NO:02 or a fragment thereof. Disclosed herein is an isolated nucleotide sequence encoding an AAV capsid protein. Disclosed herein is an isolated nucleotide sequence encoding an AAV capsid protein, wherein the encoded capsid protein can comprise the sequence set forth in SEQ ID NO:01 or SEQ ID NO:02.

Disclosed herein is a recombinant AAV vector comprising a disclosed AAV capsid protein. Disclosed herein is a recombinant AAV vector comprising a disclosed AAV capsid variant protein. In an aspect, a disclosed recombinant AAV vector can comprise a vector genome. A vector genome can be encapsidated by a disclosed AAV capsid comprising a disclosed AAV capsid protein or a disclosed AAV capsid protein variant. In an aspect, a disclosed vector genome can comprise a first inverted terminal repeat (ITR) and a second ITR. In an aspect, a disclosed vector genome can comprise a transgene located between the first ITR and the second ITR. In an aspect, a transgene can comprise a therapeutic RNA, a therapeutic protein, or a gene-editing molecule. In an aspect, a gene-editing molecule can comprise a nuclease. In an aspect, a nuclease can comprise Cas9. In an aspect, a gene-editing molecule can be a single guide RNA (sgRNA). Disclosed herein is a AAV capsid protein variant comprising a peptide having the sequence of any one of SEQ ID NO:05-SEQ ID NO:545. Disclosed in an AAV capsid protein variant comprising an amino acid sequence having at least 90% identity to the sequence of SEQ ID NO:01, wherein the amino acids corresponding to amino acids 454-460 of SEQ ID NO:01 are substituted with a peptide having a sequence of any one of SEQ ID NO:05-SEQ ID NO:545. Disclosed in an AAV capsid protein variant comprising an amino acid sequence having at least 90% identity to the sequence of SEQ ID NO:01, wherein one or more amino acids corresponding to amino acids 454-460 of SEQ ID NO:01 are substituted with a peptide having a sequence of any one of SEQ ID NO:05-SEQ ID NO:545. Disclosed herein is an AAV capsid protein variant comprising an amino acid sequence of SEQ ID NO:02 or a sequence with at least 90% or at least 95% identity thereto. In an aspect, a disclosed AAV capsid can comprise a disclosed AAV capsid protein variant.

### C. Pharmaceutical Compositions

In an aspect, any of the disclosed AAV vectors, virus capsids, and/or AAV viral particles disclosed herein can be formulated to form a pharmaceutical composition. In an aspect, pharmaceutical compositions herein can further include a pharmaceutically acceptable carrier, diluent or excipient. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” can refer to molecular entities and other ingredients of compositions comprising such that are physiologically tolerable and do not typically produce untoward reactions when administered to a mammal (e.g., a human, a mouse). In an aspect, the “pharmaceutically acceptable” carrier used in the pharmaceutical compositions disclosed herein can be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and can comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In an aspect, the pharmaceutical compositions or formulations herein are for parenteral administration, such as intravenous, intracerebroventricular injection, intra-cisterna magna injection, intra-parenchymal injection, or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein can further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. Aqueous solutions can be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating the active (e.g., AAV vectors virus capsids, and/or AAV viral particles) in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

The pharmaceutical compositions disclosed herein can also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

### D. Methods of Use

1. Methods of Alleviatinm and/or Treating a Disease or a Condition

Disclosed herein is a method of alleviating and/or treating a disease or a condition comprising administering to a subject in need thereof a therapeutically effectively amount of a disclosed AAV vector, a disclosed AAV particle, a disclosed AAV genome, a disclosed AAV viral capsid, a disclosed AAV viral capsid protein, or any combination thereof.

Disclosed herein is a method of alleviating and/or treating a disease or a condition comprising administering to a subject in need thereof a therapeutically effectively amount of a pharmaceutical composition comprising a disclosed AAV vector, a disclosed AAV particle, a disclosed AAV genome, a disclosed AAV viral capsid, a disclosed AAV viral capsid protein, or any combination thereof.

Disclosed herein is a method of alleviating and/or treating a disease or a condition comprising administering to a subject in need thereof a therapeutically effectively amount of cells that have been generated using a disclosed AAV vector, a disclosed AAV particle, a disclosed AAV genome, a disclosed AAV viral capsid, a disclosed AAV viral capsid protein, or any combination thereof. For example, in an aspect, a disclosed method can comprise administering CAR T cells made by using compositions (e.g., one or more of a disclosed AAV vector, disclosed AAV particle, disclosed AAV genome, disclosed viral capsid, a disclosed viral capsid protein, or any combination thereof) disclosed herein. For example, in an aspect, CAR T cells can be made using a disclosed AAV capsid protein comprising the sequence set forth in SEQ ID NO:01, wherein amino acids 454-460 of the capsid protein comprise the sequence set forth in any one of SEQ ID NO:05-SEQ ID NO:545. In an aspect, CAR T cells can be made using a disclosed AAV capsid protein comprising the sequence set forth in SEQ ID NO:02.

Any of the compositions (e.g., a disclosed AAV vector, a disclosed AAV particle, a disclosed AAV genome, a disclosed AAV viral capsid, a disclosed AAV viral capsid protein, or any combination thereof) described herein can be used for alleviating and/or treating a disease or a condition. In an aspect, any of the compositions (e.g., a disclosed AAV vector, a disclosed AAV particle, a disclosed AAV genome, a disclosed AAV viral capsid, a disclosed AAV viral capsid protein, or any combination thereof) described herein can be used for alleviating and/or treating a disease or a condition by systemic administration. In an aspect, any of the compositions (e.g., a disclosed AAV vector, a disclosed AAV particle, a disclosed AAV genome, a disclosed AAV viral capsid, a disclosed AAV viral capsid protein, or any combination thereof) described herein can be used for alleviating and/or treating a disease or a condition by genetically modifying a subject's immune cells ex vivo. In an aspect, any of the compositions (e.g., a disclosed AAV vector, a disclosed AAV particle, a disclosed AAV genome, a disclosed AAV viral capsid, a disclosed AAV viral capsid protein, or any combination thereof) described herein can be used for alleviating and/or treating a disease or a condition by modifying an immune cell to have one or more genetic modifications to enable expression of chimeric antigen receptors (CARs).

In an aspect, a disclosed AAV capsid protein can comprise the sequence set forth in SEQ ID NO:01, wherein amino acids 454-460 of the capsid protein comprise the sequence set forth in any one of SEQ ID NO:05-SEQ ID NO:545. In an aspect, a disclosed AAV capsid protein can comprise the sequence set forth in SEQ ID NO:02.

Thus, in an aspect, the present disclosure provides methods for alleviating one or more symptoms and/or for treating a disease or a condition in a subject in need of treatment by compositions disclosed herein, as well as a pharmaceutical composition comprising such. In an aspect, a subject of the methods herein can be a human subject. In an aspect, the subject can be a subject that has not been previously exposed to wild-type AAV or a recombinant (rAAV) vector. In an aspect, the subject can be a subject that has not been previously administered a rAAV vector. In an aspect, the subject is a subject that has been previously administered a rAAV vector, e.g., a rAAV vector described herein. A subject that has been exposed or administered an AAV or rAAV can be identified using methods known in the art, e.g., by PCR detection of viral DNA or by measuring antibody titer to AAV or rAAV, either the capsid or the transgene. In an aspect, the subject can be a subject that has not been administered an enzyme replacement therapy (e.g., by administration of the enzyme protein).

A subject that has been administered an enzyme replacement therapy can be identified using methods known in the art, e.g., by measuring antibody titer to the enzyme. However, in an aspect the subject has previously been treated with an enzyme replacement therapy. In an aspect, the subject is a subject that has undergone one or more approaches to clear neutralizing antibodies (NAbs) (e.g., plasmapheresis, immunosuppression, enzymatic degradation). In an aspect, a subject suitable of methods of use herein cannot need to clear neutralizing antibodies (NAbs) before administration of any of the compositions (e.g., a disclosed AAV vector, a disclosed AAV particle, a disclosed AAV genome, a disclosed AAV viral capsid, a disclosed AAV viral capsid protein, or any combination thereof) described herein.

In an aspect, the subject has or is suspected of having a disease that can be treated with gene therapy. Illustrative diseases or a conditions that can be treated using the methods disclosed herein can include, but are not limited to: cystic fibrosis (cystic fibrosis transmembrane regulator protein) and other diseases of the lung, hemophilia A (Factor VIII), hemophilia B (Factor IX), thalassemia (0-globin), anemia (erythropoietin) and other blood disorders, Alzheimer's disease (GDF; neprilysin), multiple sclerosis (0-interferon), Parkinson's disease (glial-cell line derived neurotrophic factor GDNF), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a, e.g., for hepatocellular carcinoma), diabetes mellitus (insulin), muscular dystrophies including Duchenne (dystrophin, mini-dystrophin, insulin-like growth factor I, a sarcoglycan e.g., β, γ, RNAi against myostatin, myostatin propeptide, follistatin, activin type II soluble receptor, anti-inflammatory polypeptides such as the I-kappa B dominant mutant, sarcospan, utrophin, mini-utrophin, antisense or RNAi against splice junctions in the dystrophin gene to induce exon skipping (see, e.g., WO/2003/095647), antisense against U7 snRNAs to induce exon skipping (see, e.g., WO/2006/021724), and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease α-galactosidase and Pompe disease lysosomal acid α-glucosidase) and other metabolic disorders, congenital emphysema (al-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease GDNF, astrocytomas endostatin, angiostatin and/or RNAi against VEGF, glioblastomas endostatin, angiostatin and/or RNAi against VEGF), liver, kidney, heart including congestive heart failure or peripheral artery disease (PAD) (e.g., by delivering protein phosphatase inhibitor I (I-1) and fragments thereof (e.g., IIC), serca2a, zinc finger proteins that regulate the phospholamban gene, Barkct, P2-adrenergic receptor, p2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCAl, SCA2 and SCA3, phenylketonuria (phenylalanine hydroxylase), autoimmune diseases, and the like.

To perform the methods disclosed herein, an effective amount of the compositions (e.g., a disclosed AAV vector, a disclosed AAV particle, a disclosed AAV genome, a disclosed AAV viral capsid, a disclosed AAV viral capsid protein, or any combination thereof) or a pharmaceutical composition comprising a disclosed AAV vector, a disclosed AAV particle, a disclosed AAV genome, a disclosed AAV viral capsid, a disclosed AAV viral capsid protein, or any combination thereof, or cells generated by using one or more of a disclosed AAV vector, a disclosed AAV particle, a disclosed AAV genome, a disclosed AAV viral capsid, a disclosed AAV viral capsid protein, or any combination thereof can be administered to a subject who needs treatment via a suitable route (e.g., oral, intramuscular, intravenous, intracerebroventricular injection, intra-cisterna magna injection, intravitreal, subretinal, subconjuctival, retrobulbar, intracameral, suprachoroidal, intracoronary injection, intraarterial injection, and/or intra-parenchymal injection) at a suitable amount as disclosed herein.

In an aspect, the present disclosure also provides for methods of introducing one or more AAV vectors to a cell, comprising contacting the cell with a composition disclosed herein. In an aspect, methods herein can include delivering one or more AAV vectors herein to a cell, comprising contacting the cell or layer with a viral vector wherein the viral vector can comprise an AAV capsid protein variant disclosed herein. In an aspect of this method, AAV vectors herein can deliver one or more heterologous molecules to a cell. In an aspect, AAV vectors herein can deliver one or more therapeutic heterologous molecules to a cell. In an aspect, one or more therapeutic heterologous molecules delivered to a cell using the methods herein can be a therapeutic protein, a therapeutic DNA, and/or therapeutic RNA. In an aspect, the therapeutic protein can be a monoclonal antibody or a fusion protein. In an aspect, the therapeutic DNA and/or RNA can be an antisense oligonucleotide, siRNA, shRNA, mRNA, a DNA oligonucleotide, and the like.

In an aspect, the present disclosure also provides for methods of introducing an AAV vector to a hematopoietic progenitor cell, a T-cell (CD4 T cell and/or CD8 T cell), a B-cell, natural killer (NK) cell, a dendritic cell, a macrophage or any combination thereof, comprising contacting the cell with a virus vector and/or composition disclosed herein. In an aspect, AAV vectors herein can be delivered to a specific tissue by administering AAV particles having one or more AAV capsid protein variants disclosed herein with enhanced tropism to a hematopoietic progenitor cell, a T-cell (CD4 T cell and/or CD8 T cell), a B-cell, a natural killer (NK) cell, a dendritic cell, a macrophage, or any combination thereof.

In an aspect, methods of administering at least one of a disclosed AAV vector, a disclosed AAV particle, a disclosed AAV genome, a disclosed AAV viral capsid, a disclosed AAV viral capsid protein, or any combination thereof having one or more nucleic acid molecules herein to a tissue substantially modulates expression of the at least one protein and/or gene as compared to baseline. As used herein, “baseline” refers to the expression of the at least one transgene (and the encoded product of the transgene) before the AAV vectors herein were administered. As used herein, “substantially modulates expression” refers to at least a 1-fold change in expression (e.g., increased expression, decreased expression) as compared to baseline. In an aspect, methods of administering at least one of a disclosed AAV vector, a disclosed AAV particle, a disclosed AAV genome, a disclosed AAV viral capsid, a disclosed AAV viral capsid protein, or any combination thereof disclosed herein to a tissue modulates expression of the at least one protein and/or gene as compared to baseline by at least about 2-fold to about 50-fold (e.g., about 2-, 4-, 6-, 8-, 10-, 20-, 30-, 40-, 50-fold). In an aspect, methods of administering at least one AAV particle or AAV vector having one or more AAV capsid protein variants disclosed herein to a tissue modulates expression of the at least one protein and/or gene as compared to baseline by at least about 2-fold to about 50-fold (e.g., about 2-, 4-, 6-, 8-, 10-, 20-, 30-, 40-, 50-fold) when the at least one AAV particle or AAV vector is delivered to a hematopoietic progenitor cell, a T-cell (CD4 T cell and/or CD8 T cell), a B-cell, a natural killer (NK) cell, a dendritic cell, a macrophage or any combination thereof.

In any of the methods disclosed herein, an effective amount of the compositions (e.g., a disclosed AAV vector, a disclosed AAV particle, a disclosed AAV genome, a disclosed AAV viral capsid, a disclosed AAV viral capsid protein, or any combination thereof) described herein can be given to a subject in need thereof to alleviate one or more symptoms associated with a disease and or condition. “An effective amount” as used herein refers to a dose of a disclosed composition which is sufficient to confer a therapeutic effect on a subject having a disease and or condition. In an aspect, an effective amount can be an amount that reduces at least one symptom of disease or condition in the subject.

In an aspect, methods of administering at least one AAV as disclosed herein can have increased gene transfer efficiency in a cell compared to naturally isolated AAV vectors. In an aspect, methods of administering at least one AAV vector as disclosed herein can have at least about 2-fold to about 50-fold (e.g., about 2-, 4-, 6-, 8-, 10-, 20-, 30-, 40-, 50-fold) increased gene transfer efficiency in a cell compared to naturally isolated AAV vectors. In an aspect, methods of administering at least one AAV vector as disclosed herein can have increased gene transfer efficiency in a tissue compared to naturally isolated AAV vectors. In an aspect, methods of administering at least one AAV vector as disclosed herein can have at least about 2-fold to about 50-fold (e.g., about 2-, 4-, 6-, 8-, 10-, 20-, 30-, 40-, 50-fold) increased gene transfer efficiency in a tissue compared to naturally isolated AAV vectors. In an aspect, methods of administering at least one AAV vector as disclosed herein can have increased gene transfer efficiency in a subject compared to naturally isolated AAV vectors. In an aspect, methods of administering at least one AAV vector as disclosed herein can have at least about 2-fold to about 50-fold (e.g., about 2-, 4-, 6-, 8-, 10-, 20-, 30-, 40-, 50-fold) increased gene transfer efficiency in a subject compared to naturally isolated AAV vectors.

In an aspect, methods herein can include administering at least one AAV vector to a subject at least once. In an aspect, methods herein can include administering at least one AAV particle and/or at least one AAV vector to a subject more than once. In an aspect, methods herein can include administering at least one AAV vector herein to a subject between at least once to at least 10 times (e.g., about 1, 2, 3, 4, 5, 6, 7, 8, 9, 10 times). In an aspect, methods herein can include administering at least one AAV vector herein to a subject at least twice, at least 3 times, at least 4 times, or at least 5 times. In an aspect, methods herein can include administering at least one AAV vector herein to a subject once a day, once every other day, once a week, once every two weeks, once every three weeks, once a month, once every other month, once every three months, once every four months, once a year, or twice a year. In an aspect, methods herein can include administering at least one AAV vector herein to a subject at many times as needed to see the desired response. In an aspect, the desired response can be attenuation of at least one symptom of a disease and/or condition in a subject after administration of a dose of an AAV vector herein compared to before administration of the AAV vector. One of skill in the art will appreciate that dosing regimens can be optimized according to disease/condition, disease/condition severity, characteristics of the subject (e.g., age, gender, weight), and the like.

In an aspect, an AAV vector herein can be used for the delivery of cre-recombinase. In an aspect, an AAV vector herein can be used for the delivery of cre-recombinase to result in a conditional activation, a conditional inactivation, an activation, an inactivation, or any combination thereof of one or more genes in a cell, tissue, and/or subject. In an aspect, an AAV vector herein can deliver cre-recombinase to one or more specific cell and/or tissue types (e.g., immune cells such as T cells and NK cells).

In an aspect, an AAV vector herein can be used for the delivery of a CRISPR-Cas system. The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined ˜20 nucleotide “spacer” or “targeting” sequence which defines the genomic target to be modified. The genomic target of Cas9 can be changed by changing the targeting sequence present in the sgRNA.

In an aspect, an AAV vector can comprise a vector genome, wherein the vector genome encodes a gene-editing molecule. In an aspect, the gene-editing molecule is a nuclease. In an aspect, the nuclease is a Cas9 nuclease. In an aspect, the nuclease is a Casl2a nuclease. In an aspect, the gene editing molecule is a sgRNA.

In an aspect, methods provided herein can include generating a cell to express any of the polynucleotides and/or vectors described herein. In an aspect, cells for use herein can be one or more immune cells. As used herein an “immune cell” can refer to a cell of the immune system. Immune cells can be categorized as lymphocytes, neutrophils, granulocytes, mast cells, monocytes/macrophages, and dendritic cells. In an aspect, cells for use herein can be one or more lymphocytes. In an aspect, lymphocytes can be T-cells (CD4 T cells and/or CD8 T cells), B-cells, and/or natural killer (NK) cells. In an aspect, cells for use herein can be one or more cytotoxic lymphocytes. As used herein, a “cytotoxic lymphocyte” refers to a lymphocyte capable cytolysis. For example, but not limited to, a cytotoxic lymphocyte can be capable of killing cancer cells, cells that are infected (particularly with viruses), and cells that are damaged in one or more other ways.

In an aspect, cells for use herein can be isolated from a subject. In an aspect, cells for use herein can be isolated from peripheral blood, umbilical cord blood, and/or bone marrow.

In an aspect, cells for use herein can be isolated from peripheral blood mononuclear cells (PBMCs). In an aspect, cells for use herein can be isolated from a leukapheresis sample. In an aspect, cells for use herein can be isolated from tumor-infiltrated lymphocytes, tissue-infiltrated lymphocytes, lymph nodes, thymus, and/or secondary lymphoid organs. In an aspect, cells for use herein can be isolated from autologous peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “autologous” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from the same subject to be treated with the compositions disclosed herein. In an aspect, cells for use herein can be isolated from allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “allogeneic” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from a different subject of the same species as the subject to be treated with the compositions disclosed herein. In an aspect, cells for use herein can be isolated from haploidentical allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs.

In an aspect, gene expression of an immune cell as disclosed herein can be modulated by any of the compositions (e.g., a disclosed AAV vector, a disclosed AAV particle, a disclosed AAV genome, a disclosed AAV viral capsid, a disclosed AAV viral capsid protein, or any combination thereof) herein to alter expression of at least one gene native to an immune cell.

In an aspect, modulating gene expression of an immune cell as disclosed herein can alter expression of at least one gene native to an immune cell by about 1% to about 100%, about 5% to about 95%, about 10% to about 90%, about 15% to about 85%, or about 20% to about 80%.

In an aspect, modulating gene expression of an immune cell as disclosed herein can prevent expression of at least one gene native to the immune cell. In an aspect, modulating gene expression of an immune cell as disclosed herein can lower expression of at least one gene native to the immune cell. In an aspect, modulating gene expression of an immune cell as disclosed herein can increase expression of at least one gene native to the immune cell.

In an aspect, gene expression of an immune cell as disclosed herein can be modulated by any of the compositions (e.g., a disclosed AAV vector, a disclosed AAV particle, a disclosed AAV genome, a disclosed AAV viral capsid, a disclosed AAV viral capsid protein, or any combination thereof) herein to have one or more genetic modifications to enable expression of chimeric antigen receptors (CARs). In an aspect, immune cells with modulated gene expression can express at least one CAR with one or more genetic modifications to an extracellular antigen recognition domain of the single-chain Fragment variant (scFv) of the CAR, a transmembrane domain of the CAR, an intracellular activation domain of the CAR, or a combination thereof.

In an aspect, gene expression of an immune cell as disclosed herein can be modulated by any of the compositions (e.g., a disclosed AAV vector, a disclosed AAV particle, a disclosed AAV genome, a disclosed AAV viral capsid, a disclosed AAV viral capsid protein, or any combination thereof) herein to have one or more genetic modifications to T-cell receptors (TCRs). In an aspect, immune cells with modulated gene expression and/or native immune cells can have one or more genetic modifications to an alpha-chain of a TCR, a beta-chain of a TCR, or a combination thereof. In an aspect, immune cells with modulated gene expression and/or native immune cells according to methods disclosed herein can have one or more genetic modifications to increase secretion of one or more antibodies, one or more cytokines, one or more proteins, or a combination thereof.

### 2. Methods of Generating an Immune Cell Therapy

Disclosed herein is a method of generating an immune cell therapy comprising administering to a subject in need thereof a therapeutically effectively amount of a disclosed AAV vector, a disclosed AAV particle, a disclosed AAV genome, a disclosed AAV viral capsid, a disclosed AAV viral capsid protein, or any combination thereof.

Disclosed herein is a method of generating an immune cell therapy comprising administering to a subject in need thereof a therapeutically effectively amount of a pharmaceutical composition comprising a disclosed AAV vector, a disclosed AAV particle, a disclosed AAV genome, a disclosed AAV viral capsid, a disclosed AAV viral capsid protein, or any combination thereof.

Disclosed herein is a method of generating an immune cell therapy comprising administering to a subject in need thereof a therapeutically effectively amount of cells that have been generated using a disclosed AAV vector, a disclosed AAV particle, a disclosed AAV genome, a disclosed AAV viral capsid, a disclosed AAV viral capsid protein, or any combination thereof. For example, in an aspect, a disclosed method can comprise administering CAR T cells made by using compositions (e.g., one or more of a disclosed AAV vector, a disclosed AAV particle, a disclosed AAV genome, a disclosed AAV viral capsid, a disclosed AAV viral capsid protein, or any combination thereof) disclosed herein. For example, in an aspect, CAR T cells can be made using a disclosed AAV capsid protein comprising the sequence set forth in SEQ ID NO:01, wherein amino acids 454-460 of the capsid protein comprise the sequence set forth in any one of SEQ ID NO:05-SEQ ID NO:545. In an aspect, CAR T cells can be made using a disclosed AAV capsid protein comprising the sequence set forth in SEQ ID NO:02.

In an aspect, a disclosed AAV capsid protein can comprise the sequence set forth in SEQ ID NO:01, wherein amino acids 454-460 of the capsid protein comprise the sequence set forth in any one of SEQ ID NO:05-SEQ ID NO:545. In an aspect, a disclosed AAV capsid protein can comprise the sequence set forth in SEQ ID NO:02.

In an aspect, compositions (e.g., a disclosed AAV vector, a disclosed AAV particle, a disclosed AAV genome, a disclosed AAV viral capsid, a disclosed AAV viral capsid protein, or any combination thereof) herein can be used in methods of generating an immune cell therapy composition. In an aspect, an immune cell therapy composition disclosed herein can include at least one immune cell with modulated gene expression. As used herein, the term “immune cell therapy” or “immunotherapy” refers to a therapeutic approach of activating or suppressing the immune system for the treatment of disease. In an aspect, an immune cell therapy composition disclosed herein encompasses adoptive cell therapy. As used herein, the term “adoptive cell therapy” refers to the transfer of ex vivo grown immune cells into a subject for treatment of a disease. In an aspect, immune cell therapy compositions disclosed herein include at least one lymphocyte with modulated gene expression. In an aspect, a lymphocyte with modulated gene expression for use in an immune cell therapy composition can be a cytotoxic lymphocyte. In an aspect, a cytotoxic lymphocyte for use in an immune cell therapy composition can be a NK cell, a CD4 T cell, and/or a CD8 T cell.

In an aspect, immune cell therapy compositions disclosed herein can be administered to a subject in need thereof. A suitable subject includes a mammal, a human, a livestock animal, a companion animal, a lab animal, or a zoological animal. In an aspect, the subject can be a rodent, e.g., a mouse, a rat, a guinea pig, etc. In an aspect, the subject can be a livestock animal. Non-limiting examples of suitable livestock animals can include pigs, cows, horses, goats, sheep, llamas and alpacas. In an aspect, the subject can be a companion animal. Non-limiting examples of companion animals can include pets such as dogs, cats, rabbits, and birds. In an aspect, the subject can be a zoological animal. As used herein, a “zoological animal” refers to an animal that can be found in a zoo. Such animals can include non-human primates, large cats, wolves, and bears. In an aspect, the animal is a laboratory animal. Non-limiting examples of a laboratory animal can include rodents, canines, felines, and non-human primates. In an aspect, the animal is a rodent. Non-limiting examples of rodents can include mice, rats, guinea pigs, etc. In an aspect, the subject is a human.

In an aspect, a subject in need thereof can have been diagnosed with a cancer. By example, but not limited to, a subject can have been diagnosed with nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, tonsil cancer, or a combination thereof.

In an aspect, a subject in need thereof can have been diagnosed with an infectious disease. By example, but not limited to, a subject can have been diagnosed with chickenpox, common cold, diphtheria, E. coli, giardiasis, HIV/AIDS, infectious mononucleosis, influenza, Lyme disease, malaria, measles, meningitis, mumps, poliomyelitis (polio), pneumonia, Rocky mountain spotted fever, rubella (German measles), Salmonella infections, severe acute respiratory syndrome (SARS), sexually transmitted diseases, shingles (herpes zoster), tetanus, toxic shock syndrome, tuberculosis, viral hepatitis, West Nile virus, whooping cough (pertussis), or a combination thereof.

In an aspect, a subject in need thereof can have been diagnosed with an autoimmune disease. By example, but not limited to, a subject can have been diagnosed with diabetes (Type 1), lupus, multiple sclerosis, rheumatoid arthritis, celiac disease, or a combination thereof.

In an aspect, a subject in need thereof can have been diagnosed with an immune deficiency disease. By example, but not limited to, a subject can have been diagnosed with autoimmune lymphoproliferative syndrome (ALPS), autoimmune polyglandular syndrome type 1 (APS-1), BENTA disease, caspase eight deficiency state (CEDS), CARD9 deficiency and other syndromes of susceptibility to Candidiasis, chronic granulomatous disease (CGD), common variable immunodeficiency (CVID), congenital neutropenia syndromes, CTLA4 deficiency, DOCK8 deficiency, GATA2 deficiency, hyper-immunoglobulin E syndrome (HIES), hyper-immunoglobulin M (IgM) syndrome, leukocyte adhesion deficiency (LAD), LRBA deficiency, PI3 kinase disease, PLAID and/or PLAID-like disease, severe combined immunodeficiency (SCID), STAT3 gain-of-function disease, Warts, Hypogammaglobulinemia, Infections, and Myelokathexis (WHIM) Syndrome, Wiskott-Aldrich syndrome (WAS), X-linked agammaglobulinemia (XLA), XMEN disease, or a combination thereof.

In an aspect, compositions (e.g., a disclosed AAV vector, a disclosed AAV particle, a disclosed AAV genome, a disclosed AAV viral capsid, a disclosed AAV viral capsid protein, or any combination thereof) herein can be used in methods of generating an immune cell therapy composition that can increase cytolytic activity in immune cells with modulated gene expression as disclosed herein compared to cytolytic activity of native immune cells. In an aspect, an immune cell therapy composition disclosed herein can increase cytolytic activity immune cells with modulated gene expression as disclosed herein by about 1% to about 100%, about 10% to about 90%, or about 20% to about 80% compared to native immune cells. In an aspect, an immune cell therapy composition disclosed herein can increase cytolytic activity in immune cells with modulated gene expression as disclosed herein by about 1%, about 5%, about 10%, about 20%, about 30%, about 40%, about 50%, about 60%, about 70%, about 80%, about 90%, or about 100% compared to native immune cells. In an aspect, an immune cell therapy composition disclosed herein can increase cytolytic activity of immune cells with modulated gene expression as disclosed herein against leukemia cells, lymphoma cells, tumor cells, metastasizing cells of solid tumors compared to cytolytic activity of native immune cells. In an aspect, an immune cell therapy composition disclosed herein can increase cytolytic activity of immune cells with modulated gene expression as disclosed herein from subjects with viral, mycotic or bacterial infectious diseases compared to cytolytic activity of native immune cells.

In an aspect, compositions (e.g., a disclosed AAV vector, a disclosed AAV particle, a disclosed AAV genome, a disclosed AAV viral capsid, a disclosed AAV viral capsid protein, or any combination thereof) herein can be used in methods of generating chimeric antigen receptor (CAR) T cells. As used herein, “chimeric antigen receptor” or “CAR” or “chimeric T cell receptor” refers herein to a synthetically designed receptor having a ligand binding domain of an antibody or another peptide sequence that binds to a molecule associated with the disease or disorder and is linked via a spacer domain to one or more intracellular signaling domains of a T cell or other receptors, such as a costimulatory domain. Chimeric receptor can also be referred to as artificial T cell receptors, chimeric T cell receptors, chimeric immunoreceptors, and chimeric antigen receptors (CARs). Generally, a CAR is designed for a T cell and is a chimera of a signaling domain of the T-cell receptor (TCR) complex and an antigen-recognizing domain (e.g., an antibody single chain variable fragment (scFv) or other antigen binding fragment) (Enblad et al., Human Gene Therapy. 2015; 26(8):498-505). A T cell that expresses a CAR is referred to as a CAR T cell. CARs can redirect T-cell specificity and reactivity toward a selected target in a non-MHC-restricted manner. The non-MHC-restricted antigen recognition gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

There are four generations of CARs, each of which contains different components. First generation CARs join an antibody-derived scFv to the CD3zeta ((or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains can include, e.g., a combination of CD3ζ, CD27, CD28, 4-1BB, ICOS, or OX40. In an aspect, CARs can contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3z and/or co-stimulatory molecules (Maude et al., Blood. 2015; 125(26):4017-4023; Kakarla and Gottschalk, Cancer J. 2014; 20(2):151-155).

CARs typically differ in their functional properties. The CD3ζ signaling domain of the T-cell receptor, when engaged, activates and induces proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs. Clinical trials suggest that both second-generation CARs are capable of inducing substantial T-cell proliferation in vivo. Third generation CARs combine multiple signaling domains (costimulatory) to augment potency.

In an aspect, a chimeric antigen receptor for use herein is a first-generation CAR. In an aspect, a chimeric antigen receptor for use herein is a second-generation CAR. In an aspect, a chimeric antigen receptor for use herein is a third generation CAR. In an aspect, a CAR can comprise an extracellular (ecto) domain comprising an antigen binding domain (e.g., an antibody, such as an scFv), a transmembrane domain, and a cytoplasmic (endo) domain.

### 3. Methods of Making CAR T Cells

Disclosed herein is a method of making CAR T cells using a therapeutically effectively amount of a disclosed AAV vector, a disclosed AAV particle, a disclosed AAV genome, a disclosed AAV viral capsid, a disclosed AAV viral capsid protein, or any combination thereof.

The present disclosure also provides methods of making CAR T cells using compositions (e.g., one or more of a disclosed AAV vector, a disclosed AAV particle, a disclosed AAV genome, a disclosed AAV viral capsid, a disclosed AAV viral capsid protein, or any combination thereof) disclosed herein. For example, in an aspect, a disclosed AAV capsid protein can comprise the sequence set forth in SEQ ID NO:01, wherein amino acids 454-460 of the capsid protein comprise the sequence set forth in any one of SEQ ID NO:05-SEQ ID NO:545. In an aspect, a disclosed AAV capsid protein can comprise the sequence set forth in SEQ ID NO:02.

In an aspect, CRISPR-Cas9 gene-editing components can be used to introduce a site-specific disruption at a gene sequence that is associated with diseases and/or conditions of interest, such as the TCR and/or MCH. In an aspect, the gene-sequence is selected from a component of the TCR. In an aspect, the TCR component is a TRAC. In an aspect, the site-specific disruption is a permanent deletion of at least a portion of the gene. In an aspect, the site-specific disruption is a small deletion in the gene. In an aspect, the site-specific disruption is a small insertion in the gene. In an aspect, the site-specific disruption is an insertion of a nucleic acid encoding a CAR in the gene. In an aspect, a site-specific disruption of the TRAC gene provides a T cell without a functional TCR. In an aspect, a DNA double-stranded break at the TRAC locus can be repaired by homology directed repair with any of the AAV vectors (e.g., AAV6, Ark313) disclosed herein. In an aspect, a DNA double-stranded break at the TRAC locus can be repaired by homology directed repair with any of the AAV vectors (e.g., AAV6, Ark313) herein wherein the AAV vector can comprise a nucleotide sequence containing right and left homology arms to the TRAC locus flanking a chimeric antigen receptor (CAR) cassette.

In an aspect, the present disclosure relates to an administration of a population of engineered T cells (e.g., CAR T cells) with a disrupted TCR and MHC as generated by any of the AAV vectors (e.g., AAV6, Ark313) disclosed herein. In an aspect, the present disclosure relates to administration of a population of cells comprising engineered T cells (e.g., engineered human CAR T cells) with a reduced risk of inducing an AAV-mediated immune response in the recipient patient. In an aspect, CRISPR-Cas9 gene-editing components are used to introduce a site-specific disruption at a TRAC locus. In an aspect, a site-specific disruption in the TRAC locus is an insertion of a nucleic acid encoding a CAR in the gene. In an aspect, a site-specific disruption in the TRAC locus provides a population of engineered T cells (e.g., engineered human CAR T cells) wherein at least 90%, 91%, 92%, 93%, 94%, 95%, 96%, 97%, 98%, or 100% of donor T cells lack expression of a functional TCR. In an aspect, a site-specific disruption in the TRAC locus provides engineered T cells wherein at least 90%, 91%, 92%, 93%, 94%, 95%, 96%, 97%, 98%, or 100% of engineered T cells lack expression of a functional TCR. In an aspect, a site-specific disruption in the TRAC locus and a purification step provides a cell population of engineered T cells (e.g., engineered human CAR T cells) wherein at least 97%, 97.5%, 98%, 98.5%, 99%, 99.5%, or 100% of engineered T cells lack expression of a functional TCR. In an aspect, a site-specific disruption in the TRAC locus and a purification step provides engineered T cells wherein at least 97%, 97.5%, 98%, 98.5%, 99%, 99.5%, or 100% of engineered T cells lack expression of a functional TCR. In an aspect, administration of a population of engineered T cells, wherein at least 97%, 97.5%, 98%, 98.5%, 99%, 99.5%, or 100% of engineered T cells (e.g., engineered human CAR T cells) lack expression of a functional TCR, reduces the risk of an AAV-mediated immune response following administration to a recipient patient. In an aspect, administration of engineered T cells, wherein at least 97%, 97.5%, 98%, 98.5%, 99%, 99.5%, or 100% of engineered T cells lack expression of a functional TCR, reduces the risk of an AAV-mediated immune response following administration to a recipient patient.

### 4. Methods of Treating a Genetic Disease or Disorder

Disclosed herein is a method of treating a genetic disease or disorder comprising administering to a subject in need thereof a therapeutically effective amount of a disclosed AAV vector, a disclosed AAV particle, a disclosed AAV genome, a disclosed AAV viral capsid, a disclosed AAV viral capsid protein, or any combination thereof.

Disclosed herein is a method of treating a genetic disease or disorder comprising administering to a subject in need thereof a therapeutically effective amount of a pharmaceutical composition comprising a disclosed AAV vector, a disclosed AAV particle, a disclosed AAV genome, a disclosed AAV viral capsid, a disclosed AAV viral capsid protein, or any combination thereof.

Disclosed herein is a method of treating a genetic disease or disorder comprising administering to a subject in need thereof a therapeutically effective amount of one or more cells, wherein the one or more cells have been contacted with a disclosed AAV vector, a disclosed AAV particle, a disclosed AAV genome, a disclosed AAV viral capsid, a disclosed AAV viral capsid protein, or any combination thereof.

Disclosed herein is a method of treating a genetic disease or disorder comprising administering to a subject in need thereof a therapeutically effective amount of a pharmaceutical composition comprising one or more cells, wherein the one or more cells have been contacted with a disclosed AAV vector, a disclosed AAV particle, a disclosed AAV genome, a disclosed AAV viral capsid, a disclosed AAV viral capsid protein, or any combination thereof.

In an aspect, the one or more cells have been contacted ex vivo. For example, in an aspect, a disclosed method can comprise administering CAR T cells made by using compositions (e.g., one or more of a disclosed AAV vector, a disclosed AAV particle, a disclosed AAV genome, a disclosed AAV viral capsid, a disclosed AAV viral capsid protein, or any combination thereof) disclosed herein. For example, in an aspect, CAR T cells can be made using a disclosed AAV capsid protein comprising the sequence set forth in SEQ ID NO:01, wherein amino acids 454-460 of the capsid protein comprise the sequence set forth in any one of SEQ ID NO:05-SEQ ID NO:545. In an aspect, CAR T cells can be made using a disclosed AAV capsid protein comprising the sequence set forth in SEQ ID NO:02.

In an aspect, a subject can have or be suspected of having a disease or disorder that can be treated with gene therapy. In an aspect, a subject can have a genetic disease or disorder that affects the immune system.

In an aspect, a subject in need thereof can be diagnosed with an autoimmune disease. By example, but not limited to, a subject can be diagnosed with diabetes (Type 1), lupus, multiple sclerosis, rheumatoid arthritis, celiac disease, or a combination thereof.

In an aspect, a subject in need thereof can be diagnosed with an immune deficiency disease. By example, but not limited to, a subject can be diagnosed with autoimmune lymphoproliferative syndrome (ALPS), autoimmune polyglandular syndrome type 1 (APS-1), BENTA disease, caspase eight deficiency state (CEDS), CARD9 deficiency and other syndromes of susceptibility to Candidiasis, chronic granulomatous disease (CGD), common variable immunodeficiency (CVID), congenital neutropenia syndromes, CTLA4 deficiency, DOCK8 deficiency, GATA2 deficiency, hyper-immunoglobulin E syndrome (HIES), hyper-immunoglobulin M (IgM) syndrome, leukocyte adhesion deficiency (LAD), LRBA deficiency, PI3 kinase disease, PLAID and/or PLAID-like disease, severe combined immunodeficiency (SCID), STAT3 gain-of-function disease, Warts, Hypogammaglobulinemia, Infections, and Myelokathexis (WHIM) Syndrome, Wiskott-Aldrich syndrome (WAS), X-linked agammaglobulinemia (XLA), XMEN disease, or a combination thereof.

Other genetic diseases and disorders include, but are not limited to, diseases and disorders due to a defect in the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF1O, ARHGEF18, ARID1A, ARID1B, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNAlC, CACNAlD, CACNA1E, CACNA1F, CACNAlG, CACNAlH, CACNAlS, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICERI, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNCIHI, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEPI, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCHI, NOTCH2, NOTCH3, NPHP4, NRXN1,NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIl, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAPI, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAMI, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect, a disclosed method of treating a genetic disease or disorder can restore one or more aspects of cellular homeostasis and/or cellular functionality and/or metabolic dysregulation in the subject (such as, for example, homeostasis and/or cellular function and/or metabolic dysregulation relating to the immune system). In an aspect, a disclosed method of treating a genetic disease or disorder can restore the functionality and/or structural integrity of a missing, deficient, and/or mutant protein or enzyme (such as, for example, a protein or enzyme in the immune system). In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity in the subject.

In an aspect, restoring the activity and/or functionality of a missing, deficient, and/or mutant protein or enzyme (such as those, for example, contributing to immune system function) can comprise a 10%, 20%, 30%, 40%, 50%, 60%, 70%, 80%, 90%, 100%, or any amount of restoration when compared to a pre-existing level such as, for example, a pre-treatment level.

In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme (such as those contributing to immune system function). In an aspect, restoration can be a partial or incomplete restoration. In an aspect, restoration can be complete or near complete restoration such that the level of expression, activity, and/or functionality is similar to that of a wild-type or control level.

In an aspect, a therapeutically effective amount of disclosed AAV vector can comprise a range of about 1×10¹⁰ vg/kg to about 2×10¹⁴-vg/kg. In an aspect, for example, a disclosed AAV vector can be administered at a dose of about 1×10¹¹ to about 8×10¹³ vg/kg or about 1×10¹² to about 8×10¹³ vg/kg. In an aspect, a disclosed AAV vector can be administered at a dose of about 1×10¹³ to about 6×10¹³ vg/kg. In an aspect, a disclosed AAV vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed AAV vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed AAV vector can be administered at a dose of about 1×10¹² vg/kg. In an aspect, a disclosed AAV vector can be administered at a dose of about 1×10¹¹ vg/kg. In an aspect, a disclosed AAV vector can be administered in a single dose, or in multiple doses (such as 2, 3, 4, 5, 6, 7, 8, 9 or 10 doses) as needed for the desired therapeutic results. In an aspect, a therapeutically effective amount of disclosed AAV vector can comprise a range determined by a skilled person.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, administering can comprise oral, intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed composition, a disclosed pharmaceutical formulation, and/or a disclosed vector can be concurrently and/or serially administered to a subject via multiple routes of administration. For example, in an aspect, administering a disclosed vector and/or a disclosed pharmaceutical formulation can comprise intravenous administration and intra-cistern magna (ICM) or intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect, a disclosed method of treating and/or preventing a genetic disease or disorder can further comprise administering to the subject a therapeutically effective amount of a therapeutic agent. A therapeutic agent can be any disclosed agent that effects a desired clinical outcome.

In an aspect, a disclosed method of treating and/or preventing a genetic disease or disorder can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of treating and/or preventing a genetic disease or disorder can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed method of treating and/or preventing a genetic disease or disorder can further comprise administering one or more proteasome inhibitors (e.g., bortezomib, carfilzomib, marizomib, ixazomib, and oprozomib). In an aspect, a disclosed method of treating and/or preventing a genetic disease or disorder can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof.

In an aspect, a disclosed method of treating a genetic disease or disorder can comprise repeating a disclosed administering step one or more times such as, for example, repeating the administering of a disclosed AAV vector, a disclosed AAV particle, a disclosed AAV genome, a disclosed AAV viral capsid, a disclosed AAV viral capsid protein, or any combination thereof.

In an aspect, a disclosed method of treating a genetic disease or disorder can comprise repeating a disclosed administering step one or more times such as, for example, repeating the administering of a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of treating a genetic disease or disorder can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed AAV vector, a disclosed AAV particle, a disclosed AAV genome, a disclosed AAV viral capsid, a disclosed AAV viral capsid protein, or any combination thereof administered to a subject, or by changing the frequency of administration of one or more of a disclosed AAV vector, a disclosed AAV particle, a disclosed AAV genome, a disclosed AAV viral capsid, a disclosed AAV viral capsid protein, or any combination thereofto a subject, or by changing the duration of time one or more of a disclosed AAV vector, a disclosed AAV particle, a disclosed AAV genome, a disclosed AAV viral capsid, a disclosed AAV viral capsid protein, or any combination thereof are administered to a subject.

In an aspect, a method can be altered by changing the amount of a disclosed pharmaceutical composition comprising a disclosed AAV vector, a disclosed AAV particle, a disclosed AAV genome, a disclosed AAV viral capsid, a disclosed AAV viral capsid protein, or any combination thereof administered to a subject, or by changing the frequency of administration of a disclosed pharmaceutical composition comprising one or more of a disclosed AAV vector, a disclosed AAV particle, a disclosed AAV genome, a disclosed AAV viral capsid, a disclosed AAV viral capsid protein, or any combination thereof to a subject, or by changing the duration of time one or more of a disclosed pharmaceutical composition comprising a disclosed AAV vector, a disclosed AAV particle, a disclosed AAV genome, a disclosed AAV viral capsid, a disclosed AAV viral capsid protein, or any combination thereof are administered to a subject.

In an aspect, a method can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more of a disclosed AAV vector, a disclosed AAV particle, a disclosed AAV genome, a disclosed AAV viral capsid, a disclosed AAV viral capsid protein, or any combination thereof, a disclosed pharmaceutical composition comprising a disclosed AAV vector, a disclosed AAV particle, a disclosed AAV genome, a disclosed AAV viral capsid, a disclosed AAV viral capsid protein, or any combination thereof, cells generated by using one or more of a disclosed AAV vector, a disclosed AAV particle, a disclosed AAV genome, a disclosed AAV viral capsid, a disclosed AAV viral capsid protein, or any combination thereof, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of treating and/or preventing a genetic disease or disorder can further comprise generating a disclosed AAV vector, a disclosed AAV particle, a disclosed AAV genome, a disclosed AAV viral capsid, a disclosed AAV viral capsid protein, or any combination thereof. In an aspect, a disclosed method can further comprise generating a disclosed viral vector. In an aspect, generating a disclosed viral vector can comprise generating an AAV vector or a recombinant AAV (such as those disclosed herein). In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect, a disclosed method can further reprogram NK cell antitumor activity. In an aspect, a disclosed method can further reducing T cell exhaustion.

In an aspect, a disclosed AAV vector, a disclosed AAV particle, a disclosed AAV genome, a disclosed AAV viral capsid, a disclosed AAV viral capsid protein, or any combination thereof can be used for the delivery of a CRISPR-Cas system.

### 5. Miscellaneous

Disclosed herein are methods of manipulating immune cells using one or more of a disclosed AAV vector, a disclosed AAV particle, a disclosed AAV genome, a disclosed AAV viral capsid, a disclosed AAV viral capsid protein, or any combination thereof.

Disclosed herein are methods of delivering CRISPR to immune cells to generate CAR sequences using one or more of a disclosed AAV vector, a disclosed AAV particle, a disclosed AAV genome, a disclosed AAV viral capsid, a disclosed AAV viral capsid protein, or any combination thereof.

Disclosed herein are methods of genetically reprogramming immune cells to reduce T cell exhaustion using one or more of a disclosed AAV vector, a disclosed AAV particle, a disclosed AAV genome, a disclosed AAV viral capsid, a disclosed AAV viral capsid protein, or any combination thereof.

Disclosed herein are methods of enhancing antitumor activity of immune cells using one or more of a disclosed AAV vector, a disclosed AAV particle, a disclosed AAV genome, a disclosed AAV viral capsid, a disclosed AAV viral capsid protein, or any combination thereof.

Disclosed herein is a preclinical model of engineering cell therapies in immunocompetent hosts using one or more of a disclosed AAV vector, a disclosed AAV particle, a disclosed AAV genome, a disclosed AAV viral capsid, a disclosed AAV viral capsid protein, or any combination thereof.

Disclosed herein is a preclinical model of T cell function in an autoimmune disease using one or more of a disclosed AAV vector, a disclosed AAV particle, a disclosed AAV genome, a disclosed AAV viral capsid, a disclosed AAV viral capsid protein, or any combination thereof.

Disclosed herein is a method of performing homology directed repair in cells using one or more of a disclosed AAV vector, a disclosed AAV particle, a disclosed AAV genome, a disclosed AAV viral capsid, a disclosed AAV viral capsid protein, or any combination thereof.

Disclosed herein is a method of enhancing transduction efficiency in murine T cells using one or more of a disclosed AAV vector, a disclosed AAV particle, a disclosed AAV genome, a disclosed AAV viral capsid, a disclosed AAV viral capsid protein, or any combination thereof.

Disclosed herein is a method of precise genome engineering in murine T cells using one or more of a disclosed AAV vector, a disclosed AAV particle, a disclosed AAV genome, a disclosed AAV viral capsid, a disclosed AAV viral capsid protein, or any combination thereof.

Disclosed herein is a method of performing nucleofection-free DNA delivery using one or more of a disclosed AAV vector, a disclosed AAV particle, a disclosed AAV genome, a disclosed AAV viral capsid, a disclosed AAV viral capsid protein, or any combination thereof.

In an aspect, disclosed immune cells can comprise memory/effector T cells, naive T cells, NK cells, or any combination thereof. In an aspect, a disclosed method can comprise contacting the disclosed immune cells vitro, ex vivo, or in vivo. In an aspect, immune cells can be contacted with a disclosed viral vector comprising an AAV capsid protein comprising the sequence set forth in SEQ ID NO:01, wherein amino acids 454-460 of the capsid protein comprise the sequence set forth in any one of SEQ ID NO:05-SEQ ID NO:545. In an aspect, immune cells can be contacted with a disclosed viral vector comprising an AAV capsid protein comprising the sequence set forth in SEQ ID NO:02. In an aspect, a disclosed viral vector can comprise the nucleic acid sequence set forth in SEQ ID NO:04. In an aspect, immune cells can be contacted with one or more a disclosed AAV vector, a disclosed AAV particle, a disclosed AAV genome, a disclosed AAV viral capsid, a disclosed AAV viral capsid protein, or any combination thereof. In an aspect, a disclosed method can reduce tumor size in a subject and/or improve survival of a subject. In an aspect, a disclosed method can be used to screen one or more libraries of genes in human T cells.

Disclosed herein is a nucleotide sequence encoding an adeno-associated virus (AAV) capsid protein variant, wherein the encoded AAV capsid protein variant has at least 90% identity to the sequence of SEQ ID NO:01, wherein the amino acids corresponding to amino acids 454-460 of SEQ ID NO:01 are substituted with a peptide having a sequence of any one of SEQ ID NO:05-SEQ ID NO:545. Disclosed herein is a recombinant AAV capsid protein variant, wherein the capsid protein variant comprises a peptide having the sequence of any one SEQ ID NO:05-SEQ ID NO:545. Disclosed herein is an AAV capsid protein variant, wherein the AAV capsid protein variant comprises the sequence of SEQ ID NO:02 or a sequence with at least 90% or at least 95% identity thereto. Disclosed herein is a recombinant AAV vector comprising a disclosed AAV capsid protein variant, wherein the AAV vector comprises a vector genome. In an aspect, a disclosed vector genome is encapsidated by an AAV capsid comprising a disclosed AAV capsid protein variant. In an aspect, a disclosed vector genome comprises a first inverted terminal repeat (ITR) and a second ITR. In an aspect, a disclosed vector genome comprises a transgene located between the first ITR and the second ITR. In an aspect, a disclosed transgene encodes a therapeutic RNA. In an aspect, a disclosed transgene encodes a therapeutic protein. In an aspect, a disclosed transgene encodes a gene-editing molecule. In an aspect, a disclosed gene-editing molecule is a nuclease. In an aspect, a disclosed nuclease is a Cas9 nuclease. In an aspect, a disclosed gene-editing molecule is a single guide RNA (sgRNA). Disclosed herein is an AAV capsid protein variant comprising a peptide having the sequence of any one of SEQ ID NO:05-SEQ ID NO:545. Disclosed herein is an AAV capsid protein variant comprising an amino acid sequence having at least 90% identity to the sequence of SEQ ID NO:01, wherein the amino acids corresponding to amino acids 454-460 of SEQ ID NO:01 are substituted with a peptide having a sequence of any one of SEQ ID NO:05-SEQ ID NO:545. Disclosed herein is an AAV capsid protein variant comprising an amino acid sequence of SEQ ID NO: 2 or a sequence with at least 90% or at least 95% identity thereto. Disclosed herein is an AAV capsid comprising a disclosed AAV capsid protein variant. In an aspect, a disclosed AAV capsid comprises about 60 copies of the AAV capsid protein variant, or fragments thereof. In an aspect, a disclosed AAV capsid comprises one or more copies of the AAV capsid protein variant and wherein the AAV capsid protein variants are arranged with T=1 icosahedral symmetry. Disclosed herein is a recombinant AAV vector comprising a disclosed AAV capsid protein variant or a disclosed AAV capsid. Disclosed herein is a pharmaceutical composition comprising a disclosed recombinant AAV vector or a disclosed pharmaceutical composition. Disclosed herein is a method of introducing a recombinant AAV vector into a target cell, the method comprising contacting the target cell with a disclosed recombinant AAV vector or a disclosed pharmaceutical composition. A method of delivering a transgene to a target cell in a subject, the method comprising administering to the subject a disclosed recombinant AAV vector or a disclosed pharmaceutical composition. In an aspect, the target cell is an immune cell. In an aspect, a disclosed immune cell comprises a T cell, a NK cell, or a combination thereof. In an aspect, contacting of the cell is performed in vitro, ex vivo, or in vivo. Disclosed herein is a method of treating a subject in need thereof, comprising administering to the subject an effective amount of a disclosed recombinant AAV vector or a disclosed pharmaceutical composition. Disclosed herein is a method of treating a subject in need thereof, comprising administering to the subject a cell that has been contacted ex vivo with a disclosed recombinant AAV vector or a disclosed pharmaceutical composition. In an aspect, the subject comprises a mammal. In an aspect, the subject is a human or a mouse.

### E. Kits

Disclosed herein is a kit comprising one or more of a disclosed AAV vector, a disclosed AAV particle, a disclosed AAV genome, a disclosed AAV viral capsid, a disclosed AAV viral capsid protein, or any combination thereof. Disclosed herein is a kit comprising cells generated by using one or more of a disclosed AAV vector, a disclosed AAV particle, a disclosed AAV genome, a disclosed AAV viral capsid, a disclosed AAV viral capsid protein, or any combination thereof. Disclosed herein is a kit comprising CAR T cells generated by using one or more of a disclosed AAV vector, a disclosed AAV particle, a disclosed AAV genome, a disclosed AAV viral capsid, a disclosed AAV viral capsid protein, or any combination thereof.

In an aspect, a disclosed kit can be used to prepare one or more of a disclosed AAV vector, a disclosed AAV particle, a disclosed AAV genome, a disclosed AAV viral capsid, a disclosed AAV viral capsid protein, or any combination thereof. In an aspect, a disclosed AAV capsid protein can comprise the sequence set forth in SEQ ID NO:02. In an aspect, a disclosed AAV capsid protein can be encoded by the sequence set forth in SEQ ID NO:04.

Disclosed herein is a kit comprising a pharmaceutical formulation comprising one or more of a disclosed AAV vector, a disclosed AAV particle, a disclosed AAV genome, a disclosed AAV viral capsid, a disclosed AAV viral capsid protein, or any combination thereof.

In an aspect, a disclosed kit can comprise at least two components constituting the kit. Together, the components constitute a functional unit for a given purpose (such as, for example, treating a subject in need thereof). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise (i) one or more of a disclosed AAV vector, a disclosed AAV particle, a disclosed AAV genome, a disclosed AAV viral capsid, a disclosed AAV viral capsid protein, or any combination thereof, and (ii) a label or package insert with instructions for use. In an aspect, suitable containers include, for example, bottles, vials, syringes, blister pack, etc. The containers can be formed from a variety of materials such as glass or plastic. The container can hold comprising one or more of a disclosed AAV vector, a disclosed AAV particle, a disclosed AAV genome, a disclosed AAV viral capsid, a disclosed AAV viral capsid protein, or any combination thereof a disclosed pharmaceutical formulation, or any combination thereof, and can have a sterile access port (for example the container may be an intravenous solution bag or a vial having a stopper pierceable by a hypodermic injection needle). The label or package insert can indicate one or more a disclosed AAV vector, a disclosed AAV particle, a disclosed AAV genome, a disclosed AAV viral capsid, a disclosed AAV viral capsid protein, or any combination thereof can be used for delivering gene therapy, for delivering CAR gene therapy, for delivering CRISPR to engineer long CAR sequences, for genetically reprogramming T cells to, for example, reduce exhaustion and/or enhance NK cell antitumor activity. A disclosed kit can comprise additional components necessary for administration such as, for example, other buffers, diluents, filters, needles, and syringes

As stated above, a disclosed kit can comprise instructions relating to the use, dosage, dosing schedule, and/or route of administration of one or more of a disclosed AAV vector, a disclosed AAV particle, a disclosed AAV genome, a disclosed AAV viral capsid, a disclosed AAV viral capsid protein, or any combination thereof.

In an aspect, a disclosed kit can provide additional components such as buffers and other interpretive information. In an aspect, the disclosure can provide articles of manufacture comprising contents of the kits described above.

### IX. EXAMPLES

While the present disclosure has been described with reference to the specific embodiments thereof, it should be understood by those skilled in the art that various changes can be made and equivalents can be substituted without departing from the true spirit and scope of the disclosure. In addition, many modifications can be made to adapt a particular situation, material, composition of matter, process, process step or steps, to the objective, spirit, and scope of the present disclosure. All such modifications are intended to be within the scope of the disclosure.

### A. PRELIMINARY EXAMPLES

Precise targeting of large transgenes to T cells using homology-directed repair has been transformative for adoptive cell therapies and T cell biology. Non-toxic delivery of large DNA templates via adeno-associated virus (AAV) has greatly improved knock-in efficiencies, but the tropism of current AAV serotypes restricts their use to human T cells employed in immunodeficient mouse models. As described below, to enable targeted knock-ins in murine T cells, structure-guided evolution on the AAV6 capsid was performed to generate Ark313 (which is a synthetic AAV that exhibits high transduction efficiency in murine T cells). As outline in the ensuing Examples, Ark313 can be used for nucleofection-free DNA delivery, CRISPR/Cas9-mediated gene knockouts, and targeted integration of large transgenes with efficiencies up to 50%. Moreover, Ark313 enabled pre-clinical modeling of Trac-targeted CAR- and transgenic TCR-T cells in immunocompetent models. Efficient gene targeting in murine T cells holds great potential for improved T cell therapies and opens new avenues in experimental T cell immunology.

### Example 1

### AAV6 Mutant Improved Mouse T Cell Transduction

The method for generating AAV capsid protein variants was as follows. The first step involved identification of conformational 3D antigenic epitopes on the AAV6 capsid surface using cryo-electron microscopy. AAV6 libraries were then engineered through saturation mutagenesis of amino acid residues identified within the surface loops. Specifically, amino acid residues within 454-460 (VP1 numbering; 454-GSAQNKD-460 (SEQ ID NO:01)) were selected for saturation mutagenesis and generation an AAV6 parental library.

Selected residues within the antigenic motifs were subjected to mutagenesis using degenerate primers with each codon substituted by nucleotides NNK and gene fragments combined together by Gibson assembly (a sequence overlap-based method). Specifically, to generate the AAV6 parental library, oligonucleotides containing a 21-mer and homology arms to AAV6 Cap gene were synthesized through Integrated DNA Technologies. The resulting capsid-encoding genes containing a degenerate library of mutated antigenic motifs were cloned into a wild type AAV genome to replace the original Cap encoding DNA sequence, yielding a plasmid library. Specifically, the plasmid contained genes encoding AAV2 Rep and AAV6 Cap flanked by AAV2 ITRs, with amino acids in the AAV6 Cap mutated to stop codons to reduce wild-type AAV6 plasmid contamination.

The AAV6 parental plasmid library was then transfected into HEK 293 producer cell lines with an adenoviral helper plasmid to generate an AAV6 capsid parental library. In brief, HEK293 cells were transfected at 70 to 80% confluence with polyethylenimine with equal molar ratios of pTR-AAV6-Library and adenovirus helper plasmid pXX680. HuH7 (human hepato cellular carcinoma) cells were cultured to ˜75% confluence and infected overnight with the AAV6 libraries at 5,000 viral genomes per cell. The following day, the culture medium was replaced with medium containing Ad5 at a multiplicity of infection (MOI) of 0.5. At 50% to 75% cytopathic effect, the supernatant was collected and incubated at 55° C. for 30 minutes to inactivate the Ad5. DNase I-resistant viral genomes in the media were quantified and served as the inoculum for the subsequent round of infection.

To select for new AAV6 strains that can escape neutralizing antibodies (NAbs) with tropism toward T cells, and/or act in a more potent manner than naturally occurring AAV6, the AAV6 library prepared as described above was subjected to multiple rounds or “cycles” of infection in mice (C57BL/6J mice).

In the first cycle, the AAV6 library prepared as described above was intravenously (i.v.) injected into 8-week C57/B6 mice at about 3×10¹³ to 5×10¹³ vg/kg. Mice were sacrificed 6 days post injection and viral DNA was amplified via PCR from genomic DNA extracted from T cells isolated from mouse spleens using oligonucleotides targeting the AAV6 flanking DNA sequences to amplify the AAV library sequences as described above. In brief, isolation of mouse T cells from spleens was performed using negative selection T cell isolation (Stem Cell Technologies), followed by activation using CD3/CD28 Dynabeads (Gibco). The resulting amplicons were then cloned back into vectors to generate another evolved plasmid library using the same method as generating the first evolved plasmid library above. This time the viral genomes in the media were quantified and served as the inoculum for the next cycle. In total, three cycles were performed in mice. After the last cycle, viral DNA was amplified from genomic DNA extracted from various T cells harvested from mouse spleens as described above. Amplified viral DNA was subjected to high-throughput sequencing using the lllumina MiSeq platform and the resulting data was analyzed as follows. Demultiplexed reads were subjected to a quality control check using FastQC (v.0.11.5), with no sequences flagged for poor quality, and analyzed via a custom Perl script using methods similar to those described in Tse et al. (2017) PNAS. 114(24):E4812-E4821, the disclosure of which is incorporated herein in its entirety. In brief, raw sequencing files were probed for mutagenized regions of interest, and the frequencies of different nucleotide sequences in this region were counted and ranked for each library. Nucleotide sequences were also translated, and these amino acid sequences were similarly counted and ranked. Amino acid sequence frequencies across libraries were then plotted in the R graphics package v3.5.2. A second Perl script was used to calculate the amino acid representation at each position in each library, taking into account the contribution of each mutant in the library.

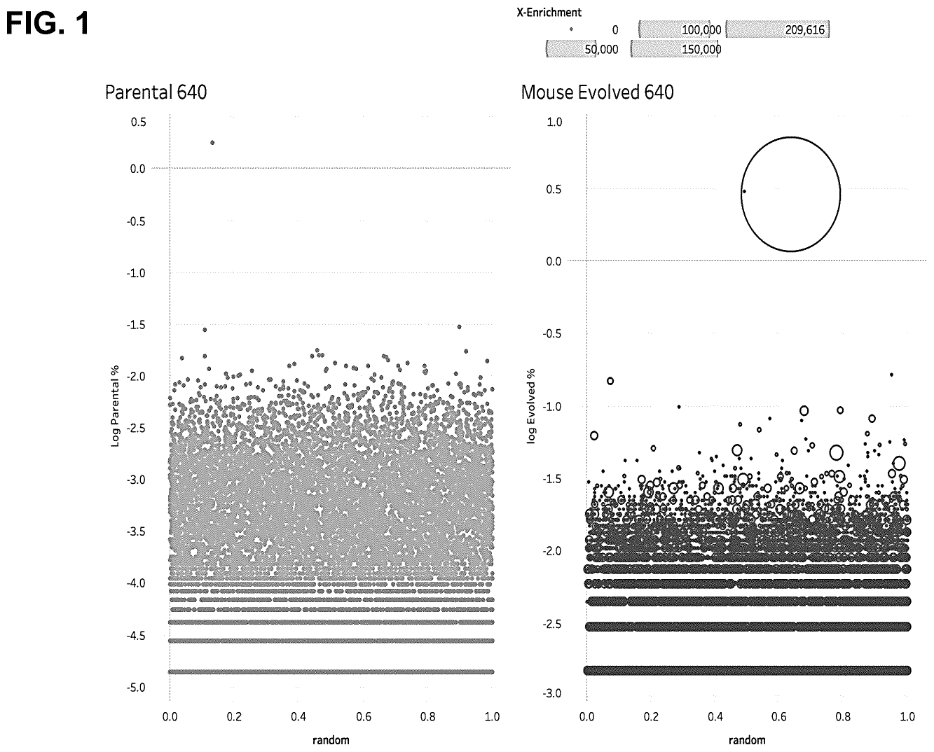

Subjecting these libraries to multiple rounds of evolution yielded several AAV6 capsid variants. The AAV6 capsid variants resulting from the cross-species in vivo screening with the highest frequency were sequenced. Bubble plots showed library diversity, directed evolution and enrichment of novel antigenic footprints in the 454-460 amino acid region between the parental libraries (FIG. 1). Substitutions present in these AAVs in the region (454-GSAQNKD-460 (SEQ ID NO:01)) are shown in Table 4. One variant was highly selected for AAV6 VP1 (Ark313) wherein its capsid variant had the following amino acid substitutions: G454V S455V A456N Q457P N458A K459E D460G (i.e., 454-VVNPAEG-460; SEQ ID NO:05).

### Example 2

### Ark313 Enhanced Transfection Efficiency Compared to Wild-Type AAV6

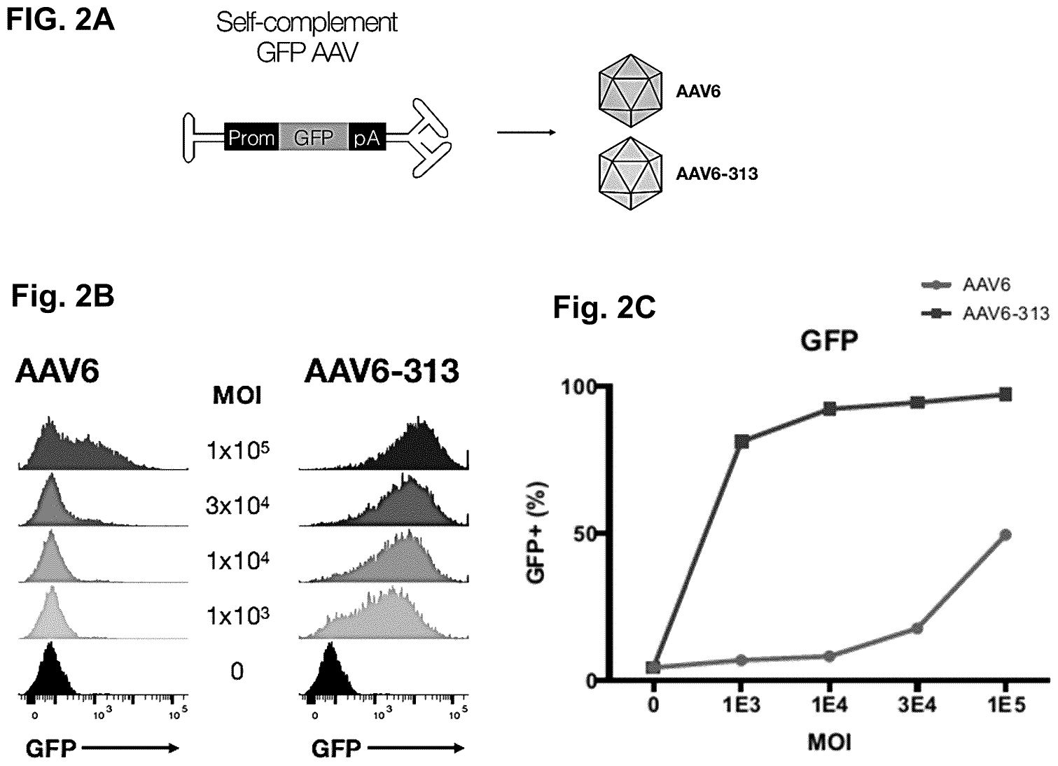

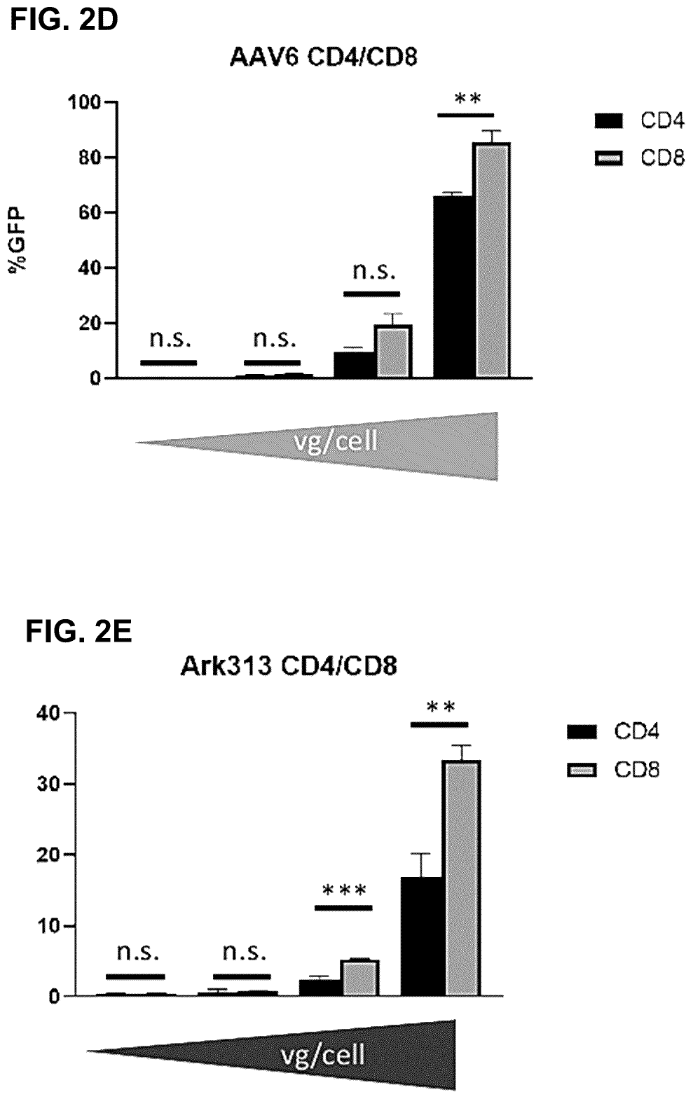

Two capsid proteins, AAV6 WT (SEQ ID NO:01) and Ark313 (SEQ ID NO:02) were selected for ex vivo characterization in T cells harvested from mice. AAVs comprising these capsid proteins and packaging of a fluorescent transgene were generated (i.e., GFP). In brief, recombinant AAV vectors were produced by transfecting HEK293 cells at 70 to 80% confluence with polyethylenimine using the triple-plasmid transfection protocol. Recombinant vectors packaging a self-complementary AAV6 driven by either CBh-eGFP were generated using this method. (FIG. 2A). Subsequent steps involving the harvesting of recombinant AAV vectors and downstream purification were carried out. In brief, vector purification was carried out using iodaxinol gradient ultracentrifugation protocol, buffer exchange and concentration using vivaspin2 100 kDa molecular weight cut-off (MWCO) centrifugation columns (F-2731-100 Bioexpress). Recombinant AAV vector titers were determined by quantitative PCR with primers amplifying AAV2 inverted terminal repeat regions (ITRs) (5′-AACATGCTACGCAGAGAGGGAGTGG-3′ (SEQ ID NO:546) and 5′-CATGAGACAAGGAACCCCTAGTGATGGAG-3′ (SEQ ID NO:547)).

Mouse T cells were harvested from spleens. In brief, isolation of mouse T cells from spleens was performed using negative selection T cell isolation (Stem Cell Technologies), followed by activation using CD3/CD28 Dynabeads (Gibco). Cells were counted and used for experiments after 24 hours of activation. T cells were maintained at a cell density of 2×10⁶ cells per mL unless specified otherwise. For incubation with the GFP containing AAVs, 1×10⁵ to 2×10⁵ activated mouse T cells were incubated in 96 well plates with a range of AAV MOIs at 1×10⁶ cells per mL for knock-in or 2×10⁶ cells per mL for transient GFP expression. GFP expression was analyzed by flow cytometry using a BD LSRFortessa X-50 analyzer. Flow cytometry was performed 48 hours after AAV addition for transient GFP expression. FIG. 2B and FIG. 2C show increased amounts of GFP positive cells at each MOI for T cells transfected with Ark313 compared to wildtype AAV6 demonstrating that Ark313 has enhanced transfection efficiency compared to wildtype AAV6. When flow cytometry was performed to determine the amounts of CD4 T cells infected compared to CD8 T cells, both WT AAV6 and Ark313 showed a higher percentage of GFP in CD8 T cells indicating an ex vivo bias toward this T cell type (FIG. 2D-FIG. 2E).

### Example 3

### Delivery of Donor Template Ark313 Improved Gene Targeting in Mouse T Cells

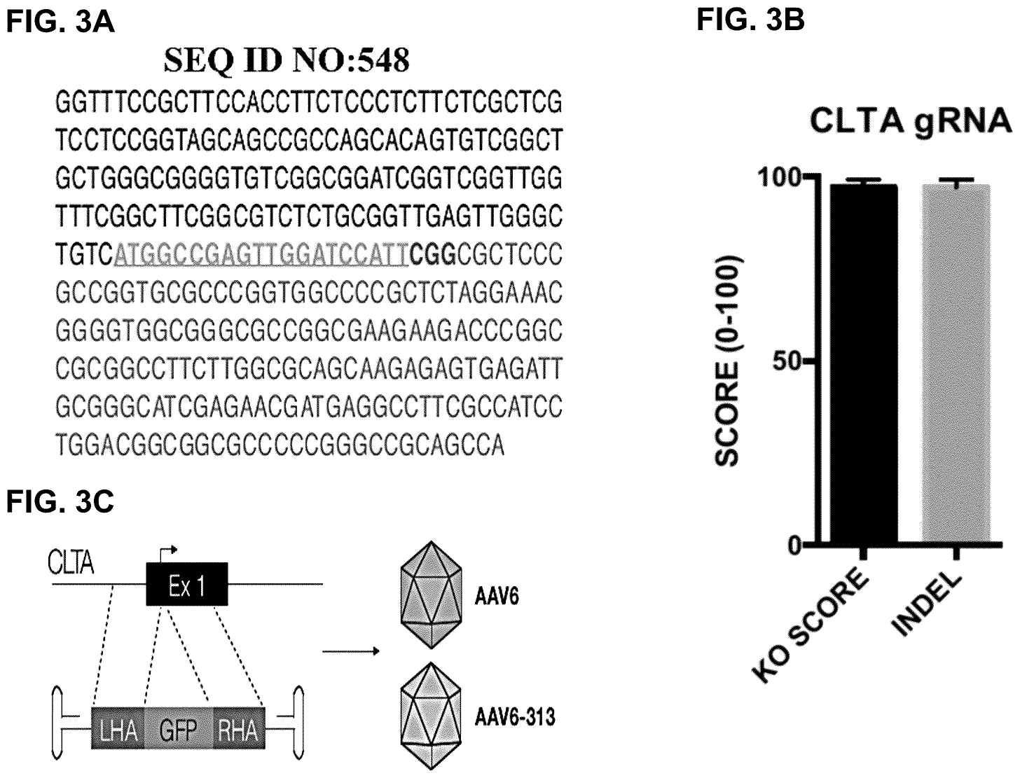

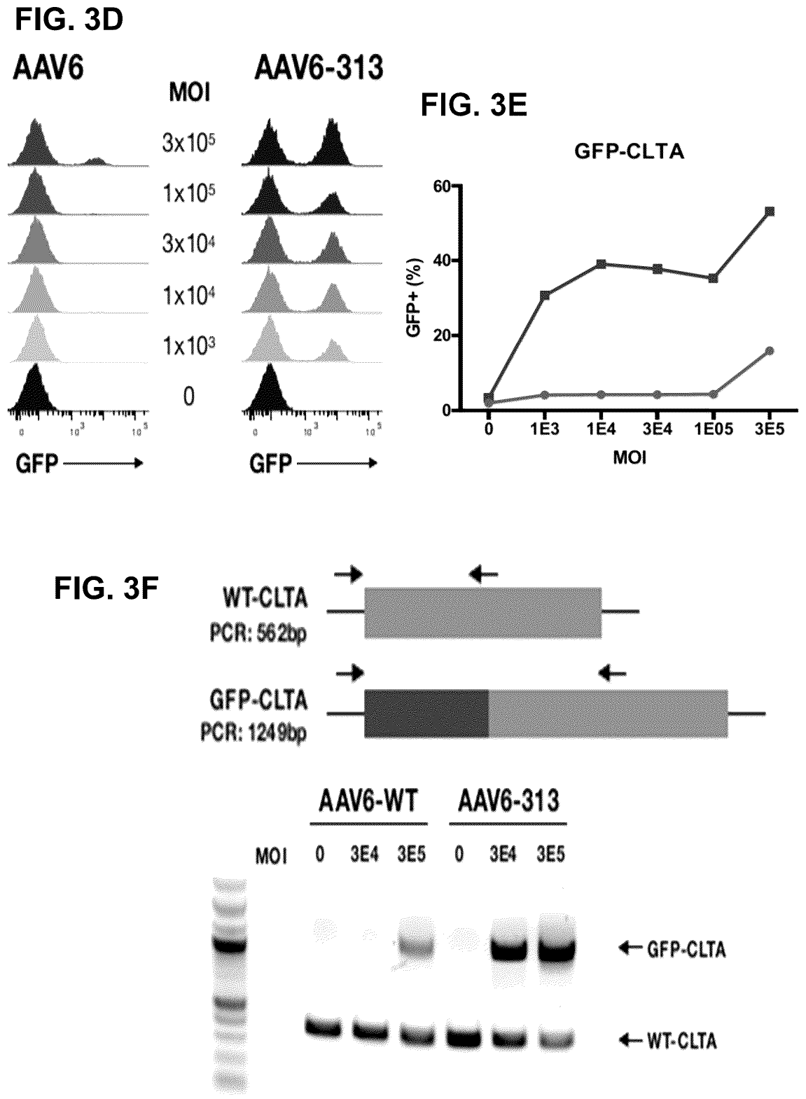

To determine if Ark313 can be used for homology directed repair (HDR) to correct DNA double-strand breaks in T cells ex vivo, AAV mediated knock-in of GFP was assessed. First, a vector having N-terminal fusion of GFP to Clta locus was generated (FIG. 3C) where the genomic sequence of Clta exon1 was targeted by a gRNA (gRNA is underlined and marked in orange followed by a PAM sequence marked in red in FIG. 3A; SEQ ID NO:548).

To perform CRISPR/Cas9 genomic targeting in mouse T cells, ribonucleoproteins (RNP) were generated by combining 60 nmol of Cas9 (Berkeley, QB3) with 120 nmol sgRNA (Synthego) (Clta gRNA: 5′-AUGGCCGAGUUGGAUCCAUU-3′; SEQ ID NO:549) and incubated for 15 minutes at 37° C. RNPs were then combined with 2E6 T cells in 20 μL Amaxa buffer P3 and electroporated using an Amaxa 96 Shuttle System (Lonza) using the electroporation program DN-100. FIG. 3B shows mouse T cells electroporated with Cas9 and Clta gRNA (Clta RNP).

For AAV mediated knock-in, 2×10⁶mouse T cells were electroporated with Clta RNP followed by addition of AAV6 (WT or Ark313) at a range of multiplicity of infections (MOI) 30 minutes after electroporation of the cells at a cell density of 2×10⁶ cells per mL. Cells were then incubated over night before being replaced by fresh cell culture media. Genomic DNA was isolated at 48 hours post electroporation and knock out efficiency was assessed by sanger sequencing and analysis using ICE software (Synthego) with primers flanking the GFP insert (Forward 5′-TTGTGGCTCACCAACCCAACCG-3′ (SEQ ID NO:550), Reverse 5′-CACTCAGAAGCCGGCAGTCTGC-3′ (SEQ ID NO:551)). Additionally, mouse T cells were electroporated with Clta RNP and incubated with either AAV6 WT or Ark313 overnight before GFP expression was analyzed 72 hours after AAV addition by flow cytometry (FIG. 3D). FIG. 3E shows the percentage of GFP positive mouse T cells at each MOI. FIG. 3F shows validation of the Clta gene targeting by PCR analysis with primers flanking the integration site.

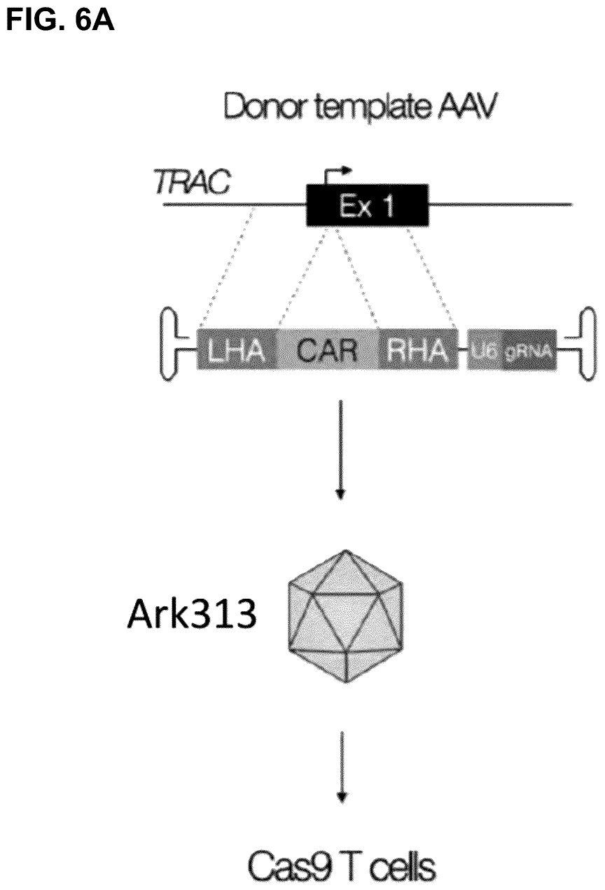

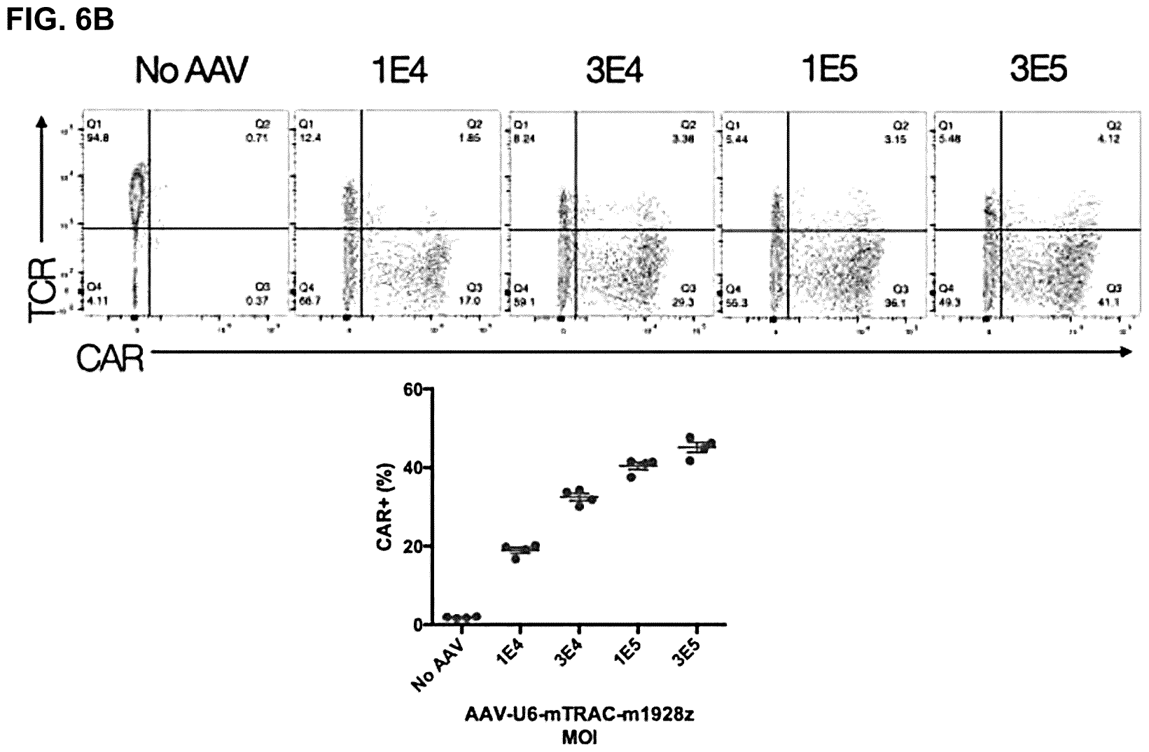

Similarly, the above experiments were repeated using a vector having a CAR for insertion into the TRAC locus (FIG. 6A). For AAV mediated knock-in of the CAR into TRAC, 2×10⁶ mouse T cells were electroporated with CAR RNP followed by addition of Ark313 at a range of multiplicity of infections (MOI) 30 minutes after electroporation at a cell density of 2×10⁶ cells per mL. Cells were then incubated over night before being replaced by fresh cell culture media. FIG. 6B shows increasing percentages of CAR was transduced in the T cells ex vivo using Ark313.

### Example 4

### Ark313 Infected Mouse T-Cells In Vivo

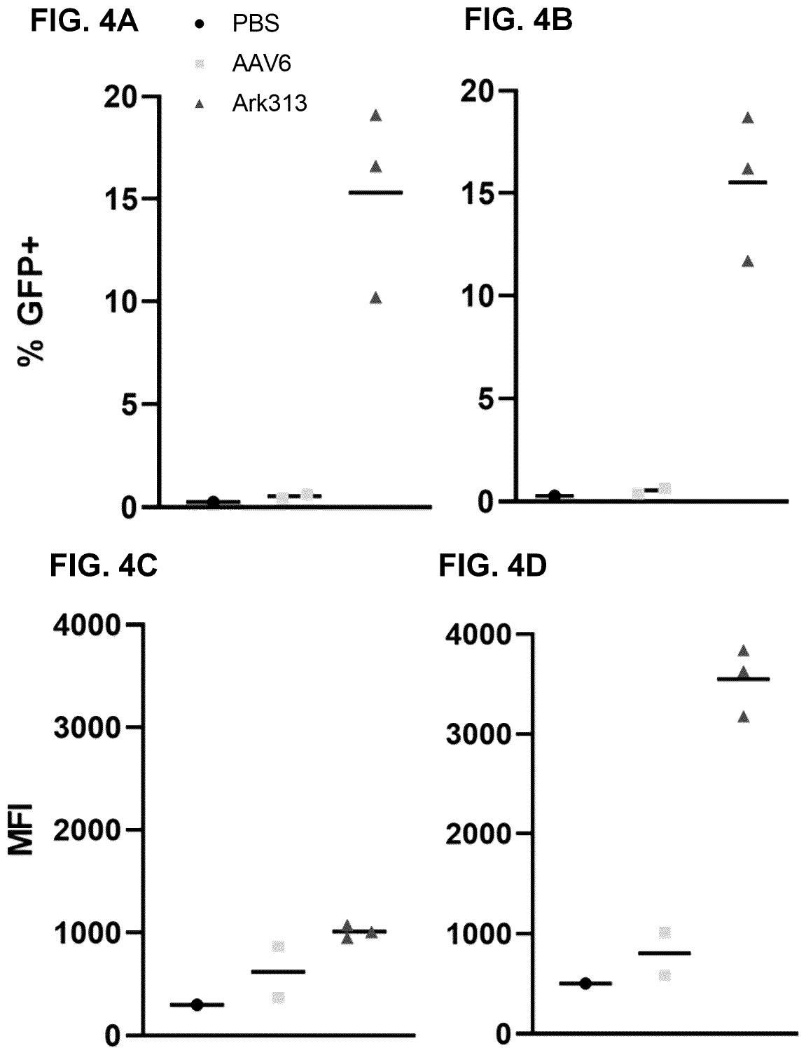

To determine if Ark313 can infect T cells in vivo, WT AAV6 or Ark313 were systemically administered to mice. In brief, mice (8-week-old, C57/B6 mice) were injected intravenously with 2.5×10¹¹ vg of either AAV6 or Ark313 encoding a scCBh-GFP cassette which was prepared as described herein. The mice were sacrificed one-week post injection. T-cells were isolated from splenocytes and either were or were not activated with CD3/CD28 dynabeads. Two days after activation/non-activation, T cells were analyzed by flow cytometry and the percentage of GFP positive cells was measured for unstimulated (FIG. 4A) or activated (FIG. 4B) cells. MFI for unstimulated (FIG. 4E) or activated (FIG. 4D) was also determined.

### Example 5

### Ark313 Infected Mouse T-Cells In Vivo

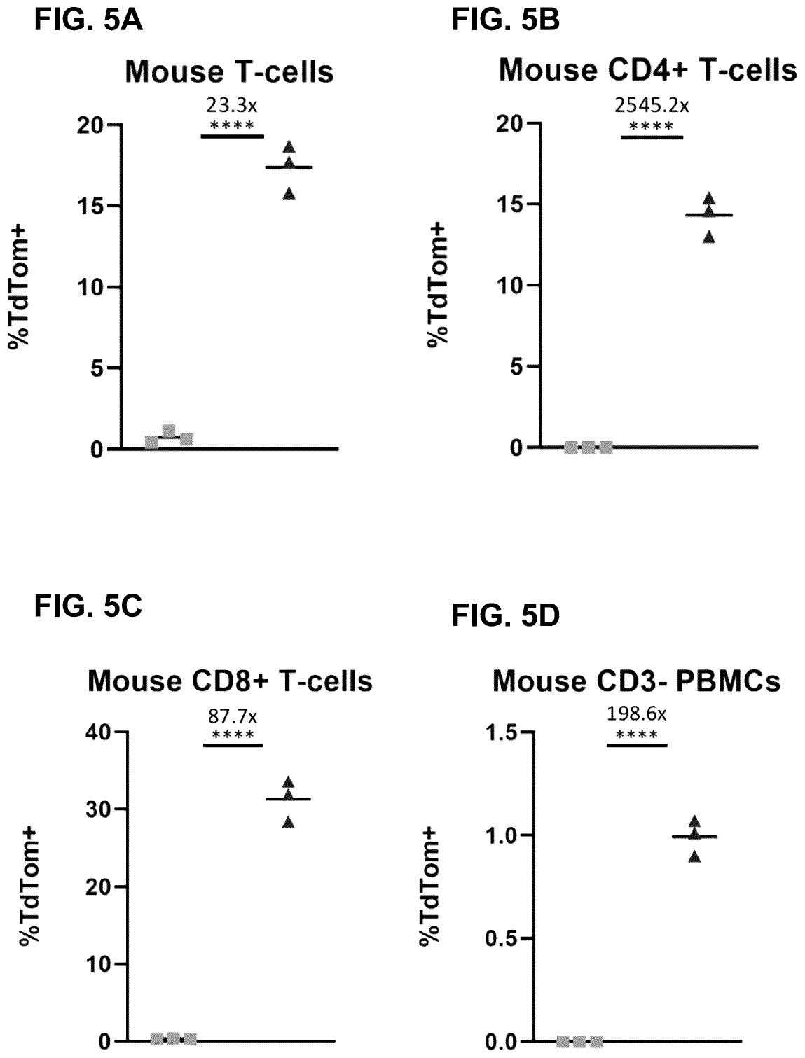

Ai9 male and female mice were injected intravenously at a dose of 1×10¹² vg/kg with a single stranded AAV6 WT or Ark313 vector to deliver Cre Recombinase. Animals were bleed 4 weeks post injection, PBMCs were harvested and transduction of immune cells was evaluated by flow cytometry. FIG. 5A-FIG. 5D show amounts of native tdTomato fluorescence following i.v. administration of AAV6 or Ark313 vectors in mouse T-cells.

### B. ADDITIONAL EXAMPLES

In Example 6—Example 10 described below, a structure-guided evolution approach was used to evolve a novel AAV variant dubbed Ark313. Ark313 was derived from AAV6 and exhibited high transduction efficiency in murine T cells. As detailed below, Ark313 can be used for transient gene delivery and precise genome engineering in primary murine T cells. Ark313 can be used to model various engineering strategies from human T cells in the murine context. These examples present new gene targeting strategies that expand the use of genetically engineered T cells for in vivo studies. Furthermore, through a genome-wide knockout screen, an essential murine host factor and the mechanism for Ark313 cellular entry. Ark313 opens new avenues in experimental T cell immunology and the preclinical modeling of precision-engineered cell therapies in immunocompetent hosts.

### Materials and Methods for Example 6—Example 10

### (a) Plasmids

Two scAAV vectors were produced for transiently expressing GFP. The first was scAAV-CBh-GFP and the second was a CMV enhancer chicken R-actin intron (CAG) promoter (scAAV-CAG-GFP, Addgene #83279). The two vectors are distinguished in the text and figure legends.

To generate a GFP fusion at the Clta N-terminus, the GFP gene was cloned into an AAV plasmid containing homology arms targeting the Clta exon 1 start codon; LHA (351 bp) (SEQ ID NO:594) and RHA (303 bp) (SEQ ID NO:595) sequences. For nucleofection-free knock-in, a U6 promoter for expressing a Clta-targeting sgRNA (AUGGCCGAGUUGGAUCCAUU) (SEQ ID NO:549) was introduced upstream of the LHA.

For integrating genes at the Trac locus, homology arms targeting Trac exon 1 were cloned into an AAV plasmid; LHA (497 bp) (SEQ ID NO:596) and RHA (500 bp) (SEQ ID NO:597) sequences. Genes for either 1928z CAR (flanked by P2A sequences), hCD19-targeting HIT, or OT-I TCR were cloned between the homology arms. To generate a TCR rescue construct, a gene fragment to rescue Trac was introduced after the 1928z P2A. For nucleofection-free knock-in, a U6 promoter for expressing a Trac-targeting sgRNA (UAUGGAUUCCAAGAGCAAUG) (SEQ ID NO:584) was introduced upstream of the LHA.

For retroviral expression under the 5′ LTR promoter, the 1928z CAR was cloned into an MSCV plasmid, and a P2A sequence and the Thy1.1 gene were included downstream of the CAR. Knockouts with Ark313 used a U6 promotor for expressing either a scrambled (SCR) negative control sgRNA or a Trac-targeting sgRNA (UAUGGAUUCCAAGAGCAAUG) (SEQ ID NO:582).

### (b) AAV Production

AAV2-ITR containing plasmids were utilized to package vector genomes into different AAV capsids by transfection of HEK293 cells together with Adenovirus Helper and AAV Rep-Cap plasmids using Polyethylenimine. AAV vectors were further purified following media harvest and PEG precipitation using iodixanol gradient ultracentrifugation. AAV vector titers were determined by qPCR on DNaseI (NEB #B0303S) treated, Proteinase K (Qiagen #1114886) digested AAV samples post-purification, using primers against the vector genome. qPCR was performed with SsoFast Eva Green Supermix (Bio-Rad #1725201) on a StepOnePlus Real-Time PCR System (Applied Biosystems #4376600). Relative quantity was determined by a serial dilution standard of known quantity for each vector plasmid.

### (c) AAV Transduction

Unless otherwise specified, AAV transduction of T cells was performed as follows. Activated T cells, 24 hr for murine cells and 48 hr for human cells, were seeded at 2×10⁶ cells per mL in T cell medium. AAV was added at a specified MOI. It was ensured that the volume of AAV added never exceeded 20% of the culture volume. After incubating the culture overnight, the AAV-containing medium was exchanged for fresh medium, and the T cells were subsequently cultured in standard conditions.

### (d) Generating the AAV6 Capsid Library

The AAV6 capsid library was generated by performing saturation mutagenesis of seven residues in the VR-IV region as reported previously (Tse L V, et al. (2017). Proc Natl Acad Sci USA. 114:E4812-E4821). To generate the library, an overlap extension PCR was performed using two amplicons amplified from a modified AAV6 backbone containing tandem stop codons replacing the randomized region to prohibit potential amplification of the wild type sequence. The randomized region, from the start of AAV6 Cap up to the SbfI site and an overlap, were encoded on one amplicon while a second amplicon encoded the remaining portion of the AAV6 Cap up to the BsiWI site. The two resulting amplicons were combined in equimolar ratio in a second overlap extension PCR step. This final assembled amplicon was digested using BsiWI and SbfI and ligated into the pITR2-Rep2-dead(GFP)Cap6 backbone, which contains AAV2 ITRs and Rep along with the AAV6 Cap gene interrupted by a filler sequence derived from GFP, inserted out of frame into the cognate BsiWI and SbfI site, thus eliminating any potential wild type AAV6 from the library ligation.

AAV6 capsid libraries by co-transfection of HEK293T cells with Adenovirus Helper plasmids with the Rep Cap plasmid library. Activated murine T cells were seeded at 10⁶ cells per mL and the pooled AAV6 capsid library was transduced for 6 hr at an MOI of 10⁴. Transduced cells were washed twice with PBS to remove any unbound AAV, cellular and viral DNA was extracted from cells using an IBI genomic DNA extraction kit (IBI Scientific #IB47280). The Cap region was amplified by PCR before being digested and ligated back into the pITR2-Rep2-dead(GFP)Cap6 backbone to generate the next-round library. Ligation products were concentrated and purified by ethanol precipitation. Purified products were electroporated into DH10B ElectroMax cells (Invitrogen #18290015) and directly plated on multiple 5,245-mm² bioassay dishes (Corning #431111) with ampicillin LB agar to maintain library diversity. Plasmid DNA from AAV6 capsid libraries was purified from pooled colonies grown on LB agar plates with ampicillin using a ZymoPURE II Plasmid Maxiprep kit (Zymo Research #D4203). The process of library production, AAV packaging, T cell transduction, and viral DNA extraction was performed three times to generate the evolved library.

### (e) AAV6 Capsid Library Sequencing and Analysis

Parental and evolved libraries were processed for Illumina NovaSeq sequencing. Briefly, parental and third-round evolved libraries were each treated with DNase I and purified by iodixanol gradient centrifugation. To dissociate the capsid, virus was heated in a PCR tube (95° C., 15 min) with Tween-20, which prohibits capsid reassembly that would interfere with amplification. Round 1 PCR was performed with primer sets -(i) Forward is 5′-CCCTACACGACGCTCTTCCGATCTNNNNNCTGGACCGGCTGATGAATCCTCTC-3′ (SEQ ID NO:574) and (ii) Reverse is 5′-GACTGGAGTTCAGACGTGTGCTCTTCCGATCTNNNNNTATACGTCTCTGTCTTGC CACACCATTCC-3′ (SEQ ID NO:575) for 18 cycles using Q5 polymerase (NEB #M0492S), and amplicons were PCR-purified (IBI Scientific #IB47010).

In Round 2, indices for demultiplexing and the P5 and P7 flow cell adaptor sequences were added in a 15-cycle PCR, and amplicons were run on and purified from a 1% agarose gel. The amplicon band was gel-purified, amplicon quality was verified using a Bioanalyzer, and concentrations were quantified by Qubit. Libraries were prepared with the Illumina NovaSeq 6000 S-Prime reagent kit (300 cycles) following the manufacturer's instructions and sequenced by Illumina NovaSeq.

De-multiplexed reads were analyzed using an in-house Perl script as done previously (Havlik L P, et al. (2021). J Virol. 95:e0058721). Reads were probed for the nucleotide sequences corresponding the library region, and the occurrence of each nucleotide sequence was counted and ranked. These sequences were converted to amino acid sequences and pooled by like-sequence, counted, and ordered by percentage rank. A second Perl script was used to calculate enrichment between the evolved library and parental library as done previously (Havlik L P, et al. (2021). J Virol. 95:e0058721). Sequences were plotted in Tableau with y-axis as log percentage, x-axis as a random dimensionless number, and bubble diameter correlating to enrichment. The frequency of each randomized amino acid in the library was calculated, and heatmaps were generated in GraphPad Prism. To generate the amino acid position-specific scoring matrix, sequences that were in the top 1,000 reads of the evolved library and enriched by over 500-fold from the parental library were selected and run through PSSMSearch (http://slim.icr.ac.uk/pssmsearch/).

### (f) Animal Work

Mice between 6-12 weeks of age were used following a protocol approved by the UCSF Institutional Animal Care and Use Committee. The following mouse strains were obtained from The Jackson Laboratory: C57BL/6J (#000664), BALB/cJ (#000651), H11-Cas9 on C57BL/6J (#028239), and Rosa26-Cas9 knock-in on C57BL/6J (#026179). NOD mice were bred and provided by the Qizhi Tang laboratory (UCSF).

C57BL/6J mice between 6-8 weeks of age were injected subcutaneously with 5×10⁵ hCD19-expressing LL2/Luc2 cells. Nine days later, mice were injected retro-orbitally with 1.5×10⁶ CAR-T cells. Mice with small (<20 mm³) or ulcerated tumors at the day of T cell injections were excluded from the experiment, the experimental groups were randomly allocated among the remaining mice. Tumors were measured using a caliper, and tumor size was estimated using the formula V=(L×W×W)/2. Alternatively, the formula of V=1/6×PI×L×W×(L+W)/2 can be used, which formula weighs L and W equally and treats the tumor as a three-dimensional oval shape.

### (g) Cell Culture

Retroviruses and AAVs were packaged in HEK 293T cells (ATCC #CRL-3216). LL2-Luc2 cells (ATCC #CRL-1642-LUC2) were transduced with an MSCV retrovirus expressing hCD19 and a puromycin resistance gene. Transduced cells were selected with puromycin (2 g/mL) for three days. Puromycin was subsequently maintained in the culture medium for these cells. OVA-expressing mCherry-positive B78 cells were provided by the Matthew Krummel laboratory (UCSF) and were used for assays with OT-I TCR T cells. These cell lines were cultured in GlutaMAX DMEM (Gibco #10566024) supplemented with FBS (10%; Corning #35016CV), streptomycin (0.1 mg/mL; ThermoFisher Scientific #15140122), penicillin-streptomycin (100 U/mL; ThermoFisher Scientific #15140122), sodium pyruvate (1 mM; Gibco #11360070), and HEPES (10 mM; Corning #25-060-CI).

721.221 Human HLA Negative B Cell Line (Millipore Sigma #SCC275) were cultured in RPMI 1640 (Gibco #11875093) supplemented with FBS (10%), penicillin-streptomycin (100 U/mL), sodium pyruvate (1 mM), HEPES (10 mM), β-mercaptoethanol (Gibco #21985-023), MEM non-essential amino acids (1×; Gibco #11140050). HLA-G expressing 721.221 were kindly provided by the Lewis Lanier laboratory (UCSF) and cultured under the same conditions as its parental cell line.

### (h) T Cell Isolation and Culture

Spleens from mice were crushed and strained prior to isolating T cells using an EasySep mouse T cell isolation kit (STEMCELL Technologies #19851). T cells were activated for at least 24 hr using Dynabeads Mouse T-Expander CD3/CD28 (Gibco #11452D). Murine T cells were cultured in RPMI 1640 (Gibco #11875093) supplemented with FBS (10%), penicillin-streptomycin (100 U/mL), sodium pyruvate (1 mM), HEPES (10 mM), β-mercaptoethanol (Gibco #21985-023), MEM non-essential amino acids (1×; Gibco #11140050) and 200 U/mL hIL-2 (Peprotech #200-02).

Human T cells were isolated from leukopaks with peripheral blood mononuclear cells were obtained from STEMCELL Technologies (#70500.1). T lymphocytes were then purified using the EasySep Human T Cell Isolation Kit (STEMCELL Technologies #17951). T Cells were activated with Dynabeads Human T-Activator CD3/CD28 (ThermoFisher #11131D) in X-vivo 15 medium (Lonza #BPO4-744Q) supplemented with 5% human serum (Gemini Bioproducts #100-512), IL-7 5 ng/mL (Miltenyi Biotec #130-095-367) and IL-15 5 ng/mL (Miltenyi Biotec #130-095-760) at a density of 10⁶ cells per mL.

### (i) Flow Cytometry

Cells were stained in FACS buffer (2% FBS and 1 mM EDTA in PBS) using the following reagents: 7-AAD (eBioscience #00-6993-50), propidium iodide (MilliporeSigma #P4170), PE-Vio770 anti-mouse QA2 (Miltenyi Biotec #130-103-909), APC-Cy7 anti-mouse TCRβ (BD #560656), Alexa Fluor 647 anti-mouse F(ab′)₂ for the CAR (Jackson ImmunoResearch #115-606-003), BV421 anti-mouse TCRVα2 (BioLegend #127825), and APC anti-mouse TCRP5.1 (BioLegend #139506). Cells were stained in PBS when Zombie Violet (BioLegend #423114) was used.

For CAR detection, T cells were stained with Alexa Fluor 647 anti-mouse F(ab′)₂ (Jackson ImmunoResearch #115-606-003), then blocked with normal mouse serum (MilliporeSigma #NSO3L) before further antibody staining was performed.

### (j) Cytotoxicity Assays

To evaluate the cytotoxicity of T cells expressing hCD19-targeting receptors, 10⁴ hCD19-LL2-Luc2 cells were plated in 100 μL culture media in a 96-well plate. 24 hours after plating the cells, 10⁴ effector T cells in 50 μL culture media were added to the wells. After 24 hr of co-culture, luminescence was measured with a GloMax Explorer microplate reader (Promega #GM3500) by adding D-Luciferin (Goldbio #LUCK-1G) at a final concentration of 0.375 mg/mL to each well. The cytotoxicity for each sample was determined by the formula: 100%×(1-(sample-minimum)/(maximum-minimum)). The minimum signal was a condition with tumor cells and Tween-20 (2%), and the maximum signal was a condition with tumor cells only.

To evaluate the cytotoxicity of OT-I TCR T cells, 10⁴ OVA.mCherry.B78 cells were plated in 100 μL culture media in a 96-well plate. 24 hr after plating the cells, 2.5×10⁵ effector T cells in 50 μL culture media were added to the wells. Cells were co-cultured for five days with imaging at 2 hr intervals using an IncuCyte live-cell analysis instrument (Sartorius). The mCherry signal intensity from each well and time point was normalized to the average of the first time point in the control wells containing tumor cells only.

### (k) Assays for Viral Binding and Cellular Uptake

Murine T cells were activated for three days with CD3/CD28 Dynabeads in murine T cell medium. For the cell surface binding assay, prior to infection, cells were cooled (4° C., 30 min) to arrest cellular uptake. Cooled cells were infected with AAV6 or Ark313 containing a scCBh-GFP cassette at 10¹ vg/cell (1 hr, 4° C.) to promote viral binding but not uptake. Unbound virions were washed out with ice cold PBS three times, and viral and cellular DNA was extracted using an IBI genomic DNA extraction kit. Uptake was assayed using a similar stepwise process as for binding, except that after washing out unbound virions, cells were warmed with medium at 37° C. and incubated in a 5% CO₂ incubator (37° C., 1 hr) to facilitate uptake of bound virions. After incubation, cells were washed and trypsinized with 0.05% TrypLE Express (ThermoFisher Scientific #12605036) for 5 min to remove uninternalized virus, and viral and cellular DNA was extracted. qPCR analysis was performed on the DNA extracts for GFP to detect viral recombinant DNA, and for Lamin-B1 to detect cellular DNA. Relative quantity of each amplicon was determined by a serial dilution of either vector plasmid or mouse genomic DNA of known concentration.

### (l) Assays for Phosphoinositide Phospholipase C GPI Cleavage

To cleave GPI-anchored proteins, 10⁵ activated C57BL6/J T cells in 100 μL of murine T cell medium were pre-treated with bacterial phosphoinositide phospholipase C (PI-PLC; 1 U/mL; ThermoFisher Scientific #P6466) for 1 hr at 37° C. prior to assays for viral binding or transduction. Binding studies were carried out as described in the previous method section. Transduction assays were performed at an MOI of 10⁵ vg/cell for 48 hr before flow cytometry analysis for GFP expression.

### (m) Nucleofection

After 24 hr of T cell activation, CD3/CD28 Dynabeads were magnetically removed, and T cells were nucleofected in P3 buffer (Lonza #V4SP-3096) with ribonucleoprotein (RNP) using a 4D-Nucleofector 96-well unit (Lonza #AAF-1003S). An amount of RNP for one reaction was generated by incubating 60 pmol Cas9 protein (QB3 MacroLab) and 120 pmol sgRNA (Synthego) at 37° C. 2×10⁶ cells were electroporated with RNP per well. Lonza program code DN-100 was used for murine T cells, and EH-115 was used for human T cells. After nucleofection, cells were diluted in culture medium and incubated (37° C., 5% CO₂). For making knock-ins, AAV was added to the culture between 30-60 min after nucleofection at the indicated MOI, and the culture were incubated overnight. The next day, the medium was exchanged for fresh T cell medium, and cells were expanded using standard culture conditions and maintained at a density of approximately 2×10⁶ cells per mL.

### (n) Retroviral Production and Transduction

3.5×10⁶ HEK 293T cells were seeded in a 10-cm dish. After approximately 24 hr, the medium was replaced with 5 mL cDMEM, and cells were transfected with 7.5 μg pCL-ECO plasmid and 7.5 μg MSCV plasmid using Lipofectamine LTX with PLUS reagent (Invitrogen #15338030). The transfection mix was prepared in 3 mL Opti-MEM medium (Gibco #31985062) and incubated for at least 30 min at room temperature before being pipetted dropwise onto the cell culture. At 24 hr after transfection, the medium was exchanged for 6 mL cDMEM collection medium. Retrovirus was harvested, sterile filtered, and frozen down at 24 hr and at 48 hr.

Transductions were performed on murine T cells at least 24 hr after activation. 6-well plates were coated with 15 μg/mL Retronectin (Takara #T100B) overnight at 4° C. The wells were gently rinsed with PBS prior to adding 3×10⁶ activated murine T cells. Retrovirus was added to the cells together to bring the total per-well volume to 2 mL, with 10 μg/mL polybrene. Cells were spinfected (2000× g, 30° C., 60 min) and then incubated overnight in a 37° C. CO₂ incubator. The next day, the medium was exchanged for fresh T cell medium.

LL2-Luc2 cells were seeded and cultured for 24 hr before transduction with retrovirus and 10 μg/mL polybrene, with overnight incubation. Transduced cells were selected with puromycin (2 μg/mL) for three days. Puromycin was subsequently maintained in the culture medium for these cells.

### (o) Genome-Wide CRISPR/Cas9 Screening

A genome-wide sgRNA knockout library targeting 18,424 genes (a total of 90,230 sgRNAs) in an MSCV plasmid was obtained from Addgene (#104861) and amplified following the attached instructions to maintain library representation (PMID 30639098). Viral packaging and transductions were performed using methods described in the previous section. The screen was performed with two technical replicates, each maintaining 500-fold coverage throughout the experimental procedure. For each replicate, 9×10⁷ activated Cas9-expressing murine T cells were transduced with the retroviral library by spinfection and incubated overnight. Transduction efficiencies of at least 50% were confirmed for each replicate by flow cytometry for BFP expression. The cells were cultured and expanded for 48 hr before being re-activated using CD3/CD28 Dynabeads at a 1:1 ratio. At 24 hr after re-activation (i.e., 96 hr after the initial spinfection), 1.5×10⁸ cells per replicate were transduced with Ark313 scAAV-CAG-GFP at an MOI of 3×10⁴. At 48 hr after AAV transduction, cells were prepared for sorting by 7-AAD live-dead staining (eBioscience #00-6993-50), followed by fixation in 4% formaldehyde in PBS (15 min, 4° C.) with cells at a concentration of 10⁷ cells per mL. Fixed, BFP positive, cells were sorted in to four bins based on GFP expression in FACS buffer (2% FBS and 1 mM EDTA in PBS); a total output of >4.5×10⁷ cells were sorted per replicate at the UCSF Parnassus Flow Cytometry Core (PFCC).

After sorting, genomic DNA was isolated as described previously (Jacob W. Freimer, 2021), sgRNA barcodes were PCR-amplified using Ex Taq DNA polymerase (Clontech #RROO1A) for 28 cycles, and amplicons were purified using SPRI beads. PCR primers were designed for Illumina sequencing with barcoded P7 primers. The barcode binding region was 5′-TTGTGGAAAGGACGAAACACCG-3′ for the P5 adapter (SEQ ID NO:600) and 5′-CTAAAGCGCATGCTCCAGACTG-3′ for the P7 adapter (SEQ ID NO:601). Amplicon libraries were sequenced with Illumina NextSeq500 using the NextSeq 500/550 high output kit v2.5 (Illumina #20024906), with 500-fold coverage as the targeted depth of sequencing.

### (p) Analysis of FACS-Based Screen

Sequencing data were mapped to the reference library using MAGeCK count with the argument—trim-5 22,23,24,25,26,28,29,30 to remove a staggered 5′ adapter (Li W, et al. (2014). Genome Biol. 15:554). The resulting raw counts were input into a Bayesian hierarchical model called waterbear. The model treats each sgRNA from each replicate as a draw from a four-dimensional Dirichlet-multinomial distribution, with each dimension corresponding to a bin from cell sorting. Effects were modeled by a spike-and-slab approach, similar to Bayesian sparse linear mixed models (Zhou X, et al. (2013). PLoS Genet. 9:e1003264). During each MCMC sample, if a gene is included in the model, all of its guides are allowed to have correlated effect sizes conditionally independent given the overall gene-level effect size. If the gene is not included in the model, then all of the guides are modeled to have an effect size of zero. The model was implemented in NIMBLE (de Valpine P, et al. (2017) J Comput Graphical Stats. 26:403-413) and four chains were run, each with 10,000 burn-in samples and 10,000 additional samples that were kept for posterior summaries. The genes that had a posterior inclusion probability (PIP) >0.9 were interpreted to have an effect,

TABLE 5
Listing of Primer Sequences
for AAV Titration

| SEQ ID NO | Target |
|---|---|
| 552 | Trac LHA forward |
| 553 | Trac LHA reverse |
| 554 | sfGFP forward |
| 555 | sfGFP reverse |
| 556 | U6 forward |
| 557 | U6 reverse |
| 558 | AAV2-ITR forward |
| 559 | AAV2-ITR reverse |

TABLE 6
Listing of Sequences for Genomic
DNA Knock-In Validation

| SEQ ID NO | Target |
|---|---|
| 560 | Clta forward |
| 561 | Clta reverse |

TABLE 7
Listing of Sequences for
Genomic DNA Indel Frequency

| SEQ ID NO | Target |
|---|---|
| 562 | Clta forward |
| 563 | Clta reverse |
| 564 | B2m forward |
| 565 | B2m reverse |
| 566 | Aavr forward |
| 567 | Aavr reverse |
| 568 | Gpr108 forward |
| 569 | Gpr108 reverse |

TABLE 8
Listing of Sequences for Capsid Library Primers

| SEQ ID NO | Target |
|---|---|
| 570 | AAV6 mutagenesis and SbfI site forward |
| 571 | AAV6 mutagenesis and SbfI site reverse |
| 572 | AAV6 Cap BsiWi site forward |
| 573 | AAV6 Cap BsiWi site reverse |

TABLE 9
Listing of Illumina Sequencing
Primers for Capsid Library

| SEQ ID NO | Target |
|---|---|
| 574 | AAV6 Illumina amplification forward |
| 575 | AAV6 Illumina amplification reverse |

TABLE 10
Listing of Illumina Sequencing
Primers for Knock-Out Screen

| SEQ ID NO | Target |
|---|---|
| 576 | P5 |
| 577 | P7 (index) |

TABLE 11
Listing of Sequences for Binding and Uptake

| SEQ ID NO | Target |
|---|---|
| 578 | Lmnb1 gDNA forward |
| 579 | Lmnb1 gDNA reverse |
| 580 | GFP forward |
| 581 | GFP reverse |

TABLE 12
Listing of sgRNA
Sequences for Examples 6-10

| SEQ ID NO | sgRNA Sequences |
|---|---|
| 582 | Trac |
| 583 | Trbc |
| 584 | Clta |
| 585 | B2m_1 |
| 586 | B2m_2 |
| 587 | H2-Q7_1 |
| 588 | H2-Q7_2 |
| 589 | Aavr_1 |
| 590 | Aavr_2 |
| 591 | Gpr108_1 |
| 592 | Gpr108_2 |
| 593 | B2M |
| 602 | NTC |

TABLE 13
Listing of Homology Arm
Sequences for Examples 6-10

| SEQ ID NO | Homology Arms |
|---|---|
| 594 | Clta LHA |
| 595 | Clta RHA |
| 596 | Trac LHA |
| 597 | Trac RHA |

TABLE 14
Listing of Insert Sequences

| SEQ ID NO | Target |
|---|---|
| 598 | sfGFP Insert Sequence |
| 599 | 1928z Insert Sequence |

### Introduction to Example 6—Example 10

Decades of research have placed T lymphocytes at the center of adaptive immunity and tolerance. Advances in the ability to engineer the T cell genome and modulate gene expression have been fundamental to our understanding of the regulation of T cell development and function in both health and disease. Recently, T cells engineered to express a Chimeric Antigen Receptor (CAR) have been transformative in treating hematological malignancies (June CH, et al. (2018). Science. 359:1361-1365; June C H, et al. (2018). N Engl J Med. 379:64-73; Sadelain M et al. (2017). Nature. 545:423-431), and there is great interest in extending this modality to the treatment of solid tumors (June CH, et al. (2018). N Engl J Med. 379:64-73).

The most common recombinant gene delivery vectors for T cells are replication-defective retroviruses such as γ-retroviruses or lentiviruses, which result in semi-random integration and variable transgene expression due to variegation. Position effects can lead to heterogeneous T cell function, transgene silencing, and insertional oncogenesis, which limit the efficacy and safety of these therapeutic products (Shah NN, et al. (2019). Blood Adv. 3:2317-2322; Fraietta J A, et al. (2018). Nature. 558:307-312).

Advances in gene editing technologies have enabled the precise integration of transgenes in primary human T cells (Eyquem J, et al. (2017). Nature. 543:113-117; Sather B D, et al. (2015). Sci Transl Med. 7:307) and opened new avenues for experimental and clinical T cell engineering. The targeted integration of a CAR under the control of the endogenous TCR alpha (TRAC) promoter conferred physiologic receptor expression and yields T cells with superior anti-tumor activity compared to γ-retroviral delivery in xenograft mouse models. The expression offered by the TRAC locus has also been reported to_improve the activity of transgenic TCRs (Muller TR, et al. (2021). Cell Rep Med. 2:100374; Roth TL, et al. (2018). Nature. 559:405-409; Schober K, et al. (2019). Nat Biomed Eng. 3:974-984). Recently, using gene targeting, the TCR specificity was remodeled to target cell surface antigens in an HLA-independent manner, and this HLA-independent TCR (HIT) benefited from the physiologic signal transduction and antigen sensitivity of the TCR locus and architecture (Mansilla-Soto J, et al. (2022). Nat Med. 28:345-352).

In the context of T cells, only immunocompetent models can recapitulate the complexity of a tumor microenvironment or autoimmune niche. However, the identification of an efficient and non-toxic method for targeting large DNA cargo to murine T cells has remained elusive. Electroporation of TALEN mRNA (Menger L, et al. (2016). Cancer Res. 76:2087-2093) or Cas9 ribonucleoprotein (RNP) (Seki A, et al. (2018). J Exp Med. 215:985-997) have been used to make knockouts, but homology directed repair template (HDRT) delivery has been a bottleneck for making knock-ins (KI). HDRTs have been delivered to human T cells using adeno-associated virus serotype 6 (AAV6) (Eyquem J, et al. (2017). Nature. 543:113-117; Sather B D, et al. (2015). Sci Transl Med. 7:307) or DNA (Nguyen DN, et al. (2020). Nat Biotechnol. 38: 44-49; Roth TL, et al. (2018). Nature. 559:405-409), with AAV6 remaining the most efficient and least toxic method. In contrast to human T cells, attempts to edit murine T cells by electroporating a short ssDNA or dsDNA HDRT (for making a single-nucleotide mutation) resulted in low targeting (<10%) and high toxicity (50-85% cell death) (Kornete M, et al. (2018). J Immunol. 200: 2489-2501). AAV could potentially solve these issues, but to date there is no AAV serotype that can efficiently transduce murine T cells, and thus there is a crucial unmet need for AAV variants with this tropism. To this end, multiple approaches including rational engineering of surface epitopes on AAV capsids, directed evolution through DNA shuffling, peptide insert libraries, 3D structure-guided evolution, and more recently, machine learning have been utilized to generate novel AAV variants with altered tropism, improved transduction efficiency and/or the ability to evade neutralizing antisera (Bryant DH, et al. (2021). Nat Biotechnol. 39:691-696; Challis R C, et al. (2022). Annu Rev Neurosci. 10:1146; Li W, et al. (2014). Genome Biol. 15:554; Madigan V J, et al. (2016). Curr Opin Virol. 18:89-96). Notably, the feasibility of achieving receptor switching in newly evolved AAV variants through infectious cycling of AAV capsid libraries with modified surface footprints was established (Havlik LP, et al. (2021). J Virol. 95:e0058721).

As described below, a structure-guided evolution approach was employed to evolve a novel AAV variant that was named Ark313. Ark313 was derived from AAV6 and exhibited high transduction efficiency in murine T cells. The data show that Ark313 can be used for transient gene delivery and precise genome engineering in primary murine T cells. Ark313 can be used to model various engineering strategies from human T cells in the murine context, and new gene targeting strategies that expand the use of genetically engineered T cells for in vivo studies are now possible. Furthermore, through a genome-wide knockout screen, an essential murine host factor was identified and the mechanism for Ark313 cellular entry was elucidated. Ark313 opens new avenues in experimental T cell immunology and the preclinical modeling of precision-engineered cell therapies in immunocompetent hosts.

### Example 6

Structure-Guided Evolution Evolved an AAV Capsid Variant with Murine T Cell Tropism

To identify an AAV variant enabling efficient DNA delivery to murine T cells, an AAV capsid library based on AAV serotype 6 was generated. This serotype was chosen as a template for mutagenesis and evolution due to its established ability to transduce and facilitate HDRT knock-in in human T lymphocytes, NK cells, and hematopoietic stem cells (Pomeroy EJ, et al. (2020). Mol Ther. 28:52-63; Sather B D, et al. (2015). Sci Transl Med. 7:307; Wang J, et al. (2015). Nat Biotechnol. 33:1256-1263). Saturation mutagenesis was performed on a pseudotyped AAV2/6 wild-type genome composed of the AAV2 Rep gene and AAV6 Cap gene flanked by AAV2 inverted terminal repeats (ITRs). Saturation mutagenesis was performed on variable region IV (VR-IV) (amino acids 454-460) of the VP3 capsid protein subunit. This surface epitope has been implicated in host cell entry and antibody-mediated neutralization of different AAV serotypes. Targeting this region in other AAV serotypes for structure-guided evolution yielded new and improved variants (Havlik LP, et al. (2021). J Virol. 95:e0058721; Tse LV, et al. (2017). Proc Natl Acad Sci USA. 114:E4812-E4821).

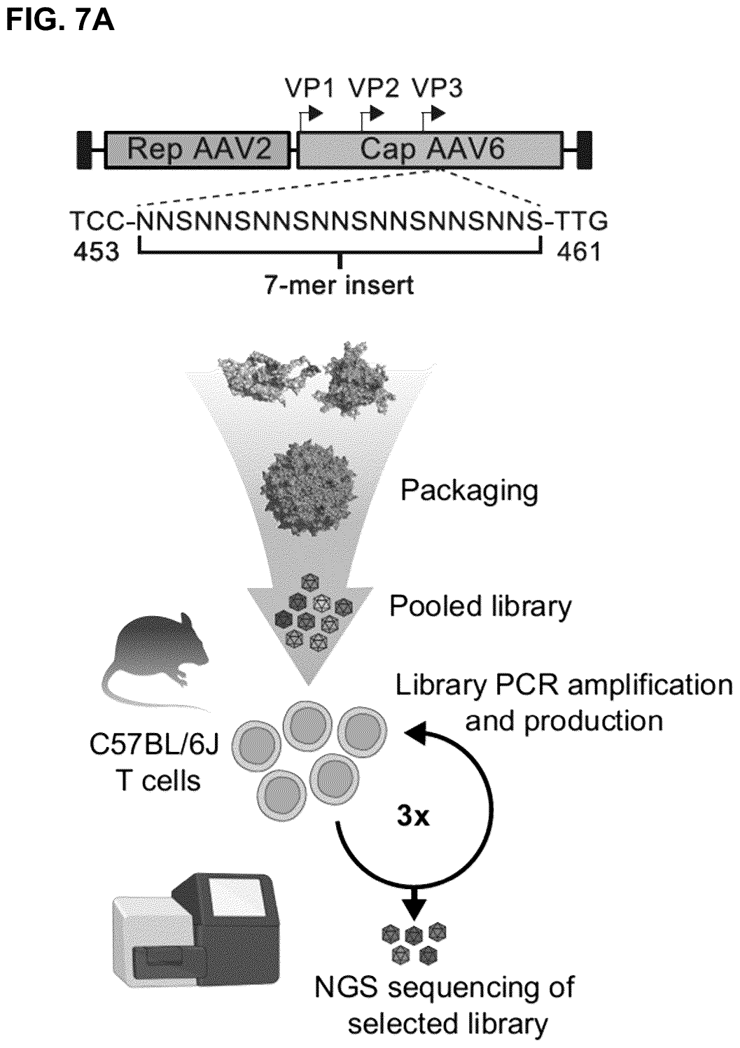

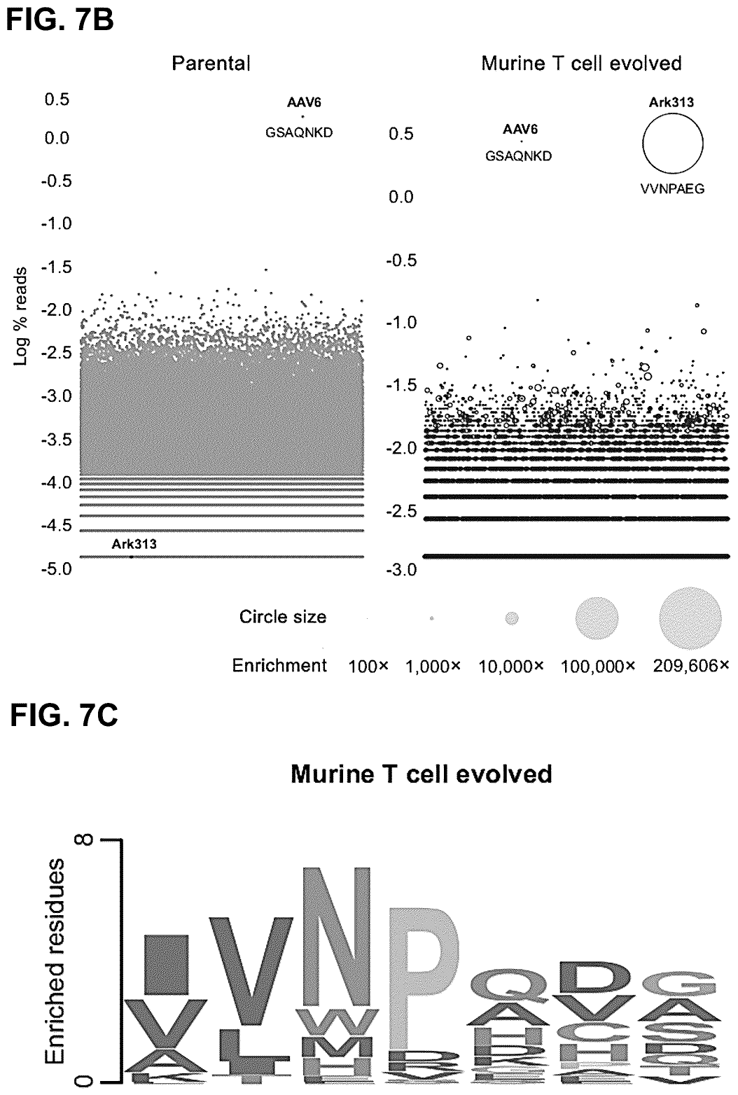

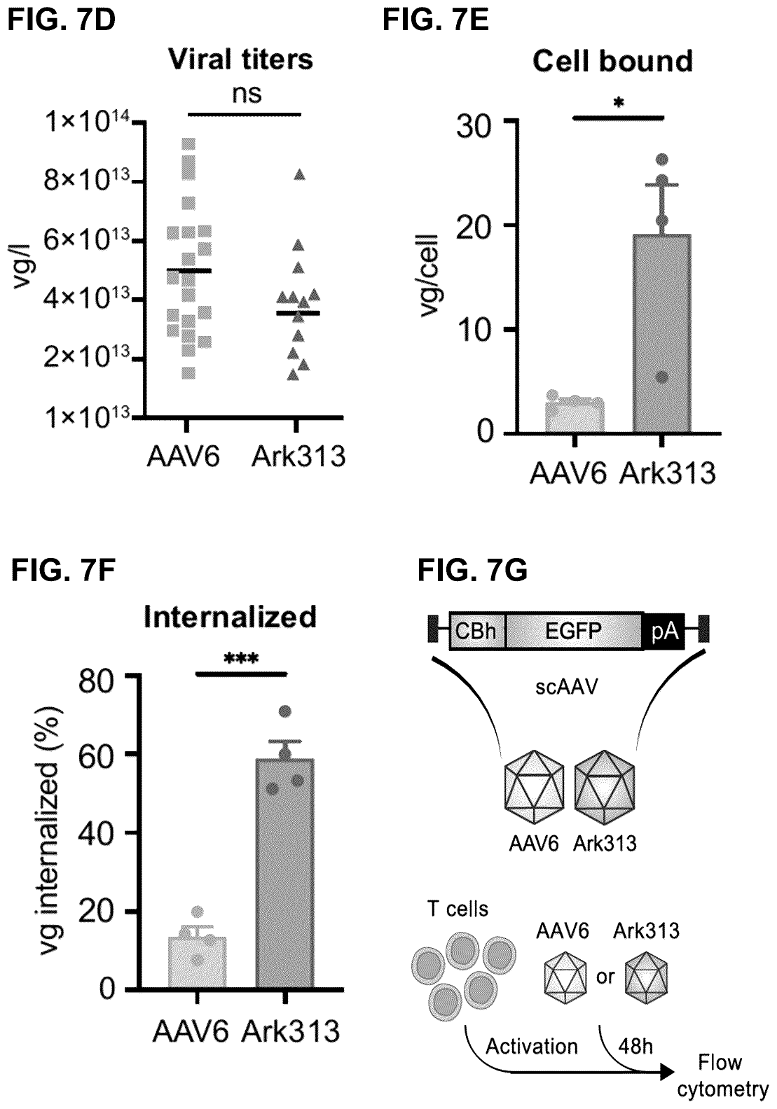

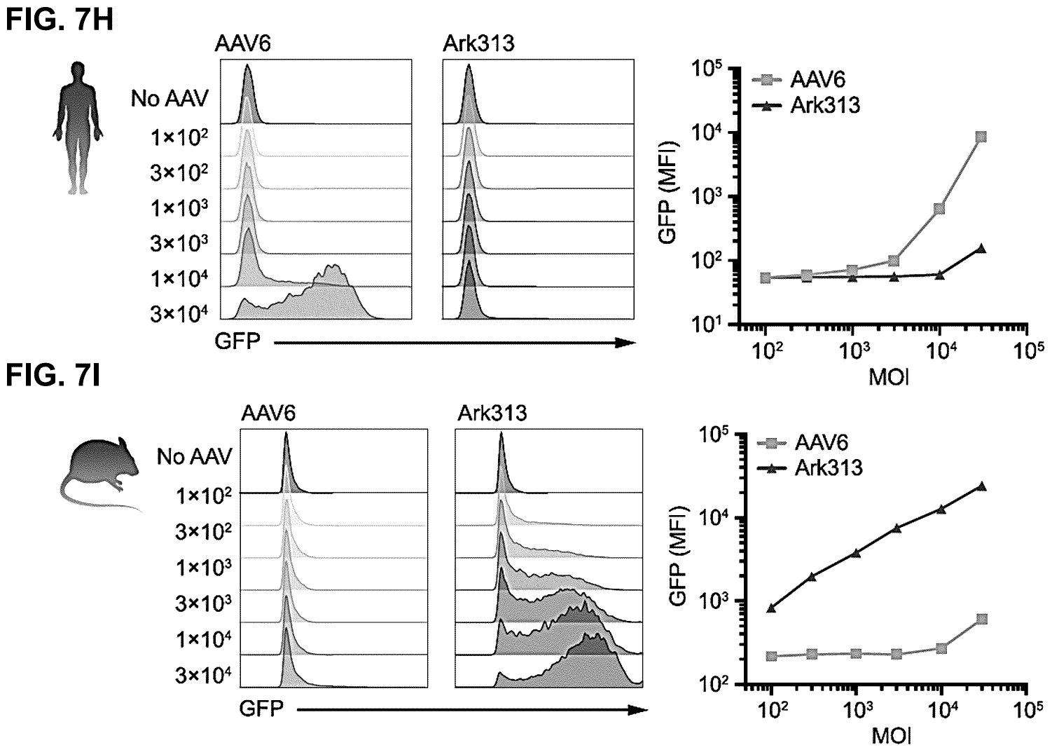

A screening strategy to identify AAVs that could efficiently deliver donor DNA to murine T cells for CRISPR/Cas9 genome editing was developed. Primary splenocyte T cells isolated from C57BL/6J mice were activated with CD3/CD28 beads and recombinant IL-2, and then co-cultured with the capsid library at a relatively low multiplicity of infection (MOI of 10⁴) for 6 hours (FIG. 7A). To enrich for mutants that underwent cellular uptake, T cells were washed post-infection to remove residual surface-bound virus. Viral DNA was then purified, PCR-amplified, and re-cloned into the wild type AAV plasmid backbone to generate a capsid library for a subsequent round of evolution (FIG. 7A). After three infection cycles, the parental and evolved libraries were analyzed by next-generation sequencing. Remarkably, a single dominant variant was identified -Ark313. Ark313 carried the amino acid substitution 454-VVNPAEG-460 (SEQ ID NO:02) and displayed ˜200,000-fold enrichment (FIG. 7B). Evaluation of the top-ranked sequences and most highly enriched reads (top 1,000 reads, >500-fold enrichment) indicated a consensus motif [I/V][IL/V][N][P] for the first four amino acids (FIG. 7C).

Next, recombinant AAV6 and Ark313 were produced and no significant difference for viral titer between AAV6 and Ark313 was observed. This indicated that the mutations did not affect packaging efficiency (FIG. 7D). The AAVs were then assessed for cell surface binding and uptake. Murine T cells were cooled to 4° C. prior to and during AAV incubation to arrest cellular uptake. After washing cells to remove unbound virus, viral DNA was extracted and the number of vector genomes per cell was quantitated. Compared to AAV6, a significantly higher amount of Ark313 was bound to murine T cells (FIG. 7E). To analyze cellular uptake, the same process was performed to allow AAV binding, followed by a short incubation at 37° C. to promote uptake. A significantly higher percentage of Ark313 particles compared to AAV6 was internalized by murine T cells (FIG. 7F). Motivated by Ark313 enhanced binding and update, the ability of Ark313 to transduce primary murine T cells was evaluated. A self-complementary AAV (scAAV) encoding GFP was packaged under a hybrid chicken β-actin (CBh) promoter in either AAV6 or Ark313 capsids (FIG. 7G). In human T cells, high transduction efficiency was observed with AAV6 as expected at MOIs≥10⁴, and GFP expression was not observed with Ark313 (FIG. 7H, FIG. 8C). These data showed that the VR-IV of the AAV6 capsid was critical for AAV6 entry in human T cells. Remarkably, in murine T cells, Ark313 improved transduction efficiency with a 30-fold increase in MFI at low Multiplicity of Infection (MOI) (FIG. 7I, FIG. 8D). At the highest MOIs, Ark313 showed even greater improvement with transduction efficiencies up to ˜40-fold higher than AAV6 (FIG. 7I, FIG. 8D). Together, these data showed that Ark313 exhibited high transduction efficiency in murine T cells and indicated the involvement of a murine T cell-specific host factor in Ark313 binding and uptake.

### Example 7

### Genome-Wide CRISPR/Cas9 Screen Identified Ark313 Primary Receptor

While the primary cellular entry mechanisms for AAV6 in human T cells have not been specifically explored, the role of heparan sulfate and N-linked sialylated glycoproteins (Huang LY, et al. (2016). J Virol. 90:5219-5230; Wu Z, et al. (2006). J Virol. 80:11393-11397; Wu Z, et al. (2006). J Virol. 80:9093-9103) and the cognate AAV receptor (AAVR) (Pillay S, et al. (2016). Nature. 530:108-112) in cell surface binding and uptake in general are understood. To determine the entry mechanism of Ark313, a flow cytometry-based genome-wide CRISPR knockout screen was optimized to identify host factors required for Ark313 transduction in primary murine T cells (FIG. 9A). Activated Cas9-expressing murine T cells were transduced with a γ-retroviral pool containing a genome-wide sgRNA library (90,230 sgRNAs) (Henriksson J, et al. (2019). Cell. 176:882-896) and transduced with an Ark313 scAAV for GFP expression at an MOI of 3×10⁴. Cells were sorted into four bins for low-to-high GFP expression (FIG. 9B). Genomic DNA was extracted from each sorted population, and sgRNA barcodes were PCR-amplified and sequenced. The sgRNAs in each of the four bins were compared using waterbear analysis and ranked the sgRNAs based on enrichment in lower GFP bins. This comparison revealed 15 genes as positive regulators of Ark313 transduction (lfsr <0.1). (FIG. 9C). The top hits included genes that are known to be involved in AAV transduction. As expected, AU040320, which encodes the previously described AAVR, was found to be highly enriched (FIG. 9C, FIG. 9D) (Pillay S, et al. (2016). Nature. 530:108-112). Gpr108, which has been identified together with AAVR as an important regulator of AAV processing, was identified (FIG. 9C, FIG. 9D) (Dudek AM, et al. (2020). Mol Ther. 28:367-381; Pillay S, et al. (2016). Nature. 530:108-112. Both hits provided confidence in the sensitivity of the screen. Among the remaining top hits identified were B2m, H2-Q7, and H2-Q6, all of which are components of major histocompatibility complex (MHC) class I (FIG. 9E). H2-Q7 and H2-Q6, together with H2-Q8 and H2-Q9, encode proteins that are classified as QA2 (Devlin JJ, et al. (1985). EMBO J. 4:3203-3207). Interestingly, H2-Q7 is a GPI-anchored cell surface protein (Stroynowski I, et al. (1987). Cell. 50:759-768; Stroynowski I, et al. (1996). Res Immunol. 147:290-301; da Silva IL, et al. (2018). Front Immunol. 9:2894) and multiple GPI-processing genes such as Gpaal were also identified as top hits (FIG. 9D, FIG. 9E). In summary, the screen identified known regulators of AAV transduction and nominated the MHC class I molecule QA2 as a necessary receptor for Ark313 transduction.

To validate the requirement of QA2 expression for Ark313 transduction, T cells from mouse strains that express various levels of QA2 were isolated and activated. In addition to C57BL/6J mice, BALB/cJ mice were also included as a control strain that expresses low QA2 as a result of Q8/Q9 genetic deletions (Das G, et al. (2000). J Exp Med. 192:1521-1528; Mellor A L, et al. (1985). Proc Natl Acad Sci USA. 82:5920-5924; Stroynowski I, et al. (1996). Res Immunol. 147:290-301) as well as NOD mice (that express intermediate QA2 levels) (FIG. 9F). T cells were transduced with scAAV-GFP packaged in either AAV6 or Ark313 and analyzed by flow cytometry. GFP expression was correlated with QA2 expression across strains. The highest transduction occurred in C57BL/6J and the lowest transduction occurred in BALB/cJ (FIG. 9F, FIG. 10A). Within each strain, GFP expression was higher in the QA2-high subpopulation especially for C57BL/6J and NOD (FIG. 9F, FIG. 10B). In contrast, AAV6 transduction efficiency was low and equivalent regardless of QA2 expression (FIG. 9F, FIG. 10A-FIG. 10B). These results indicated that QA2 is a critical factor for Ark313 transduction of murine T cells.

To further validate the importance of our screen hits, activated T cells were nucleofected in arrayed format with RNPs containing two separate sgRNAs for knocking out each gene (FIG. 10C, FIG. 10D). The nucleofected cells were then re-activated and transduced with scAAV-GFP in Ark313. Nearly complete prevention of GFP expression and thus transduction was observed in murine T cells that had undergone knockout of B2m, H2-Q7, or Aavr (FIG. 9G, FIG. 10E). GFP expression was also reduced in Gpr108-KO cells (FIG. 9G, FIG. 10E), though to a lower extent than in other KO cells, tracking with the gene effect observed in the screen (FIG. 9D). Finally, as H2-Q7 is a GPI-anchored protein (FIG. 9E), the ability of Ark313 to transduce murine T cells pre-treated with recombinant GPI-cleaving enzyme phosphoinositide phospholipase C (PI-PLC) was assessed. First, the binding of AAV6 and Ark313 to T cells with or without PI-PLC pre-treatment was assessed. AAV6 was not affected in its ability to bind C57BL/6J T cells, but Ark313 experienced a ˜10-fold reduction in bound virus per cell (based on quantification of viral genomes) following PI-PLC treatment (FIG. 9H). Whether GPI cleavage could ablate Ark313 transduction was next examined. Following PI-PLC pre-treatment, the Ark313 condition showed a ˜5-fold decrease in the percentage of GFP-positive cells, exhibiting similar transduction as the parental AAV6, whereas AAV6 remained unaffected (FIG. 9I). These data corroborated the finding that the GPI-anchored protein H2-Q7 was essential for Ark313 binding and transduction of murine T cells.

### Example 8

### Ark313 Permitted Efficient Gene Targeting in Primary Murine T Cells

Whether the superior transduction of primary murine T cells by Ark313 could be utilized for a range of gene editing applications was examined. First, whether Ark313 could deliver a sgRNA expressing cassette was assessed. T cells from Cas9-expressing mice were isolated and activated and transduced them with Ark313 expressing either a Trac-targeting sgRNA or a scrambled (SCR) control sgRNA (FIG. 11A). TCR-expression in the transduced T cells was analyzed by flow cytometry and up to 83.5% TCR KO was observed in the Trac-transduced cells (FIG. 11B, FIG. 12A). Even at the lowest MOI tested, the KO level was superior to 40% (FIG. 12A). Whether Ark313 could deliver larger DNA cargo such as an HDRT for knock-in and target a GFP to a broadly expressed vesicle-coating protein (e.g., the clathrin light chain A (Clta)), was determined. Murine T cells were nucleofected with Cas9-RNP targeting the Clta gene, and cells were transduced with either AAV6 or Ark313 containing an HDRT for fusing GFP to the Clta N-terminus (FIG. 11C). AAV6 was inefficient at delivering HDRT with less than 10% knock-in at the highest AAV dose. Remarkably, Ark313 yielded much higher knock-in (i.e., >30% at the lowest MOI tested and greater than 50% at the highest MOI) (FIG. 11D). The targeted integrations were further validated by PCR-amplifying genomic DNA flanking the clta locus (FIG. 12B).

While remarkably improved knock-in was observed with Ark313, the reduction of the cell loss associated with RNP nucleofection was pursued to further improve the yield of edited cells. Since Ark313 showed high gene editing efficiency when delivering a sgRNA (FIG. 11B, FIG. 12A), whether co-delivery of sgRNA and HDRT in a single vector to T cells with constitutively expressed Cas9 would result in efficient knock-in and low toxicity was interrogated. A U6 promoter expressing a Clta-targeting sgRNA was incorporated into the construct containing the HDRT for the GFP-Clta fusion and packaged this into either Ark313 or AAV6 (FIG. 11E). Cas9-expressing T cells were transduced with a range AAV6 MOIs. No detectable knock-in was observed. Strikingly, Ark313-mediated co-delivery of sgRNA and HDRT resulted knock-in as high as with Cas9-RNP nucleofection (FIG. 11F). No difference in proliferation or cell numbers between non-treated cells and Ark313-treated cells was observed (FIG. 11G), and the nucleofection-free method increased the knock-in cell yield by over five-fold (FIG. 11H). These data underscore the superior performance of Ark313 for both transient gene delivery and targeted integration in primary murine T cells. The high transduction efficiency of Ark313, which allowed for co-delivery of an HDRT and sgRNA, enabled one-step manufacturing of knock-in cells with high viability and facilitated large production of T cells for in vitro and in vivo applications

### Example 9

### Trac was an Ideal Locus for Experimental T Cell Immunology

As Ark313-mediated gene delivery unlocked knock-in capacities in T cells, the use of Ark313 to engineer T cells was expanded for the study of adoptive cell therapies against cancers. An HDRT targeting Trac exon 1 was designed for expression of a transgene under the endogenous promoter and integrated multiple recombinant receptors relevant to immunotherapy such as a murine CAR targeting human CD19 (hCD19) (1928z), a HIT targeting hCD19, or a transgenic OT-I TCR (FIG. 13A). A construct in which the Trac gene was re-introduced to generate TCR-positive Trac-1928z-T cells was also designed (FIG. 13A). All vectors were designed with a U6 promotor-driven Trac sgRNA for nucleofection-free knock-in and were packaged in Ark313.

Activated T cells from Cas9-expressing mice were transduced with a Trac-1928z Ark313 at several MOIs. CAR and TCR expression was analyzed by flow cytometry and observed high knock-in rates (up to 46.6% at the highest MOI) and efficient TCR KO (FIG. 14A, FIG. 14B). The yield of edited cells using Cas9-RNP electroporation was compared to edited cells using electroporation-free knock-in by co-delivery of sgRNA and HDRT. There was a ˜10-fold increase in Trac-CAR cell yield using the electroporation-free approach (FIG. 14C, FIG. 14D).

To validate the functionality of the antigen-specific Trac-T cells (FIG. 13C), a cytotoxicity assay against hCD19-expressing murine LL2 lung cancer cells (LL2-hCD19) was performed. Compared to non-transduced T cells, each of the hCD19-targeting T cell conditions exhibited significant cytotoxicity against antigen-expressing cells (FIG. 13D). Finally, the functionality of the Trac-OT-I TCR-T cells was validated by performing a cytotoxicity assay against OVA-expressing B78 cells and observed that Trac-OT-I TCR knock-in cells exhibited similar cytotoxicity as transgenic (Trg) OT-I TCR-T cells (FIG. 13E). As the field of synthetic immunology is demanding larger cassettes and multiplex edits, the potential of Ark313 in facilitating multiple genetic modifications in a single step was interrogated. Cas9-expressing T cells were transduced with two separate Ark313 all-in-one HDRTs targeting two separate genes, Clta and Trac. Over 8% of cells underwent dual knock-in (FIG. 13F), further highlighting the range of use for Ark313 in engineering complex gene-edited T cell therapies.

### Example 10

Targeting a CAR to the Trac Locus with Ark313 Enhanced Tumor Control in an Immunocompetent Solid Tumor Mouse Model

Human TRAC-CAR T cells were superior to retrovirally engineered CAR T cells in controlling a B-ALL xenografted tumor. To explore whether the superiority of TRAC-CAR T cells was maintained in an immunocompetent solid tumor model, the panel of CAR T cells previously tested (Eyquem J, et al. (2017). Nature. 543:113-117) was reproduced in a murine setting. Trac-1928z-T cells with Ark313 and conventional 1928z with γ-retrovirus (gRV) were generated. Since Trac-1928z-T cells were TCR-KO, the gRV-expressing 1928z-T cells were co-transduced with either Ark313 expressing a Trac-targeting sgRNA or a SCR sgRNA (FIG. 15A). This procedure generated TCR-KO gRV-1928z-T cells and TCR-intact gRV-1928z-T cells. The expression of the TCR and CAR was assessed by flow cytometry prior to T cell injections (FIG. 15B). LL2-hCD19 cells were injected subcutaneously in C57BL/6J mice. Nine days later, mice were injected retro-orbitally with a single dose of either Trac-1928z-T cells or gRV-transduced 1928z-T cells (FIG. 15A). Although the gRV CAR-T cells were highly cytotoxic in vitro (FIG. 16A), their control of the tumor was limited in vivo (FIG. 15C), with no significant improvement in survival compared to non-treated mice (FIG. 15D). However, the Trac-1928z CAR-T cells reduced tumor size and significantly improved survival compared to non-treated mice in this highly aggressive solid tumor model (FIG. 15C, FIG. 15D). These results represented the first time that adoptively transferred knock-in T cells had been tested in a syngeneic cancer model. The findings highlighted the utility of Ark313 in testing next generation T cell therapies immunocompetent settings.

### Summary of Example 6—Example 10

A structure-guided evolution was used to construct an AAV that has tropism for murine T lymphocytes. This methodological approach can now be extended to any AAV capsid to achieve targeting of any cell type of interest. The work described herein showed that murine T cells enabled targeted manipulation in immunocompetent mouse models. Many clinical trials use adoptive T cell therapies, and more recently precisely edited T cells (NCT03666000, NCT04035434, NCT04629729, NCT04637763). But, to date, murine T cell engineering has relied on the use of transgenic mice or semi-randomly integrating viral vectors, as gene targeting has been inefficient and HDRT DNA delivery can be toxic. The inability to do gene targeting in mouse models has been a roadblock for T cell immunology and preclinical modeling in immunocompetent mice. Thus, Ark313 is a potentially transformative tool for T cell immunology and cancer immunotherapy.

Genome-wide screens have been used to identify essential host factors and restriction factors for viruses. In the case of AAV, the first genome-wide perturbation screen for infection host factors was with AAV2 on the HAP1 human cell line, which identified AAVR and GPR108 (Pillay S, et al. (2016). Nature. 530:108-112). Subsequent efforts identified host restriction factors such as Crb3 (Madigan V J, et al. (2019). J Virol. 93(21):e00943-19), and also receptor switching mechanisms in newly evolved AAV variants (Havlik LP, et al. (2021). J Virol. 95:e0058721). The VR-IV region in AAV8 can be evolved to a new variant, Hum.8, which uses integrin beta-1 (ITGB1) in lieu of the cognate AAVR. This demonstrates the evolutionary plasticity in AAV tropism.

As described herein, the essential cell surface binders of Ark313 were identified. This work employed the first genome-wide knockout screen on primary cells to identify an AAV entry mechanism. The screen highlighted known essential genes for AAV processing, Aavr and Gpr108 (Pillay S, et al. (2016). Nature. 530:108-112), and B2m and H2-Q7 were among the top hits (FIG. 9C, FIG. 9D, FIG. 9G). B2M is a protein that associates with MHC class I, and H2-Q7 is a protein that along with Q5, Q6, Q9 and Q10 is referred to as QA2. In view of the connection between Ark313 and MHC class I, whether the human ortholog for H2-Q7, HLA-G (Comiskey M, et al. (2003). Hum Immunol. 64:999-1004; da Silva IL, et al. (2018). Front Immunol. 9:2894) mediates AAV6 uptake in human T cells was examined. B2M KO in human T cells or HLA-G expression did not affect AAV6 transduction (FIG. 16B, FIG. 16C).

Ark313 transduction correlated with QA2 expression using cells from different mouse strains (FIG. 9F). With this correlation, gene expression databases were used to identify those cell types that might be amenable to Ark313 transduction. For example, NK and NKT cells express H2-Q7/QA2 (Heng TS, et al. (2008). Nat Immunol. 9:1091-1094) and therefore might be good candidates, thus providing possibilities to study engineered NK cells in immunocompetent mice. Conversely, certain B cell subsets, neutrophils, monocytes, and macrophages express low levels of H2-Q7/QA2 (Dietz S, et al. (2021). Front Immunol. 12:787468; Heng TS, et al. (2008). Nat Immunol. 9:1091-1094), so low transduction efficiency is expected. Both the AAV library approach and knockout screen can be extended to the aforementioned primary cell types to generate new AAV variants and interrogate the biology of virus-host interactions.

The work described herein shows that Ark313 was an efficient vector for transient transgene expression in murine T cells by expressing GFP or an sgRNA. This approach required minimal cell handling, was non-toxic, easily scalable, and can now be applied to any transgene within packaging capacity such as Cre, compact Cas proteins or any gene that might modulate T cell function or fate.

Ark313 permitted non-toxic HDRT delivery for efficient gene targeting in primary murine T cells. Greater than a 50% knock-in was observed at the Clta locus by delivering the HDRT with Ark313 to RNP-nucleofected cells. This is the first instance in which knock-in has been performed in primary murine T cells at such high efficiency. There was on average a ˜60% reduction in cell viability and slower proliferation in the days after murine T cell nucleofection, which is substantially worse than the analogous viability reduction for human T cells. While this cell loss potentially limits the use of edited mouse T cells in large-scale experiments, such as those with libraries; this technical hurdle was overcome by successfully co-delivering HDRT and sgRNA in a single AAV to Cas9-expressing cells. Nucleofection-free knock-in yielded similar efficiencies as with nucleofection but without the negative impact on cell yield. Although the packaging capacity of AAV vectors imposes limits on cargo delivery, dual KI can be readily performed by dual AAV infection, thus enabling a broad spectrum of engineering and screening applications.

Using the mouse Trac locus, receptors can now be integrated to further explore their functionality and identify potential limitations in immunocompetent model systems. The Ark313 nucleofection-free approach can be used to target a CAR, HIT or a transgenic TCR to the Trac locus. These three families of receptors, when delivered to the human TRAC locus, are in clinical or in pre-clinical stages of development but have yet to be tested in complex immunocompetent models that recapitulate the challenges that T cell therapies face in solid tumors. Human TRAC-CAR-T cells have been demonstrated to improve tumor clearance in xenograft models compared to conventional virally expressed CAR-T cells (Eyquem J, et al. (2017). Nature. 543:113-117). Murine Trac-CAR-T cells and gRV CAR-T cells were compared in an immunocompetent solid tumor mouse mode. Only the Trac-CAR-T cells showed significant improvement in survival. This is the first instance in which Trac-CAR-T cells have been used in an immunocompetent mouse model. In addition to survival, this model should enable in-depth interrogation of the biology of Trac-CAR-T cells and how the cells interact with the endogenous immune system upon adoptive transfer. The crosstalk between CARs and TCRs in solid tumors is currently unknown. The TCR is known to contribute to T cell fitness through tonic signaling and interaction with specific intra-tumoral DC populations, providing co-stimulation. TCRs can also drive polyclonal antitumor response and address tumor heterogeneity. In TRAC-CAR T cell, the absence of a TCR can be beneficial, as co-activation of T cells through the CAR and the TCR has been shown to negatively affect CD8-T cells in a leukemia model (Yang Y, et al. (2017). Sci Transl Med. 9(417):eaagl209). The ability to generate a panel of TCR-expressing Trac-CAR-T cells by either KO of the TCR, rescuing the Trac gene or co-deliver a recombinant TCR provides a path to study the interplay between CAR and TCR signaling in vivo. These results further highlight the potential impact of Ark313 as a tool in the field of T cell immunology.

The homogenous and monoallelic expression conferred by the TRAC locus has been demonstrated to be ideal for screening pooled libraries of genes in human T cells (Roth TL, et al. (2020). Cell. 181:728-744). However, the relevance of the elucidated genetic effects depends entirely on the biological context. In this study, similar homogenous and predictable expression at the Trac locus of murine T cells (FIG. 16D) was shown. Thus, Ark313 offers the possibility to perform knock-in screening at Trac in immunocompetent models. Finally, while this study focused on cancer immunotherapy, the ability to re-direct T cell specificity is not limited to cancer mouse models. The potential to knock in any TCR to replace the endogenous TCR, without the need to breed transgenic TCR mice, opens possibilities to study T cells in autoimmunity. Ark313 is expected to be a fundamental tool to accelerate the discovery of cell therapy modalities in immunocompetent models and clinical translation.

### Introduction to Example 11—Example 15

Manipulation of T-cells in mouse models in vivo has allowed for interrogation of immune mechanisms and pathways. However, generation of conditional T lymphocyte specific genetic changes in mouse models can be labor intensive, requiring embryonic manipulation and challenging to control temporally. To better study and manipulate T-cell biology in mouse models for gene therapy and immunotherapy applications, a novel AAV capsid mutant Ark313, which was capable of efficient and targeted gene transfer to murine T-cells in vivo, was generated (described supra). Delivery of transiently expressed genes was feasible as demonstrated by a self-complementary GFP cassette delivered using Ark313. Here, ˜10% GFP+of CD3+ splenocytes was observed, but there was no significant transduction of CD3-splenocytes at a systemic vector dose of 5E12 vg/kg. A slight bias for CD8+ over CD4+ T-cell resident splenocytes was also noted. At a single IV dose of 5E13 vg/kg in the Ai9 fluorescent reporter mouse model, Ark313 expressing Cre recombinase was generated and that Ark313 could achieve permanent genetic changes in ˜25% of murine T-cells in vivo.

Additionally, Ark313 appeared to display a liver de-targeted phenotype relative to parental AAV6. Analysis of T-cell subtypes shows Ark313 significantly transduced naive, effector, and memory with slight preference for effector and memory T-cells.

### Example 11

### Evolution of Capsid Mutant Ark313 for Transduction of Murine T-cells

To develop AAVs capable of targeting murine T-cells in vivo, a capsid evolution was performed using the AAV6 serotype preforming saturation mutagenesis on variable region IV (VR-IV). This region was chosen due to its location at the 3-fold spike of assembled capsids. FIG. 22A shows the monomer, FIG. 22B shows the trimer, and FIG. 22C shows the assembled capsid, which has a demonstrated importance for tissue tropism and cell entry. AAV6 was chosen as a parent serotype for this evolution due to its known ability to broadly infect human immune cell lineages greater than other serotypes. Importantly, VR-IV of not known to overlap with AAV6's sialic acid (SA) or heparin sulfate (HS) dual glycan binding motifs. In FIG. 22A-FIG. 22C, VR-IV is shown in dark grey, SA is shown in white, and HS shown is in black). This library was then cycled on C57Bl/6J T-cells ex vivo for three rounds and then high throughput sequenced to generate mutants highly capable of transducing murine T-cells (see, e.g., FIG. 1 or FIG. 7B). Four of the top sequenced and highly enriched variants, dubbed Ark313 (sequence 454-VVNPAEG-460) (SEQ ID NO:05), Ark483 (sequence 454-LLNREAT-460) (SEQ ID NO:41), Ark485 (sequence 454-IVNPGCG-460) (SEQ ID NO:45), and Ark486 (sequence 454-KLLPVGE-460) (SEQ ID NO:47) as well as all-natural serotypes AAV1-Rh.10 (not including AAV7 and including Rh32.33) were then assessed on C57Bl/6J T-cells ex vivo packaging a self-complementary CBH driven GFP (scCBh-GFP) at a high 1×10′ vg/cell to determine which AAVs displayed murine T-cell tropism. Ark313, the most enriched variant within from the evolution, outperformed all other serotypes whether natural of engineered by both % GFP+(FIG. 22D) and median fluorescence intensity (FIG. 22E). Other engineered serotypes such as Ark483, Ark485, and Ark486, which all contained all or part of a consensus motif of two neutrally charged branched residues followed by an asparagine and proline, all outperformed the parental AAV6 strain. Notably serotypes AAV1, AAV2, and AAV5 could all appreciably infect murine T-cells with AAV5 performing the best among the natural serotypes. Accordingly, AAV5 and Ark313 was selected for in vivo testing as well as the parental AAV6 serotype.

### Example 12

Evolved Capsid Mutant Ark313 Efficiently Transduced T-cells in vivo

Packaging a scCBh-GFP cassette in AAV5, AAV6 and Ark313 were injected into 8-week-old mice intravenously by tail vein with at 1×10¹¹ vg/mouse (roughly 5E¹² vg/kg). As T-cells are a dividing cell population, animals were sacrificed 7-days post injection to minimize any loss of transient AAV expression and analyzed by splenocyte populations by flow cytometry. While neither AAV5 nor AAV6 were able to appreciably transduce CD3+ splenocytes at this dose, Ark313 could transduce up to 10.2% of spleen resident T-cells. Further, Ark13 did not significantly transduce the CD3-splenocyte population any greater than AAV5 or AAV6, which were both only negligibly do so. When comparing the CD4+ and CD8+ T-cells subset, Ark 313 did not significantly either population more so than the other although CD8+ T-cell transduction trended higher.

Next, the stability of transient AAV expression in T-cells transduced in vivo was next assessed. To do this, the same dose was injected and submandibular bleeds were performed every week for 4 weeks. The peripheral blood leukocyte (PBL) CD3+ population was performed. Ark313 was able to attain detectable transduction in circulating PBLs by week one and was stable at week two. By week three, the % GFP+ signal was non-significantly reduced and was stable until week 4. When examining the splenocyte population from these same mice, up to 9.3% of GFP+ splenocyte CD3+ T-cells was transduced and there was negligible transduction of the CD3-population. These data demonstrated that transient delivery of self-complementary AAV genomes for the transduction of both circulating and spleen resident T-cells was possible when using the Ark313 capsid and that effect lasted for at least 4 weeks post-injection. This can be without significantly transducing non-T-cell immune populations and efficiently transducing both CD4+ and CD8+ T-cell populations.

### Example 13

### Single Stranded AAV Genomes Do Not Efficiently Transduce T-Cells In Vivo

Some of the various aspects of AAV biology in vivo were next explored. To do this, the Ai9 mouse model (which contains a cre activatable TdTomato fluorescent reporter), was employed. As the resultant reporter activity was dependent on a permanent genetic change within the host genome, reporter signal strength would be conserved within a dividing T-cell population. Additionally, as continuous expression from AAV genome was not needed to maintain reporter signal, lower limits of detection and be attained. The efficacies of both a self-complementary CBh driven cre (scCBh-cre) cassette and a single-stranded CBA driven cre (ssCBA-cre) packaged in both Ark313 and the parental AAV6 serotype were compared. Mice were dosed at a 1E12 vg/mouse (5E13/vg/kg) given intravenously by tail vein injection and sacrificed 6 weeks later. In the self-complementary cohort, mice injected with Ark313 had up to 22.8% of spleen resident CD3+ cells being TdTomato+, a 20-fold increase over the parental AAV6 serotype, while again not significantly targeting the CD3-splenocyte population. Using a transient GFP reporter, CD4+ T-cells and CD8+ T-cells were both effectively transduced by Ark313 in vivo. However, when looking at single-stranded transgenes, expression was markedly reduced in T-cells. Mice injected with AAV6 packaging a ssCBA-cre transgene at the same dose as the self-complementary cohort did not have any detectable reporter activity 6 weeks post injection. Ark313 injected mice had up to 1.6% TdTomato+CD3+ splenocytes and was 14-fold less than the self-comp cohort. Interestingly, single stranded transgenes had a slight but significant bias for CD4+ T-cells over CD8+ T-cells in vivo. Together, this data indicated a defect in second strand synthesis in murine T-cells.

### Example 14

### Biodistribution of Ark313 was Liver De-Targeted

The biodistribution of Ark313 in vivo was examined relative to the parental AAV6 serotype. Using the tissue from the Ai9 study comparing single-stranded vs. self-comp genomes, the liver and heart for native TdTomato+ fluorescence was examined as AAV6 is used clinically to target these organs. In this self-comp cohort, no discernible differences were observed between AAV6 and Ark313 in the heart or liver by native fluorescence. However, when looking at single-stranded vectors, Ark313 showed drastically reduced expression in the liver relative to AAV6 (FIG. 21A). FIG. 21B shows the effect of the single-stranded vector in the heart. This could represent an additional advantage in limiting off-target effects while attempting to manipulate T cells in vivo using Ark313. The vector genomes per lamin beta 2 genome copy were quantified in various target organs including the liver, heart, muscle, brain and spleen by qPCR. Ark313 vector genomes were significantly reduced in liver with no significant difference in any other tested organ for both the self-complementary and single-stranded cohorts. As observed with other liver-de-targeted AAV vectors, it is plausible that such is concomitant with longer residence time in circulation, which in turn could promote increased opportunity to interact with circulating and splenic T-cell populations.

### Example 15

### Ark313 Preferentially Targeted Memory and Effector Over Naive T-Cells

The types of T-cells being transduced in vivo were interrogated by looking at CD44 and CD62L staining, markers associated with memory/effector and naive T-cell subsets respectively. Using Ai9 mice and a scCBh-Cre cassette, mice were injected intravenously by tail vein at IE11 vg/mouse with both Ark313 and AAV6 and sacrificed 4 weeks post-injection.

Ark313 again vastly outperformed AAV6 in transducing T-cells in vivo, with up to 10% CD3+ Splenocytes being TdTomato+ and no significant difference in CD3-splenocytes transduced between the two groups, which was negligible. There was no significant difference in amount of CD8+ over CD4+ T-cells transduced by Ark313. Within the CD4+ population, upon injection with either AAV6 or Ark313, there was an increase in total amount of both memory and effector CD4+ T-cells withing the spleen. Interestingly, Ark313 showed a significantly higher transduction of both memory and effector CD4+ T-cells over naive CD4+ T-cells while AAV6 was unable appreciably transduce any CD4+ population. In the CD8+ T-cell population, an expansion of effector subsets following injection with either AAV6 or Ark313 was noted relative to the PBS control. Notably while AAV6 was unable to transduce any CD8+ T-cell subset at this dose, Ark313 transduced up to 10% of spleen naive and memory CD8+ T-cells. Interestingly effector CD8+ T-cell populations had highly variable transduction by Ark313, with up to 45% being TdTomato+ in some mice. This indicated either an increased preference of Ark313 for transduction of effector and memory T-cells or represented an expansion of these T-cell subsets following injection by AAV likely representing an anti-AAV immunes response.

### Summary of Example 11-Example 15

As described in Examples 11-15, the data presented herein show a novel capsid mutant Ark313 can be used for the in vivo transduction of murine T-cells. When packaging a self-complementary GFP transgene, Ark313 can transduce circulating T-cells for at least 4 weeks post injection. In the cre based reporter Ai9 mouse model, Ark313 can transduce up to ˜25% of T-cells when packaging a self-complementary transgene and ˜1.5% when packaging a single-stranded transgene indicating potential defects in second-strand synthesis. Interestingly, Ark313 was shown to be a liver de-targeted vector, highlighting the importance of the mutated region for determining cell tropism. Finally, Ark313 displayed a bias for transducing different T-cell subtypes. Both memory and effector CD4+ T-cells were more significantly transduced over naive CD4+ T-cells. CD8+ effector T-cells were significantly transduced over naive and memory CD8+ T-cells, with up to ˜45% of CD8+ effector T-cells being TdTom+ in some experiments. This could potentially indicate a bias towards transducing effector T-cells or may represent a clonally expanded T-cell population in response to AAV. Collectively, described herein is a novel tool to interrogate T-cell biology, which can be used alone for transient gene delivery to T-cells either ex vivo or in vivo or in combination with mouse models that employ gene editing tools.

Gene edited T-cells are fast becoming a new platform for cell-based therapies. However, the inability of current AAV serotypes to transduce murine T-cells limits the pre-clinical studies that can be performed within the field. As the first AAV serotype to be described to target murine T-cells, Ark313 allows for pre-clinical modeling of adoptive cell transfers therapies, and potentially in vivo gene editing by both knock-out and knock-in. As a tool to study the limits and safety aspect of in vivo gene editing, Ark313 promises to accelerate the use of gene edited T-cells for clinical applications.

With Ark313, a synthetic AAV with a defined murine cellular entry mechanism through H2-Q7/MHC-I was provided. H2-Q7 has a very specific expression pattern in mice and is also not expressed at all in cells of human origin. That affords the skilled person with the potential to engineer cells that normally do not express H2-Q7 to express the H2-Q7 at the cell surface. Doing so can generate cells that can be specifically targeted by Ark313 in an environment that lacks natural target cells.

In sum, Ark313 is a tool that allows for interrogation of T-cell biology with broad applications that span from the basic biology of T cells to pre-clinical modelling of adoptive cell therapies to novel AAV targeting approaches in vivo.

1. A nucleic acid molecule, comprising: a sequence encoding an adeno-associated virus (AAV) capsid protein, wherein the encoded AAV capsid protein comprises the sequence of SEQ ID NO:01, wherein amino acids 454-460 of the capsid protein comprise the sequence set forth in any one of SEQ ID NO:05 SEQ ID NO:545.

2-4. (canceled)

5. An AAV capsid protein comprising a sequence having at least 90% identity to the sequence set forth in SEQ ID NO:01, wherein amino acids 454-460 of the capsid protein comprise the sequence set forth in any one of SEQ ID NO:05-SEQ ID NO:545.

6. The AAV capsid protein of claim 5, wherein amino acids 454-460 of the capsid protein comprise the sequence set forth in SEQ ID NO:05.

7-9. (canceled)

10. A recombinant AAV (rAAV) vector, comprising: a vector genome, wherein the vector genome is encapsidated by an AAV capsid comprising the AAV capsid protein of claim 5.

11-12. (canceled)

13. The rAAV vector of claim 10, wherein the vector genome comprises a first inverted terminal repeat (ITR) and a second ITR, and a transgene located between the first ITR and the second ITR.

14. (canceled)

15. The rAAV vector of claim 13, wherein the transgene encodes a therapeutic RNA or a therapeutic protein: a missing, deficient, and/or mutant protein or enzyme: or a gene-editing molecule.

16-17. (canceled)

18. The rAAV vector of claim 15, wherein the gene-editing molecule comprises a nuclease or a single guide RNA (sgRNA).

19. The rAAV vector of claim 18, wherein the nuclease comprises a Cas9 nuclease.

20. (canceled)

21. The rAAV vector of claim 18, wherein the single guide RNA (sgRNA) targets a gene in a T cell or NK cell.

22. A pharmaceutical composition comprising the rAAV vector of claim 10 and at least one pharmaceutically acceptable carrier.

23. A method of delivering a transgene to a target cell in a subject, the method comprising: administering to the subject having a disease or condition a therapeutically effective amount of the pharmaceutical composition of claim 22.

24. The method of claim 23, wherein the target cell is an immune cell, wherein the immune cell is a T cell, a NK cell, or a combination thereof.

25-26. (canceled)

27. A method of alleviating and/or treating a disease or a condition in a subject in need thereof, the method comprising: administering to the subject one or more cells that have been contacted ex vivo with the rAAV vector of claim 10.

28. The method of claim 24, wherein the disease or condition comprises an autoimmune disease or an immune deficiency disease.

29. The method of claim 24, wherein following the administering of the pharmaceutical composition, one or more aspects of (i) T cell and/or NK cell cellular homeostasis and/or ii) T cell and/or NK cell cellular functionality in the subject is improved and/or restored.

30-37. (canceled)

38. An AAV capsid library, comprising: a first AAV capsid protein comprising the sequence set forth in SEQ ID NO:01, and one or more capsid proteins comprising the sequence set forth in SEQ ID NO:01, wherein amino acids 454-460 of the capsid protein comprise the sequence set forth in any one of SEQ ID NO:05-SEQ ID NO:545.

39. The nucleic acid molecule of claim 1, wherein amino acids 454-460 of the capsid protein comprise the sequence set forth in SEQ ID NO:05.

40. The method of claim 27, wherein the disease or condition comprises an autoimmune disease or an immune deficiency disease.

41. The method of claim 27, wherein the one or more cells comprises a T cell, a NK cell, or a combination thereof.

42. The method of claim 27, wherein following the administering of the one or more cells, one or more aspects of (i) T cell and/or NK cell cellular homeostasis and/or (ii) T cell and/or NK cell cellular functionality in the subject is improved and/or restored.
